(19) United States
(12) Patent Application Publication
(10) Pub. No.: US 2018/0148744 A1
(43) Pub. Date: May 31, 2018

(54) BIOLOGICAL METHODS FOR PREPARING 3-HYDROXYPROPIONIC ACID

(71) Applicant: VERDEZYNE, INC., Carlsbad, CA (US)

(72) Inventor: Eric Michael KNIGHT, Carlsbad, CA (US)

(21) Appl. No.: 15/558,863

(22) PCT Filed: Mar. 18, 2016
(86) PCT No.: PCT/US2016/023243

§ 371 Date: Sep. 15, 2017

Related U.S. Application Data
(60) Provisional application No. 62/136,350, filed on Mar. 20, 2015.

(51) Int. Cl. C12P 7/52; C12N 9/04; C12N 9/02; C12P 7/42; C12N 15/81; C12N 15/52

(52) U.S. Cl. CPC C12P 7/52; C12N 9/0006; C12N 9/0008; C12P 7/42; C12Y 101/01059; C12N 2510/02; C12N 15/81; C12N 15/52; C12N 2330/50; C12N 2511/00; C12Y 102/01018

(57) ABSTRACT

This technology relates in part to biological methods for producing 3-hydroxypropionic acid and engineered microorganisms capable of such production.

### CROSS-REFERENCE

This application claims the benefit of U.S. Provisional Application No. 62/136,350, filed Mar. 20, 2015, which application is incorporated herein by reference in its entirety.

### TECHNICAL FIELD

The technology relates in part to biological methods for producing 3-hydroxypropionic acid and to engineered microorganisms capable of such production.

### BACKGROUND OF THE INVENTION

3-hydroxypropionic acid (3-HP) is a 3-carbon chemical that is a precursor to a number of valuable products, including acrylic acid. Microorganisms employ various enzyme-driven biological pathways to support their own metabolism and growth. A cell synthesizes native proteins, including enzymes, in vivo from deoxyribonucleic acid (DNA). DNA first is transcribed into a complementary ribonucleic acid (RNA) that comprises a ribonucleotide sequence encoding the protein. RNA then directs translation of the encoded protein by interaction with various cellular components, such as ribosomes. The resulting enzymes participate as biological catalysts in pathways involved in producing a variety of organic molecules by the organism.

These pathways can be exploited for the harvesting of naturally produced organic molecules, such as 3-HP. The pathways also can be altered to increase production of 3-HP, which has commercially valuable applications as noted above. Advances in recombinant molecular biology methodology allow researchers to isolate DNA from one organism and insert it into another organism, thus altering the cellular synthesis of enzymes or other proteins. Advances in recombinant molecular biology methodology also allow endogenous genes, carried in the genomic DNA of a microorganism, to be increased or decreased in copy number, thus altering the cellular synthesis of enzymes or other proteins. Such genetic engineering can change the biological pathways within the host organism, causing it to produce a desired product. Microorganic industrial production can minimize the use of caustic chemicals and the production of toxic byproducts, thus providing a “clean” source for certain compounds.

### SUMMARY OF THE INVENTION

Disclosed herein a genetically modified yeast, comprising one or more genetic modifications that reduce or abolish the activity of 3-hydroxypropionate dehydrogenase (HPD1) or malonate semialdehyde dehydrogenase (acetylating) (ALD6). In one embodiment, the genetically modified yeast comprises one or more genetic modifications that reduce or abolish the activity of 3-hydroxypropionate dehydrogenase (HPD1). In another embodiment, the genetically modified yeast comprises one or more genetic modifications that reduce or abolish the activity of malonate semialdehyde dehydrogenase (acetylating) (ALD6). In another embodiment, the one or more genetic modifications reduce or abolish the activity of 3-hydroxypropionate dehydrogenase (HPD1) and increase the activity of malonate semialdehyde dehydrogenase (acetylating) (ALD6).

In another embodiment, the HPD1 activity of the genetically modified yeast is reduced or abolished, and the one or more genetic modifications comprise a disruption, deletion or knockout of (i) a polynucleotide that encodes a HPD1 polypeptide, or (ii) a promoter operably linked to a polynucleotide that encodes a HPD1 polypeptide.

In another embodiment, the ALD6 activity of the genetically modified yeast is reduced or abolished, and the one or more genetic modifications comprise a disruption, deletion or knockout of (i) a polynucleotide that encodes a ALD6 polypeptide or (ii) a promoter operably linked to a polynucleotide that encodes a ALD6 polypeptide.

In another embodiment, the genetically modified yeast further comprises one or more genetic modifications that increase the activity of one or more enzymes selected from the group consisting of a cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase, and 3-hydroxypropionyl-CoA hydrolase. In another embodiment, the genetically modified yeast further comprises one or more genetic modifications that decrease the activity of one or more enzymes selected from the group consisting of a cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase, and 3-hydroxypropionyl-CoA hydrolase.

In another embodiment, the genetically modified yeast is of a strain selected from the group consisting of Yarrowia yeast, Candida albicans, Candida revkaufi, Candida pulcherrima, Candida tropicalis, Candida maltosa, Candida utilis, Candida viswanathii, Candida strain ATCC20336, Rhodotorula yeast, Rhodosporidium yeast, Saccharomyces yeast, Cryptococcus yeast, Trichosporon yeast, Pichia yeast, Kluyveromyces yeast and Lipomyces yeast. In some cases, the genetically modified yeast is a Candida tropicalis strain or a Candida strain ATCC20336. In some cases, the genetically modified yeast is a Candida strain ATCC20336. In some cases, the genetically modified yeast is selected from the group consisting of sAA5405, sAA5526, sAA5600, AA5679, sAA5710 and sAA5733. In some cases, the genetically modified yeast is sAA5600. In some cases, the genetically modified yeast is sAA5733.

In another embodiment, a HPD1 polypeptide of the genetically modified yeast comprises a polypeptide 60% or more identical to SEQ ID NO: 1. In another embodiment, the HPD1 polypeptide of the genetically modified yeast comprises a polypeptide 65% or more identical to SEQ ID NO: 1. In another embodiment, the HPD1 polypeptide of the genetically modified yeast comprises a polypeptide 70% or more identical to SEQ ID NO: 1. In another embodiment, the HPD1 polypeptide of the genetically modified yeast comprises a polypeptide 75% or more identical to SEQ ID NO: 1. In another embodiment, the HPD1 polypeptide of the genetically modified yeast comprises a polypeptide 80% or more identical to SEQ ID NO: 1. In another embodiment, the HPD1 polypeptide of the genetically modified yeast comprises a polypeptide 85% or more identical to SEQ ID NO: 1. In another embodiment, the HPD1 polypeptide of the genetically modified yeast comprises a polypeptide 90% or more identical to SEQ ID NO: 1. In another embodiment, the HPD1 polypeptide of the genetically modified yeast comprises a polypeptide 95% or more identical to SEQ ID NO: 1. In another embodiment, the HPD1 polypeptide of the genetically modified yeast comprises a polypeptide 100% identical to SEQ ID NO: 1.

In another embodiment, a ALD6 polypeptide of the genetically modified yeast comprises a polypeptide 60% or more identical to SEQ ID NO: 17. In another embodiment, the ALD6 polypeptide of the genetically modified yeast comprises a polypeptide 65% or more identical to SEQ ID NO: 17. In another embodiment, the ALD6 polypeptide of the genetically modified yeast comprises a polypeptide 70% or more identical to SEQ ID NO: 17. In another embodiment, the ALD6 polypeptide of the genetically modified yeast comprises a polypeptide 75% or more identical to SEQ ID NO: 17. In another embodiment, the ALD6 polypeptide of the genetically modified yeast comprises a polypeptide 80% or more identical to SEQ ID NO: 17. In another embodiment, the ALD6 polypeptide of the genetically modified yeast comprises a polypeptide 85% or more identical to SEQ ID NO: 17. In another embodiment, the ALD6 polypeptide of the genetically modified yeast comprises a polypeptide 90% or more identical to SEQ ID NO: 17. In another embodiment, the ALD6 polypeptide of the genetically modified yeast comprises a polypeptide 95% or more identical to SEQ ID NO: 17. In another embodiment, the ALD6 polypeptide of the genetically modified yeast comprises a polypeptide 100% identical to SEQ ID NO: 17.

In another embodiment, the HPD1 or ALD6 activity of the genetically modified yeast is abolished. In another embodiment, the HPD1 and ALD6 activity of the genetically modified yeast is abolished.

In another embodiment, the genetically modified yeast is adapted to produce 3-hydroxypropionic acid, 3-hydroxypropionate or a salt thereof from a feedstock. In another embodiment, the feedstock comprises one or more alkane hydrocarbons. For example, the feedstock can comprise one or more alkane hydrocarbons with odd carbon numbered chains. In another embodiment, the feedstock comprises one or more fatty acids or esters. For example, the feedstock can comprise one or more fatty acids or esters with odd carbon numbered chains. In another embodiment, the odd carbon numbered chains have at least 3 carbon atoms. In another embodiment, the odd carbon numbered chains have at least 5, 7, 9, 11, 13, 15, 17, 19, 21, 23, 25, 27, 29, 31, 33, or 35 carbon atoms. In another embodiment, the odd carbon numbered chains have less than 35 carbon atoms. In another embodiment, the odd carbon numbered chains have at most 5, 7, 9, 11, 13, 15, 17, 19, 21, 23, 25, 27, 29, 31, 33, or 35 carbon atoms. In another embodiment, the odd carbon numbered chains have 3 to 35 carbon atoms. In another embodiment, the odd carbon numbered chains have 3 to 5, 3 to 7, 3 to 9, 3 to 11, 3 to 13, 3 to 15, 3 to 17, 3 to 19, 3 to 21, 3 to 23, 3 to 25, 3 to 27, 3 to 29, 3 to 31, 3 to 33, 3 to 35, 5 to 7, 5 to 9, 5 to 11, 5 to 13, 5 to 15, 5 to 17, 5 to 19, 5 to 21, 5 to 23, 5 to 25, 5 to 27, 5 to 29, 5 to 31, 5 to 33, 5 to 35, 7 to 9, 7 to 11, 7 to 13, 7 to 15, 7 to 17, 7 to 19, 7 to 21, 7 to 23, 7 to 25, 7 to 27, 7 to 29, 7 to 31, 7 to 33, 7 to 35, 9 to 11, 9 to 13, 9 to 15, 9 to 17, 9 to 19, 9 to 21, 9 to 23, 9 to 25, 9 to 27, 9 to 29, 9 to 31, 9 to 33, 9 to 35, 11 to 13, 11 to 15, 11 to 17, 11 to 19, 11 to 21, 11 to 23, 11 to 25, 11 to 27, 11 to 29, 11 to 31, 11 to 33, 11 to 35, 13 to 15, 13 to 17, 13 to 19, 13 to 21, 13 to 23, 13 to 25, 13 to 27, 13 to 29, 13 to 31, 13 to 33, 13 to 35, 15 to 17, 15 to 19, 15 to 21, 15 to 23, 15 to 25, 15 to 27, 15 to 29, 15 to 31, 15 to 33, 15 to 35, 17 to 19, 17 to 21, 17 to 23, 17 to 25, 17 to 27, 17 to 29, 17 to 31, 17 to 33, 17 to 35, 19 to 21, 19 to 23, 19 to 25, 19 to 27, 19 to 29, 19 to 31, 19 to 33, 19 to 35, 21 to 23, 21 to 25, 21 to 27, 21 to 29, 21 to 31, 21 to 33, 21 to 35, 23 to 25, 23 to 27, 23 to 29, 23 to 31, 23 to 33, 23 to 35, 25 to 27, 25 to 29, 25 to 31, 25 to 33, 25 to 35, 27 to 29, 27 to 31, 27 to 33, 27 to 35, 29 to 31, 29 to 33, 29 to 35, 31 to 33, 31 to 35, or 33 to 35 carbon atoms. In another embodiment, the feedstock comprises one or more fatty acids or esters selected from the group consisting of propionic acid, propionate, valeric acid, valerate, heptanoic acid, heptanoate, nonanoic acid, nonanoate, undecanoic acid, undecanoate, tridecanoic acid, tridecanoate, pentadecanoic acid, pentadecanoate, heptadecanoic acid, heptadecanoate, nonadecanoic acid, nonadecanoate, heneicosanoic acid, heneisocanoate, tricosanoic acid, tricosanoate, pentacosanoic acid, pentacosanoate, heptacosanoic acid, heptacosanoate, nonacosanoic acid, nonacosanoate, hentriacontanoic acid, and hentriacontanoate. In another embodiment, the feedstock comprises one or more fatty acids selected from the group consisting of propionic acid, valeric acid, heptanoic acid, nonanoic acid, undecanoic acid, tridecanoic acid, pentadecanoic acid, heptadecanoic acid, nonadecanoic acid, heneicosanoic acid, tricosanoic acid, pentacosanoic acid, heptacosanoic acid, nonacosanoic acid, and hentriacontanoic acid. In another embodiment, the feedstock comprises one or more esters selected from the group consisting of propionate, valerate, heptanoate, nonanoate, undecanoate, tridecanoate, pentadecanoate, heptadecanoate, nonadecanoate, heneisocanoate, tricosanoate, pentacosanoate, heptacosanoate, nonacosanoate, and hentriacontanoate. In another embodiment, the feedstock comprises propane, n-pentane, or n-nonane. In another embodiment, the feedstock comprises pentadecanoic acid or pentadecanoate. In another embodiment, the pentadecanoate is methyl-pentadecanoate. In another embodiment, the source of the feedstock comprises one or more of petroleum, plants, chemically synthesized alkane hydrocarbons, alkane hydrocarbons produced by fermentation of a microorganism, animals, microorganisms, plants, plant oils, chemically synthesized fatty acids or fatty acids produced by fermentation of a microorganism.

In another embodiment, the yield or titer of 3-hydroxypropionic acid, 3-hydroxypropionate or a salt thereof is about 0.1 g/L to 25 g/L, for example, about 0.1 g/L to 0.5 g/L, about 0.1 g/L to 1 g/L, about 0.1 g/L to 2 g/L, about 0.1 g/L to 5 g/L, about 0.1 g/L to 10 g/L, about 0.1 g/L to 15 g/L, about 0.1 g/L to 20 g/L, about 0.1 g/L to 25 g/L, about 0.5 g/L to 1 g/L, about 0.5 g/L to 2 g/L, about 0.5 g/L to 5 g/L, about 0.5 g/L to 10 g/L, about 0.5 g/L to 15 g/L, about 0.5 g/L to 20 g/L, about 0.5 g/L to 25 g/L, about 1 g/L to 2 g/L, about 1 g/L to 5 g/L, about 1 g/L to 10 g/L, about 1 g/L to 15 g/L, about 1 g/L to 20 g/L, about 1 g/L to 25 g/L, about 2 g/L to 5 g/L, about 2 g/L to 10 g/L, about 2 g/L to 15 g/L, about 2 g/L to 20 g/L, about 2 g/L to 25 g/L, 5 g/L to 10 g/L, about 5 g/L to 15 g/L, about 5 g/L to 20 g/L, about 5 g/L to 25 g/L, about 10 g/L to 15 g/L, about 10 g/L to 20 g/L, about 10 g/L to 25 g/L, about 15 g/L to 20 g/L, about 15 g/L to 25 g/L, or about 20 g/L to 25 g/L. In another embodiment, the yield or titer of 3-hydroxypropionic acid, 3-hydroxypropionate or a salt thereof is at least about 0.1 g/L, for example, at least about 0.2 g/L, 0.3 g/L, 0.4 g/L, 0.5 g/L, 1 g/L, 2 g/L, 3 g/L, 4 g/L, 5 g/L, 6 g/L, 7 g/L, 8 g/L, 9 g/L, 10 g/L, 11 g/L, 12 g/L, 13 g/L, 14 g/L, 15 g/L, 16 g/L, 17 g/L, 18 g/L, 19 g/L, 20 g/L, 21 g/L, 22 g/L, 23 g/L, 24 g/L, or 25 g/L.

In another aspect, disclosed is an expression vector, comprising the one or more genetic modifications described herein. In another embodiment, also disclosed is an expression vector, comprising a nucleic acid sequence which is at least about 70% identical, for example, at least about 75%, at least about 80%, at least about 85%, at least about 90%, or at least about 95% identical to SEQ ID NO:6 or SEQ ID NO:19. In another embodiment, the nucleic acid sequence is at least about 80% identical to SEQ ID NO:6 or SEQ ID NO:19. In another embodiment, the nucleic acid sequence is at least about 90% identical to SEQ ID NO:6 or SEQ ID NO:19.

In another aspect, disclosed is a cell, comprising the expression vector described herein. In another embodiment, the cell is a bacterium. In another embodiment, the cell is a yeast. In another embodiment, the yeast is of a strain selected from the group consisting of Yarrowia yeast, Candida albicans, Candida revkaufi, Candida pulcherrima, Candida tropicalis, Candida maltosa, Candida utilis, Candida viswanathii, Candida strain ATCC20336, Rhodotorula yeast, Rhodosporidium yeast, Saccharomyces yeast, Cryptococcus yeast, Trichosporon yeast, Pichia yeast, Kluyveromyces yeast and Lipomyces yeast. In another embodiment, the yeast is a Candida tropicalis strain or a Candida strain ATCC20336. In another embodiment, the yeast is a Candida strain ATCC20336.

In another aspect, disclosed is a method for producing 3-hydroxypropionic acid, 3-hydroxypropionate or a salt thereof. In another embodiment, the method comprises: (a) contacting the genetically modified yeast described herein with a feedstock; and (b) culturing the genetically modified yeast under a condition in which the 3-hydroxypropionic acid, 3-hydroxypropionate or salt thereof is produced. In another embodiment, the method further comprises isolating the 3-hydroxypropionic acid, 3-hydroxypropionate or salt thereof.

In another aspect, disclosed is a method of producing acrylic acid, acrylate or a salt or derivative thereof. In another embodiment, the method comprises: (a) producing 3-hydroxypropionic acid, 3-hydroxypropionate or a salt thereof by performing any method described herein; and (b) subjecting the 3-hydroxypropionic acid, 3-hydroxypropionate or salt thereof to a condition under which acrylic acid, acrylate or a salt or derivative thereof is produced. In another embodiment, the condition comprises dehydration of the 3-hydroxypropionic acid, 3-hydroxypropionate or a salt thereof. In another embodiment, the method further comprises dehydrating of the 3-hydroxypropionic acid, 3-hydroxypropionate or a salt thereof.

Also provided in certain aspects is an engineered microorganism capable of producing 3-hydroxypropionic acid (3-HP), which microorganism includes one or more altered enzyme activities selected from the group consisting of cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase, 3-hydroxypropionyl-CoA hydrolase, 3-hydroxypropionate dehydrogenase and malonate semialdehyde dehydrogenase activity.

In certain aspects, one or more of the enzyme activities of cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase, 3-hydroxypropionyl-CoA hydrolase and malonate semialdehyde dehydrogenase is increased with respect to the activity level of the same enzyme in a naturally occurring or unmodified parental or host strain from which the engineered microorganism is derived. In some embodiments, a 3-hydroxypropionate dehydrogenase activity and/or a malonate semialdehyde dehydrogenase activity is reduced or abolished relative to the activity level of the same enzyme in a naturally occurring or unmodified parental or host strain from which the engineered microorganism is derived.

Also provided in certain aspects is an engineered microorganism that produces 3-hydroxypropionic acid, 3-hydroxypropionate or a salt thereof (collectively and interchangeably referred to herein as 3-HP).

Provided in certain aspects is a method for producing 3-hydroxypropionic acid, including culturing an engineered microorganism described herein under conditions in which 3-hydroxypropionic acid is produced. In some embodiments, the 3-hydroxypropionic acid is further converted to acrylic acid and/or other downstream products. In certain embodiments, the 3-hydroxypropionic acid is isolated and in some embodiments, the isolated 3-hydroxypropionic acid is further converted to acrylic acid and/or other downstream products.

Also provided in certain aspects is a method for preparing a microorganism that produces 3-HP, which includes: (a) introducing one or more genetic modifications to a host organism that decreases (reduces) or eliminates (abolishes) a 3-hydroxypropionate dehydrogenase (HPD1) activity and/or a malonate semialdehyde dehydrogenase (ALD6) activity and (b) selecting for engineered microorganisms that produce 3-HP. Also provided in certain aspects are nucleic acids, plasmids and expression vectors for preparing a microorganism that produces 3-HP. In some embodiments, the method further comprises introducing one or more genetic modifications to a host organism, whereby one or more of the following enzymatic activities are increased in the resulting engineered microorganism: cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase and 3-hydroxypropionyl-CoA hydrolase. In one embodiment, provided herein is a method for preparing a microorganism that produces 3-HP, which includes (a) introducing one or more genetic modifications to a host organism that decreases (reduces) or eliminates (abolishes) a 3-hydroxypropionate dehydrogenase (HPD1); (b) introducing one or more genetic modifications to a host organism that increases malonate semialdehyde dehydrogenase (ALD6) activity and (c) selecting for engineered microorganisms that produce 3-HP. Also provided in certain aspects are nucleic acids, plasmids and expression vectors for preparing a microorganism that produces 3-HP.

Provided also in certain aspects is a method for producing 3-HP that includes: contacting an engineered microorganism with a feedstock comprising one or more odd chain alkanes, and/or one or more odd chain fatty acids, wherein the engineered microorganism includes at least a genetic alteration that: (a) partially or completely blocks (reduces or abolishes) a HPD1 activity or (b) partially or completely blocks (reduces or abolishes) an ALD6 activity, and culturing the engineered microorganism under conditions in which 3-HP is produced. In some embodiments, the engineered microorganism includes a genetic alteration that partially or completely blocks (reduces or abolishes) a HPD1 activity and a genetic alteration that partially or completely blocks (reduces or abolishes) an ALD6 activity. In certain embodiments, the engineered microorganism includes a genetic alteration that increases the activity of one or more of the following enzymes: cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase and 3-hydroxypropionyl-CoA hydrolase. In some embodiments, the engineered microorganism includes one or more genetic alterations that reduce or abolish a HPD1 activity and increase an ALD6 activity.

In certain embodiments of the method, the engineered microorganism includes an enzymatic pathway for the ω-oxidation of alkanes. In some embodiments, the engineered microorganism includes an enzymatic pathway for the β-oxidation of aliphatic carboxylic acid compounds. In some embodiments, the engineered microorganism includes an enzymatic pathway for the ω-oxidation of alkanes and an enzymatic pathway for the β-oxidation of aliphatic carboxylic acid compounds. In certain embodiments, the 3-HP is isolated. In some embodiments, the 3-HP is used to manufacture acrylic acid and/or other downstream products.

Certain embodiments are described further in the following description, examples, claims and drawings.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows a schematic diagram of the ω-oxidation pathway for producing odd chain fatty acids from odd chain alkanes.

FIG. 2 shows a schematic diagram of a biological pathway for production of 3-HP (3-hydroxypropionic acid or 3-hydroxypropionate) from odd chain alkanes or odd chain fatty acids. The source material can be an odd chain fatty acid. Alternately, the source material can be an odd chain alkane, which can be converted to an odd chain fatty acid by ω-oxidation, as illustrated in FIG. 1. An exemplary odd chain fatty acid, as illustrated in the Figure, is propanoic acid (same as propionic acid). An exemplary odd chain alkane, as illustrated in the Figure, is propane.

FIG. 3 depicts the biological pathway for production of 3-HP in a Candida strain ATCC20336 HPD1 mutant. As shown in the figure, reducing or abolishing the activity of 3-hydroxypropionate dehydrogenase (HPD1) reduces or prevents the conversion of 3-HP to malonate semialdehyde, thereby leading to a build-up of 3-HP and increasing its production.

FIG. 4 depicts the biological pathway for production of 3-HP in a Candida strain ATCC20336 ALD6 mutant. As shown in the figure, reducing or abolishing the activity of malonate semialdehyde dehydrogenase (acetylating) (ALD6) reduces or prevents the conversion of 3-HP to downstream products acetyl-CoA and/or acetaldehyde, thereby leading to a build-up of 3-HP and increasing its production.

FIG. 5 depicts a HPD1 deletion cassette.

FIG. 6 depicts an ALD6 deletion cassette.

### DETAILED DESCRIPTION OF THE INVENTION

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The numerical ranges as used herein are inclusive. For example, an odd carbon numbered chain have “3 to 35 carbon atoms” includes odd carbon numbered chains with 3 or 35 carbon atoms. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

### Overview

3-hydroxypropionic acid (3-HP or 3HP, used interchangeably herein, which collectively refers to 3-hydroxypropionic acid, a 3-hydroxypropionate salt or ester thereof, or mixtures thereof in any proportion) is a platform chemical that can readily be converted into a variety of valuable products such as poly(hydroxypropionate), 1,3-propanediol, ethyl 3-ethoxypropionate (EEP), malonic acid and acrylic acid. For example, 3-HP can be dehydrated to produce acrylic acid, which in turn can be esterified to produce methyl acrylate or aminated to produce acrylamide. Acrylamide can further be converted by dehydration to acrylonitrile, acrylonitrile can be condensed to produce adiponitrile and adiponitrile can be hydrolysed to produce hexamethylenediamine (HMDA). In addition, polymerized acrylic acid (with itself or with other monomers such as acrylamide, acrylonitrile, vinyl, styrene, or butadiene) can produce a variety of homopolymers and copolymers that are used in the manufacture of various plastics, coatings, adhesives, elastomers, latex applications, emulsions, leather finishings, and paper coating, as well as floor polishes and paints. Acrylic acid also can be used as a chemical intermediate for the production of acrylic esters such as ethyl acrylate, butyl acrylate, methyl acrylate, and 2-ethyl hexyl acrylate and superabsorbent polymers (glacial acrylic acid).

Provided herein are methods for producing 3-HP, using biological systems. Such production systems may have significantly less environmental impact and could be economically competitive with current manufacturing systems. Thus, provided in part herein are methods for manufacturing 3-HP using engineered microorganisms. In some embodiments, microorganisms are engineered to contain at least one modified gene encoding an enzyme. In certain embodiments, an organism may be selected for elevated or decreased activity of a native enzyme.

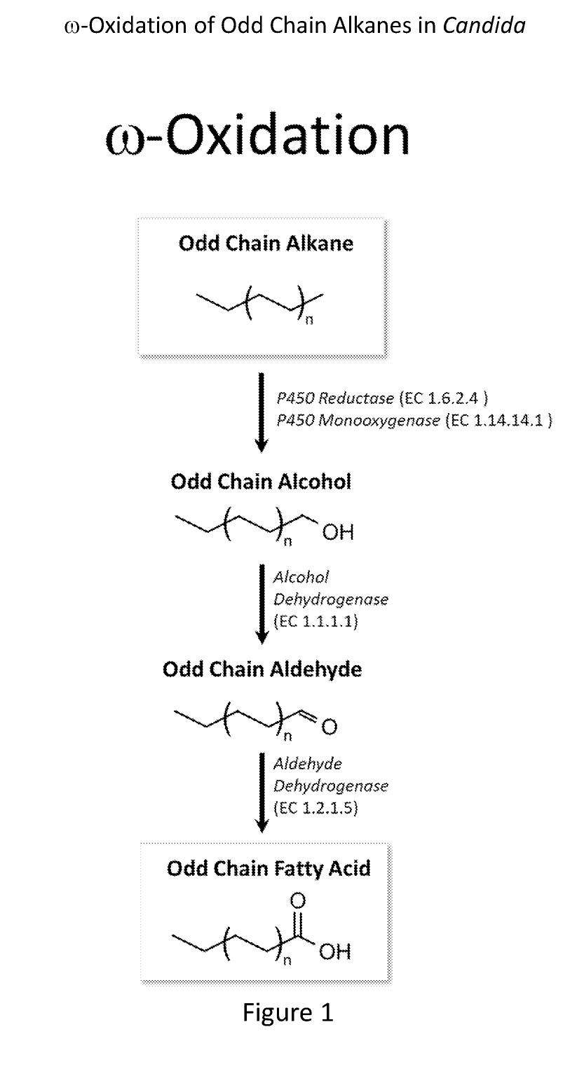

An exemplary embodiment of a method for manufacturing 3-HP using an engineered microorganism is as follows: A feedstock containing one or more odd chain alkanes is subjected to ω-oxidation in a microorganism, such as yeast, which is depicted in FIG. 1. During ω-oxidation, odd chain alkanes can be converted to odd chain alcohols, and the conversion is catalyzed by a cytochrome P450 reductase (e.g., EC 1.6.2.4; CPRA and CPRB genes of Candida strain ATCC20336 yeast strain; SEQ ID NOS: 28-31) and a cytochrome P-450 monooxygenase (e.g., EC 1.14.14.1; CYP52A12, CYP52A13, CYP52A14, CYP52A15, CYP52A16, CYP52A17, CYP52A18, CYP52A19, CYP52A20 and CYP52D2 genes of Candida strain ATCC20336 yeast strain; SEQ ID NOS: 32-51). The odd chain alcohols can then be converted to odd chain aldehydes, a reaction that is catalyzed by an alcohol dehydrogenase (e.g., EC 1.1.1.1; ADH1-1 short, ADH1-2 short, ADH1-2, ADH2a, ADH2b, ADH3, ADH4, ADH5, ADH7 and ADH8 genes of Candida strain ATCC20336 yeast strain; SEQ ID NOS: 52-71). The resulting odd chain aldehydes can be converted to odd chain fatty acids by catalysis using an aldehyde dehydrogenase (e.g., EC 1.2.1.5; ALDH genes of Candida strain ATCC20336 yeast strain; SEQ ID NOS: 72 and 73).

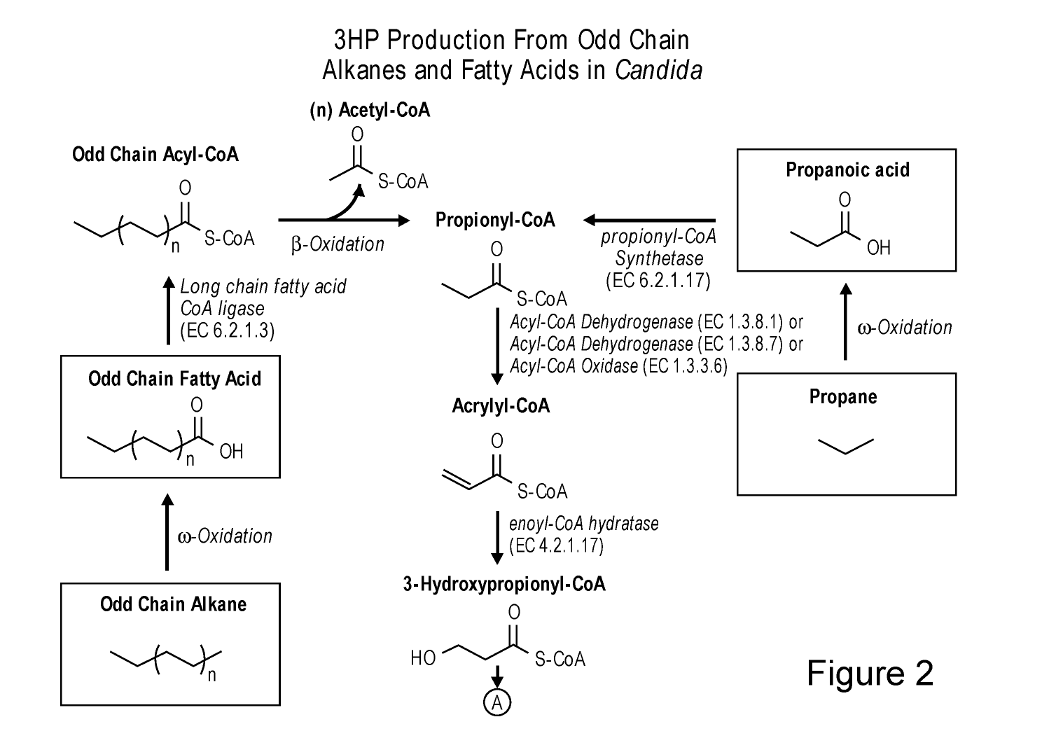

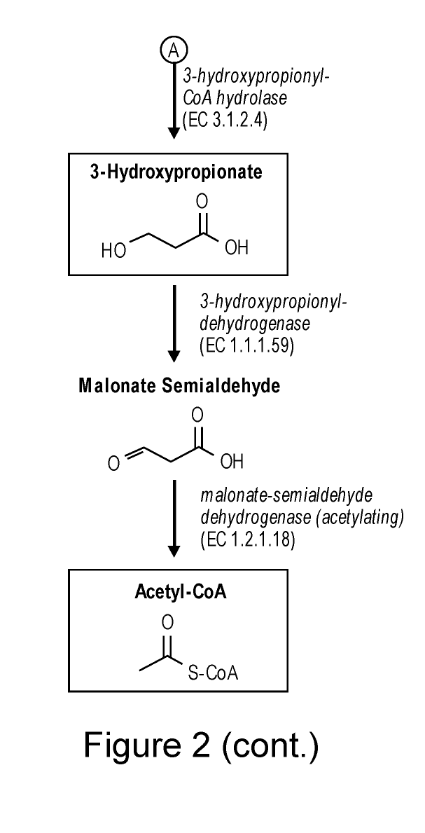

The odd chain fatty acids that are the products of ω-oxidation can then undergo β-oxidation and, through a further series of steps, be converted to 3-HP. Alternately, the source material in the feedstock can include one or more odd chain fatty acids, in which case their prior production through ω-oxidation of odd chain alkanes would not be needed. As the exemplary embodiment illustrates in FIG. 2, fatty acid CoA ligase (e.g., EC 6.2.1.3; FAT1/ACS1 genes of Candida strain ATCC20336 yeast strain; SEQ ID NOS: 74-77) can catalyze the conversion of odd chain fatty acids to odd chain fatty acyl-CoA (“CoA” being coenzyme A). An acetyl-CoA C-acyltransferase enzyme (e.g., beta-ketothiolase or POT1/FOX3/POX3 in S. cerevisiae or Candida, EC 2.3.1.16; SEQ ID NOS: 78-85) can catalyze the formation of a fatty acyl-CoA shortened by 2 carbons, by cleavage of 3-ketoacyl-CoA with the thiol group of another molecule of CoA. The thiol is inserted between C-2 and C-3, which yields an acetyl CoA molecule and an acyl CoA molecule that is two carbons shorter. The resulting shortened fatty acyl-CoA can progressively be shortened, two carbon atoms at a time, catalyzed by the acetyl-CoA C-acyltransferase enzyme, until propionyl-CoA is obtained. Alternately, if propionic acid is used as the starting material (source material in the feedstock, e.g.), the enzyme propionyl-CoA synthetase (e.g., EC 6.2.1.17; PRPE gene; SEQ ID NOS: 86-91) can catalyze its conversion to propionyl-CoA.

As illustrated in FIG. 2, propionyl-CoA can then be converted to acrylyl-CoA, and this conversion can be catalyzed by an acyl-CoA dehydrogenase (e.g., EC 1.3.8.1 from Pseudomonas putida (H8234), SEQ ID NOS: 92 and 93, encoded by gene L483 29890, or EC 1.3.8.7 from Pseudomonas putida (KT2440), SEQ ID NOS: 94 and 95, encoded by gene PP2216) or an acyl-CoA oxidase (e.g., EC 1.3.3.6; POX4 and POX5 genes of Candida strain ATCC20336 yeast strain; SEQ ID NOS: 96-99). The enzyme enoyl-CoA hydratase (e.g., EC 4.2.1.17; FOX2 gene of Candida strain ATCC20336 yeast strain; SEQ ID NOS: 100 and 101) can catalyze the conversion of acrylyl-CoA to 3-hydroxypropionyl-CoA. 3-hydroxypropionyl-CoA can then be converted to the desired end product, 3-hydroxypropionate (referred to interchangeably with 3-hydroxypropionic acid and depicted as 3-HP or 3HP). The conversion of 3-hydroxypropionyl-CoA to 3-HP can be catalyzed by the enzyme 3-hydroxypropionyl-CoA hydrolase (e.g., EC 3.1.2.4; EHD3 gene of Candida; SEQ ID NOS: 102 and 103). As described in detail below, the activities of one or more of any of the aforementioned enzymes can be increased to increase the production of 3-HP.

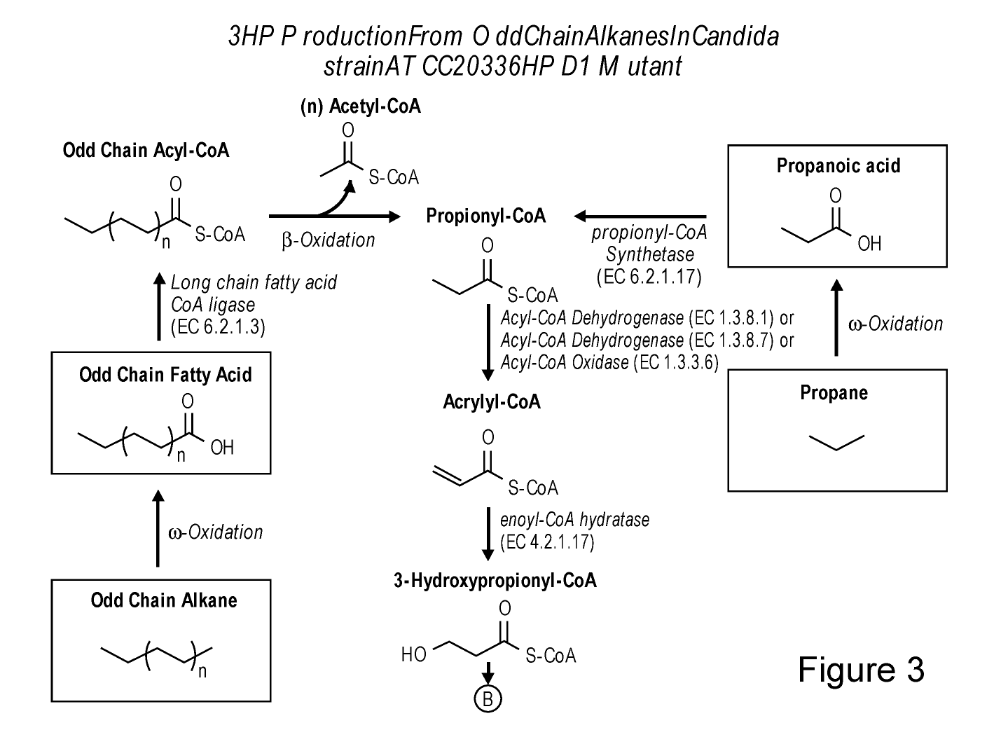

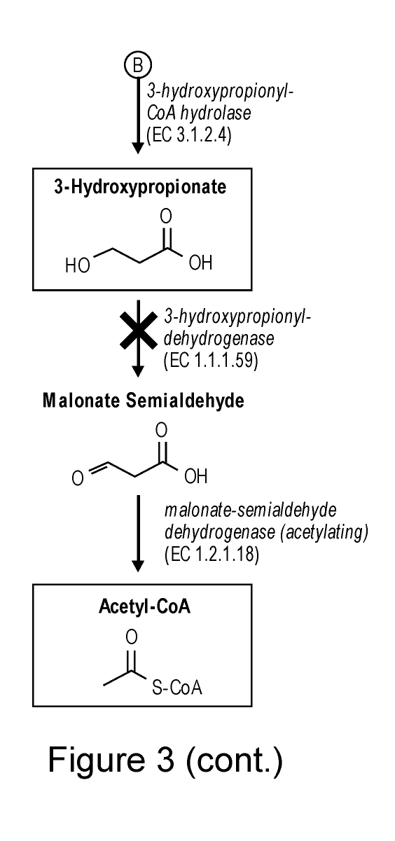

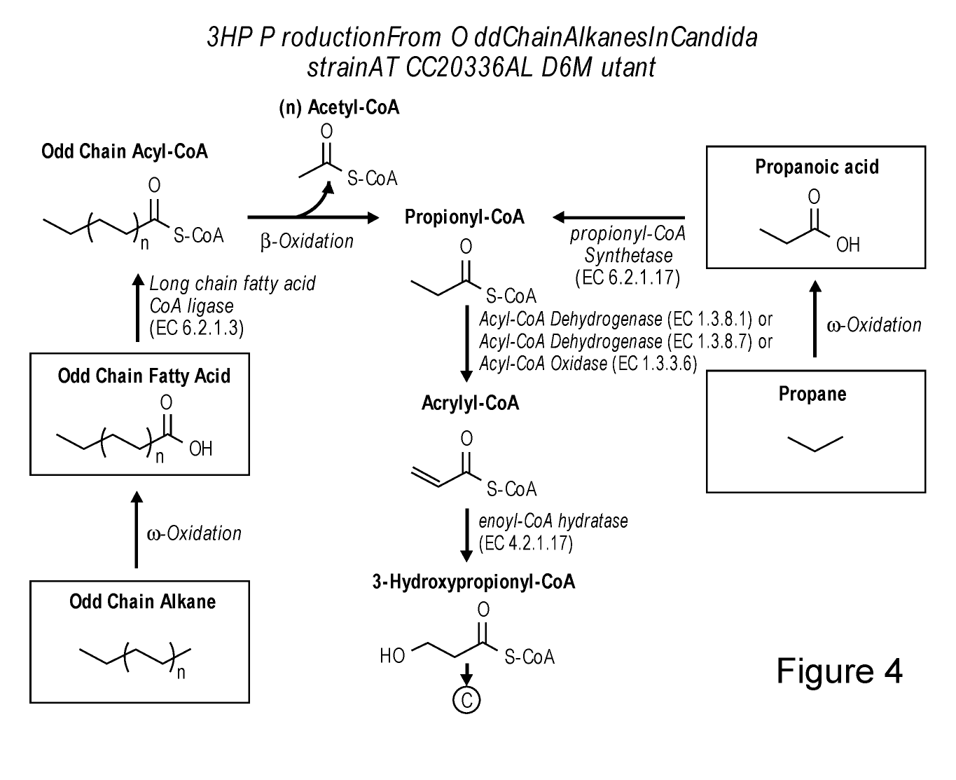

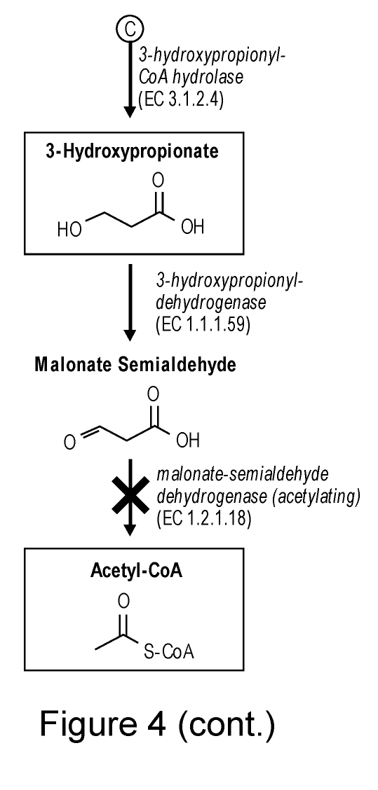

FIGS. 3 and 4 depict an embodiment of a pathway for producing 3-HP in a yeast strain, as also described in FIG. 2, and additionally depicts the downstream conversion of 3-HP, by the yeast, to other products. For example, as shown in FIGS. 3 and 4, 3-HP can further be converted to malonate semialdehyde in the yeast, and this conversion can be catalyzed by 3-hydroxypropionate dehydrogenase, also referred to herein as HPD1 (e.g., EC 1.1.1.59; SEQ ID NO: 1 (polynucleotide encoding HPD1) and SEQ ID NO: 2 (HPD1 polypeptide). The malonate semialdehyde can further be converted to acetyl-CoA, and this conversion can be catalyzed by the enzyme malonate-semialdehyde dehydrogenase (acetylating), also referred to herein as ALD6 (e.g., EC 1.2.1.18; SEQ ID NO: 17 (polynucleotide encoding ALD6) and SEQ ID NO: 18 (ALD6 polypeptide). As depicted in FIGS. 3 and 4, reducing or abolishing the activity of HPD1 (see FIG. 3) and/or ALD6 can lead to a build-up of the 3-HP product by inhibiting the formation of downstream products of 3-HP. In some embodiments, the activity of HPD1 can be reduced or abolished and the activity of ALD6 can be increased, thereby helping to clear the microorganism of residual amount of the toxic intermediate, malonate semialdehyde, while building up 3-HP production in the microorganism.

The 3-HP generated according to the methods provided herein, an embodiment of which is exemplified above, can further be isolated from the microorganism and/or be used to generate valuable downstream chemicals, such as acrylic acid. Microrganisms, including methods of genetically engineering the microorganisms, the enzymes and enzymatic pathways involved in the generation of 3-HP, source chemicals and feedstocks and other aspects of the genetically engineered organisms, nucleic acids, vectors and methods provided herein are described in further detail below.

### Microorganisms

A microorganism can be selected to be suitable for genetic manipulation and often can be cultured at cell densities useful for industrial production of a target product. A selected microorganism often can be maintained in a fermentation device.

The term “engineered microorganism” as used herein refers to a modified microorganism that includes one or more activities distinct from an activity present in a microorganism utilized as a starting point (hereafter a “host microorganism”). An engineered microorganism includes a heterologous polynucleotide in some embodiments, and in certain embodiments, an engineered organism has been subjected to selective conditions that alter an activity, or introduce an activity, relative to the host microorganism. Thus, an engineered microorganism has been altered directly or indirectly by a human being. A host microorganism sometimes is a native microorganism, and at other times is a microorganism that has been engineered to a point that can serve as a starting point for further modifications to produce the engineered microorganism that generates the compound of interest (e.g., 3-HP) in a higher yield relative to the host microorganism.

In some embodiments an engineered microorganism is a single cell organism, often capable of dividing and proliferating. A microorganism can include one or more of the following features: aerobe, anaerobe, filamentous, non-filamentous, monoploid, dipoid, polyploid, auxotrophic and/or non-auxotrophic. In certain embodiments, an engineered microorganism is a prokaryotic microorganism (e.g., bacterium), and in certain embodiments, an engineered microorganism is a non-prokaryotic microorganism. In some embodiments, an engineered microorganism is a eukaryotic microorganism (e.g., yeast, fungi, amoeba).

In some embodiments, any suitable yeast may be selected as a host microorganism, engineered microorganism or source for a heterologous polynucleotide. Yeast microorganisms can include, but are not limited to, Yarrowia yeast (e.g., Y. lipolytica (formerly classified as Candida lipolytica)), Candida yeast (e.g., C. revkaufi, C. pulcherrima, C. viswanathii, C. tropicalis, C. maltosa, C. utilis, Candida strain ATCC20336, C. albicans), Rhodotorula yeast (e.g., R. glutinus, R. graminis), Rhodosporidium yeast (e.g., R. toruloides), Saccharomyces yeast (e.g., S. cerevisiae, S. bayanus, S. pastorianus, S. carlsbergensis), Cryptococcus yeast, Trichosporon yeast (e.g., T. pullans, T. cutaneum), Pichia yeast (e.g., P. pastoris) and Lipomyces yeast (e.g., L. starkeyii, L. lipoferus). In some embodiments, a yeast is a Y. lipolytica strain that includes, but is not limited to, ATCC20962, ATCC8862, ATCC18944, ATCC20228, ATCC76982 and LGAM S(7)1 strains (Papanikolaou S., and Aggelis G., Bioresour. Technol. 82(1):43-9 (2002)). In certain embodiments, a yeast is a Candida strain that includes, but is not limited to, ATCC20336, ATCC20913, ATCC20962, sAA002, sAA5526, sAA5405, sAA5679, sAA5710, SU-2 (ura3-/ura3-), ATCC20962, H5343 (beta oxidation blocked; U.S. Pat. No. 5,648,247) strains.

Any suitable fungus may be selected as a host microorganism, engineered microorganism or source for a heterologous polynucleotide. Non-limiting examples of fungi include, but are not limited to, Aspergillus fungi (e.g., A. parasiticus, A. nidulans), Thraustochytrium fungi, Schizochytrium fungi and Rhizopus fungi (e.g., R. arrhizus, R. oryzae, R. nigricans). In some embodiments, a fungus is an A. parasiticus strain that includes, but is not limited to, strain ATCC24690, and in certain embodiments, a fungus is an A. nidulans strain that includes, but is not limited to, strain ATCC38163.

Any suitable prokaryote may be selected as a host microorganism, engineered microorganism or source for a heterologous polynucleotide. A Gram negative or Gram positive bacteria may be selected. Examples of bacteria include, but are not limited to, Bacillus bacteria (e.g., B. subtilis, B. megaterium), Acinetobacter bacteria, Norcardia baceteria, Xanthobacter bacteria, Escherichia bacteria (e.g., E. coli (e.g., strains DH10B, Stb12, DH5-alpha, DB3, DB3.1), DB4, DB5, JDP682 and ccdA-over (e.g., U.S. application Ser. No. 09/518,188)), Streptomyces bacteria, Erwinia bacteria, Klebsiella bacteria, Serratia bacteria (e.g., S. marcessans), Pseudomonas bacteria (e.g., P. aeruginosa), Salmonella bacteria (e.g., S. typhimurium, S. typhi), Megasphaera bacteria (e.g., Megasphaera elsdenii). Bacteria also include, but are not limited to, photosynthetic bacteria (e.g., green non-sulfur bacteria (e.g., Choroflexus bacteria (e.g., C. aurantiacus), Chloronema bacteria (e.g., C. gigateum)), green sulfur bacteria (e.g., Chlorobium bacteria (e.g., C. limicola), Pelodictyon bacteria (e.g., P. luteolum), purple sulfur bacteria (e.g., Chromatium bacteria (e.g., C. okenii)), and purple non-sulfur bacteria (e.g., Rhodospirillum bacteria (e.g., R. rubrum), Rhodobacter bacteria (e.g., R. sphaeroides, R. capsulatus), and Rhodomicrobium bacteria (e.g., R. vanellii)).

Cells from non-microbial organisms can be utilized as a host microorganism, engineered microorganism or source for a heterologous polynucleotide. Examples of such cells, include, but are not limited to, insect cells (e.g., Drosophila (e.g., D. melanogaster), Spodoptera (e.g., S. frugiperda Sf9 or Sf21 cells) and Trichoplusa (e.g., High-Five cells); nematode cells (e.g., C. elegans cells); avian cells; amphibian cells (e.g., Xenopus laevis cells); reptilian cells; mammalian cells (e.g., NIH3T3, 293, CHO, COS, VERO, C127, BHK, Per-C6, Bowes melanoma and HeLa cells); and plant cells (e.g., Arabidopsis thaliana, Nicotania tabacum, Cuphea acinifolia, Cuphea aequipetala, Cuphea angustifolia, Cuphea appendiculata, Cuphea avigera, Cuphea avigera var. pulcherrima, Cuphea axilliflora, Cuphea bahiensis, Cuphea baillonis, Cuphea brachypoda, Cuphea bustamanta, Cuphea calcarata, Cuphea calophylla, Cuphea calophylla subsp. mesostemon, Cuphea carthagenensis, Cuphea circaeoides, Cuphea confertiflora, Cuphea cordata, Cuphea crassiflora, Cuphea cyanea, Cuphea decandra, Cuphea denticulata, Cuphea disperma, Cuphea epilobiifolia, Cuphea ericoides, Cuphea flava, Cuphea flavisetula, Cuphea fuchsiifolia, Cuphea gaumeri, Cuphea glutinosa, Cuphea heterophylla, Cuphea hookeriana, Cuphea hyssopifolia (Mexican-heather), Cuphea hyssopoides, Cuphea ignea, Cuphea ingrata, Cuphea jorullensis, Cuphea lanceolata, Cuphea linarioides, Cuphea llavea, Cuphea lophostoma, Cuphea lutea, Cuphea lutescens, Cuphea melanium, Cuphea melvilla, Cuphea micrantha, Cuphea micropetala, Cuphea mimuloides, Cuphea nitidula, Cuphea palustris, Cuphea parsonsia, Cuphea pascuorum, Cuphea paucipetala, Cuphea procumbens, Cuphea pseudosilene, Cuphea pseudovaccinium, Cuphea pulchra, Cuphea racemosa, Cuphea repens, Cuphea salicifolia, Cuphea salvadorensis, Cuphea schumannii, Cuphea sessiliflora, Cuphea sessilifolia, Cuphea setosa, Cuphea spectabilis, Cuphea spermacoce, Cuphea splendida, Cuphea splendida var. viridiflava, Cuphea strigulosa, Cuphea subuligera, Cuphea teleandra, Cuphea thymoides, Cuphea tolucana, Cuphea urens, Cuphea utriculosa, Cuphea viscosissima, Cuphea watsoniana, Cuphea wrightii, Cuphea lanceolata)

Microorganisms or cells used as host organisms or source for a heterologous polynucleotide are commercially available. Microorganisms and cells described herein, and other suitable microorganisms and cells are available, for example, from Invitrogen Corporation, (Carlsbad, Calif.), American Type Culture Collection (Manassas, Va.), and Agricultural Research Culture Collection (NRRL; Peoria, Ill.).

Host microorganisms and engineered microorganisms may be provided in any suitable form. For example, such microorganisms may be provided in liquid culture or solid culture (e.g., agar-based medium), which may be a primary culture or may have been passaged (e.g., diluted and cultured) one or more times. Microorganisms also may be provided in frozen form or dry form (e.g., lyophilized). Microorganisms may be provided at any suitable concentration.

In some embodiments, host microorganisms are capable of ω-oxidation of alkanes. In certain embodiments, host microorganisms are capable of β-oxidation of aliphatic carboxylic acid compounds, where such compounds can also have alcohol, aldehyde, ester or additional caboxy functional groups. Such compounds can include for example fatty alcohols, fatty acids, monocarboxylic acids, dicarboxylic acids, and polycarboxylic acids. In some embodiments, the host microorganisms are capable of ω-oxidation of alkanes and are capable of β-oxidation of odd chain aliphatic carboxylic acid compounds. In certain embodiments, the host microorganisms are capable of producing 3-HP. The activities utilized to metabolize aliphatic carboxylic acids to 3-HP may include, but are not limited to, enzymatic activities of a cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase, an enoyl-CoA dehydrogenase and 3-hydroxypropionyl-CoA hydrolase.

The term “ω-oxidation activity” refers to any of the activities in the omega oxidation pathway utilized to metabolize alkanes, fatty alcohols, fatty acids, dicarboxylic acids, or sugars. The activities utilized to metabolize fatty alcohols, fatty acids, or dicarboxylic acids include, but are not limited to, monooxygenase activity (e.g., cytochrome P450 activity), monooxygenase reductase activity (e.g., cytochrome P450 reductase activity), alcohol dehydrogenase activity (e.g., fatty alcohol dehydrogenase activity, or long-chain alcohol dehydrogenase activity), fatty alcohol oxidase activity, fatty aldehyde dehydrogenase activity, and thioesterase activity.

The term “β oxidation activity” refers to any of the activities in the beta oxidation pathway utilized to metabolize aliphatic carboxylic acids. The host organisms having beta oxidation activity may possess such activity endogenously, or such activity may be engineered into the host organism via genetic manipulation, protoplast fusion or other means.

### Engineered Pathways

FIGS. 1-4 depict certain biological pathways useful for making 3-HP from odd chain alkanes and/or odd chain aliphatic carboxylic acid compounds (e.g., fatty acids, esters or salts thereof). Any suitable animal, microorganism, plant, including higher plant, plant oil, kerosene, diesel oil, fuel oil, petroleum jelly, paraffin wax, motor oil, asphalt, chemically synthesized alkane, alkane hydrocarbons produced by fermentation of a microorganism, or the like can be used as a source or feedstock for the odd chain alkanes. Any natural or chemically synthesized fatty acid, fatty ester, fatty alcohol, plant based oil, seed based oil, non-petroleum derived soap stock, animal source, microorganism source or the like can be used as the feedstock (starting material or carbon source) for odd chain fatty acids, esters or salts thereof. The feedstock can contain only one or more odd chain alkanes, only one or more odd chain fatty acids/esters, or a mixture of one or more odd chain alkanes and one or more odd chain fatty acids/esters.

As used herein, an “alkane” is a compound containing only carbon atoms and hydrogen atoms, where the atoms are all connected by single bonds. Alkanes are of the formula, CₙH₂ₙ₊₂, where “n” is the number of carbon atoms in the molecule. An alkane can be linear, i.e., a straight chain where each carbon atom in the chain is linked to one or two other carbon atoms in the chain. Alternately, an alkane can be a branched chain where at least one non-terminal carbon atom in a linear configuration is further linked to one or two alkyl groups by replacing one or two of its carbon-hydrogen bonds with a carbon-alkyl bond. As used herein, an “alkyl” group is of the formula CₙH₂ₙ₊₁, i.e., a group which, when bonded to a hydrogen atom, forms an alkane or when bonded to an existing alkane, forms an alkane with a higher number of carbon atoms. An “odd chain alkane,” used interchangeably herein with “odd carbon numbered alkane chains,” is an alkane having an odd number of linearly arranged carbon atoms. The odd chain alkanes used in the methods provided herein can have 3, 5, 7, 9, 11, 13, 15, 17, 19, 21, 23, 25, 27, 29, 31, 33, 35 or higher number of carbon atoms. Exemplary odd chain alkanes can include, but are not limited to, propane, n-pentane (also referred to herein as pentane), n-heptane (also referred to herein as heptane), n-nonane (also referred to herein as nonane), n-undecane, n-tridecane, n-pentadecane, n-heptadecane, n-nonadecane, n-henicosane, n-tricosane, n-pentacosane, n-heptacosane, n-nonacosane, n-hentriacontane, n-tritriacontane, n-pentatriacontane and the like, including higher carbon chain alkanes.

As used herein, a “fatty acid” is an aliphatic carboxylic acid that includes a hydrocarbon chain and a terminal carboxyl group. Fatty acids often are present as esters in fats and oils, and the term “fatty acid” as used herein includes esters of fatty acids. Fatty acid esters can be formed by the reaction of a fatty acid with an alcohol. For example, the reaction of a fatty acid with methanol produces a methyl ester of the fatty acid and the reaction of a fatty acid with glycerol produces a glyceride (mono-, di- or tri-glyceride, depending on whether one, two or three alcohol groups from the glycerol, respectively, react with a fatty acid). An “odd chain” fatty acid, used interchangeably herein with “odd carbon numbered fatty acid chains,” is a fatty acid that has an odd number of carbon atoms in a linear (i.e., not branched) configuration, the number of carbon atoms not including the carbon atoms forming an ester on the carboxyl function. The odd chain fatty acids used in the methods provided herein can have 3, 5, 7, 9, 11, 13, 15, 17, 19, 21, 23, 25, 27, 29, 31, 33, 35 or higher number of carbon atoms. Exemplary odd chain fatty acids (and their corresponding esters, e.g., methyl, ethyl, propyl, glyceride or other suitable ester) include, but are not limited to, propionic acid (also referred to herein as propanoic acid), valeric acid, heptanoic acid, nonanoic acid, undecanoic acid, tridecanoic acid, pentadecanoic acid, heptadecanoic acid, nonadecanoic acid, heneicosanoic acid, tricosanoic acid, pentacosanoic acid, heptacosanoic acid, nonacosanoic acid, henatriacontanoic acid, tritriacontanoic acid, pentatriacontanoic acid and the like, including higher carbon chain fatty acids.

As used herein, the term “3-hydroxypropionic acid” refers to the carboxylic acid C₃H₆O₃, having a molecular mass of about 90.08 g/mol and a pKa of about 4.5. 3-hydroxypropionic acid also is known in the art as hydracrylic acid or ethylene lactic acid. The terms “3-HP,” “3HP,” “3-hydroxypropionate” or “3-hydroxypropionic acid,” as used herein, can refer interchangeably to the aforementioned carboxylic acid, C₃H₆O₃, or any of its various 3-hydroxypropionate salt or ester forms, or mixtures thereof. Chemically, 3-hydroxypropionate generally corresponds to a salt or ester of 3-hydroxypropionic acid. Therefore, 3-hydroxypropionic acid and 3-hydroxypropionate refer to the same compound, which can be present in either of the two forms depending on the pH of the solution. Therefore, the terms 3-hydroxypropionic acid, 3-hydroxypropionate, 3-HP, 3HP, as well as other art recognized names such as hydracrylic acid and ethylene lactic acid are used interchangeably herein.

In certain embodiments, one or more activities in one or more metabolic pathways can be engineered to increase carbon flux through the engineered pathways to produce a desired product, i.e., 3-HP. The engineered activities can be chosen to allow increased production of metabolic intermediates that can be utilized in one or more other engineered pathways to achieve increased production of 3-HP, relative to the unmodified host organism. The engineered activities also can be chosen to allow decreased activity of enzymes that reduce production of a desired intermediate or end product (e.g., reverse activities). This “carbon flux management” can be optimized for any chosen feedstock, by engineering the appropriate activities in the appropriate pathways. The process of “carbon flux management” through engineered pathways produces 3-HP at a level and rate closer to the calculated maximum theoretical yield for any given feedstock, in certain embodiments. The terms “theoretical yield” or “maximum theoretical yield” as used herein refer to the yield of product of a chemical or biological reaction that can be formed if the reaction went to completion. Theoretical yield is based on the stoichiometry of the reaction and ideal conditions in which starting material is completely consumed, undesired side reactions do not occur, the reverse reaction does not occur, and there no losses in the work-up procedure.

A microorganism can be modified and engineered to include or regulate one or more activities in a 3-HP pathway. The term “activity” as used herein refers to the functioning of a microorganism's natural or engineered biological pathways to yield various products, including 3-HP and its precursors. 3-HP producing activity can be provided by any source, in certain embodiments. Such sources include, without limitation, eukaryotes such as yeast and fungi and prokaryotes such as bacteria. In some embodiments, an activity (e.g., HPD1, ALD6) in a pathway described herein can be altered (e.g., disrupted, reduced) to increase carbon flux through a 3-HP producing pathway, which renders such activity undetectable.

The term “undetectable” as used herein refers to an amount of an analyte that is below the limits of detection, using detection methods or assays known (e.g., described herein). In certain embodiments, a genetic modification partially reduces an enzyme activity. The term “partially reduced activity” as used here refers to a level of activity in an engineered organism that is lower than the level of activity found in the starting organism not containing such a genetic modification.

In some embodiments, a 3-HP pathway enzyme activity can be modified to alter the catalytic specificity of the chosen activity. In some embodiments, the altered catalytic specificity can be found by screening naturally occurring variant or mutant populations of a host organism. In certain embodiments, the altered catalytic specificity can be generated by various mutagenesis techniques in conjunction with selection and/or screening for the desired activity.

An engineered microorganism provided herein can include one or more of the following activities: a cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase, an enoyl-CoA dehydrogenase, 3-hydroxypropionyl-CoA hydrolase, 3-hydroxypropionate dehydrogenase and malonate semialdehyde dehydrogenase. In certain embodiments, one or more of the foregoing activities can be altered by way of one or more genetic modifications. In some embodiments, one or more of the foregoing activities is altered by way of (i) adding a heterologous polynucleotide that encodes a polypeptide having the activity, or (ii) altering or adding a regulatory sequence that regulates the expression of a polypeptide having the activity. In certain embodiments, one or more of the foregoing activities is altered by way of (i) disrupting an endogenous polynucleotide that encodes a polypeptide having the activity (e.g., insertional mutagenesis), (ii) deleting a regulatory sequence that regulates the expression of a polypeptide having the activity, or (iii) deleting the coding sequence that encodes a polypeptide having the activity (e.g., knock out mutagenesis).

In some situations, it is desirable for a gene to be expressed only during a certain phase or phases of the life cycle of the host production organism. For example, some gene(s) must be expressed for cells to grow and divide, but it may be desirable to turn the same gene(s) off during the phase in which the organism is producing the product of interest, namely, 3-HP. Such transient expression of a gene or genes only during the growth phase of the engineered host cell's life cycle can be accomplished by placing the gene under the control of a promoter that is on and active in the presence of a media component(s) that are included in the media only during the growth phase; when that same component(s) is removed from the media, the promoter is no longer functional and thus the gene that it controls is no longer expressed. One such useful promoter is the promoter for the HXT6 gene. This gene encodes a low-affinity hexose transporter and the HTX6 promoter is functional (and thus the gene is only expressed) in the presence of dextrose. Dextrose is typically a component of a fermentation medium that is used during growth phase but not during the 3-HP production phase. The HXT5 promoter can be fused to the open reading frame and terminator of the gene to be transiently expressed.

For those gene(s) that preferably are expressed only during production phase, each gene can be placed under the control of a strong promoter that is active when cultured in the presence of the feedstock of choice, such as, for example, fatty acids or oils. Examples of promoters that are highly expressed when Candida yeast species are cultured in the presence of fatty acids include, but are not limited to, POX4, PEX11 and ICL1.

### Exemplary Enzymes of the 3-HP Pathway

ω-Oxidation—Monooxygenases

A cytochrome P450 monooxygenase enzyme (e.g., EC 1.14.14.1), as used herein, often catalyzes the insertion of one atom of oxygen into an organic substrate (RH) while the other oxygen atom is reduced to water. Insertion of the oxygen atom near the omega carbon of a substrate yields an alcohol derivative of the original starting substrate (e.g., yields a fatty alcohol). A cytochrome P450 monooxygenase sometimes is encoded by the host organism and sometimes can be added to generate an engineered organism.

In certain embodiments, the monooxygenase activity is unchanged in a host or engineered organism. In some embodiments, the host monooxygenase activity can be increased by increasing the number of copies of a cytochrome P450 monooxygenase gene, or by increasing the activity of a promoter that regulates transcription of a cytochrome P450 monooxygenase gene, thereby increasing the production of the target product, 3-HP, due to increased carbon flux through the pathway. In certain embodiments, the cytochrome P450 monooxygenase gene can be isolated from any suitable organism. Non-limiting examples of organisms that include, or can be used as donors for, cytochrome P450 monooxygenase enzymes include yeast (e.g., Candida, Saccharomyces, Debaryomyces, Meyerozyma, Lodderomyces, Scheffersomyces, Clavispora, Yarrowia, Pichia, Kluyveromyces, Eremothecium, Zygosaccharomyces, Lachancea, Nakaseomyces), animals (e.g., Homo, Rattus), bacteria (e.g., Escherichia, Pseudomonas, Bacillus), or plants (e.g., Arabidopsis, Nictotania, Cuphea).

The activity of cytochrome P450 monooxgenase in the engineered microorganism, relative to the host microorganism, can be measured using a variety of known assays. An exemplary assay is described, for example, in Donato et al., J. Tiss. Cult. Methods, 14(3):153-157, (1992).

ω-Oxidation—Reductases

A cytochrome P450 reductase (e.g., EC 1.6.2.4), as used herein, can catalyze the reduction of the heme-thiolate moiety in cytochrome P450 by transferring an electron to the cytochrome P450. A cytochrome P450 reductase sometimes is encoded by the host organism and sometimes is added to generate an engineered organism. In certain embodiments, the cytochrome P450 reductase activity is unchanged in a host or engineered organism. In some embodiments, the host cytochrome P450 reductase activity can be increased by increasing the number of copies of a cytochrome P450 reductase gene, or by increasing the activity of a promoter that regulates transcription of a cytochrome P450 reductase gene, thereby increasing the production of the target product, 3-HP, due to increased carbon flux through the pathway. In certain embodiments, the cytochrome P450 reductase gene can be isolated from any suitable organism. Non-limiting examples of organisms that include, or can be used as donors for, cytochrome P450 reductase enzymes include yeast (e.g., Candida, Saccharomyces, Debaryomyces, Meyerozyma, Lodderomyces, Scheffersomyces, Clavispora, Yarrowia, Pichia, Kluyveromyces, Eremothecium, Zygosaccharomyces, Lachancea, Nakaseomyces), animals (e.g., Homo, Rattus), bacteria (e.g., Escherichia, Pseudomonas, Bacillus), or plants (e.g., Arabidopsis, Nictotania, Cuphea).

The activity of cytochrome P450 reductase in the engineered microorganism, relative to the host microorganism, can be measured using a variety of known assays. Exemplary assays are described, for example, in Yim et al., J. Biochem. Mol. Biol., 38(3):366-369, (2005); Guengerich et. al., Nat. Protoc., 4(9):1245-1251, (2009))

ω-Oxidation-Alcohol Dehydrogenases

An alcohol dehydrogenase (e.g., EC 1.1.1.1; long-chain alcohol dehydrogenase), as used herein, can catalyze the removal of a hydrogen from an alcohol to yield an aldehyde or ketone and a hydrogen atom and NADH. An alcohol dehydrogenase sometimes is encoded by the host organism and sometimes can be added to generate an engineered organism. In certain embodiments, the alcohol dehydrogenase activity is unchanged in a host or engineered organism. In some embodiments, the host alcohol dehydrogenase activity can be increased by increasing the number of copies of an alcohol dehydrogenase gene, or by increasing the activity of a promoter that regulates transcription of an alcohol dehydrogenase gene, thereby increasing the production of target product, 3-HP, due to increased carbon flux through the pathway. In certain embodiments, the alcohol dehydrogenase gene can be isolated from any suitable organism. Non-limiting examples of organisms that include, or can be used as donors for, alcohol dehydrogenase enzymes include yeast (e.g., Candida, Saccharomyces, Debaryomyces, Meyerozyma, Lodderomyces, Scheffersomyces, Clavispora, Yarrowia, Pichia, Kluyveromyces, Eremothecium, Zygosaccharomyces, Lachancea, Nakaseomyces), animals (e.g., Homo, Rattus), bacteria (e.g., Escherichia, Pseudomonas, Bacillus), or plants (e.g., Arabidopsis, Nictotania, Cuphea).

The activity of alcohol dehydrogenase in the engineered microorganism, relative to the host microorganism, can be measured using a variety of known assays. An exemplary assay is described, for example, in Walker, Biochem. Education, 20(1): published online 30 June, 2010.

ω-Oxidation—Aldehyde Dehydrogenases

A fatty aldehyde dehydrogenase enzyme (e.g., EC 1.2.1.5; long chain aldehyde dehydrogenase), as used herein, can catalyze the oxidation of long chain aldehydes to a long chain carboxylic acid, NADH and H⁺. A fatty aldehyde dehydrogenase sometimes is encoded by the host organism and sometimes can be added to generate an engineered organism. In certain embodiments, the fatty aldehyde dehydrogenase activity is unchanged in a host or engineered organism. In some embodiments, the host fatty aldehyde dehydrogenase activity can be increased by increasing the number of copies of a fatty aldehyde dehydrogenase gene, or by increasing the activity of a promoter that regulates transcription of a fatty aldehyde dehydrogenase gene, thereby increasing the production of target product, 3-HP, due to increased carbon flux through the pathway. In certain embodiments, the fatty aldehyde dehydrogenase gene can be isolated from any suitable organism. Non-limiting examples of organisms that include, or can be used as donors for, fatty aldehyde dehydrogenase enzymes include yeast (e.g., Candida, Saccharomyces, Debaryomyces, Meyerozyma, Lodderomyces, Scheffersomyces, Clavispora, Yarrowia, Pichia, Kluyveromyces, Eremothecium, Zygosaccharomyces, Lachancea, Nakaseomyces), animals (e.g., Homo, Rattus), bacteria (e.g., Escherichia, Pseudomonas, Bacillus), or plants (e.g., Arabidopsis, Nictotania, Cuphea).

The activity of aldehyde dehydrogenase in the engineered microorganism, relative to the host microorganism, can be measured using a variety of known assays. An exemplary assay is described, for example, in Duellman et al., Anal. Biochem., 434(2):226-232, (2013).

β-oxidation—Long Chain Fatty Acid/Acyl CoA Ligases

An acyl-CoA ligase enzyme (e.g., EC 6.2.1.3), as used herein, can catalyze the conversion of a long chain fatty acid to a long chain fatty acyl-CoA. An acyl-CoA ligase sometimes is encoded by the host organism and can be added to generate an engineered organism. In some embodiments, host acyl-CoA ligase activity can be increased by increasing the number of copies of an acyl-CoA ligase gene, by increasing the activity of a promoter that regulates transcription of an acyl-CoA ligase gene, or by increasing the number copies of the gene and by increasing the activity of a promoter that regulates transcription of the gene, thereby increasing production of the target product, 3-HP, due to increased carbon flux through the pathway. In certain embodiments, the acyl-CoA ligase gene can be isolated from any suitable organism. Non-limiting examples of organisms that include, or can be used as donors for, acyl-CoA ligase enzymes include Candida, Saccharomyces, or Yarrowia.

The activity of acyl-CoA ligase in the engineered microorganism, relative to the host microorganism, can be measured using a variety of known assays. An exemplary assay is described, for example, in Watkins et al., J. Biol. Chem., 273:18210-18219, (1998).

β-oxidation—Acyl-CoA Synthetase

Fatty acids can be converted into fatty-acyl-CoA intermediates by the activity of an acyl-CoA synthetase (e.g., ACS1, ACS2; EC 6.2.1.3; also referred to as acyl-CoA synthetase, acyl-CoA ligase), in many organisms. Acyl-CoA synthetase has six isoforms encoded by ACS1, FAT1, ACS2A, ACS2B, ACS2C and ACS2D, respectively, in Candida spp. (e.g., homologous to FAA1, FAT1, and FAA2 in S. cerevisiae). Acyl-CoA synthetase is a member of the ligase class of enzymes and catalyzes the reaction,

ATP+Fatty Acid+CoA<=>AMP+Pyrophosphate+Fatty-Acyl-CoA.

Fatty acids and Coenzyme A often are utilized in the activation of fatty acids to fatty-acyl-CoA intermediates for entry into various cellular processes. In some embodiments, host acyl-CoA synthetase activity can be increased by increasing the number of copies of an acyl-CoA synthetase gene, by increasing the activity of a promoter that regulates transcription of an acyl-CoA synthetase gene, or by increasing the number copies of the gene and by increasing the activity of a promoter that regulates transcription of the gene, thereby increasing production of the target product, 3-HP, due to increased carbon flux through the pathway.

The presence, absence or amount of acyl-CoA synthetase activity can be detected by any suitable method known in the art. Non-limiting examples of suitable detection methods include enzymatic assays (e.g., Lageweg et al “A Fluorometric Assay for Acyl-CoA Synthetase Activity”, Analytical Biochemistry, 197(2):384-388 (1991)), PCR based assays (e.g., qPCR, RT-PCR), immunological detection methods (e.g., antibodies specific for acyl-CoA synthetase), the like and combinations thereof. Non-limiting examples of organisms that include, or can be used as donors for, acyl-CoA ligase enzymes include Candida, Saccharomyces, or Yarrowia.

β-oxidation—Acetyl-CoA C-Acyltransferases

An Acetyl-CoA C-acyltransferase enzyme (e.g., a beta-ketothiolase, EC 2.3.1.16), as used herein, can catalyze the formation of a fatty acyl-CoA shortened by 2 carbon atoms, by cleavage of the 3-ketoacyl-CoA by the thiol group of another molecule of CoA. The thiol is inserted between C-2 and C-3, which yields an acetyl CoA molecule and an acyl CoA molecule that is two carbons shorter. An Acetyl-CoA C-acyltransferase sometimes is encoded by the host organism and sometimes can be added to generate an engineered organism. In certain embodiments, the acetyl-CoA C-acyltransferase activity is unchanged in a host or engineered organism. In some embodiments, the host acetyl-CoA C-acyltransferase activity can be increased by increasing the number of copies of an acetyl-CoA C-acyltransferase gene, or by increasing the activity of a promoter that regulates transcription of an acetyl-CoA C-acyltransferase gene, thereby increasing the production of the target product, 3-HP, due to increased carbon flux through the pathway. In certain embodiments, the acetyl-CoA C-acyltransferase gene can be isolated from any suitable organism. Non-limiting examples of organisms that include, or can be used as donors for, acetyl-CoA C-acyltransferase enzymes include Candida, Saccharomyces, or Yarrowia. One type of acetyl-CoA C-acyltransferase is an acetoacetyl CoA thiolase (e.g., “acoat”).

The activity of acetyl-CoA C-acyl transferase in the engineered microorganism, relative to the host microorganism, can be measured using a variety of known assays. An exemplary assay is described, for example, in Miyazawa et al., J. Biochem., 90(2):511-519, (1981).

β-oxidation—Propionyl-CoA Synthetase

A propionyl-CoA synthetase enzyme (e.g., EC 6.2.1.17), as used herein, can catalyze the conversion of propionic acid to propionyl-CoA. A propionyl-CoA synthetase sometimes is encoded by the host organism and sometimes can be added to generate an engineered organism. In certain embodiments, the propionyl-CoA synthetase activity is unchanged in a host or engineered organism. In some embodiments, the host propionyl-CoA synthetase activity can be increased by increasing the number of copies of a propionyl-CoA synthetase gene, or by increasing the activity of a promoter that regulates transcription of a propionyl-CoA synthetase gene, thereby increasing the production of the target product, 3-HP, due to increased carbon flux through the pathway. In certain embodiments, the propionyl-CoA synthetase gene can be isolated from any suitable organism. Non-limiting examples of organisms that include, or can be used as donors for propionyl-CoA synthetase enzymes include E. Coli K-12 MG1655, Metallosphaera sedula, S. typhimurium, Candida, Saccharomyces, or Yarrowia.

The activity of propionyl-CoA synthetase in the engineered microorganism, relative to the host microorganism, can be measured using a variety of known assays. Exemplary assays are described, for example, in Valentin et al., Appl. Env. Microbiol., 66(12):5253-5258, (2000) and Rajashekara et al., FEBS Lett., 556:143-147, (2004).

β-oxidation—Acyl-CoA Dehydrogenases

An acyl-CoA dehydrogenase enzyme (e.g., EC 1.3.8.1 or EC 1.3.8.7), as used herein, can catalyze the formation of a 2,3-enoyl-CoA (or trans-2,3-dehydroacyl-CoA) from its corresponding acyl-CoA (e.g., acrylyl-CoA from propionyl-CoA). In some embodiments, the activity is encoded by the host organism and sometimes can be added or increased to generate an engineered organism. In certain embodiments, the acyl-CoA dehydrogenase activity is unchanged in a host or engineered organism. In some embodiments, the host acyl-CoA dehydrogenase activity can be increased by increasing the number of copies of an acyl-CoA dehydrogenase gene, by increasing the activity of a promoter that regulates transcription of an acyl-CoA dehydrogenase gene, or by increasing the number copies of the gene and by increasing the activity of a promoter that regulates transcription of the gene, thereby increasing production of the target product, 3-HP, due to increased carbon flux through the pathway. In certain embodiments, the acyl-CoA dehydrogenase gene can be isolated from any suitable organism. Non-limiting examples of organisms that include, or can be used as donors for, acyl-CoA dehydrogenase enzymes include mammals, bacteria, e.g., Pseudomonas putida, Candida, Saccharomyces, or Yarrowia.

The activity of acyl-CoA dehydrogenase in the engineered microorganism, relative to the host microorganism, can be measured using a variety of known assays. An exemplary assay is described, for example, in Dommes et al., Anal. Biochem., 71(2):571-578, (1976).

β-oxidation—Acyl-CoA Oxidases

An acyl-CoA oxidase enzyme (e.g., EC 1.3.3.6), as used herein, like acyl-CoA dehydrogenases, can catalyze the oxidation of an acyl-CoA to a 2,3-enoyl-CoA (e.g., propionyl-CoA to acrylyl-CoA). In some embodiments the acyl-CoA oxidase activity is encoded by the host organism and sometimes can be altered to generate an engineered organism. An acyl-CoA oxidase activity is encoded, for example, by the POX4 and POX5 genes of Candida strain ATCC20336. In certain embodiments, endogenous acyl-CoA oxidase activity can be increased. In certain embodiments, host acyl-CoA oxidase activity of one or more of the PDX genes can be increased by genetically altering (e.g., increasing) the amount of the polypeptide produced (e.g., a strongly transcribed or constitutively expressed heterologous promoter is introduced in operable linkage with a polynucleotide that encodes the polypeptide; the copy number of a polynucleotide that encodes the polypeptide is increased (e.g., by introducing a plasmid that includes the polynucleotide, integration of additional copies in the host genome). Nucleic acid sequences encoding POX4 and POX5 can be obtained from a number of sources, including Candida tropicalis, for example.

The activity of acyl-CoA oxidase in the engineered microorganism, relative to the host microorganism, can be measured using a variety of known assays. An exemplary assay is described, for example, in Gopalan et al., Anal. Biochem., 250(1):44-50, (1997).

β-oxidation—Enoyl-CoA Hydratases

An enoyl-CoA hydratase enzyme (e.g., EC 4.2.1.17), as used herein, can catalyze the addition of a hydroxyl group and a proton to the unsaturated β-carbon on a fatty-acyl CoA (e.g., can facilitate the conversion of acrylyl-CoA to 3-hydroxypropionyl-CoA) and sometimes is encoded by the host organism and sometimes can be added to generate an engineered organism. In certain embodiments, the enoyl-CoA hydratase activity is unchanged in a host or engineered organism. In some embodiments, the host enoyl-CoA hydratase activity can be increased by increasing the number of copies of an enoyl-CoA hydratase gene, by increasing the activity of a promoter that regulates transcription of an enoyl-CoA hydratase gene, or by increasing the number copies of the gene and by increasing the activity of a promoter that regulates transcription of the gene, thereby increasing the production of the target product, 3-HP, due to increased carbon flux through the pathway. In certain embodiments, the enoyl-CoA hydratase gene can be isolated from any suitable organism. Non-limiting examples of organisms that include, or can be used as donors for, enoyl-CoA hydratase enzymes include Candida, Saccharomyces, or Yarrowia.

The activity of enoyl-CoA hydratase in the engineered microorganism, relative to the host microorganism, can be measured using a variety of known assays. An exemplary assay is described, for example, in Tsuge et al., FEMS Microbiol. Lett., 184(2):193-198, (2000).

β-oxidation—3-hydroxypropionyl-CoA hydrolases

A 3-hydroxypropionyl-CoA hydrolase enzyme (e.g., EC 3.1.2.4), as used herein, can catalyze the conversion of 3-hydroxypropionyl-CoA to 3-hydroxypropionate and sometimes is encoded by the host organism and sometimes can be added to generate an engineered organism. In certain embodiments, the enoyl-CoA hydratase activity is unchanged in a host or engineered organism. In some embodiments, the host 3-hydroxypropionyl-CoA hydrolase activity can be increased by increasing the number of copies of a 3-hydroxypropionyl-CoA hydrolase gene, by increasing the activity of a promoter that regulates transcription of a 3-hydroxypropionyl-CoA hydrolase gene, or by increasing the number copies of the gene and by increasing the activity of a promoter that regulates transcription of the gene, thereby increasing the production of the target product, 3-HP, due to increased carbon flux through the pathway. In certain embodiments, the 3-hydroxypropionyl-CoA hydrolase gene can be isolated from any suitable organism. Non-limiting examples of organisms that include, or can be used as donors for, 3-hydroxypropionyl-CoA hydrolase enzymes include Candida, Saccharomyces, or Yarrowia.

The activity of 3-hydroxypropionyl-CoA hydrolase in the engineered microorganism, relative to the host microorganism, can be measured using a variety of known assays. An exemplary assay is described, for example, in Shimomura et al., J. Biol. Chem., 269(19):14248-14253, (1994).

β-oxidation—3-hydroxypropionate dehydrogenase (HPD1)

A 3-hydroxypropionate dehydrogenase enzyme (e.g., EC 1.1.1.59), as used herein, can catalyze the conversion of 3-hydroxypropionate to malonate semialdehyde and sometimes is encoded by the host organism and sometimes can be disrupted to generate an engineered organism. In certain embodiments, the 3-hydroxypropionate dehydrogenase activity is unchanged in a host or engineered organism. In some embodiments, the host 3-hydroxypropionate dehydrogenase activity can be decreased by decreasing the number of copies of a 3-hydroxypropionate dehydrogenase gene, by decreasing the activity of a promoter that regulates transcription of a 3-hydroxypropionate dehydrogenase gene, or by decreasing the number copies of the gene and by decreasing the activity of a promoter that regulates transcription of the gene, thereby increasing the build-up and net production of the target product, 3-HP, due to decreasing the carbon flux through pathways involving the conversion of 3-HP to downstream products.

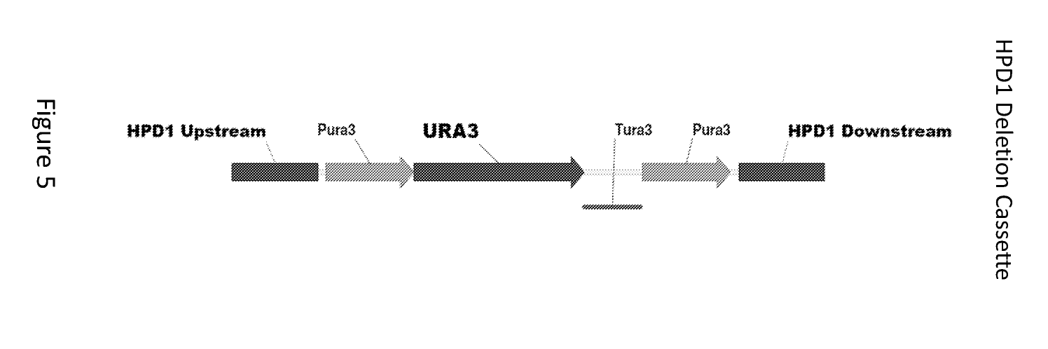

In some embodiments, the host 3-hydroxypropionate dehydrogenase activity can be decreased by disruption (e.g., knockout, insertion mutagenesis, the like and combinations thereof) of a 3-hydroxypropionate dehydrogenase gene, or by decreasing the activity of the promoter (e.g., addition of repressor sequences to the promoter or 5′UTR) that transcribes a 3-hydroxypropionate dehydrogenase gene. In some embodiments, the nucleotide sequence of the 3-hydroxypropionate dehydrogenase (HPD1) gene is disrupted with a URA3 nucleotide sequence encoding a selectable marker, and introduced to a host microorganism, thereby generating an engineered organism deficient in HPD1 activity. Nucleic acid sequences encoding HPD1 can be obtained from a number of sources, including Candida tropicalis and Candida strain ATCC20336, for example. Described in the examples are experiments conducted to decrease the activity encoded by the HPD1 gene (e.g., generating HPD1 deletion mutants, an embodiment of which is depicted in FIG. 5). Non-limiting examples of organisms that include 3-hydroxypropionate dehydrogenase enzymes include Candida, Saccharomyces, or Yarrowia.

The activity of 3-hydroxypropionate dehydrogenase in the engineered microorganism, relative to the host microorganism, can be measured using a variety of known assays. An exemplary assay is provided in the examples section. Another exemplary assay is described, for example, in U.S. Pat. No. 8,728,788.

β-oxidation—Malonate Semialdehyde Dehydrogenases (acetylating) (ALD6)

A malonate semialdehyde dehydrogenase (ALD6) enzyme (e.g., EC 1.2.1.18), as used herein, can catalyze the conversion of malonate semialdehyde to acetyl-CoA and sometimes is encoded by the host organism and sometimes can be added or disrupted to generate an engineered organism. In certain embodiments, ALD6 activity is unchanged in a host or engineered organism. In some embodiments, the host ALD6 activity can be increased by increasing the number of copies of a ALD6 gene, by increasing the activity of a promoter that regulates transcription of a ALD6 gene, or by increasing the number copies of the gene and by increasing the activity of a promoter that regulates transcription of the gene, thereby removing residual amounts of the toxic intermediate, malonate semialdehyde. For example, in some embodiments, the microorganism can be engineered to have disrupted HPD1 activity and increased ALD6 activity, thereby removing residual amounts of the toxic intermediate, malonate semialdehyde, while building 3-HP production in the microorganism. In certain embodiments, the ALD6 gene can be isolated from any suitable organism. Non-limiting examples of organisms that include, or can be used as donors for, ALD6 enzymes include Candida, Saccharomyces, or Yarrowia.

In some embodiments, the host ALD6 activity can be decreased by decreasing the number of copies of a ALD6 gene, by decreasing the activity of a promoter that regulates transcription of a ALD6 gene, or by decreasing the number copies of the gene and by decreasing the activity of a promoter that regulates transcription of the gene, thereby increasing the build-up and net production of the target product, 3-HP, due to decreasing the carbon flux through pathways involving the conversion of 3-HP to downstream products.

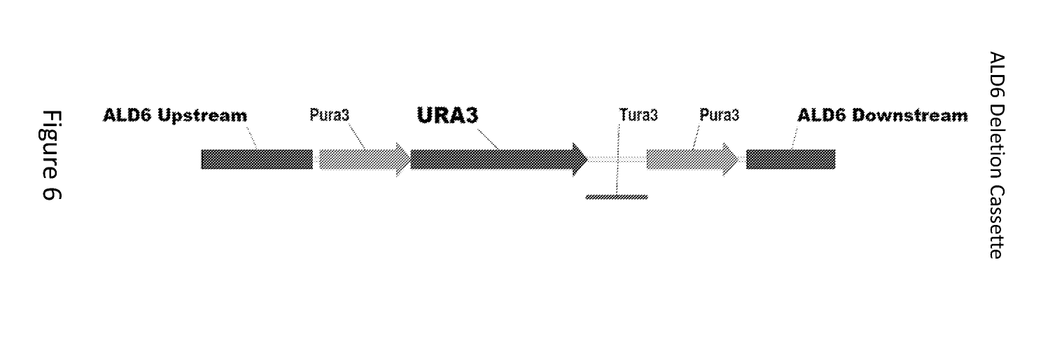

In some embodiments, the host ALD6 activity can be decreased by disruption (e.g., knockout, insertion mutagenesis, the like and combinations thereof) of a ALD6 gene, or by decreasing the activity of the promoter (e.g., addition of repressor sequences to the promoter or 5′UTR) that transcribes a ALD6 gene. In some embodiments, the nucleotide sequence of the ALD6 gene is disrupted with a URA3 nucleotide sequence encoding a selectable marker, and introduced to a host microorganism, thereby generating an engineered organism deficient in ALD6 activity. Nucleic acid sequences encoding ALD6 can be obtained from a number of sources, including Candida tropicalis and Candida strain ATCC20336, for example. Described in the examples are experiments conducted to decrease the activity encoded by the ALD6 gene (e.g., generating ALD6 deletion mutants, an embodiment of which is depicted in FIG. 6). Non-limiting examples of organisms that include ALD6 enzymes include Candida, Saccharomyces, or Yarrowia.

The activity of malonate semialdehyde dehydrogenase in the engineered microorganism, relative to the host microorganism, can be measured using a variety of known assays. An exemplary assay is described, for example, in Bannerjee et al., J. Biol. Chem., 245:1828-1835, (1970). Another exemplary assay is provided, for example, in Hayaishi et al., J. Biol. Chem., 236:781-790, (1961).

### Polynucleotides and Polypeptides for Genetic Engineering of Microorganisms

A nucleic acid (e.g., also referred to herein as nucleic acid reagent, target nucleic acid, target nucleotide sequence, nucleic acid sequence of interest or nucleic acid region of interest) can be from any source or composition, such as DNA, cDNA, gDNA (genomic DNA), RNA, siRNA (short inhibitory RNA), RNAi, tRNA or mRNA, for example, and can be in any form (e.g., linear, circular, supercoiled, single-stranded, double-stranded, and the like). A nucleic acid can also comprise DNA or RNA analogs (e.g., containing base analogs, sugar analogs and/or a non-native backbone and the like). It is understood that the term “nucleic acid” does not refer to or infer a specific length of the polynucleotide chain, thus polynucleotides and oligonucleotides are also included in the definition. Deoxyribonucleotides include deoxyadenosine, deoxycytidine, deoxyguanosine and deoxythymidine. For RNA, the uracil base is uridine.

A nucleic acid sometimes is a plasmid, phage, autonomously replicating sequence (ARS), centromere, artificial chromosome, yeast artificial chromosome (e.g., YAC) or other form of expression vector able to replicate or be replicated in a host cell. In certain embodiments, a nucleic acid can be from a library or can be obtained from enzymatically digested, sheared or sonicated genomic DNA (e.g., fragmented) from an organism of interest. In some embodiments, nucleic acid subjected to fragmentation or cleavage may have a nominal, average or mean length of about 5 to about 10,000 base pairs, about 100 to about 1,000 base pairs, about 100 to about 500 base pairs, or about 10, 15, 20, 25, 30, 35, 40, 45, 50, 55, 60, 65, 70, 75, 80, 85, 90, 95, 100, 200, 300, 400, 500, 600, 700, 800, 900, 1000, 2000, 3000, 4000, 5000, 6000, 7000, 8000, 9000 or 10000 base pairs. Fragments can be generated by any suitable method in the art, and the average, mean or nominal length of nucleic acid fragments can be controlled by selecting an appropriate fragment-generating procedure by the person of ordinary skill. In some embodiments, the fragmented DNA can be size selected to obtain nucleic acid fragments of a particular size range.

Nucleic acids can be fragmented by various methods known to the person of ordinary skill, which include without limitation, physical, chemical and enzymatic processes. Examples of such processes are described in U.S. Patent Application Publication No. 20050112590 (published on May 26, 2005, entitled “Fragmentation-based methods and systems for sequence variation detection and discovery,” naming Van Den Boom et al.). Certain processes can be selected by the person of ordinary skill to generate non-specifically cleaved fragments or specifically cleaved fragments. Examples of processes that can generate non-specifically cleaved fragment sample nucleic acid include, without limitation, contacting sample nucleic acid with apparatus that expose nucleic acid to shearing force (e.g., passing nucleic acid through a syringe needle; use of a French press); exposing sample nucleic acid to irradiation (e.g., gamma, x-ray, UV irradiation; fragment sizes can be controlled by irradiation intensity); boiling nucleic acid in water (e.g., yields about 500 base pair fragments) and exposing nucleic acid to an acid and base hydrolysis process.

Nucleic acids may be specifically cleaved by contacting the nucleic acid with one or more specific cleavage agents. The term “specific cleavage agent” as used herein refers to an agent, sometimes a chemical or an enzyme that can cleave a nucleic acid at one or more specific sites. Specific cleavage agents often will cleave specifically according to a particular nucleotide sequence at a particular site. Examples of enzymic specific cleavage agents include without limitation endonucleases (e.g., DNase (e.g., DNase I, II); RNase (e.g., RNase E, F, H, P); Cleavase™ enzyme; Taq DNA polymerase; E. coli DNA polymerase I and eukaryotic structure-specific endonucleases; murine FEN-1 endonucleases; type I, II or III restriction endonucleases such as Acc I, Afl III, Alu I, Alw44 I, Apa I, Asn I, Ava I, Ava II, BamH I, Ban II, Bcl I, Bgl I. Bgl II, Bln I, Bsm I, BssH II, BstE II, Cfo I, CIa I, Dde I, Dpn I, Dra I, EcIX I, EcoR I, EcoR I, EcoR II, EcoR V, Hae II, Hae II, Hind II, Hind III, Hpa I, Hpa II, Kpn I, Ksp I, Mlu I, MIuN I, Msp I, Nci I, Nco I, Nde I, Nde II, Nhe I, Not I, Nru I, Nsi I, Pst I, Pvu I, Pvu II, Rsa I, Sac I, Sal I, Sau3A I, Sca I, ScrF I, Sfi I, Sma I, Spe I, Sph I, Ssp I, Stu I, Sty I, Swa I, Taq I, Xba I, Xho I); glycosylases (e.g., uracil-DNA glycolsylase (UDG), 3-methyl adenine DNA glycosylase, 3-methyladenine DNA glycosylase II, pyrimidine hydrate-DNA glycosylase, FaPy-DNA glycosylase, thymine mismatch-DNA glycosylase, hypoxanthine-DNA glycosylase, 5-Hydroxymethyluracil DNA glycosylase (HmUDG), 5-Hydroxymethyl-cytosine DNA glycosylase, or 1,N6-etheno-adenine DNA glycosylase); exonucleases (e.g., exonuclease III); ribozymes, and DNAzymes. Sample nucleic acids may be treated with a chemical agent, or synthesized using modified nucleotides, and the modified nucleic acid may be cleaved. In non-limiting examples, sample nucleic acid may be treated with (i) alkylating agents such as methylnitrosourea that generate several alkylated bases, including N3-methyladenine and N3-methylguanine, which are recognized and cleaved by alkyl purine DNA-glycosylase; (ii) sodium bisulfite, which causes deamination of cytosine residues in DNA to form uracil residues that can be cleaved by uracil N-glycosylase; and (iii) a chemical agent that converts guanine to its oxidized form, 8-hydroxyguanine, which can be cleaved by formamidopyrimidine DNA N-glycosylase. Examples of chemical cleavage processes include without limitation alkylation, (e.g., alkylation of phosphorothioate-modified nucleic acid); cleavage of acid lability of P3′-N5′-phosphoroamidate-containing nucleic acid; and osmium tetroxide and piperidine treatment of nucleic acid.

A nucleic acid suitable for use in the embodiments described herein sometimes is amplified by any amplification process known in the art (e.g., PCR, RT-PCR and the like). Nucleic acid amplification may be particularly beneficial when using organisms that are typically difficult to culture (e.g., slow growing, require specialize culture conditions and the like). The terms “amplify”, “amplification”, “amplification reaction”, or “amplifying” as used herein refer to any in vitro processes for multiplying the copies of a target sequence of nucleic acid. Amplification sometimes refers to an “exponential” increase in target nucleic acid. However, “amplifying” as used herein can also refer to linear increases in the numbers of a select target sequence of nucleic acid, but is different than a one-time, single primer extension step. In some embodiments, a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may also limit inaccuracies associated with depleted reactants in standard PCR reactions.

In some embodiments, a nucleic acid reagent sometimes is stably integrated into the chromosome of the host organism, or a nucleic acid reagent can be a deletion of a portion of the host chromosome, in certain embodiments (e.g., genetically modified organisms, where alteration of the host genome confers the ability to selectively or preferentially maintain the desired organism carrying the genetic modification). Such nucleic acid reagents (e.g., nucleic acids or genetically modified organisms whose altered genome confers a selectable trait to the organism) can be selected for their ability to guide production of a desired protein or nucleic acid molecule. When desired, the nucleic acid reagent can be altered such that codons encode for (i) the same amino acid, using a different tRNA than that specified in the native sequence, or (ii) a different amino acid than is normal, including unconventional or unnatural amino acids (including detectably labeled amino acids). As described herein, the term “native sequence” refers to an unmodified nucleotide sequence as found in its natural setting (e.g., a nucleotide sequence as found in an organism).

A nucleic acid or nucleic acid reagent can comprise certain elements often selected according to the intended use of the nucleic acid. Any of the following elements can be included in or excluded from a nucleic acid reagent. A nucleic acid reagent, for example, may include one or more or all of the following nucleotide elements: one or more promoter elements, one or more 5′ untranslated regions (5′UTRs), one or more regions into which a target nucleotide sequence may be inserted (an “insertion element”), one or more target nucleotide sequences, one or more 3′ untranslated regions (3′UTRs), and one or more selection elements. A nucleic acid reagent can be provided with one or more of such elements and other elements may be inserted into the nucleic acid before the nucleic acid is introduced into the desired organism. In some embodiments, a provided nucleic acid reagent comprises a promoter, 5′UTR, optional 3′UTR and insertion element(s) by which a target nucleotide sequence is inserted (i.e., cloned) into the nucleotide acid reagent. In certain embodiments, a provided nucleic acid reagent comprises a promoter, insertion element(s) and optional 3′UTR, and a 5′ UTR/target nucleotide sequence is inserted with an optional 3′UTR. The elements can be arranged in any order suitable for expression in the chosen expression system (e.g., expression in a chosen organism, or expression in a cell free system, for example), and in some embodiments a nucleic acid reagent comprises the following elements in the 5′ to 3′ direction: (1) promoter element, 5′UTR, and insertion element(s); (2) promoter element, 5′UTR, and target nucleotide sequence; (3) promoter element, 5′UTR, insertion element(s) and 3′UTR; and (4) promoter element, 5′UTR, target nucleotide sequence and 3′UTR.

A promoter element typically is required for DNA synthesis and/or RNA synthesis. A promoter element often comprises a region of DNA that can facilitate the transcription of a particular gene, by providing a start site for the synthesis of RNA corresponding to a gene. Promoters generally are located near the genes they regulate, are located upstream of the gene (e.g., 5′ of the gene), and are on the same strand of DNA as the sense strand of the gene, in some embodiments. In some embodiments, a promoter element can be isolated from a gene or organism and inserted in functional connection with a polynucleotide sequence to allow altered and/or regulated expression. A non-native promoter (e.g., promoter not normally associated with a given nucleic acid sequence) used for expression of a nucleic acid often is referred to as a heterologous promoter. In certain embodiments, a heterologous promoter and/or a 5′UTR can be inserted in functional connection with a polynucleotide that encodes a polypeptide having a desired activity as described herein. The terms “operably linked” and “in functional connection with” as used herein with respect to promoters, refer to a relationship between a coding sequence and a promoter element. The promoter is operably linked or in functional connection with the coding sequence when expression from the coding sequence via transcription is regulated, or controlled by, the promoter element. The terms “operably linked” and “in functional connection with” are utilized interchangeably herein with respect to promoter elements.

A promoter often interacts with a RNA polymerase. A polymerase is an enzyme that catalyzes synthesis of nucleic acids using a preexisting nucleic acid reagent. When the template is a DNA template, an RNA molecule is transcribed before protein is synthesized. Enzymes having polymerase activity suitable for use in the present methods include any polymerase that is active in the chosen system with the chosen template to synthesize protein. In some embodiments, a promoter (e.g., a heterologous promoter) also referred to herein as a promoter element, can be operably linked to a nucleotide sequence or an open reading frame (ORF). Transcription from the promoter element can catalyze the synthesis of an RNA corresponding to the nucleotide sequence or ORF sequence operably linked to the promoter, which in turn leads to synthesis of a desired peptide, polypeptide or protein.

Promoter elements sometimes exhibit responsiveness to regulatory control. Promoter elements also sometimes can be regulated by a selective agent. That is, transcription from promoter elements sometimes can be turned on, turned off, up-regulated or down-regulated, in response to a change in environmental, nutritional or internal conditions or signals (e.g., heat inducible promoters, light regulated promoters, feedback regulated promoters, hormone influenced promoters, tissue specific promoters, oxygen and pH influenced promoters, promoters that are responsive to selective agents (e.g., kanamycin) and the like, for example). Promoters influenced by environmental, nutritional or internal signals frequently are influenced by a signal (direct or indirect) that binds at or near the promoter and increases or decreases expression of the target sequence under certain conditions.

Non-limiting examples of selective or regulatory agents that can influence transcription from a promoter element used in embodiments described herein include, without limitation, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like). In some embodiments, the regulatory or selective agent can be added to change the existing growth conditions to which the organism is subjected (e.g., growth in liquid culture, growth in a fermenter, growth on solid nutrient plates and the like for example).

In some embodiments, regulation of a promoter element can be used to alter (e.g., increase, add, decrease or substantially eliminate) the activity of a peptide, polypeptide or protein (e.g., enzyme activity for example). For example, a microorganism can be engineered by genetic modification to express a nucleic acid reagent that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent that can decrease expression of an activity by decreasing or substantially eliminating transcription from a homologous or heterologous promoter operably linked to a nucleotide sequence of interest, in certain embodiments.

In some embodiments the activity can be altered using recombinant DNA and genetic techniques known to the artisan. Methods for engineering microorganisms are further described herein. For example, yeast transcriptional repressors and their associated genes, including their DNA binding motifs, can be determined using the MEME sequence analysis software. Potential regulator binding motifs can be identified using the program MEME to search intergenic regions bound by regulators for overrepresented sequences. For each regulator, the sequences of intergenic regions bound with p-values less than 0.001 can be extracted to use as input for motif discovery.

In some embodiments, the altered activity can be found by screening the organism under conditions that select for the desired change in activity. For example, certain microorganisms can be adapted to increase or decrease an activity by selecting or screening the organism in question on a media containing substances that are poorly metabolized or even toxic. An increase in the ability of an organism to grow on a substance that is normally poorly metabolized may result in an increase in the measured growth rate on that substance, for example. A decrease in the sensitivity to a toxic substance might be manifested by growth on higher concentrations of the toxic substance, for example. Genetic modifications that are identified in this manner sometimes are referred to as naturally occurring mutations or the organisms that carry them can sometimes be referred to as naturally occurring mutants. Modifications obtained in this manner are not limited to alterations in promoter sequences. That is, screening microorganisms by selective pressure, as described above, can yield genetic alterations that can occur in non-promoter sequences, and sometimes also can occur in sequences that are not in the nucleotide sequence of interest, but in a related nucleotide sequences (e.g., a gene involved in a different step of the same pathway, a transport gene, and the like). Naturally occurring mutants sometimes can be found by isolating naturally occurring variants from unique environments, in some embodiments.

In addition to the regulated promoter sequences, regulatory sequences, and coding polynucleotides provided herein, a nucleic acid reagent may include a polynucleotide sequence 80% or more identical to the foregoing (or to the complementary sequences). That is, a nucleotide sequence that is at least 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more identical to a nucleotide sequence described herein can be utilized. The term “identical” as used herein refers to two or more nucleotide sequences having substantially the same nucleotide sequence when compared to each other. One test for determining whether two nucleotide sequences or amino acids sequences are substantially identical is to determine the percent of identical nucleotide sequences or amino acid sequences shared.

Calculations of sequence identity can be performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences.

Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the World Wide Web URL http address gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at World Wide Web URL http address gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Sequence identity can also be determined by hybridization assays conducted under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

As noted above, nucleic acid reagents may also comprise one or more 5′ UTR's, and one or more 3′UTR's. A 5′ UTR may comprise one or more elements endogenous to the nucleotide sequence from which it originates, and sometimes includes one or more exogenous elements. A 5′ UTR can originate from any suitable nucleic acid, such as genomic DNA, plasmid DNA, RNA or mRNA, for example, from any suitable organism (e.g., virus, bacterium, yeast, fungi, plant, insect or mammal). The artisan may select appropriate elements for the 5′ UTR based upon the chosen expression system (e.g., expression in a chosen organism, or expression in a cell free system, for example). A 5′ UTR sometimes comprises one or more of the following elements known to the artisan: enhancer sequences (e.g., transcriptional or translational), transcription initiation site, transcription factor binding site, translation regulation site, translation initiation site, translation factor binding site, accessory protein binding site, feedback regulation agent binding sites, Pribnow box, TATA box, -35 element, E-box (helix-loop-helix binding element), ribosome binding site, replicon, internal ribosome entry site (IRES), silencer element and the like. In some embodiments, a promoter element may be isolated such that all 5′ UTR elements necessary for proper conditional regulation are contained in the promoter element fragment, or within a functional subsequence of a promoter element fragment.

A 5′UTR in the nucleic acid reagent can comprise a translational enhancer nucleotide sequence. A translational enhancer nucleotide sequence often is located between the promoter and the target nucleotide sequence in a nucleic acid reagent. A translational enhancer sequence often binds to a ribosome, sometimes is an 18S rRNA-binding ribonucleotide sequence (i.e., a 40S ribosome binding sequence) and sometimes is an internal ribosome entry sequence (IRES). An IRES generally forms an RNA scaffold with precisely placed RNA tertiary structures that contact a 40S ribosomal subunit via a number of specific intermolecular interactions. Examples of ribosomal enhancer sequences are known and can be identified by the artisan (e.g., Mignone et al., Nucleic Acids Research 33: D141-D146 (2005); Paulous et al., Nucleic Acids Research 31: 722-733 (2003); Akbergenov et al., Nucleic Acids Research 32: 239-247 (2004); Mignone et al., Genome Biology 3(3): reviews0004.1-0001.10 (2002); Gallie, Nucleic Acids Research 30: 3401-3411 (2002); Shaloiko et al., World Wide Web URL http address interscience.wiley.com, DOI: 10.1002/bit.20267; and Gallie et al., Nucleic Acids Research 15: 3257-3273 (1987)).

A translational enhancer sequence sometimes is a eukaryotic sequence, such as a Kozak consensus sequence or other sequence (e.g., hydroid polyp sequence, GenBank accession no. U07128). A translational enhancer sequence sometimes is a prokaryotic sequence, such as a Shine-Dalgarno consensus sequence. In certain embodiments, the translational enhancer sequence is a viral nucleotide sequence. A translational enhancer sequence sometimes is from a 5′ UTR of a plant virus, such as Tobacco Mosaic Virus (TMV), Alfalfa Mosaic Virus (AMV); Tobacco Etch Virus (ETV); Potato Virus Y (PVY); Turnip Mosaic (poty) Virus and Pea Seed Borne Mosaic Virus, for example. In certain embodiments, an omega sequence about 67 bases in length from TMV is included in the nucleic acid reagent as a translational enhancer sequence (e.g., devoid of guanosine nucleotides and includes a 25 nucleotide long poly (CAA) central region).

A 3′ UTR may comprise one or more elements endogenous to the nucleotide sequence from which it originates and sometimes includes one or more exogenous elements. A 3′ UTR may originate from any suitable nucleic acid, such as genomic DNA, plasmid DNA, RNA or mRNA, for example, from any suitable organism (e.g., a virus, bacterium, yeast, fungi, plant, insect or mammal). The artisan can select appropriate elements for the 3′ UTR based upon the chosen expression system (e.g., expression in a chosen organism, for example). A 3′ UTR sometimes comprises one or more of the following elements known to the artisan: transcription regulation site, transcription initiation site, transcription termination site, transcription factor binding site, translation regulation site, translation termination site, translation initiation site, translation factor binding site, ribosome binding site, replicon, enhancer element, silencer element and polyadenosine tail. A 3′ UTR often includes a polyadenosine tail and sometimes does not, and if a polyadenosine tail is present, one or more adenosine moieties may be added or deleted from it (e.g., about 5, about 10, about 15, about 20, about 25, about 30, about 35, about 40, about 45 or about 50 adenosine moieties may be added or subtracted).

In some embodiments, modification of a 5′ UTR and/or a 3′ UTR can be used to alter (e.g., increase, add, decrease or substantially eliminate) the activity of a promoter. Alteration of the promoter activity can in turn alter the activity of a peptide, polypeptide or protein (e.g., enzyme activity for example), by a change in transcription of the nucleotide sequence(s) of interest from an operably linked promoter element comprising the modified 5′ or 3′ UTR. For example, a microorganism can be engineered by genetic modification to express a nucleic acid reagent comprising a modified 5′ or 3′ UTR that can add a novel activity (e.g., an activity not normally found in the host organism) or increase the expression of an existing activity by increasing transcription from a homologous or heterologous promoter operably linked to a nucleotide sequence of interest (e.g., homologous or heterologous nucleotide sequence of interest), in certain embodiments. In some embodiments, a microorganism can be engineered by genetic modification to express a nucleic acid reagent comprising a modified 5′ or 3′ UTR that can decrease (reduce or abolish) the expression of an activity by decreasing or substantially eliminating transcription from a homologous or heterologous promoter operably linked to a nucleotide sequence of interest, in certain embodiments.

A nucleotide reagent sometimes can comprise a target nucleotide sequence. A “target nucleotide sequence” as used herein encodes a nucleic acid, peptide, polypeptide or protein of interest, and may be a ribonucleotide sequence or a deoxyribonucleotide sequence. A target nucleic acid sometimes is an untranslated ribonucleic acid and sometimes is a translated ribonucleic acid. An untranslated ribonucleic acid may include, but is not limited to, a small interfering ribonucleic acid (siRNA), a short hairpin ribonucleic acid (shRNA), other ribonucleic acid capable of RNA interference (RNAi), an antisense ribonucleic acid, or a ribozyme. A translatable target nucleotide sequence (e.g., a target ribonucleotide sequence) sometimes encodes a peptide, polypeptide or protein, which are sometimes referred to herein as “target peptides,” “target polypeptides” or “target proteins.”

Any peptides, polypeptides or proteins, or an activity catalyzed by one or more peptides, polypeptides or proteins may be encoded by a target nucleotide sequence and may be selected by a user. Representative proteins include enzymes, e.g., cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase, 3-hydroxypropionyl-CoA hydrolase, 3-hydroxypropionate dehydrogenase and malonate semialdehyde dehydrogenase. The term “enzyme” as used herein refers to a protein which can act as a catalyst to induce a chemical change in other compounds, thereby producing one or more products from one or more substrates.

Specific polypeptides (e.g., enzymes) useful for embodiments described herein are listed herein. The term “protein” as used herein refers to a molecule having a sequence of amino acids linked by peptide bonds. This term includes fusion proteins, oligopeptides, peptides, cyclic peptides, polypeptides and polypeptide derivatives, whether native or recombinant, and also includes fragments, derivatives, homologs, and variants thereof. A protein or polypeptide sometimes is of intracellular origin (e.g., located in the nucleus, cytosol, or interstitial space of host cells in vivo) and sometimes is a cell membrane protein in vivo. In some embodiments (described above, and in further detail hereafter in Engineering and Alteration Methods), a genetic modification can result in a modification (e.g., increase, substantially increase, decrease or substantially decrease) of a target activity.

A translatable nucleotide sequence generally is located between a start codon (AUG in ribonucleic acids and ATG in deoxyribonucleic acids) and a stop codon (e.g., UAA (ochre), UAG (amber) or UGA (opal) in ribonucleic acids and TAA, TAG or TGA in deoxyribonucleic acids), and sometimes is referred to herein as an “open reading frame” (ORF). A translatable nucleotide sequence (e.g., ORF) sometimes is encoded differently in one organism (e.g., most organisms encode CTG as leucine) than in another organism (e.g., C. tropicalis encodes CTG as serine). In some embodiments, a translatable nucleotide sequence is altered to correct alternate genetic code (e.g., codon usage) differences between a nucleotide donor organism and an nucleotide recipient organism (e.g., engineered organism). In certain embodiments, a translatable nucleotide sequence is altered to improve; (i) codon usage, (ii) transcriptional efficiency, (iii) translational efficiency, (iv) the like, and combinations thereof.

A nucleic acid reagent sometimes comprises one or more ORFs. An ORF may be from any suitable source, sometimes from genomic DNA, mRNA, reverse transcribed RNA or complementary DNA (cDNA) or a nucleic acid library comprising one or more of the foregoing, and is from any organism species that contains a nucleic acid sequence of interest, protein of interest, or activity of interest. Non-limiting examples of organisms from which an ORF can be obtained include bacteria, yeast, fungi, human, insect, nematode, bovine, equine, canine, feline, rat or mouse, for example.

A nucleic acid reagent sometimes comprises a nucleotide sequence adjacent to an ORF that is translated in conjunction with the ORF and encodes an amino acid tag. The tag-encoding nucleotide sequence is located 3′ and/or 5′ of an ORF in the nucleic acid reagent, thereby encoding a tag at the C-terminus or N-terminus of the protein or peptide encoded by the ORF. Any tag that does not abrogate in vitro transcription and/or translation may be utilized and may be appropriately selected by the artisan. Tags may facilitate isolation and/or purification of the desired ORF product from culture or fermentation media.

A tag sometimes specifically binds a molecule or moiety of a solid phase or a detectable label, for example, thereby having utility for isolating, purifying and/or detecting a protein or peptide encoded by the ORF. In some embodiments, a tag comprises one or more of the following elements: FLAG (e.g., DYKDDDDKG), V5 (e.g., GKPIPNPLLGLDST), c-MYC (e.g., EQKLISEEDL), HSV (e.g., QPELAPEDPED), influenza hemaglutinin, HA (e.g., YPYDVPDYA), VSV-G (e.g., YTDIEMNRLGK), bacterial glutathione-S-transferase, maltose binding protein, a streptavidin- or avidin-binding tag (e.g., pcDNA™6 BioEase™ Gateway® Biotinylation System (Invitrogen)), thioredoxin, β-galactosidase, VSV-glycoprotein, a fluorescent protein (e.g., green fluorescent protein or one of its many color variants (e.g., yellow, red, blue)), a polylysine or polyarginine sequence, a polyhistidine sequence (e.g., His6) or other sequence that chelates a metal (e.g., cobalt, zinc, copper), and/or a cysteine-rich sequence that binds to an arsenic-containing molecule. In certain embodiments, a cysteine-rich tag comprises the amino acid sequence CC-Xn-CC, wherein X is any amino acid and n is 1 to 3, and the cysteine-rich sequence sometimes is CCPGCC. In certain embodiments, the tag comprises a cysteine-rich element and a polyhistidine element (e.g., CCPGCC and His6).

A tag often conveniently binds to a binding partner. For example, some tags bind to an antibody (e.g., FLAG) and sometimes specifically bind to a small molecule. For example, a polyhistidine tag specifically chelates a bivalent metal, such as copper, zinc and cobalt; a polylysine or polyarginine tag specifically binds to a zinc finger; a glutathione S-transferase tag binds to glutathione; and a cysteine-rich tag specifically binds to an arsenic-containing molecule. Arsenic-containing molecules include LUMIO™ agents (Invitrogen, California), such as FlAsH™ (EDT2[4′,5′-bis(1,3,2-dithioarsolan-2-yl)fluorescein-(1,2-ethanedithiol)2]) and ReAsH reagents (e.g., U.S. Pat. No. 5,932,474 to Tsien et al., entitled “Target Sequences for Synthetic Molecules;” U.S. Pat. No. 6,054,271 to Tsien et al., entitled “Methods of Using Synthetic Molecules and Target Sequences;” U.S. Pat. Nos. 6,451,569 and 6,008,378; published U.S. Patent Application 2003/0083373, and published PCT Patent Application WO 99/21013, all to Tsien et al. and all entitled “Synthetic Molecules that Specifically React with Target Sequences”). Such antibodies and small molecules sometimes are linked to a solid phase for convenient isolation of the target protein or target peptide.

A tag sometimes comprises a sequence that localizes a translated protein or peptide to a component in a system, which is referred to as a “signal sequence” or “localization signal sequence” herein. A signal sequence often is incorporated at the N-terminus of a target protein or target peptide, and sometimes is incorporated at the C-terminus. Examples of signal sequences are known to the artisan, are readily incorporated into a nucleic acid reagent, and often are selected according to the organism in which expression of the nucleic acid reagent is performed. A signal sequence in some embodiments localizes a translated protein or peptide to a cell membrane. Examples of signal sequences include, but are not limited to, a nucleus targeting signal (e.g., steroid receptor sequence and N-terminal sequence of SV40 virus large T antigen); mitochondrial targeting signal (e.g., amino acid sequence that forms an amphipathic helix); peroxisome targeting signal (e.g., C-terminal sequence in YFG from S. cerevisiae); and a secretion signal (e.g., N-terminal sequences from invertase, mating factor alpha, PHO5 and SUC2 in S. cerevisiae; multiple N-terminal sequences of B. subtilis proteins (e.g., Tjalsma et al., Microbiol. Molec. Biol. Rev. 64: 515-547 (2000)); alpha amylase signal sequence (e.g., U.S. Pat. No. 6,288,302); pectate lyase signal sequence (e.g., U.S. Pat. No. 5,846,818); precollagen signal sequence (e.g., U.S. Pat. No. 5,712,114); OmpA signal sequence (e.g., U.S. Pat. No. 5,470,719); lam beta signal sequence (e.g., U.S. Pat. No. 5,389,529); B. brevis signal sequence (e.g., U.S. Pat. No. 5,232,841); and P. pastoris signal sequence (e.g., U.S. Pat. No. 5,268,273).

A tag sometimes is directly adjacent to the amino acid sequence encoded by an ORF (i.e., there is no intervening sequence) and sometimes a tag is substantially adjacent to an ORF encoded amino acid sequence (e.g., an intervening sequence is present). An intervening sequence sometimes includes a recognition site for a protease, which is useful for cleaving a tag from a target protein or peptide. In some embodiments, the intervening sequence is cleaved by Factor Xa (e.g., recognition site I (E/D)GR), thrombin (e.g., recognition site LVPRGS), enterokinase (e.g., recognition site DDDDK), TEV protease (e.g., recognition site ENLYFQG) or PreScission™ protease (e.g., recognition site LEVLFQGP), for example.

An intervening sequence sometimes is referred to herein as a “linker sequence,” and may be of any suitable length selected by the artisan. A linker sequence sometimes is about 1 to about 20 amino acids in length, and sometimes about 5 to about 10 amino acids in length. The artisan may select the linker length to substantially preserve target protein or peptide function (e.g., a tag may reduce target protein or peptide function unless separated by a linker), to enhance disassociation of a tag from a target protein or peptide when a protease cleavage site is present (e.g., cleavage may be enhanced when a linker is present), and to enhance interaction of a tag/target protein product with a solid phase. A linker can be of any suitable amino acid content, and often comprises a higher proportion of amino acids having relatively short side chains (e.g., glycine, alanine, serine and threonine).

A nucleic acid reagent sometimes includes a stop codon between a tag element and an insertion element or ORF, which can be useful for translating an ORF with or without the tag. Mutant tRNA molecules that recognize stop codons (described above) suppress translation termination and thereby are designated “suppressor tRNAs.” Suppressor tRNAs can result in the insertion of amino acids and continuation of translation past stop codons (e.g., U.S. Patent Application No. 60/587,583, filed Jul. 14, 2004, entitled “Production of Fusion Proteins by Cell-Free Protein Synthesis,”; Eggertsson, et al., (1988) Microbiological Review 52(3):354-374, and Engleerg-Kukla, et al. (1996) in Escherichia coli and Salmonella Cellular and Molecular Biology, Chapter 60, pps 909-921, Neidhardt, et al. eds., ASM Press, Washington, D.C.). A number of suppressor tRNAs are known, including but not limited to, supE, supP, supD, supF and supZ suppressors, which suppress the termination of translation of the amber stop codon; supB, g1T, supL, supN, supC and supM suppressors, which suppress the function of the ochre stop codon and glyT, trpT and Su-9 suppressors, which suppress the function of the opal stop codon. In general, suppressor tRNAs contain one or more mutations in the anti-codon loop of the tRNA that allows the tRNA to base pair with a codon that ordinarily functions as a stop codon. The mutant tRNA is charged with its cognate amino acid residue and the cognate amino acid residue is inserted into the translating polypeptide when the stop codon is encountered. Mutations that enhance the efficiency of termination suppressors (i.e., increase stop codon read-through) have been identified. These include, but are not limited to, mutations in the uar gene (also known as the prfA gene), mutations in the ups gene, mutations in the sueA, sueB and sueC genes, mutations in the rpsD (ramA) and rpsE (spcA) genes and mutations in the rplL gene.

Thus, a nucleic acid reagent comprising a stop codon located between an ORF and a tag can yield a translated ORF alone when no suppressor tRNA is present in the translation system, and can yield a translated ORF-tag fusion when a suppressor tRNA is present in the system. Suppressor tRNA can be generated in cells transfected with a nucleic acid encoding the tRNA (e.g., a replication incompetent adenovirus containing the human tRNA-Ser suppressor gene can be transfected into cells, or a YAC containing a yeast or bacterial tRNA suppressor gene can be transfected into yeast cells, for example). Vectors for synthesizing suppressor tRNA and for translating ORFs with or without a tag are available to the artisan (e.g., Tag-On-Demand™ kit (Life Technolgies, a Thermo Fisher Scientific company, California; Capone et al., Amber, ochre and opal suppressor tRNA genes derived from a human serine tRNA gene. EMBO J. 4:213, 1985).

Any convenient cloning strategy known in the art may be utilized to incorporate an element, such as an ORF, into a nucleic acid reagent. Known methods can be utilized to insert an element into the template independent of an insertion element, such as (1) cleaving the template at one or more existing restriction enzyme sites and ligating an element of interest and (2) adding restriction enzyme sites to the template by hybridizing oligonucleotide primers that include one or more suitable restriction enzyme sites and amplifying by polymerase chain reaction (described in greater detail herein). Other cloning strategies take advantage of one or more insertion sites present or inserted into the nucleic acid reagent, such as an oligonucleotide primer hybridization site for PCR, for example, and others described herein. In some embodiments, a cloning strategy can be combined with genetic manipulation such as recombination (e.g., recombination of a nucleic acid reagent with a nucleic acid sequence of interest into the genome of the organism to be modified, as described further herein). In some embodiments, the cloned ORF(s) can produce (directly or indirectly) 3-HP, by engineering a microorganism with one or more ORFs of interest.

In some embodiments, the nucleic acid reagent includes one or more recombinase insertion sites. A recombinase insertion site is a recognition sequence on a nucleic acid molecule that participates in an integration/recombination reaction by recombination proteins. For example, the recombination site for Cre recombinase is loxP, which is a 34 base pair sequence comprised of two 13 base pair inverted repeats (serving as the recombinase binding sites) flanking an 8 base pair core sequence (e.g., FIG. 1 of Sauer, B., Curr. Opin. Biotech. 5:521-527 (1994)). Other examples of recombination sites include attB, attP, attL, and attR sequences, and mutants, fragments, variants and derivatives thereof, which are recognized by the recombination protein X, Int and by the auxiliary proteins integration host factor (IHF), FIS and excisionase (Xis) (e.g., U.S. Pat. Nos. 5,888,732; 6,143,557; 6,171,861; 6,270,969; 6,277,608; and 6,720,140; U.S. patent application Ser. No. 09/517,466, filed Mar. 2, 2000, and Ser. No. 09/732,914, filed Aug. 14, 2003, and in U.S. patent publication no. 2002-0007051-A1; Landy, Curr. Opin. Biotech. 3:699-707 (1993)).

Examples of recombinase cloning nucleic acids are in Gateway® systems (Life Technologies, a Thermo Fisher Scientific company, California), which include at least one recombination site for cloning a desired nucleic acid molecules in vivo or in vitro. In some embodiments, the system utilizes vectors that contain at least two different site-specific recombination sites, often based on the bacteriophage lambda system (e.g., att1 and att2), and are mutated from the wild-type (att0) sites. Each mutated site has a unique specificity for its cognate partner att site (i.e., its binding partner recombination site) of the same type (for example attB1 with attP1, or attL1 with attR1) and will not cross-react with recombination sites of the other mutant type or with the wild-type att0 site. Different site specificities allow directional cloning or linkage of desired molecules thus providing desired orientation of the cloned molecules. Nucleic acid fragments flanked by recombination sites are cloned and subcloned using the Gateway® system by replacing a selectable marker (for example, ccdB) flanked by att sites on the recipient plasmid molecule, sometimes termed the Destination Vector. Desired clones are then selected by transformation of a ccdB sensitive host strain and positive selection for a marker on the recipient molecule. Similar strategies for negative selection (e.g., use of toxic genes) can be used in other organisms such as thymidine kinase (TK) in mammals and insects.

A recombination system useful for engineering yeast is outlined briefly. The system makes use of the URA3 gene (e.g., for S. cerevisieae and C. albicans, for example) or URA4 and URA5 genes (e.g., for S. pombe, for example) and toxicity of the nucleotide analogue 5-Fluoroorotic acid (5-FOA). The URA3 or URA4 and URA5 genes encode orotine-5′-monophosphate (OMP) decarboxylase. Yeast with an active URA3 or URA4 and URA5 gene (phenotypically Ura+) convert 5-FOA to fluorodeoxyuridine, which is toxic to yeast cells. Yeast carrying a mutation in the appropriate gene(s) or having a knock out of the appropriate gene(s) can grow in the presence of 5-FOA, if the media is also supplemented with uracil.

A nucleic acid engineering construct can be made which may comprise the URA3 gene or cassette, flanked on either side by the same nucleotide sequence in the same orientation. The URA3 cassette comprises a promoter, the URA3 gene and a functional transcription terminator. Target sequences which direct the construct to a particular nucleic acid region of interest in the organism to be engineered are added such that the target sequences are adjacent to and about the flanking sequences on either side of the URA3 cassette. Yeast can be transformed with the engineering construct and plated on minimal media without uracil. Colonies can be screened by PCR to determine those transformants that have the engineering construct inserted in the proper location in the genome. Checking insertion location prior to selecting for recombination of the URA3 cassette may reduce the number of incorrect clones carried through to later stages of the procedure. Correctly inserted transformants can then be replica plated on minimal media containing 5-FOA to select for recombination of the URA3 cassette out of the construct, leaving a disrupted gene and an identifiable footprint (e.g., nucleic acid sequence) that can be used to verify the presence of the disrupted gene. The technique described is useful for disrupting or “knocking out” gene function, but also can be used to insert genes or constructs into a host organisms genome in a targeted, sequence specific manner.

A nucleic acid reagent sometimes contains one or more origin of replication (ORI) elements. In some embodiments, a template comprises two or more ORIs, where one reagent functions efficiently in one organism (e.g., a bacterium) and another reagent functions efficiently in another organism (e.g., a eukaryote, like yeast for example). In some embodiments, an ORI may function efficiently in one species (e.g., S. cerevisieae, for example) and another ORI may function efficiently in a different species (e.g., S. pombe, for example). A nucleic acid reagent also sometimes includes one or more transcription regulation sites.

A nucleic acid reagent can include one or more selection elements (e.g., elements for selection of the presence of the nucleic acid reagent, and not for activation of a promoter element which can be selectively regulated). Selection elements often are utilized using known processes to determine whether a nucleic acid reagent is included in a cell. In some embodiments, a nucleic acid reagent includes two or more selection elements, where one reagent functions efficiently in one organism and another reagent functions efficiently in another organism. Examples of selection elements include, but are not limited to, (1) nucleic acid segments that encode products that provide resistance against otherwise toxic compounds (e.g., antibiotics); (2) nucleic acid segments that encode products that are otherwise lacking in the recipient cell (e.g., essential products, tRNA genes, auxotrophic markers); (3) nucleic acid segments that encode products that suppress the activity of a gene product; (4) nucleic acid segments that encode products that can be readily identified (e.g., phenotypic markers such as antibiotics (e.g., β-lactamase), β-galactosidase, green fluorescent protein (GFP), yellow fluorescent protein (YFP), red fluorescent protein (RFP), cyan fluorescent protein (CFP), and cell surface proteins); (5) nucleic acid segments that bind products that are otherwise detrimental to cell survival and/or function; (6) nucleic acid segments that otherwise inhibit the activity of any of the nucleic acid segments described in Nos. 1-5 above (e.g., antisense oligonucleotides); (7) nucleic acid segments that bind products that modify a substrate (e.g., restriction endonucleases); (8) nucleic acid segments that can be used to isolate or identify a desired molecule (e.g., specific protein binding sites); (9) nucleic acid segments that encode a specific nucleotide sequence that can be otherwise non-functional (e.g., for PCR amplification of subpopulations of molecules); (10) nucleic acid segments that, when absent, directly or indirectly confer resistance or sensitivity to particular compounds; (11) nucleic acid segments that encode products that either are toxic or convert a relatively non-toxic compound to a toxic compound (e.g., Herpes simplex thymidine kinase, cytosine deaminase) in recipient cells; (12) nucleic acid segments that inhibit replication, partition or heritability of nucleic acid molecules that contain them; and/or (13) nucleic acid segments that encode conditional replication functions, e.g., replication in certain hosts or host cell strains or under certain environmental conditions (e.g., temperature, nutritional conditions, and the like).

A nucleic acid reagent is of any form useful as an expression vector for in vivo transcription and/or translation. A nucleic acid sometimes is a plasmid, such as a supercoiled plasmid, sometimes is a yeast artificial chromosome (e.g., YAC), sometimes is a linear nucleic acid (e.g., a linear nucleic acid produced by PCR or by restriction digest), sometimes is single-stranded and sometimes is double-stranded. A nucleic acid reagent sometimes is prepared by an amplification process, such as a polymerase chain reaction (PCR) process or transcription-mediated amplification process (TMA). In TMA, two enzymes are used in an isothermal reaction to produce amplification products detected by light emission (see, e.g., Biochemistry 1996 Jun. 25; 35(25):8429-38 and World Wide Web URL http address devicelink.com/ivdt/archive/00/11/007.html). Standard PCR processes are known (e.g., U.S. Pat. Nos. 4,683,202; 4,683,195; 4,965,188; and 5,656,493), and generally are performed in cycles. Each cycle includes heat denaturation, in which hybrid nucleic acids dissociate; cooling, in which primer oligonucleotides hybridize; and extension of the oligonucleotides by a polymerase (i.e., Taq polymerase). An example of a PCR cyclical process is treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Multiple cycles frequently are performed using a commercially available thermal cycler. PCR amplification products sometimes are stored for a time at a lower temperature (e.g., at 4° C.) and sometimes are frozen (e.g., at −20° C.) before analysis.

In some embodiments, a nucleic acid reagent, protein reagent, protein fragment reagent or other reagent described herein is isolated or purified. The term “isolated” as used herein refers to material removed from its original environment (e.g., the natural environment if it is naturally occurring, or a host cell if expressed exogenously), and thus is altered “by the hand of man” from its original environment. The term “purified” as used herein with reference to molecules does not refer to absolute purity. Rather, “purified” refers to a substance in a composition that contains fewer substance species in the same class (e.g., nucleic acid or protein species) other than the substance of interest in comparison to the sample from which it originated. “Purified,” if a nucleic acid or protein for example, refers to a substance in a composition that contains fewer nucleic acid species or protein species other than the nucleic acid or protein of interest in comparison to the sample from which it originated. Sometimes, a protein or nucleic acid is “substantially pure,” indicating that the protein or nucleic acid represents at least 50% of protein or nucleic acid on a mass basis of the composition. Often, a substantially pure protein or nucleic acid is at least 75% on a mass basis of the composition, and sometimes at least 95% on a mass basis of the composition.

### Engineering and Alteration Methods

Methods and compositions (e.g., nucleic acid reagents) described herein can be used to generate engineered microorganisms. As noted above, the term “engineered microorganism” as used herein refers to a modified organism that includes one or more activities distinct from an activity present in a microorganism utilized as a starting point for modification (e.g., host microorganism or unmodified organism). Engineered microorganisms typically arise as a result of a genetic modification, usually introduced or selected for, by one of skill in the art using readily available techniques. Non-limiting examples of methods useful for generating an altered activity include, introducing a heterologous polynucleotide (e.g., nucleic acid or gene integration, also referred to as “knock in”), removing an endogenous polynucleotide, altering the sequence of an existing endogenous nucleic acid sequence (e.g., site-directed mutagenesis), disruption of an existing endogenous nucleic acid sequence (e.g., knock outs and transposon or insertion element mediated mutagenesis), selection for an altered activity where the selection causes a change in a naturally occurring activity that can be stably inherited (e.g., causes a change in a nucleic acid sequence in the genome of the organism or in an epigenetic nucleic acid that is replicated and passed on to daughter cells), PCR-based mutagenesis, and the like. The term “mutagenesis” as used herein refers to any modification to a nucleic acid (e.g., nucleic acid reagent, or host chromosome, for example) that is subsequently used to generate a product in a host or modified organism. Non-limiting examples of mutagenesis include deletion, insertion, substitution, rearrangement, point mutations, suppressor mutations and the like. Mutagenesis methods are known in the art and are readily available to the artisan. Non-limiting examples of mutagenesis methods are described herein and can also be found in Maniatis, T., E. F. Fritsch and J. Sambrook (1982) Molecular Cloning: a Laboratory Manual; Cold Spring Harbor Laboratory, Cold Spring Harbor, N.Y. Another non-limiting example of mutagenesis can be conducted using a Stratagene (San Diego, Calif.) “QuickChange” kit according to the manufacturer's instructions.

The term “genetic modification” as used herein refers to any suitable nucleic acid addition, removal or alteration that facilitates production of a target product (e.g., 3-HP) in an engineered microorganism. Genetic modifications include, without limitation, insertion of one or more nucleotides in a native nucleic acid of a host organism in one or more locations, deletion of one or more nucleotides in a native nucleic acid of a host organism in one or more locations, modification or substitution of one or more nucleotides in a native nucleic acid of a host organism in one or more locations, insertion of a non-native nucleic acid into a host organism (e.g., insertion of an autonomously replicating vector), and removal of a non-native nucleic acid in a host organism (e.g., removal of a vector).

The term “heterologous polynucleotide” as used herein refers to a nucleotide sequence not present in a host microorganism in some embodiments. In certain embodiments, a heterologous polynucleotide is present in a different amount (e.g., different copy number) than in a host microorganism, which can be accomplished, for example, by introducing more copies of a particular nucleotide sequence to a host microorganism (e.g., the particular nucleotide sequence may be in a nucleic acid autonomous of the host chromosome or may be inserted into a chromosome). A heterologous polynucleotide is from a different organism in some embodiments, and in certain embodiments, is from the same type of organism but from an outside source (e.g., a recombinant source).

In some embodiments, an organism engineered using the methods and nucleic acid reagents described herein can produce 3-HP. In certain embodiments, an engineered microorganism described herein that produces 3-HP may comprise one or more altered activities selected from the group consisting of cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase, 3-hydroxypropionyl-CoA hydrolase, 3-hydroxypropionate dehydrogenase (HPD1) and malonate semialdehyde dehydrogenase (ALD6) (acetylating). In some embodiments, an engineered microorganism as described herein may comprise a genetic modification that decreases or eliminates HPD1 and/or ALD6 activities. In some embodiments, an engineered microorganism as described herein may comprise a genetic modification that adds or increases a cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase or 3-hydroxypropionyl-CoA hydrolase activity.

The term “altered activity” as used herein refers to an activity in an engineered microorganism that is added or modified relative to the host microorganism (e.g., added, increased, reduced, inhibited or removed activity). An activity can be altered by introducing a genetic modification to a host microorganism that yields an engineered microorganism having added, increased, reduced, inhibited or removed activity.

An added activity often is an activity not detectable in a host microorganism. An increased activity generally is an activity detectable in a host microorganism that has been increased in an engineered microorganism. An activity can be increased to any suitable level for production of a target product (e.g., 3-HP), including but not limited to less than 1.2 fold, 1.5 fold, 2-fold, 2.5 fold, 3 fold, 3.5 fold, 4 fold, 5 fold, 6 fold, 7 fold, 8 fold, 9 fold, 10 fold, 12 fold, 13 fold, 14 fold, 15 fold, 16 fold, 17, fold 18 fold 19 fold, 20 fold or greater than 20 fold (e.g., about 0.5% increase to about 99% increase; about 20%, 30%, 40%, 50%, 60%, 70%, 80%, 90% increase). A reduced or inhibited activity generally is an activity detectable in a host microorganism that has been reduced or inhibited in an engineered microorganism. An activity can be reduced to undetectable levels in some embodiments, or detectable levels in certain embodiments. An activity can be decreased to any suitable level for production of a target product (e.g., 3-HP), including but not limited to less than 2-fold (e.g., about 10% decrease to about 99% decrease; about 20%, 30%, 40%, 50%, 60%, 70%, 80%, 90% decrease), 2-fold, 3-fold, 4-fold, 5-fold, 6-fold, 7-fold, 8-fold, 9-fold, of 10-fold decrease, or greater than about 10-fold decrease.

An altered activity sometimes is an activity not detectable in a host organism and is added to an engineered organism. An altered activity also may be an activity detectable in a host organism and is increased in an engineered organism. An activity may be added or increased by increasing the number of copies of a polynucleotide that encodes a polypeptide having a target activity, in some embodiments. In certain embodiments an activity can be added or increased by inserting into a host microorganism a heterologous polynucleotide that encodes a polypeptide having the added activity. In certain embodiments, an activity can be added or increased by inserting into a host microorganism a heterologous polynucleotide that is (i) operably linked to another polynucleotide that encodes a polypeptide having the added activity, and (ii) up regulates production of the polynucleotide. Thus, an activity can be added or increased by inserting or modifying a regulatory polynucleotide operably linked to another polynucleotide that encodes a polypeptide having the target activity. In certain embodiments, an activity can be added or increased by subjecting a host microorganism to a selective environment and screening for microorganisms that have a detectable level of the target activity. Examples of a selective environment include, without limitation, a medium containing a substrate that a host organism can process and a medium lacking a substrate that a host organism can process.

An altered activity sometimes is an activity detectable in a host organism and is reduced, inhibited or removed (i.e., not detectable) in an engineered organism. An activity may be reduced or removed by decreasing the number of copies of a polynucleotide that encodes a polypeptide having a target activity, in some embodiments. In some embodiments, an activity can be reduced or removed by (i) inserting a polynucleotide within a polynucleotide that encodes a polypeptide having the target activity (disruptive insertion), and/or (ii) removing a portion of or all of a polynucleotide that encodes a polypeptide having the target activity (deletion or knock out, respectively). In certain embodiments, an activity can be reduced or removed by inserting into a host microorganism a heterologous polynucleotide that is (i) operably linked to another polynucleotide that encodes a polypeptide having the target activity, and (ii) down regulates production of the polynucleotide. Thus, an activity can be reduced or removed by inserting or modifying a regulatory polynucleotide operably linked to another polynucleotide that encodes a polypeptide having the target activity.

An activity also can be reduced or removed by (i) inhibiting a polynucleotide that encodes a polypeptide having the activity or (ii) inhibiting a polynucleotide operably linked to another polynucleotide that encodes a polypeptide having the activity. A polynucleotide can be inhibited by a suitable technique known in the art, such as by contacting an RNA encoded by the polynucleotide with a specific inhibitory RNA (e.g., RNAi, siRNA, ribozyme). An activity also can be reduced or removed by contacting a polypeptide having the activity with a molecule that specifically inhibits the activity (e.g., enzyme inhibitor, antibody). In certain embodiments, an activity can be reduced or removed by subjecting a host microorganism to a selective environment and screening for microorganisms that have a reduced level or removal of the target activity.

In some embodiments, an untranslated ribonucleic acid, or a cDNA can be used to reduce the expression of a particular activity or enzyme. For example, a microorganism can be engineered by genetic modification to express a nucleic acid reagent that reduces the expression of an activity by producing an RNA molecule that is partially or substantially homologous to a nucleic acid sequence of interest which encodes the activity of interest. The RNA molecule can bind to the nucleic acid sequence of interest and inhibit the nucleic acid sequence from performing its natural function, in certain embodiments. In some embodiments, the RNA may alter the nucleic acid sequence of interest which encodes the activity of interest in a manner that the nucleic acid sequence of interest is no longer capable of performing its natural function (e.g., the action of a ribozyme for example).

In certain embodiments, nucleotide sequences sometimes are added to, modified or removed from one or more of the nucleic acid reagent elements, such as the promoter, 5′UTR, target sequence, or 3′UTR elements, to enhance, potentially enhance, reduce, or potentially reduce transcription and/or translation before or after such elements are incorporated in a nucleic acid reagent. In some embodiments, one or more of the following sequences may be modified or removed if they are present in a 5′UTR: a sequence that forms a stable secondary structure (e.g., quadruplex structure or stem loop stem structure (e.g., EMBL sequences X12949, AF274954, AF139980, AF152961, S95936, U194144, AF116649 or substantially identical sequences that form such stem loop stem structures); a translation initiation codon upstream of the target nucleotide sequence start codon; a stop codon upstream of the target nucleotide sequence translation initiation codon; an ORF upstream of the target nucleotide sequence translation initiation codon; an iron responsive element (IRE) or like sequence; and a 5′ terminal oligopyrimidine tract (TOP, e.g., consisting of 5-15 pyrimidines adjacent to the cap). A translational enhancer sequence and/or an internal ribosome entry site (IRES) sometimes is inserted into a 5′UTR (e.g., EMBL nucleotide sequences J04513, X87949, M95825, M12783, AF025841, AF013263, AF006822, M17169, M13440, M22427, D14838 and M17446 and substantially identical nucleotide sequences).

An AU-rich element (ARE, e.g., AUUUA repeats) and/or splicing junction that follows a non-sense codon sometimes is removed from or modified in a 3′UTR. A polyadenosine tail sometimes is inserted into a 3′UTR if none is present, sometimes is removed if it is present, and adenosine moieties sometimes are added to or removed from a polyadenosine tail present in a 3′UTR. Thus, some embodiments are directed to a process comprising: determining whether any nucleotide sequences that increase, potentially increase, reduce or potentially reduce translation efficiency are present in the elements, and adding, removing or modifying one or more of such sequences if they are identified. Certain embodiments are directed to a process comprising: determining whether any nucleotide sequences that increase or potentially increase translation efficiency are not present in the elements, and incorporating such sequences into the nucleic acid reagent.

In some embodiments, an activity can be altered by modifying the nucleotide sequence of an ORF. An ORF sometimes is mutated or modified (for example, by point mutation, deletion mutation, insertion mutation, PCR based mutagenesis and the like) to alter, enhance or increase, reduce, substantially reduce or eliminate the activity of the encoded protein or peptide. The protein or peptide encoded by a modified ORF sometimes is produced in a lower amount or may not be produced at detectable levels, and in some embodiments, the product or protein encoded by the modified ORF is produced at a higher level (e.g., codons sometimes are modified so they are compatible with tRNA's preferentially used in the host organism or engineered organism). To determine the relative activity, the activity from the product of the mutated ORF (or cell containing it) can be compared to the activity of the product or protein encoded by the unmodified ORF (or cell containing it).

In some embodiments, an ORF nucleotide sequence sometimes is mutated or modified to alter the triplet nucleotide sequences used to encode amino acids (e.g., amino acid codon triplets, for example). Modification of the nucleotide sequence of an ORF to alter codon triplets sometimes is used to change the codon found in the original sequence to better match the preferred codon usage of the organism in which the ORF or nucleic acid reagent will be expressed. The codon usage, and therefore the codon triplets encoded by a nucleic acid sequence, in bacteria may be different from the preferred codon usage in eukaryotes, like yeast or plants for example. Preferred codon usage also may be different between bacterial species. In certain embodiments an ORF nucleotide sequences sometimes is modified to eliminate codon pairs and/or eliminate mRNA secondary structures that can cause pauses during translation of the mRNA encoded by the ORF nucleotide sequence. Translational pausing sometimes occurs when nucleic acid secondary structures exist in an mRNA, and sometimes occurs due to the presence of codon pairs that slow the rate of translation by causing ribosomes to pause. In some embodiments, the use of lower abundance codon triplets can reduce translational pausing due to a decrease in the pause time needed to load a charged tRNA into the ribosome translation machinery. Therefore, to increase transcriptional and translational efficiency in bacteria (e.g., where transcription and translation are concurrent, for example) or to increase translational efficiency in eukaryotes (e.g., where transcription and translation are functionally separated), the nucleotide sequence of a nucleotide sequence of interest can be altered to better suit the transcription and/or translational machinery of the host and/or genetically modified microorganism. In certain embodiments, slowing the rate of translation by the use of lower abundance codons, which slow or pause the ribosome, can lead to higher yields of the desired product due to an increase in correctly folded proteins and a reduction in the formation of inclusion bodies.

Codons can be altered and optimized according to the preferred usage by a given organism by determining the codon distribution of the nucleotide sequence donor organism and comparing the distribution of codons to the distribution of codons in the recipient or host organism. Techniques described herein (e.g., site directed mutagenesis and the like) can then be used to alter the codons accordingly. Comparisons of codon usage can be done by hand, or using nucleic acid analysis software commercially available to the artisan.

Modification of the nucleotide sequence of an ORF also can be used to correct codon triplet sequences that have diverged in different organisms. For example, certain yeast (e.g., C. tropicalis and C. maltosa) use the amino acid triplet CUG (e.g., CTG in the DNA sequence) to encode serine. CUG typically encodes leucine in most organisms. In order to maintain the correct amino acid in the resultant polypeptide or protein, the CUG codon must be altered to reflect the organism in which the nucleic acid reagent will be expressed. Thus, if an ORF from a bacterial donor is to be expressed in either Candida yeast strain mentioned above, the heterologous nucleotide sequence must first be altered or modified to the appropriate leucine codon. Therefore, in some embodiments, the nucleotide sequence of an ORF sometimes is altered or modified to correct for differences that have occurred in the evolution of the amino acid codon triplets between different organisms. In some embodiments, the nucleotide sequence can be left unchanged at a particular amino acid codon, if the amino acid encoded is a conservative or neutral change in amino acid when compared to the originally encoded amino acid.

In some embodiments, an activity can be altered by modifying translational regulation signals, like a stop codon for example. A stop codon at the end of an ORF sometimes is modified to another stop codon, such as an amber stop codon, described above. In some embodiments, a stop codon is introduced within an ORF, sometimes by insertion or mutation of an existing codon. An ORF comprising a modified terminal stop codon and/or internal stop codon often is translated in a system comprising a suppressor tRNA that recognizes the stop codon. An ORF comprising a stop codon sometimes is translated in a system comprising a suppressor tRNA that incorporates an unnatural amino acid during translation of the target protein or target peptide. Methods for incorporating unnatural amino acids into a target protein or peptide are known, which include, for example, processes utilizing a heterologous tRNA/synthetase pair, where the tRNA recognizes an amber stop codon and is loaded with an unnatural amino acid (e.g., World Wide Web URL iupac.org/news/prize/2003/wang.pdf).

Depending on the portion of a nucleic acid reagent (e.g., Promoter, 5′ or 3′ UTR, ORI, ORF, and the like) chosen for alteration (e.g., by mutagenesis, introduction or deletion, for example) the modifications described above can alter a given activity by (i) increasing or decreasing feedback inhibition mechanisms, (ii) increasing or decreasing promoter initiation, (iii) increasing or decreasing translation initiation, (iv) increasing or decreasing translational efficiency, (v) modifying localization of peptides or products expressed from nucleic acid reagents described herein, or (vi) increasing or decreasing the copy number of a nucleotide sequence of interest, (vii) expression of an anti-sense RNA, RNAi, siRNA, ribozyme and the like. In some embodiments, alteration of a nucleic acid reagent or nucleotide sequence can alter a region involved in feedback inhibition (e.g., 5′ UTR, promoter and the like). A modification sometimes is made that can add or enhance binding of a feedback regulator and sometimes a modification is made that can reduce, inhibit or eliminate binding of a feedback regulator.

In certain embodiments, alteration of a nucleic acid reagent or nucleotide sequence can alter sequences involved in transcription initiation (e.g., promoters, 5′ UTR, and the like). A modification sometimes can be made that can enhance or increase initiation from an endogenous or heterologous promoter element. A modification sometimes can be made that removes or disrupts sequences that increase or enhance transcription initiation, resulting in a decrease or elimination of transcription from an endogenous or heterologous promoter element.

In some embodiments, alteration of a nucleic acid reagent or nucleotide sequence can alter sequences involved in translational initiation or translational efficiency (e.g., 5′ UTR, 3′ UTR, codon triplets of higher or lower abundance, translational terminator sequences and the like, for example). A modification sometimes can be made that can increase or decrease translational initiation, modifying a ribosome binding site for example. A modification sometimes can be made that can increase or decrease translational efficiency. Removing or adding sequences that form hairpins and changing codon triplets to a more or less preferred codon are non-limiting examples of genetic modifications that can be made to alter translation initiation and translation efficiency.

In certain embodiments, alteration of a nucleic acid reagent or nucleotide sequence can alter sequences involved in localization of peptides, proteins or other desired products (e.g., 3-HP, for example). A modification sometimes can be made that can alter, add or remove sequences responsible for targeting a polypeptide, protein or product to an intracellular organelle, the periplasm, cellular membranes, or extracellularly. Transport of a heterologous product to a different intracellular space or extracellularly sometimes can reduce or eliminate the formation of inclusion bodies (e.g., insoluble aggregates of the desired product).

In some embodiments, alteration of a nucleic acid reagent or nucleotide sequence can alter sequences involved in increasing or decreasing the copy number of a nucleotide sequence of interest. A modification sometimes can be made that increases or decreases the number of copies of an ORF stably integrated into the genome of an organism or on an epigenetic nucleic acid reagent. Non-limiting examples of alterations that can increase the number of copies of a sequence of interest include, adding copies of the sequence of interest by duplication of regions in the genome (e.g., adding additional copies by recombination or by causing gene amplification of the host genome, for example), cloning additional copies of a sequence onto a nucleic acid reagent, or altering an ORI to increase the number of copies of an epigenetic nucleic acid reagent. Non-limiting examples of alterations that can decrease the number of copies of a sequence of interest include, removing copies of the sequence of interest by deletion or disruption of regions in the genome, removing additional copies of the sequence from epigenetic nucleic acid reagents, or altering an ORI to decrease the number of copies of an epigenetic nucleic acid reagent.

In certain embodiments, increasing or decreasing the expression of a nucleotide sequence of interest can also be accomplished by altering, adding or removing sequences involved in the expression of an anti-sense RNA, RNAi, siRNA, ribozyme and the like. The methods described above can be used to modify expression of anti-sense RNA, RNAi, siRNA, ribozyme and the like.

Engineered microorganisms can be prepared by altering, introducing or removing nucleotide sequences in the host genome or in stably maintained epigenetic nucleic acid reagents, as noted above. The nucleic acid reagents use to alter, introduce or remove nucleotide sequences in the host genome or epigenetic nucleic acids can be prepared using the methods described herein or available to the artisan.

Nucleic acid sequences having a desired activity can be isolated from cells of a suitable organism using lysis and nucleic acid purification procedures described in a known reference manual (e.g., Maniatis, T., E. F. Fritsch and J. Sambrook (1982) Molecular Cloning: a Laboratory Manual; Cold Spring Harbor Laboratory, Cold Spring Harbor, N.Y.) or using commercially available cell lysis and DNA purification reagents and kits. In some embodiments, nucleic acids used to engineer microorganisms can be provided for conducting methods described herein after processing of the organism containing the nucleic acid. For example, the nucleic acid of interest may be extracted, isolated, purified or amplified from a sample (e.g., from an organism of interest or culture containing a plurality of organisms of interest, like yeast or bacteria for example). The term “isolated” as used herein refers to nucleic acid removed from its original environment (e.g., the natural environment if it is naturally occurring, or a host cell if expressed exogenously), and thus is altered “by the hand of man” from its original environment. An isolated nucleic acid generally is provided with fewer non-nucleic acid components (e.g., protein, lipid) than the amount of components present in a source sample. A composition comprising isolated sample nucleic acid can be substantially isolated (e.g., about 90%, 91%, 92%, 93%, 94%, 95%, 96%, 97%, 98%, 99% or greater than 99% free of non-nucleic acid components). The term “purified” as used herein refers to sample nucleic acid provided that contains fewer nucleic acid species than in the sample source from which the sample nucleic acid is derived. A composition comprising sample nucleic acid may be substantially purified (e.g., about 90%, 91%, 92%, 93%, 94%, 95%, 96%, 97%, 98%, 99% or greater than 99% free of other nucleic acid species). The term “amplified” as used herein refers to subjecting nucleic acid of a cell, organism or sample to a process that linearly or exponentially generates amplicon nucleic acids having the same or substantially the same nucleotide sequence as the nucleotide sequence of the nucleic acid in the sample, or portion thereof. As noted above, the nucleic acids used to prepare nucleic acid reagents as described herein can be subjected to fragmentation or cleavage.

Amplification of nucleic acids is sometimes necessary when dealing with organisms that are difficult to culture. Where amplification may be desired, any suitable amplification technique can be utilized. Non-limiting examples of methods for amplification of polynucleotides include, polymerase chain reaction (PCR); ligation amplification (or ligase chain reaction (LCR)); amplification methods based on the use of Q-beta replicase or template-dependent polymerase (see US Patent Publication Number US20050287592); helicase-dependent isothermal amplification (Vincent et al., “Helicase-dependent isothermal DNA amplification”. EMBO reports 5 (8): 795-800 (2004)); strand displacement amplification (SDA); thermophilic SDA nucleic acid sequence based amplification (3 SR or NASBA) and transcription-associated amplification (TAA). Non-limiting examples of PCR amplification methods include standard PCR, AFLP-PCR, Allele-specific PCR, Alu-PCR, Asymmetric PCR, Colony PCR, Hot start PCR, Inverse PCR (IPCR), In situ PCR (ISH), Intersequence-specific PCR (ISSR-PCR), Long PCR, Multiplex PCR, Nested PCR, Quantitative PCR, Reverse Transcriptase PCR (RT-PCR), Real Time PCR, Single cell PCR, Solid phase PCR, combinations thereof, and the like. Reagents and hardware for conducting PCR are commercially available.

Protocols for conducting the various types of PCR listed above are readily available to the artisan. PCR conditions can be dependent upon primer sequences, target abundance, and the desired amount of amplification, and therefore, one of skill in the art may choose from a number of PCR protocols available (see, e.g., U.S. Pat. Nos. 4,683,195 and 4,683,202; and PCR Protocols: A Guide to Methods and Applications, Innis et al., eds., 1990. PCR often is carried out as an automated process with a thermostable enzyme. In this process, the temperature of the reaction mixture is cycled through a denaturing region, a primer-annealing region, and an extension reaction region automatically. Machines specifically adapted for this purpose are commercially available. A non-limiting example of a PCR protocol that may be suitable for embodiments described herein is, treating the sample at 95° C. for 5 minutes; repeating forty-five cycles of 95° C. for 1 minute, 59° C. for 1 minute, 10 seconds, and 72° C. for 1 minute 30 seconds; and then treating the sample at 72° C. for 5 minutes. Additional PCR protocols are described in the example section. Multiple cycles frequently are performed using a commercially available thermal cycler. Suitable isothermal amplification processes known and selected by the person of ordinary skill in the art also may be applied, in certain embodiments. In some embodiments, nucleic acids encoding polypeptides with a desired activity can be isolated by amplifying the desired sequence from an organism having the desired activity using oligonucleotides or primers designed based on sequences described herein.

Amplified, isolated and/or purified nucleic acids can be cloned into the recombinant DNA vectors described herein or into suitable commercially available recombinant DNA vectors. Cloning of nucleic acid sequences of interest into recombinant DNA vectors can facilitate further manipulations of the nucleic acids for preparation of nucleic acid reagents, (e.g., alteration of nucleotide sequences by mutagenesis, homologous recombination, amplification and the like, for example). Standard cloning procedures (e.g., enzymic digestion, ligation, and the like) are known (e.g., described in Maniatis, T., E. F. Fritsch and J. Sambrook (1982) Molecular Cloning: a Laboratory Manual; Cold Spring Harbor Laboratory, Cold Spring Harbor, N.Y.).

In some embodiments, nucleic acid sequences prepared by isolation or amplification can be used, without any further modification, to add an activity to a microorganism and thereby create a genetically modified or engineered microorganism. In certain embodiments, nucleic acid sequences prepared by isolation or amplification can be genetically modified to alter (e.g., increase or decrease, for example) a desired activity. In some embodiments, nucleic acids, used to add an activity to an organism, sometimes are genetically modified to optimize the heterologous polynucleotide sequence encoding the desired activity (e.g., polypeptide or protein, for example). The term “optimize” as used herein can refer to alteration to increase or enhance expression by preferred codon usage. The term optimize can also refer to modifications to the amino acid sequence to increase the activity of a polypeptide or protein, such that the activity exhibits a higher catalytic activity as compared to the “natural” version of the polypeptide or protein.

Nucleic acid sequences of interest can be genetically modified using methods known in the art. Mutagenesis techniques are particularly useful for small scale (e.g., 1, 2, 5, 10 or more nucleotides) or large scale (e.g., 50, 100, 150, 200, 500, or more nucleotides) genetic modification. Mutagenesis allows the artisan to alter the genetic information of an organism in a stable manner, either naturally (e.g., isolation using selection and screening) or experimentally by the use of chemicals, radiation or inaccurate DNA replication (e.g., PCR mutagenesis). In some embodiments, genetic modification can be performed by whole scale synthetic synthesis of nucleic acids, using a native nucleotide sequence as the reference sequence, and modifying nucleotides that can result in the desired alteration of activity. Mutagenesis methods sometimes are specific or targeted to specific regions or nucleotides (e.g., site-directed mutagenesis, PCR-based site-directed mutagenesis, and in vitro mutagenesis techniques such as transplacement and in vivo oligonucleotide site-directed mutagenesis, for example). Mutagenesis methods sometimes are non-specific or random with respect to the placement of genetic modifications (e.g., chemical mutagenesis, insertion element (e.g., insertion or transposon elements) and inaccurate PCR based methods, for example).

Site directed mutagenesis is a procedure in which a specific nucleotide or specific nucleotides in a DNA molecule are mutated or altered. Site directed mutagenesis typically is performed using a nucleic acid sequence of interest cloned into a circular plasmid vector. Site-directed mutagenesis requires that the wild type sequence be known and used a platform for the genetic alteration. Site-directed mutagenesis sometimes is referred to as oligonucleotide-directed mutagenesis because the technique can be performed using oligonucleotides which have the desired genetic modification incorporated into the complement a nucleotide sequence of interest. The wild type sequence and the altered nucleotide are allowed to hybridize and the hybridized nucleic acids are extended and replicated using a DNA polymerase. The double stranded nucleic acids are introduced into a host (e.g., E. coli, for example) and further rounds of replication are carried out in vivo. The transformed cells carrying the mutated nucleic acid sequence are then selected and/or screened for those cells carrying the correctly mutagenized sequence. Cassette mutagenesis and PCR-based site-directed mutagenesis are further modifications of the site-directed mutagenesis technique. Site-directed mutagenesis can also be performed in vivo (e.g., transplacement “pop-in pop-out”, in vivo site-directed mutagenesis with synthetic oligonucleotides and the like, for example).

PCR-based mutagenesis can be performed using PCR with oligonucleotide primers that contain the desired mutation or mutations. The technique functions in a manner similar to standard site-directed mutagenesis, with the exception that a thermocycler and PCR conditions are used to replace replication and selection of the clones in a microorganism host. As PCR-based mutagenesis also uses a circular plasmid vector, the amplified fragment (e.g., linear nucleic acid molecule) containing the incorporated genetic modifications can be separated from the plasmid containing the template sequence after a sufficient number of rounds of thermocycler amplification, using standard electrophorectic procedures. A modification of this method uses linear amplification methods and a pair of mutagenic primers that amplify the entire plasmid. The procedure takes advantage of the E. coli Dam methylase system which causes DNA replicated in vivo to be sensitive to the restriction endonucleases DpnI. PCR synthesized DNA is not methylated and is therefore resistant to DpnI. This approach allows the template plasmid to be digested, leaving the genetically modified, PCR synthesized plasmids to be isolated and transformed into a host bacteria for DNA repair and replication, thereby facilitating subsequent cloning and identification steps. A certain amount of randomness can be added to PCR-based sited directed mutagenesis by using partially degenerate primers.

Recombination sometimes can be used as a tool for mutagenesis. Homologous recombination allows the artisan to specifically target regions of known sequence for insertion of heterologous nucleotide sequences using the host organisms natural DNA replication and repair enzymes. Homologous recombination methods sometimes are referred to as “pop in pop out” mutagenesis, transplacement, knock out mutagenesis or knock in mutagenesis. Integration of a nucleic acid sequence into a host genome is a single cross over event, which inserts the entire nucleic acid reagent (e.g., pop in). A second cross over event excises all but a portion of the nucleic acid reagent, leaving behind a heterologous sequence, often referred to as a “footprint” (e.g., pop out). Mutagenesis by insertion (e.g., knock in) or by double recombination leaving behind a disrupting heterologous nucleic acid (e.g., knock out) both server to disrupt or “knock out” the function of the gene or nucleic acid sequence in which insertion occurs. By combining selectable markers and/or auxotrophic markers with nucleic acid reagents designed to provide the appropriate nucleic acid target sequences, the artisan can target a selectable nucleic acid reagent to a specific region, and then select for recombination events that “pop out” a portion of the inserted (e.g., “pop in”) nucleic acid reagent.

Such methods take advantage of nucleic acid reagents that have been specifically designed with known target nucleic acid sequences at or near a nucleic acid or genomic region of interest. Popping out typically leaves a “foot print” of left over sequences that remain after the recombination event. The left over sequence can disrupt a gene and thereby reduce or eliminate expression of that gene. In some embodiments, the method can be used to insert sequences, upstream or downstream of genes that can result in an enhancement or reduction in expression of the gene. In certain embodiments, new genes can be introduced into the genome of a host organism using similar recombination or “pop in” methods. An example of a yeast recombination system using the ura3 gene and 5-FOA were described briefly above and further detail is presented herein.

A method for modification is described in Alani et al., “A method for gene disruption that allows repeated use of URA3 selection in the construction of multiply disrupted yeast strains”, Genetics 116(4):541-545 August 1987. The original method uses a URA3 cassette with 1000 base pairs (bp) of the same nucleotide sequence cloned in the same orientation on either side of the URA3 cassette. Targeting sequences of about 50 bp are added to each side of the construct. The double stranded targeting sequences are complementary to sequences in the genome of the host organism. The targeting sequences allow site-specific recombination in a region of interest. The modification of the original technique replaces the two 1000 bp sequence direct repeats with two 200 bp direct repeats. The modified method also uses 50 bp targeting sequences. The modification reduces or eliminates recombination of a second knock out into the 1000 bp repeat left behind in a first mutagenesis, therefore allowing multiply knocked out yeast. Additionally, the 200 bp sequences used herein are uniquely designed, self-assembling sequences that leave behind identifiable footprints. The technique used to design the sequences incorporate design features such as low identity to the yeast genome, and low identity to each other. Therefore a library of the self-assembling sequences can be generated to allow multiple knockouts in the same organism, while reducing or eliminating the potential for integration into a previous knockout.

As noted above, the URA3 cassette makes use of the toxicity of 5-FOA in yeast carrying a functional URA3 gene. Uracil synthesis deficient yeast strains can be transformed with the modified URA3 cassette, using standard yeast transformation protocols, and the transformed cells are plated on minimal media minus uracil. In some embodiments, PCR can be used to verify correct insertion into the region of interest in the host genome, and certain embodiments the PCR step can be omitted. Inclusion of the PCR step can reduce the number of transformants that need to be counter selected to “pop out” the URA3 cassette. The transformants (e.g., all or the ones determined to be correct by PCR, for example) can then be counter-selected on media containing 5-FOA, which will select for recombination out (e.g., popping out) of the URA3 cassette, thus rendering the yeast ura3 deficient again, and resistant to 5-FOA toxicity. Targeting sequences used to direct recombination events to specific regions are presented herein. A modification of the method described above can be used to integrate genes in to the chromosome, where after recombination a functional gene is left in the chromosome next to the 200 bp footprint.

In some embodiments, other auxotrophic or dominant selection markers can be used in place of URA3 (e.g., an auxotrophic selectable marker), with the appropriate change in selection media and selection agents. Auxotrophic selectable markers are used in strains deficient for synthesis of a required biological molecule (e.g., amino acid or nucleoside, for example). Non-limiting examples of additional auxotrophic markers include; HIS3, TRP1, LEU2, LEU2-d, and LYS2. Certain auxotrophic markers (e.g., URA3 and LYS2) allow counter selection to select for the second recombination event that pops out all but one of the direct repeats of the recombination construct. HIS3 encodes an activity involved in histidine synthesis. TRP1 encodes an activity involved in tryptophan synthesis. LEU2 encodes an activity involved in leucine synthesis. LEU2-d is a low expression version of LEU2 that selects for increased copy number (e.g., gene or plasmid copy number, for example) to allow survival on minimal media without leucine. LYS2 encodes an activity involved in lysine synthesis, and allows counter selection for recombination out of the LYS2 gene using alpha-amino adipate (α-amino adipate).

Dominant selectable markers can be useful because they also allow industrial and/or prototrophic strains to be used for genetic manipulations. Additionally, dominant selectable markers provide the advantage that rich medium can be used for plating and culture growth, and thus growth rates are markedly increased. Non-limiting examples of dominant selectable markers include; Tn903 kan^{r}, Cm^{r}, Hyg^{r}, CUP1, and DHFR. Tn903 kan^{r} encodes an activity involved in kanamycin antibiotic resistance (e.g., typically neomycin phosphotransferase II or NPTII, for example). Cm^{r} encodes an activity involved in chloramphenicol antibiotic resistance (e.g., typically chloramphenicol acetyl transferase or CAT, for example). Hyg^{r} encodes an activity involved in hygromycin resistance by phosphorylation of hygromycin B (e.g., hygromycin phosphotransferase, or HPT). CUP1 encodes an activity involved in resistance to heavy metal (e.g., copper, for example) toxicity. DHFR encodes a dihydrofolate reductase activity which confers resistance to methotrexate and sulfanilamde compounds.

In contrast to site-directed or specific mutagenesis, random mutagenesis does not require any sequence information and can be accomplished by a number of widely different methods. Random mutagenesis often is used to create mutant libraries that can be used to screen for the desired genotype or phenotype. Non-limiting examples of random mutagenesis include; chemical mutagenesis, UV-induced mutagenesis, insertion element or transposon-mediated mutagenesis, DNA shuffling, error-prone PCR mutagenesis, and the like.

Chemical mutagenesis often involves chemicals like ethyl methanesulfonate (EMS), nitrous acid, mitomycin C, N-methyl-N-nitrosourea (MNU), diepoxybutane (DEB), 1, 2, 7, 8-diepoxyoctane (DEO), methyl methane sulfonate (MMS), N-methyl-N′-nitro-N-nitrosoguanidine (MNNG), 4-nitroquinoline 1-oxide (4-NQO), 2-methyloxy-6-chloro-9(3-[ethyl-2-chloroethyl]-aminopropylamino)-acridinedihydrochloride (ICR-170), 2-amino purine (2AP), and hydroxylamine (HA), provided herein as non-limiting examples. These chemicals can cause base-pair substitutions, frameshift mutations, deletions, transversion mutations, transition mutations, incorrect replication, and the like. In some embodiments, the mutagenesis can be carried out in vivo. Sometimes the mutagenic process involves the use of the host organisms DNA replication and repair mechanisms to incorporate and replicate the mutagenized base or bases.

Another type of chemical mutagenesis involves the use of base-analogs. The use of base-analogs cause incorrect base pairing which in the following round of replication is corrected to a mismatched nucleotide when compared to the starting sequence. Base analog mutagenesis introduces a small amount of non-randomness to random mutagenesis, because specific base analogs can be chose which can be incorporated at certain nucleotides in the starting sequence. Correction of the mispairing typically yields a known substitution. For example, Bromo-deoxyuridine (BrdU) can be incorporated into DNA and replaces T in the sequence. The host DNA repair and replication machinery can sometime correct the defect, but sometimes will mispair the BrdU with a G. The next round of replication then causes a G-C transversion from the original A-T in the native sequence.

Ultra violet (UV) induced mutagenesis is caused by the formation of thymidine dimers when UV light irradiates chemical bonds between two adjacent thymine residues. Excision repair mechanism of the host organism correct the lesion in the DNA, but occasionally the lesion is incorrectly repaired typically resulting in a C to T transition.

Insertion element or transposon-mediated mutagenesis makes use of naturally occurring or modified naturally occurring mobile genetic elements. Transposons often encode accessory activities in addition to the activities necessary for transposition (e.g., movement using a transposase activity, for example). In many examples, transposon accessory activities are antibiotic resistance markers (e.g., see Tn903 kan^{r} described above, for example). Insertion elements typically only encode the activities necessary for movement of the nucleic acid sequence. Insertion element and transposon mediated mutagenesis often can occur randomly, however specific target sequences are known for some transposons. Mobile genetic elements like IS elements or Transposons (Tn) often have inverted repeats, direct repeats or both inverted and direct repeats flanking the region coding for the transposition genes. Recombination events catalyzed by the transposase cause the element to remove itself from the genome and move to a new location, leaving behind a portion of an inverted or direct repeat. Classic examples of transposons are the “mobile genetic elements” discovered in maize. Transposon mutagenesis kits are commercially available which are designed to leave behind a 5 codon insert (e.g., Mutation Generation System kit, Finnzymes, World Wide Web URL finnzymes.us, for example). This allows the artisan to identify the insertion site, without fully disrupting the function of most genes.

DNA shuffling is a method which uses DNA fragments from members of a mutant library and reshuffles the fragments randomly to generate new mutant sequence combinations. The fragments are typically generated using DNaseI, followed by random annealing and re-joining using self-priming PCR. The DNA overhanging ends, from annealing of random fragments, provide “primer” sequences for the PCR process. Shuffling can be applied to libraries generated by any of the above mutagenesis methods.

Error prone PCR and its derivative rolling circle error prone PCR uses increased magnesium and manganese concentrations in conjunction with limiting amounts of one or two nucleotides to reduce the fidelity of the Taq polymerase. The error rate can be as high as 2% under appropriate conditions, when the resultant mutant sequence is compared to the wild type starting sequence. After amplification, the library of mutant coding sequences must be cloned into a suitable plasmid. Although point mutations are the most common types of mutation in error prone PCR, deletions and frameshift mutations are also possible. There are a number of commercial error-prone PCR kits available, including those from Stratagene and Clontech (e.g., World Wide Web URL strategene.com and World Wide Web URL clontech.com, respectively, for example). Rolling circle error-prone PCR is a variant of error-prone PCR in which wild-type sequence is first cloned into a plasmid and then the whole plasmid is amplified under error-prone conditions.

As noted above, organisms with altered activities can also be isolated using genetic selection and screening of organisms challenged on selective media or by identifying naturally occurring variants from unique environments. For example, 2-Deoxy-D-glucose is a toxic glucose analog. Growth of yeast on this substance yields mutants that are glucose-deregulated. A number of mutants have been isolated using 2-Deoxy-D-glucose including transport mutants, and mutants that ferment glucose and galactose simultaneously instead of glucose first then galactose when glucose is depleted. Similar techniques have been used to isolate mutant microorganisms that can metabolize plastics (e.g., from landfills), petrochemicals (e.g., from oil spills), and the like, either in a laboratory setting or from unique environments.

Similar methods can be used to isolate naturally occurring mutations in a desired activity when the activity exists at a relatively low or nearly undetectable level in the organism of choice, in some embodiments. The method generally consists of growing the organism to a specific density in liquid culture, concentrating the cells, and plating the cells on various concentrations of the substance to which an increase in metabolic activity is desired. The cells are incubated at a moderate growth temperature, for 5 to 10 days. To enhance the selection process, the plates can be stored for another 5 to 10 days at a low temperature. The low temperature sometimes can allow strains that have gained or increased an activity to continue growing while other strains are inhibited for growth at the low temperature. Following the initial selection and secondary growth at low temperature, the plates can be replica plated on higher or lower concentrations of the selection substance to further select for the desired activity.

A native, heterologous or mutagenized polynucleotide can be introduced into a nucleic acid reagent for introduction into a host organism, thereby generating an engineered microorganism. Standard recombinant DNA techniques (restriction enzyme digests, ligation, and the like) can be used by the artisan to combine the mutagenized nucleic acid of interest into a suitable nucleic acid reagent capable of (i) being stably maintained by selection in the host organism, or (ii) being integrating into the genome of the host organism. As noted above, sometimes nucleic acid reagents comprise two replication origins to allow the same nucleic acid reagent to be manipulated in bacterial before final introduction of the final product into the host organism (e.g., yeast or fungus, for example). Standard molecular biology and recombinant DNA methods are known (e.g., described in Maniatis, T., E. F. Fritsch and J. Sambrook (1982) Molecular Cloning: a Laboratory Manual; Cold Spring Harbor Laboratory, Cold Spring Harbor, N.Y.).

Nucleic acid reagents can be introduced into microorganisms using various techniques. Non-limiting examples of methods used to introduce heterologous nucleic acids into various organisms include; transformation, transfection, transduction, electroporation, ultrasound-mediated transformation, particle bombardment and the like. In some instances the addition of carrier molecules (e.g., bis-benzimdazolyl compounds, for example, see U.S. Pat. No. 5,595,899) can increase the uptake of DNA in cells typically though to be difficult to transform by conventional methods. Conventional methods of transformation are known (e.g., described in Maniatis, T., E. F. Fritsch and J. Sambrook (1982) Molecular Cloning: a Laboratory Manual; Cold Spring Harbor Laboratory, Cold Spring Harbor, N.Y.).

### Culture, Production and Process Methods

Engineered microorganisms often are cultured under conditions that optimize the yield of 3-HP. In general, non-limiting examples of conditions that may be optimized include the type and amount of carbon source, the type and amount of nitrogen source, the carbon-to-nitrogen ratio, the oxygen level, growth temperature, pH, length of the biomass production phase, length of 3-HP accumulation phase, and time of cell harvest.

Culture media generally contain a suitable carbon source. Carbon sources useful for culturing microorganisms and/or fermentation processes sometimes are referred to as feedstocks. The term “feedstock” as used herein refers to a composition containing a carbon source that is provided to an organism, which is used by the organism to produce energy and metabolic products useful for growth. A feedstock (also referred to herein as a “substrate” or as a “carbon source”) can be a natural substance, a “man-made” (e.g., synthetic) substance, a purified or isolated substance, a mixture of purified substances, a mixture of unpurified substances or combinations thereof. A feedstock often is prepared by and/or provided to an organism by a person, and a feedstock often is formulated prior to administration to the organism. For the production of 3-HP, a carbon source can include, but are not limited to, odd chain alkanes, odd chain fatty acids/esters, or mixtures thereof in the presence or absence of other substances including, but not limited to, one or more of the following: even chain alkanes, alkenes, alkynes, each of which may be linear, branched, saturated, unsaturated, substituted or combinations thereof; linear or branched alcohols or aldehydes; linear (e.g., even chain) or branched fatty acids (e.g., about 6 carbons to about 60 carbons, including free fatty acids, soap stock, for example); esters of fatty acids; monoglycerides; diglycerides; triglycerides, phospholipids, mono-carboxylic acids, di-carboxylic acids, polycarboxylic acids, monosaccharides (e.g., also referred to as “saccharides,” which include 6-carbon sugars (e.g., glucose, fructose), 5-carbon sugars (e.g., xylose and other pentoses) and the like), disaccharides (e.g., lactose, sucrose), oligosaccharides (e.g., glycans, homopolymers of a monosaccharide), polysaccharides (e.g., starch, cellulose, heteropolymers of monosaccharides or mixtures thereof) and sugar alcohols (e.g., glycerol).

Carbon sources also can be selected from one or more of the following non-limiting examples: for example, for sources of odd chain alkanes, any suitable animal, microorganism, plant, including higher plant, plant oil, kerosene, diesel oil, fuel oil, gasoline, petrochemicals, petroleum jelly, paraffin wax, paraffin oil, paraffins (e.g., saturated paraffin, unsaturated paraffin, substituted paraffin, linear paraffin, branched paraffin, or combinations thereof); motor oil, asphalt, chemically synthesized alkane, alkane hydrocarbons produced by fermentation of a microorganism, or the like can be used as a feedstock. Non-limiting commercial sources of carbon feedstocks include renewable feedstocks (e.g., cheese whey permeate, cornsteep liquor, sugar beet molasses, barley malt), plants or plant products (e.g., vegetable oils (e.g., almond oil, canola oil, cocoa butter, coconut oil, corn oil, cottonseed oil, flaxseed oil, grape seed oil, illipe, olive oil, palm oil, palm kernel oil, safflower oil, peanut oil, soybean oil, sesame oil, shea nut oil, sunflower oil walnut oil, the like and combinations thereof) and animal fats (e.g., beef tallow, butterfat, lard, cod liver oil).

A carbon source also may include a metabolic product that can be used directly as a metabolic substrate in an engineered pathway described herein, or indirectly via conversion to a different molecule using engineered or native biosynthetic pathways in an engineered microorganism. In certain embodiments, metabolic pathways can be preferentially biased towards production of a desired product by increasing the levels of one or more activities in one or more metabolic pathways having and/or generating at least one common metabolic and/or synthetic substrate. In some embodiments, a metabolic byproduct (e.g., fatty acid) of an engineered activity (e.g., ω-oxidation activity) can be used in one or more metabolic pathways selected from gluconeogenesis, pentose phosphate pathway, glycolysis, fatty acid synthesis, β-oxidation, and omega oxidation, to generate a carbon source that can be converted to 3-HP.

In some embodiments, a feedstock includes a mixture of carbon sources, where each carbon source in the feedstock is selected based on the genotype of the engineered microorganism. In certain embodiments, a mixed carbon source feedstock includes one or more carbon sources selected from sugars, cellulose, alkanes, fatty acids, triacylglycerides, paraffins, the like and combinations thereof.

Nitrogen may be supplied from an inorganic (e.g., (NH₄)₂SO₄) or organic source (e.g., urea or glutamate). In addition to appropriate carbon and nitrogen sources, culture media also can contain suitable minerals, salts, cofactors, buffers, vitamins, metal ions (e.g., Mn⁺², Co⁺², Zn⁺², Mg⁺²) and other components suitable for culture of microorganisms.

Engineered microorganisms sometimes are cultured in complex media (e.g., yeast extract-peptone-dextrose broth (YPD)). In some embodiments, engineered microorganisms are cultured in a defined minimal media that lacks a component necessary for growth and thereby forces selection of a desired expression cassette (e.g., Yeast Nitrogen Base (DIFCO Laboratories, Detroit, Mich.)). Culture media in some embodiments are common commercially prepared media, such as Yeast Nitrogen Base (DIFCO Laboratories, Detroit, Mich.). Other defined or synthetic growth media may also be used and the appropriate medium for growth of the particular microorganism is known. A variety of host organisms can be selected for the production of engineered microorganisms. Non-limiting examples include yeast (e.g., Candida (e.g., ATCC20336, ATCC20913, ATCC20962), Yarrowia lipolytica (e.g., ATCC20228)) and filamentous fungi (e.g., Aspergillus nidulans (e.g., ATCC38164) and Aspergillus parasiticus (e.g., ATCC 24690)). In specific embodiments, yeast strains are cultured in YPD media (10 g/L Bacto Yeast Extract, 20 g/L Bacto Peptone, and 20 g/L Dextrose). Filamentous fungi, in particular embodiments, are grown in CM (Complete Medium) containing 10 g/L Dextrose, 2 g/L Bacto Peptone, 1 g/L Bacto Yeast Extract, 1 g/L Casamino acids, 50 mL/L 20× Nitrate Salts (120 g/L NaNO₃, 10.4 g/L KCl, 10.4 g/L MgSO₄.7 H₂O), 1 mL/L 1000× Trace Elements (22 g/L ZnSO₄.7 H₂O, 11 g/L H₃BO₃, 5 g/L MnCl₂.7 H₂O, 5 g/L FeSO₄.7 H₂O, 1.7 g/L CoCl₂.6 H₂O, 1.6 g/L CuSO₄.5 H₂O, 1.5 g/L Na₂ MoO₄.2 H₂O, and 50 g/L Na₄EDTA), and 1 mL/L Vitamin Solution (100 mg each of Biotin, pyridoxine, thiamine, riboflavin, p-aminobenzoic acid, and nicotinic acid in 100 mL water).

A suitable pH range for the fermentation often is between about pH 2.0 to about pH 9.0, where a pH in the range of about pH 6.0 to about pH 9.0 sometimes is utilized for initial culture conditions. Depending on the host organism, culturing may be conducted under aerobic or anaerobic conditions, where microaerobic conditions sometimes are maintained. A two-stage process may be utilized, where one stage promotes microorganism proliferation and another state promotes production of target molecule. In a two-stage process, the first stage may be conducted under aerobic conditions (e.g., introduction of air and/or oxygen) and the second stage may be conducted under anaerobic conditions (e.g., air or oxygen are not introduced to the culture conditions). In some embodiments, the first stage may be conducted under anaerobic conditions and the second stage may be conducted under aerobic conditions. In certain embodiments, a two-stage process may include two more organisms, where one organism generates an intermediate in one stage and another organism processes the intermediate product into a target product (e.g., 3-HP) in another stage, for example.

A variety of fermentation processes may be applied for commercial biological production of a target product. In some embodiments, commercial production of a target product from a recombinant microbial host is conducted using a batch, fed-batch or continuous fermentation process, for example.

A batch fermentation process often is a closed system where the media composition is fixed at the beginning of the process and not subject to further additions beyond those required for maintenance of pH and oxygen level during the process. At the beginning of the culturing process the media is inoculated with the desired organism and growth or metabolic activity is permitted to occur without adding additional sources (i.e., carbon and nitrogen sources) to the medium. In batch processes the metabolite and biomass compositions of the system change constantly up to the time the culture is terminated. In a typical batch process, cells proceed through a static lag phase to a high-growth log phase and finally to a stationary phase, wherein the growth rate is diminished or halted. Left untreated, cells in the stationary phase will eventually die.

A variation of the standard batch process is the fed-batch process, where the carbon source is continually added to the fermenter over the course of the fermentation process. Fed-batch processes are useful when catabolite repression is apt to inhibit the metabolism of the cells or where it is desirable to have limited amounts of carbon source in the media at any one time. Measurement of the carbon source concentration in fed-batch systems may be estimated on the basis of the changes of measurable factors such as pH, dissolved oxygen and the partial pressure of waste gases (e.g., CO₂).

Batch and fed-batch culturing methods are known in the art. Examples of such methods may be found in Thomas D. Brock in Biotechnology: A Textbook of Industrial Microbiology, 2.sup.nd ed., (1989) Sinauer Associates Sunderland, Mass. and Deshpande, Mukund V., Appl. Biochem. Biotechnol., 36:227 (1992).

In continuous fermentation process a defined media often is continuously added to a bioreactor while an equal amount of culture volume is removed simultaneously for product recovery. Continuous cultures generally maintain cells in the log phase of growth at a constant cell density. Continuous or semi-continuous culture methods permit the modulation of one factor or any number of factors that affect cell growth or end product concentration. For example, an approach may limit the carbon source and allow all other parameters to moderate metabolism. In some systems, a number of factors affecting growth may be altered continuously while the cell concentration, measured by media turbidity, is kept constant. Continuous systems often maintain steady state growth and thus the cell growth rate often is balanced against cell loss due to media being drawn off the culture. Methods of modulating nutrients and growth factors for continuous culture processes, as well as techniques for maximizing the rate of product formation, are known and a variety of methods are detailed by Brock, supra.

In some embodiments involving fermentation, the fermentation can be carried out using two or more microorganisms (e.g., host microorganism, engineered microorganism, isolated naturally occurring microorganism, the like and combinations thereof), where a feedstock is partially or completely utilized by one or more organisms in the fermentation (e.g., mixed fermentation), and the products of cellular respiration or metabolism of one or more organisms can be further metabolized by one or more other organisms to produce a desired target product (e.g., 3-HP). In certain embodiments, each organism can be fermented independently and the products of cellular respiration or metabolism purified and contacted with another organism to produce a desired target product. In some embodiments, one or more organisms are partially or completely blocked in a metabolic pathway (e.g., β-oxidation, ω-oxidation, the like or combinations thereof), thereby producing a desired product that can be used as a feedstock for one or more other organisms. Any suitable combination of microorganisms can be utilized to carry out mixed fermentation or sequential fermentation.

In various embodiments, the 3-HP produced by the genetically engineered microorganisms can be isolated or purified from the culture media or extracted from the engineered microorganisms. The terms “isolated” or “extracted” are used synonymously herein in regard to the target product generated by the engineered microorganisms (e.g., 3-HP) and refer to the target product being removed from the source (e.g., the microorganism) in which it naturally occurs. “Isolated,” as used herein, does not necessarily mean “purified.” For example, a crude lysate fraction of the microorganism can contain isolated product (e.g., 3-HP) which, in some embodiments can further be purified from the remaining contents of the lysate.

In some embodiments, fermentation of feedstocks by methods described herein can produce a target product (e.g., 3-HP) at a level of about 5% to about 100% of maximum theoretical yield (e.g., about 10%, 15%, about 20%, about 25% or more of theoretical yield (e.g., 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more of theoretical yield).

The term “theoretical yield” as used herein refers to the amount of product that could be made from a starting material if the reaction is 100% complete. For the product 3-HP, the term “theoretical yield” refers to the yield of 3-hydroxypropionic acid, 3-hydroxypropionate (salt or ester forms), or mixtures thereof in any proportion relative to one another. Theoretical yield is based on the stoichiometry of a reaction and ideal conditions in which starting material is completely consumed, undesired side reactions do not occur, the reverse reaction does not occur, and there are no losses in the work-up procedure. Culture media can be tested for target product (e.g., 3-HP) concentration and drawn off when the concentration reaches a predetermined level. Detection methods are known in the art, including but not limited to chromatographic methods (e.g., gas chromatography) or combined chromatographic/mass spectrometry (e.g., GC-MS) methods. Target product (e.g., 3-HP) may be present at a range of levels as described herein.

A target product such as 3-HP sometimes can be retained within an engineered microorganism after a culture process is completed, and in certain embodiments, the target product can be secreted out of the microorganism into the culture medium. For the latter embodiments, (i) culture media may be drawn from the culture system and fresh medium may be supplemented, and/or (ii) target product may be extracted from the culture media during or after the culture process is completed. Engineered microorganisms can be cultured on or in solid, semi-solid or liquid media. In some embodiments media is drained from cells adhering to a plate. In certain embodiments, a liquid-cell mixture is centrifuged at a speed sufficient to pellet the cells but not disrupt the cells and allow extraction of the media, as known in the art. The cells may then be resuspended in fresh media. Target product can be purified from culture media according to methods known in the art.

Provided herein are non-limiting examples of methods useful for recovering target product from fermentation broth and/or isolating/partially purifying a target product from non-target products when utilizing mixed chain length feedstocks. Recovery of 3-HP from fermentation broth can be accomplished using a variety of methods. Optionally, one can first employ a centrifugation step to separate cell mass and 3-HP from the aqueous phase. The 3-HP in the aqueous phase can then be further concentrated and purified via various chromatography, filtration and/or precipitation steps.

In certain embodiments, target product is extracted from the cultured engineered microorganisms. The microorganism cells can be concentrated by centrifugation at a speed sufficient to shear the cell membranes. In some embodiments, the cells can be physically disrupted (e.g., shear force, sonication) or chemically disrupted (e.g., contacted with detergent or other lysing agent). The phases may be separated by centrifugation or other method known in the art and target product may be isolated according to known methods.

Commercial grade target product sometimes is provided in substantially pure form (e.g., 90% pure or greater, 95% pure or greater, 99% pure or greater or 99.5% pure or greater). In some embodiments, target product may be modified into any one of a number of downstream products. 3-HP can be provided as 3-hydroxypropionic acid, an ester thereof, or a salt or other derivative thereof.

Target product can be provided within cultured microbes containing the target product (e.g., 3-HP), and cultured microbes may be supplied fresh or frozen in a liquid media or dried. Fresh or frozen microbes may be contained in appropriate moisture-proof containers that may also be temperature controlled as necessary. Target product sometimes is provided in culture medium that is substantially cell-free. In some embodiments, target product or modified target product purified from microbes is provided, and target product sometimes is provided in substantially pure form. 3-hydroxypropionic acid is an acidic viscous liquid with a pKa of 4.5, and may be transported in a variety of containers including one ton cartons, drums, and the like.

In certain embodiments, a target product (e.g., 3-HP) is produced with a yield of about 0.10 grams per gram of feedstock added, or greater; 0.20 grams of target product per gram of feedstock added, or greater; 0.30 grams of target product per gram of feedstock added, or greater; 0.40 grams of target product per gram of feedstock added, or greater; 0.50 grams of target product per gram of feedstock added, or greater; 0.55 grams of target product per gram of feedstock added, or greater; 0.56 grams of target product per gram of feedstock added, or greater; 0.57 grams of target product per gram of feedstock added, or greater; 0.58 grams of target product per gram of feedstock added, or greater; 0.59 grams of target product per gram of feedstock added, or greater; 0.60 grams of target product per gram of feedstock added, or greater; 0.61 grams of target product per gram of feedstock added, or greater; 0.62 grams of target product per gram of feedstock added, or greater; 0.63 grams of target product per gram of feedstock added, or greater; 0.64 grams of target product per gram of feedstock added, or greater; 0.65 grams of target product per gram of feedstock added, or greater; 0.66 grams of target product per gram of feedstock added, or greater; 0.67 grams of target product per gram of feedstock added, or greater; 0.68 grams of target product per gram of feedstock added, or greater; 0.69 grams of target product per gram of feedstock added, or greater; 0.70 grams of target product per gram of feedstock added or greater; 0.71 grams of target product per gram of feedstock added, or greater; 0.72 grams of target product per gram of feedstock added, or greater; 0.73 grams of target product per gram of feedstock added, or greater; 0.74 grams of target product per gram of feedstock added, or greater; 0.75 grams of target product per gram of feedstock added, or greater; 0.76 grams of target product per gram of feedstock added, or greater; 0.77 grams of target product per gram of feedstock added, or greater; 0.78 grams of target product per gram of feedstock added, or greater; 0.79 grams of target product per gram of feedstock added, or greater; 0.80 grams of target product per gram of feedstock added, or greater; 0.81 grams of target product per gram of feedstock added, or greater; 0.82 grams of target product per gram of feedstock added, or greater; 0.83 grams of target product per gram of feedstock added, or greater; 0.84 grams of target product per gram of feedstock added, or greater; 0.85 grams of target product per gram of feedstock added, or greater; 0.86 grams of target product per gram of feedstock added, or greater; 0.87 grams of target product per gram of feedstock added, or greater; 0.88 grams of target product per gram of feedstock added, or greater; 0.89 grams of target product per gram of feedstock added, or greater; 0.90 grams of target product per gram of feedstock added, or greater; 0.91 grams of target product per gram of feedstock added, or greater; 0.92 grams of target product per gram of feedstock added, or greater; 0.93 grams of target product per gram of feedstock added, or greater; 0.94 grams of target product per gram of feedstock added, or greater; 0.95 grams of target product per gram of feedstock added, or greater; 0.96 grams of target product per gram of feedstock added, or greater; 0.97 grams of target product per gram of feedstock added, or greater; 0.98 grams of target product per gram of feedstock added, or greater; 0.99 grams of target product per gram of feedstock added, or greater; 1.0 grams of target product per gram of feedstock added, or greater; 1.1 grams of target product per gram of feedstock added, or greater; 1.2 grams of target product per gram of feedstock added, or greater; 1.3 grams of target product per gram of feedstock added, or greater; 1.4 grams of target product per gram of feedstock added, or greater; or about 1.5 grams of target product per gram of feedstock added, or greater.

In certain embodiments, the 3-HP is produced with a yield of greater than about 0.15 grams per gram of the feedstock In some embodiments, the 3-HP is produced at between about 10% and about 100% of maximum theoretical yield of any introduced feedstock ((e.g., about 15%, about 20%, about 25% or more of theoretical yield (e.g., 25% or more, 26% or more, 27% or more, 28% or more, 29% or more, 30% or more, 31% or more, 32% or more, 33% or more, 34% or more, 35% or more, 36% or more, 37% or more, 38% or more, 39% or more, 40% or more, 41% or more, 42% or more, 43% or more, 44% or more, 45% or more, 46% or more, 47% or more, 48% or more, 49% or more, 50% or more, 51% or more, 52% or more, 53% or more, 54% or more, 55% or more, 56% or more, 57% or more, 58% or more, 59% or more, 60% or more, 61% or more, 62% or more, 63% or more, 64% or more, 65% or more, 66% or more, 67% or more, 68% or more, 69% or more, 70% or more, 71% or more, 72% or more, 73% or more, 74% or more, 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, or 99% or more of theoretical maximum yield).

In certain embodiments, the 3-HP is produced in a concentration range (yield or titer) of between about 0.1 g/L to about 1000 g/L of culture media (e.g., at least about 0.1 g/L, at least about 0.2 g/L, at least about 0.5 g/L, at least about 0.6 g/L, at least about 0.7 g/L, at least about 0.8 g/L, at least about 0.9 g/L, at least about 1.0 g/L, at least about 1.1 g/L, at least about 1.2 g/L, at least about 1.3 g/L, at least about 1.4 g/L, at least about 1.5 g/L, at least about 1.6 g/L, at least about 1.7 g/L, at least about 1.8 g/L, at least about 1.9 g/L, at least about 2.0 g/L, at least about 2.25 g/L, at least about 2.5 g/L, at least about 2.75 g/L, at least about 3.0 g/L, at least about 3.25 g/L, at least about 3.5 g/L, at least about 3.75 g/L, at least about 4.0 g/L, at least about 4.25 g/L, at least about 4.5 g/L, at least about 4.75 g/L, at least about 5.0 g/L, at least about 6.0 g/L, at least about 7.0 g/L, at least about 8.0 g/L, at least about 9.0 g/L, at least about 10 g/L, at least about 15 g/L, at least about 20 g/L, at least about 25 g/L, at least about 30 g/L, at least about 35 g/L, at least about 40 g/L, at least about 45 g/L, at least about 50 g/L, at least about 55 g/L, at least about 60 g/L, at least about 65 g/L, at least about 70 g/L, at least about 75 g/L, at least about 80 g/L, at least about 85 g/L, at least about 90 g/L, at least about 95 g/L, at least about 100 g/L, at least about 110 g/L, at least about 120 g/L, at least about 130 g/L, at least about 140 g/L, at least about 150 g/L, at least about 160 g/L, at least about 170 g/L, at least about 180 g/L, at least about 190 g/L, at least about 200 g/L, at least about 225 g/L, at least about 250 g/L, at least about 275 g/L, at least about 300 g/L, at least about 325 g/L, at least about 350 g/L, at least about 375 g/L, at least about 400 g/L, at least about 425 g/L, at least about 450 g/L, at least about 475 g/L, at least about 500 g/L, at least about 550 g/L, at least about 600 g/L, at least about 650 g/L, at least about 700 g/L, at least about 750 g/L, at least about 800 g/L, at least about 850 g/L, at least about 900 g/L, at least about 950 g/L, or at least about 1000 g/L

In certain, embodiments, the engineered organism comprises between about a 5-fold to about a 500-fold increase in 3-HP production when compared to wild-type or partially engineered organisms of the same strain, under identical fermentation conditions (e.g., about a 5-fold increase, about a 10-fold increase, about a 15-fold increase, about a 20-fold increase, about a 25-fold increase, about a 30-fold increase, about a 35-fold increase, about a 40-fold increase, about a 45-fold increase, about a 50-fold increase, about a 55-fold increase, about a 60-fold increase, about a 65-fold increase, about a 70-fold increase, about a 75-fold increase, about a 80-fold increase, about a 85-fold increase, about a 90-fold increase, about a 95-fold increase, about a 100-fold increase, about a 125-fold increase, about a 150-fold increase, about a 175-fold increase, about a 200-fold increase, about a 250-fold increase, about a 300-fold increase, about a 350-fold increase, about a 400-fold increase, about a 450-fold increase, or about a 500-fold increase).

In certain embodiments, the maximum theoretical yield (Yₘₐₓ) of 3-HP ranges from about 0.06 grams of 3-HP per gram of substrate (also referred to as “feedstock” or “carbon source”) to about 2.0 grams of 3-HP per gram of substrate, depending on the carbon composition of the substrate.

### Production of Acrylic Acid

The 3-HP that is generated according to the methods provided herein can further be used to produce acrylic acid. In some embodiments, the 3-HP is isolated prior to its conversion to acrylic acid and in some embodiments, the 3-HP is not isolated prior to its conversion to acrylic acid.

Acrylic acid can be generated from 3-HP according to a variety of known methods including, but not limited to, distillation, dehydration and fermentation based methods. For example, dehydration of 3-HP in the presence of a strong acid catalyst (e.g., phosphoric acid) can generate acrylic acid. Other methods are described, for example, in U.S. Pat. Nos. 3,639,466; 7,279,598; 8,338,145; 8,846,353; U.S. Appln. No. 2011/0105791 A1; and PCT publication WO 2013/185009 A1.

### EXAMPLES

The examples set forth below illustrate certain embodiments and do not limit the technology. Certain examples set forth below utilize standard recombinant DNA and other biotechnology protocols known in the art. Many such techniques are described in detail in Maniatis, T., E. F. Fritsch and J. Sambrook (1982) Molecular Cloning: a Laboratory Manual; Cold Spring Harbor Laboratory, Cold Spring Harbor, N.Y. DNA mutagenesis can be accomplished using the Stratagene (San Diego, Calif.) “QuickChange” kit according to the manufacturer's instructions.

Non-limiting examples of recombinant DNA techniques and genetic manipulation of microorganisms are described herein. In some embodiments, strains of engineered organisms described herein are mated to combine genetic backgrounds to further enhance carbon flux management through native and/or engineered pathways described herein, for the production of a desired target product (e.g., 3-hydroxypropionic acid).

The formulae for certain media used in selected examples are set forth below:

(1) TE-LiOAc (Tris/EDTA/Lithium Acetate)Solution
A 1×TE LiOAc solution is prepared by mixing together the following:
   1 part 10×TE solution (0.1 M Tris-C1, 0.01 mM EDTA, pH 7.5)
   1 part 10× LiOAc solution (1M LiOAc, pH 7.5, adjusted with diluted acetic acid)
   8 parts sterile distilled water

(2) SC Dextrose (-Ura) liquid media (SCD-ura media)

| | | |
|---|---|---|
| Glucose | 10 | g |
| Drop-out mix (-Ura) | l | g |
| Yeast nitrogen base | 3.35 | g |
| ddH₂O | 400 | mL |

In a clean 500 mL bottle, mix the glucose, SC (-ura) mix, yeast nitrogen base, and 400 mL of double-distilled water. Once the components have dissolved completely, fill to 500 mL with double-distilled water. Filter sterilize using a 0.2 micron filterware setup. Store at room temperature.

*Note: an equivalent amount of fructose may be substituted for glucose if SCFructose (-URA) media is needed.

(3) SC Dextrose (-Ura) plates (per liter) (SCD-ura plates)

| | | |
|---|---|---|
| Glucose | 20 | g |
| Drop-out mix (-Ura) | 2 | g |
| Yeast nitrogen base | 6.7 | g |
| ddH₂O | 250 | mL |

Agar solution:

| Bacto agar | 20 g |
|---|---|
| ddH₂O | 680 |

1) Mix the agar and double distilled water thoroughly, fill to 700 mL and transfer to a 1 L glass bottle. Autoclave on the liquid cycle.

2) In a separate bottle, prepare the AGAR SOLUTION. Once the components have dissolved completely, fill to 300 mL with double-distilled water. Filter sterilize with 0.2 micron filterware, then cool to about 60° C.

3) Swirl to mix thoroughly. Plate approximately 30 mL/plate. Solidify several hours/overnight at room temperature and then store at 4° C. upside-down.

(4) SC Dextrose plates with 5-FOA

| Agar solution: | | |
|---|---|---|
| Bacto agar | 20 | g |
| ddH₂O | 480 | mL |

Media mix:

| | | |
|---|---|---|
| 5-FOA | 1 | g |
| Uracil | 0.3 | g |
| Glucose | 20 | g |
| Nitrogen | 6.7 | g |
| Amino acid dropout mix | 2.14 | g |
| ddH₂O | 400 | mL |

“5-FOA” refers to 5-fluoroorotic acid.

Prepare the agar mix (final volume 500 mL) in a 2 L flask. Autoclave on liquid cycle. Fill to 500 mL total volume. Dissolve with stirring on low heat at a maximum temperature of 55° C. Filter sterilize using 0.2 micron filterware. After sterilization, cool to about 60° C. then add the media mix. Swirl to mix thoroughly.

(5) YPD Liquid Media (per liter)

To 700 ml of water in a beaker, add
10 g of Yeast Extract
20 g of Bacto Peptone

Mix until in solution. Bring volume to 900 mls. Autoclave. Add 100 ml of a sterile 20% Dextrose solution.

(6) YPD Plates (for 40 plates)

To 700 ml of water in a beaker add
10 g of Yeast Extract
20 g of Bacto Peptone

Mix until in solution. Bring volume to 900 mls and place in a 2 L Beaker. Add 20 g of Bacto Agar and mix. Autoclave. Add 100 ml of a sterile 20% Dextrose solution. Mix and pour plates.

(7) 20% Dextrose solution

To 780 mls of ddH₂O add 200 g of Dextrose. Mix until in solution and bring volume to 1000 mls with ddH₂O. Filter sterilized.

(8) YP Liquid Media (for 1 L)

To 700 ml of water in a beaker, add
10 g of Yeast Extract
20 g of Bacto Peptone

Mix until in solution. Bring volume to 1 L. Autoclave.

### Example 1: Cloning HPD1 from Candida Strain ATCC20336

The HPD1 DNA sequence (SEQ ID NO: 1), which encodes a 3-hydroxypropionate dehydrogenase (SEQ ID NO: 2), was amplified from Candida strain ATCC20336 genomic DNA using primers MMSB_FWD (SEQ ID NO: 3) and MMSB_REV (SEQ ID NO: 4). The PCR product was gel purified, ligated into a pET26b plasmid vector (Novagen), and transformed into competent TOP10 E. coli cells (Invitrogen). Clones containing PCR inserts were sequenced to confirm correct DNA sequence, exemplary of which is plasmid pAA1753 (SEQ ID NO: 5).

### Example 2: Enzyme Assay to Determine HPD1 Function

E. Coli strains containing either pAA1753 (SEQ ID NO: 5) or a pET26b vector were induced using the Novagen overnight express autoinduction system 1 with shaking at 250 rpm and 37° C. Samples were prepared by pelleting cells at 13,000 rpm, rinsed once with water, and then resuspended in buffer K containing 50 mM Tris-HCl, pH 8.0 and 1 mM MgCl₂. Cells were lysed by three rounds of sonication, consisting of 20 a second of sonication, followed by a 1 minute rest on ice. Following sonication, the insoluble debris was pelleted by centrifugation at 4° C. for 15 minutes at 16,000 rpm. Soluble cell extracts were kept cold while protein was purified using the Qiagen Ni-NTA spin kit. Samples were run through a PD10 column to remove imidazole and eluted in buffer K. total protein concentrations in eluates were determined by the Coomassie Plus (Bradford) assay.

For measuring dehydrogenase activity, each reaction contained 50 mM Tris-HCl, pH8.0, 2 mM MgCl₂, 1 mM NADP+ or 1 mM NAD+. 100 μl soluble cell extract was added to each reaction for a total volume of 270 μl. Absorbance measurements were taken for 3 minutes at 340 nm & 30° C. before and after adding 30 μl of 100 mM 3HP to each reaction (Table 1).

TABLE 1

| | specific activity (U/mg) |
|---|---|
| Control (no HPD1 protein) | −6.1E−04 |
| HPD1 | 1.1E−02 |

### Example 3: Transformation Procedure

5 mL YPD start cultures were inoculated with a single colony of Candida strain ATCC20913 and incubated overnight at 30° C., with shaking at about 200 rpm. The following day, fresh 25 mL YPD cultures were inoculated to an initial OD₆₀₀ nm of 0.4 and the culture incubated at 30° C., with shaking at about 200 rpm until an OD₆₀₀ nm of 1.0-2.0 was reached. Cells were pelleted by centrifugation at 1,000×g, 4° C. for 10 minutes. Cells were washed by resuspending in 10 mL sterile water, pelleted, resuspended in 1 mL sterile water and transferred to a 1.5 mL microcentrifuge tube. The cells were then washed in 1 mL sterile TE/LiOAC solution, pH 7.5, pelleted, resuspended in 0.25 mL TE/LiOAC solution and incubated with shaking at 30° C. for 30 minutes.

The cell solution was divided into 50 μL aliquots in 1.5 mL tubes to which was added 5-8 μg of linearized DNA and 5 μL of carrier DNA (boiled and cooled salmon sperm DNA, 10 mg/mL). 300 μL of sterile PEG solution (40% PEG 3500, 1×TE, 1× LiOAC) was added, mixed thoroughly and incubated at 30° C. for 60 minutes with gentle mixing every 15 minutes. 40 μL of DMSO was added, mixed thoroughly and the cell solution was incubated at 42° C. for 15 minutes. Cells were then pelleted by centrifugation at 1,000×g 30 seconds, resuspended in 500 μL of YPD media and incubated at 30° C. with shaking at about 200 rpm for 2 hours. Cells were then pelleted by centrifugation and resuspended in 1 mL 1×TE, cells were pelleted again, resuspended in 0.2 mL 1×TE and plated on selective media. Plates were incubated at 30° C. for growth of transformants.

### Example 4: Construction of Strain sAA5405 (HPD1/Hpd1::-P_{URA3}URA3T_{URA3}P_{URA3})

In order to create an HPD1 deletion strain, an HPD1 deletion cassette (SEQ ID NO: 6) was constructed by assembling 3 DNA fragments using overlap extension PCR. The HPD1 upstream fragment (SEQ ID NO 7) was a 400 bp DNA fragment of the HPD1 upstream region, and was amplified from Candida strain ATCC20336 genomic DNA using primers oAA7030 (SEQ ID NO: 8) and oAA7018 (SEQ ID NO: 9). The HPD1 downstream fragment (SEQ ID NO: 10) was a 400 bp DNA fragment of the HPD1 downstream region, and was amplified from Candida strain ATCC20336 genomic DNA using primers oAA7017 (SEQ ID NO: 11) and oAA7020 (SEQ ID NO: 12). The URA3 fragment was a 2.0 kb P_{URA3}URA3 T_{URA3}P_{URA3} cassette (SEQ ID NO: 13), and was amplified from plasmid pAA1860 (SEQ ID NO: 14) using primers oAA7019 (SEQ ID NO: 15) and oAA7036 (SEQ ID NO: 16). The HPD1 deletion cassette was then assembled by running a standard PCR reaction containing the HPD1 upstream, HPD1 downstream, and URA3 fragments, and primers oAA7030 and oAA7036. The HPD1 deletion cassette was purified and chemically transformed into strain sAA002; the cells were plated onto SCD-ura plates. The resultant colonies were streaked onto YPD for isolation and characterization. Colony PCR was performed to confirm the presence of the deletion cassette and one verified isolate was saved as strain sAA5405.

### Example 5: Construction of Strain sAA5526 (HPD1/Hpd1::-P_{URA3})

Strain sAA5405 was grown overnight in YPD media and plated on 5-FOA plates. Colonies that grew in the presence of 5-FOA were PCR screened for the looping out of the URA3 gene leaving behind only the URA3 promoter (P_{URA3}) in the first HPD1 allele and one verified isolate was saved as strain sAA5526.

### Example 6: Construction of Strain sAA5600 (Hpd1::-P_{URA3}URA3T_{URA3}P_{URA3}/Hpd1::-P_{URA3})

For deletion of the second HPD1 allele, the HPD1 deletion cassette (SEQ ID NO: 6) was assembled as described above. The HPD1 deletion cassette was purified and chemically transformed into strain sAA5526; the cells were plated onto SCD-ura plates. The resultant colonies were streaked onto YPD for isolation and characterization. Colony PCR was performed to confirm the presence of the deletion cassette and one verified isolate was saved as strain sAA5600.

### Example 7: Shake Flask Characterization of sAA5600 on Methyl Pentadecanoate, Nonane, and Heptane

Starter cultures (5 mL) of sAA5600 in YPD were incubated overnight at 30° C., with shaking at approximately 250 rpm. The overnight cultures were used to inoculate 25 mL fresh SP92-glycerol media (6.7 g/L yeast nitrogen base, 3.0 g/L yeast extract, 3.0 g/L (NH₄)₂SO₄, 1.0 g/L K₂HPO₄, 1.0 g/L KH₂PO₄, 75 g/L glycerol) to an initial OD₆₀₀ nm of 0.4 and incubated approximately 24 hours at 30° C., and 300 rpm shaking. Cells were pelleted by centrifugation for 10 minutes at 3,000×g, at 4° C., and then resuspended in 12.5 mL HiP-TAB media (yeast nitrogen base without amino acids and without ammonium sulfate, 1.7 g/L; yeast extract, 3.0 g/L; potassium phosphate monobasic, 10.0 g/L; potassium phosphate dibasic, 10.0 g/L) and added to 250 mL baffled shake flasks. 1.2 mL of Methyl pentadecanoate, Nonane, or Heptane was added to the shake flasks, which were shaken at approximately 300 rpm, at 30° C. Incubation of the cultures continued for 120 hours and samples were taken at 24, 48, and 120 hours for analysis of 3HP production by HPLC (Table 2).

### Example 8: Shake Flask Characterization of sAA5600 on Pentane

Starter cultures (5 mL) of sAA5600 in YPD were incubated overnight at 30° C., with shaking at approximately 250 rpm. The overnight cultures were used to inoculate 25 mL fresh SP92-glycerol media to an initial OD₆₀₀ nm of 0.4 and incubated approximately 24 hours at 30° C., and 300 rpm shaking. Cells were pelleted by centrifugation for 10 minutes at 3,000×g, at 4° C., and then resuspended in 12.5 mL HiP-TAB media and added to 250 mL baffled shake flasks. Cells were incubated approximately 24 hours at 30° C., and 300 rpm shaking. 280 μL of Pentane was added to shake flasks, which were then fitted with rubber stoppers to prevent evaporation of the Pentane feedstock. Cultures were incubated for 48 hours at 30° C., with shaking at approximately 300 rpm. Samples were taken at 48 hours for analysis of 3HP production by HPLC (Table 2).

### Example 9: Shake Flask Characterization of sAA5600 on Propane

Starter cultures (5 mL) of sAA5600 in YPD were incubated overnight at 30° C., with shaking at approximately 250 rpm. The overnight cultures were used to inoculate 25 mL fresh SP92-glycerol media to an initial OD₆₀₀ nm of 0.4 and incubated approximately 24 hours at 30° C., and 300 rpm shaking. Cells were pelleted by centrifugation for 10 minutes at 3,000×g, at 4° C., and then resuspended in 12.5 mL HiP-TAB media and added to 250 mL baffled shake flasks. Cells were incubated approximately 24 hours at 30° C., and 300 rpm shaking. In order to produce 3HP from propane, a co-feed is necessary for energy production. Therefore, 280 μL of hexane was added to shake flasks, which were then fitted with rubber stoppers. Using a syringe, the shake flasks were then filled with 100 mL of 100% propane, which were then vented to release internal pressure. Cultures were incubated for 48 hours at 30° C., with shaking at approximately 300 rpm. Samples were taken at 24 hours for analysis of 3HP production by HPLC (Table 2).

TABLE 2

| | ATCC20336 3HP | sAA5600 3HP |
|---|---|---|
| | Production(g/L) | Production(g/L) |
| Growth on Propane and | 0.00 | 0.83 |
| Hexane | | |
| Growth on Pentane | 0.00 | 2.44 |
| Growth on Heptane | 0.00 | 3.47 |
| Growth on Nonane | 0.00 | 21.60 |
| Growth on | 0.00 | 27.44 |
| Methyl-Pentadecanoate | | |

### Example 10: Construction of Strain sAA5679 (ALD6/Ald6::-P_{URA3}URA3T_{URA3}P_{URA3})

In order to delete the ALD6 gene (SEQ ID NO: 17), which encodes a malonate-semialdehyde dehydrogenase (EC 1.2.1.18) (SEQ ID NO: 18), an ALD6 deletion cassette (SEQ ID NO: 19) was constructed by assembling 3 DNA fragments using overlap extension PCR. The ALD6 upstream fragment (SEQ ID NO 20) was a 500 bp DNA fragment of the ALD6 upstream region, and was amplified from Candida strain ATCC20336 genomic DNA using primers oAA7029 (SEQ ID NO: 21) and oAA7022 (SEQ ID NO: 22). The ALD6 downstream fragment (SEQ ID NO 23) was a 400 bp DNA fragment of the ALD6 downstream region, and was amplified from Candida strain ATCC20336 genomic DNA using primers oAA7025 (SEQ ID NO: 24) and oAA7035 (SEQ ID NO: 25). The URA3 fragment was a 2.0 kb P_{URA3}URA3T_{URA3}P_{URA3} cassette (SEQ ID NO: 11), and was amplified from plasmid pAA1860 (SEQ ID NO: 12) using primers oAA7021 (SEQ ID NO: 26) and oAA7026 (SEQ ID NO: 27). The ALD6 deletion cassette was then assembled by running a standard PCR reaction containing the ALD6 upstream, ALD6 downstream, and URA3 fragments, and primers oAA7029 and oAA7035. The ALD6 deletion cassette was purified and chemically transformed into strain sAA002; the cells were plated onto SCD-ura plates. The resultant colonies were streaked onto YPD for isolation and characterization. Colony PCR was performed to confirm the presence of the deletion cassette and one verified isolate was saved as strain sAA5679.

### Example 11: Construction of Strain sAA5710 (ALD6/Ald6::-P_{URA3})

In order to loop the URA3 gene from sAA5679, the strain was grown overnight in YPD media and plated on 5-FOA plates. Colonies that grew in the presence of 5-FOA were PCR screened for the looping out of the URA3 gene leaving behind only the URA3 promoter (P_{URA3}) in the first ALD6 allele and one verified isolate was saved as strain sAA5710.

### Example 12: Construction of Strain sAA5733 (Ald6::P_{URA3}URA3T_{URA3}P_{URA3}/Ald6::-P_{URA3})

For deletion of the second ALD6 allele, the ALD6 deletion cassette (SEQ ID NO: 19) was assembled as described above. The ALD6 deletion cassette was purified and chemically transformed into strain sAA5710; the cells were plated onto SCD-ura plates. The resultant colonies were streaked onto YPD for isolation and characterization. Colony PCR was performed to confirm the presence of the deletion cassette and one verified isolate was saved as strain sAA5733.

### Example 13: Shake Flask Characterization of sAA5733 on Methyl Pentadecanoate, Nonane, and Heptane

Starter cultures (5 mL) of sAA5733 in YPD were incubated overnight at 30° C., with shaking at approximately 250 rpm. The overnight cultures were used to inoculate 25 mL fresh SP92-glycerol media (6.7 g/L yeast nitrogen base, 3.0 g/L yeast extract, 3.0 g/L (NH₄)₂SO₄, 1.0 g/L K₂HPO₄, 1.0 g/L KH₂PO₄, 75 g/L glycerol) to an initial OD600 nm of 0.4 and incubated approximately 24 hours at 30° C., and 300 rpm shaking. Cells were pelleted by centrifugation for 10 minutes at 3,000×g, at 4° C., and then resuspended in 12.5 mL HiP-TAB media (yeast nitrogen base without amino acids and without ammonium sulfate, 1.7 g/L; yeast extract, 3.0 g/L; potassium phosphate monobasic, 10.0 g/L; potassium phosphate dibasic, 10.0 g/L) media and added to 250 mL baffled shake flasks. 1.2 mL of Methyl pentadecanoate, Nonane, or Heptane was added to the shake flasks, which were shaken at approximately 300 rpm, at 30° C. Incubation of the cultures continued for 120 hours and samples were taken at 24, 48, and 120 hours for analysis of 3HP production (Table 3).

TABLE 3

| | ATCC20336 3HP | sAA5733 3HP |
|---|---|---|
| | Production(g/L) | Production(g/L) |
| 3HP from Heptane | 0.00 | 0.23 |
| 3HP from Nonane | 0.00 | 8.51 |
| 3HP from | 0.00 | 6.15 |
| Methyl-Pentadecanoate | | |

### Example 14: Shake Flask Characterization of sAA5733 on Pentane

Starter cultures (5 mL) of sAA5733 in YPD are incubated overnight between about 25° C. to about 35° C., generally at about 30° C., with shaking at about 200 rpm to 300 rpm, generally approximately 250 rpm. The overnight cultures can be used to inoculate 25 mL fresh SP92-glycerol media to an initial OD600 nm of 0.4 and then incubated approximately between 10 hours to 48 hours between about 25° C. to about 35° C., generally at about 30° C., and about 200 rpm to 400 rpm, generally about 300 rpm shaking. Cells can be pelleted by centrifugation for 10 minutes at 3,000×g, at 4° C., and then resuspended in 12.5 mL HiP-TAB media and added to 250 mL baffled shake flasks. Cells can be incubated approximately between 10 hours to 48 hours, generally about 24 hours, at a temperature between about 25° C. to about 35° C., generally at about 30° C., and about 200 rpm to 400 rpm, generally about 300 rpm shaking. 280 μL of Pentane is then added to shake flasks, which are fitted with rubber stoppers to prevent evaporation of the Pentane feedstock. Cultures are incubated for about 48 hours at about 30° C., with shaking at approximately 300 rpm. Samples can be taken at about 48 hours for analysis of 3HP production.

### Example 15: Shake Flask Characterization of sAA5733 on Propane

Starter cultures (5 mL) of sAA5733 in YPD are incubated overnight between about 25° C. to about 35° C., generally at about 30° C., with shaking at about 200 rpm to 300 rpm, generally approximately 250 rpm. The overnight cultures can be used to inoculate 25 mL fresh SP92-glycerol media to an initial OD600 nm of 0.4 and then incubated approximately between 10 hours to 48 hours between about 25° C. to about 35° C., generally at about 30° C., and about 200 rpm to 400 rpm, generally about 300 rpm shaking. Cells can be pelleted by centrifugation for 10 minutes at 3,000×g, at 4° C., and then resuspended in 12.5 mL HiP-TAB media and added to 250 mL baffled shake flasks. Cells can be incubated approximately between 10 hours to 48 hours, generally about 24 hours, at a temperature between about 25° C. to about 35° C., generally at about 30° C., and about 200 rpm to 400 rpm, generally about 300 rpm shaking. In order to produce 3HP from propane, a co-feed generally is necessary for energy production. Therefore, for example, 280 μL of hexane can be added to shake flasks, which are then fitted with rubber stoppers. Using a syringe, the shake flasks can then be filled with 100 mL of 100% propane, which are then vented to release internal pressure. Cultures are incubated for 48 hours at about 30° C., with shaking at approximately 300 rpm. Samples can be taken at 48 hours for analysis of 3HP production.

### Example 16: Measure 3HP Degradation in Strains ATCC20336 and sAA5600

Starter cultures (5 mL) of ATCC20336 and sAA5600 in YPD were incubated overnight at 30° C., with shaking at approximately 250 rpm. The overnight cultures were used to inoculate 25 mL fresh SP92-glycerol media (6.7 g/L yeast nitrogen base, 3.0 g/L yeast extract, 3.0 g/L (NH₄)₂SO₄, 1.0 g/L K₂HPO₄, 1.0 g/L KH₂PO₄, 75 g/L glycerol) to an initial OD₆₀₀ nm of 0.4 and incubated approximately 24 hours at 30° C., and 300 rpm shaking. Cells were pelleted by centrifugation for 10 minutes at 3,000×g, at 4° C., and then resuspended in 12.5 mL HiP-TAB media (yeast nitrogen base without amino acids and without ammonium sulfate, 1.7 g/L; yeast extract, 3.0 g/L; potassium phosphate monobasic, 10.0 g/L; potassium phosphate dibasic, 10.0 g/L) and added to 250 mL baffled shake flasks. 0.16 mL of 30% 3HP was added to the shake flasks, bring the 3HP concentration to 4 g/L. Cultures were then shaken at approximately 300 rpm, at 30° C. Incubation of the cultures continued for 48 hours and samples were taken at 24 and 48 hours for HPLC analysis of 3HP degradation (Table 4).

TABLE 4

| | ATCC20336 | sAA5600 |
|---|---|---|
| 3HP at 0 hours (g/L) | 4.00 | 4.00 |
| 3HP at 24 hours (g/L) | 0.16 | 4.05 |
| 3HP at 48 hours (g/L) | 0.03 | 4.01 |

### Example 17: Detection of 3HP in Fermentation Samples by HPLC

For the detection of 3HP, a Thermo Scientific UltiMate 3000 UHPLC was used. The UHPLC is equipped with a degasser, Quaternary pump with 25.6 mM Sulfuric Acid in Milli-Q water mobile phase at 0.7 mL/min, Column oven at 45C with a Phenomenex Rezex RHM Monosaccharide H+(8%) 150×7.8 column, autosampler with 20 uL injection, Refractive Index Detector, and a Variable Wavelength UV Detector at 210 nm. A 5 g/L standard was prepared and run in five levels and was detected on Refractive Index Detector with retention time of 6.29 min and UV Detector with retention time of 6.12 minutes.

### Example 18: Non-Limiting Examples of Certain Polynucleotides and Polypeptides

Listed in the following table are non-limiting examples of certain polynucleotides and polypeptides.

| SEQ | | Organism and | |
|---|---|---|---|
| ID NO: | Name | sequence type | Sequence |
| 1 | 3- | Candida sp. | atgttgagatcttcagtccgtactttctccacccagtccag |
| | hydroxypropionate | polynucleotide | agtattagccaactacggtttcgtaggcttgggtctcatgg |
| | dehydrogenase | | gccagcacatggccagacacgtctacaaccagttgcagcca |
| | (EC 1.1.1.59) | | gcagacaagttgtatgtccacgacgtcaacccccagcacac |
| | | | cacccagttcgtcaccgacgtgaccacccagaagccacaga |
| | | | acgccacacaattgacgcccttgtcctccttgaaagagttc |
| | | | accaccgagccagagtcccagttggacttcatcgtcaccat |
| | | | ggtccccgagggcaagcacgtcaaggccgttgtctccgagc |
| | | | tagtcgaccactacaatgcgtcgggaaaatacgacccatcc |
| | | | aagaagttgacctttgtggactcctccaccatcgacatccc |
| | | | cacctccagggaggtccaccagctcgttgccgacaagttac |
| | | | aaggcgccacgttcatcgacgccccggtttcgggtggtgtc |
| | | | gctggtgccaggaacggaaccttgtcgttcatggtgtcgcg |
| | | | ggacaccaaggaagacgtcgaccctaacctcgtcacgcttt |
| | | | tgaactacatgggcagcaacatcttcccatgtggtggaacc |
| | | | cacgggaccggcttggctgccaagttggcaaacaactactt |
| | | | gttggcgatcacgaacatcgccgtcgcagatagcttccagt |
| | | | tggcaaactcgttcgggttgaacttgcagaactacgccaag |
| | | | ttggtgtcgacctccacaggtaagtcctgggctagtgtcga |
| | | | taactgcccaatccccggtgtctaccctgaaaagaacttga |
| | | | cttgtgataacggatacaagggtgggtttgtcacgaagttg |
| | | | acgagaaaggatgtcgtcttggctacggagtctgctaaggc |
| | | | taacaaccagttccttatgcttggcgaagtcggtagatact |
| | | | ggtacgacaaggcttgtgaagatgaaaagtacgccaacaga |
| | | | gacttgtctgttcttttcgaattcttgggtgatcttaaaaa |
| | | | ataa |
| 2 | 3- | Candida sp. | mlissvrtfstqsrvlanygfvglglmgqhmarhvynglqp |
| | hydroxypropionate | polypeptide | adklyvhdvnpqhttqfvtdvttqkpqnatqltplsslkef |
| | dehydrogenase | | ttepesqldfivtmvpegkhvkavvselvdhynasgkydps |
| | (EC 1.1.1.59) | | kkltfvdsstidiptsrevhqlvadklqgatfidapvsggv |
| | | | agarngtlsfmvsrdtkedvdpnlvtllnymgsnifpcggt |
| | | | hgtglaaklannyllaitniavadsfqlansfglnlqnyak |
| | | | lvststgkswasvdncpipgvypeknitcdngykggfvtkl |
| | | | trkdvvlatesakannqflmlgevgrywydkacedekyanr |
| | | | dlsvlfeflgdlkk |
| 3 | PCR primer | Artificial DNA | tacccatatgttgagatcttcagtccgta |
| 4 | PCR primer | Artificial DNA | taccctcgagttttttaagatcacccaagaatt |
| 5 | pAA1753 | Artificial DNA | atccggatatagttcctcctttcagcaaaaaacccctcaag |
| | plasmid | | acccgtttagaggccccaaggggttatgctagttattgctc |
| | | | agcggtggcagcagccaactcagcttcctttcgggctttgt |
| | | | tagcagccggatctcagtggtggtggtggtggtgctcgagt |
| | | | tttttaagatcacccaagaattcgaaaagaacagacaagtc |
| | | | tctgttggcgtacttttcatcttcacaagccttgtcgtacc |
| | | | agtatctaccgacttcgccaagcataaggaactggttgtta |
| | | | gccttagcagactccgtagccaagacgacatcctttctcgt |
| | | | caacttcgtgacaaacccacccttgtatccgttatcacaag |
| | | | tcaagttcttttcagggtagacaccggggattgggcagtta |
| | | | tcgacactagcccaggacttacctgtggaggtcgacaccaa |
| | | | cttggcgtagttctgcaagttcaacccgaacgagtttgcca |
| | | | actggaagctatctgcgacggcgatgttcgtgatcgccaac |
| | | | aagtagttgtttgccaacttggcagccaagccggtcccgtg |
| | | | ggttccaccacatgggaagatgttgctgcccatgtagttca |
| | | | aaagcgtgacgaggttagggtcgacgtcttccttggtgtcc |
| | | | cgcgacaccatgaacgacaaggttccgttcctggcaccagc |
| | | | gacaccacccgaaaccggggcgtcgatgaacgtggcgcctt |
| | | | gtaacttgtcggcaacgagctggtggacctccctggaggtg |
| | | | gggatgtcgatggtggaggagtccacaaaggtcaacttctt |
| | | | ggatgggtcgtattttcccgacgcattgtagtggtcgacta |
| | | | gctcggagacaacggccttgacgtgcttgccctcggggacc |
| | | | atggtgacgatgaagtccaactgggactctggctcggtggt |
| | | | gaactctttcaaggaggacaagggcgtcaattgtgtggcgt |
| | | | tctgtggcttctgggtggtcacgtcggtgacgaactgggtg |
| | | | gtgtgctgggggttgacgtcgtggacatacaacttgtctgc |
| | | | tggctgcaactggttgtagacgtgtctggccatgtgctggc |
| | | | ccatgagacccaagcctacgaaaccgtagttggctaatact |
| | | | ctggactgggtggagaaagtacggactgaagatctcaacat |
| | | | atgtatatctccttcttaaagttaaacaaaattatttctag |
| | | | aggggaattgttatccgctcacaattcccctatagtgagtc |
| | | | gtattaatttcgcgggatcgagatctcgatcctctacgccg |
| | | | gacgcatcgtggccggcatcaccggcgccacaggtgcggtt |
| | | | gctggcgcctatatcgccgacatcaccgatggggaagatcg |
| | | | ggctcgccacttcgggctcatgagcgcttgtttcggcgtgg |
| | | | gtatggtggcaggccccgtggccgggggactgttgggcgcc |
| | | | atctccttgcatgcaccattccttgcggcggcggtgctcaa |
| | | | cggcctcaacctactactgggctgcttcctaatgcaggagt |
| | | | cgcataagggagagcgtcgagatcccggacaccatcgaatg |
| | | | gcgcaaaacctttcgcggtatggcatgatagcgcccggaag |
| | | | agagtcaattcagggtggtgaatgtgaaaccagtaacgtta |
| | | | tacgatgtcgcagagtatgccggtgtctcttatcagaccgt |
| | | | ttcccgcgtggtgaaccaggccagccacgtttctgcgaaaa |
| | | | cgcgggaaaaagtggaagcggcgatggcggagctgaattac |
| | | | attcccaaccgcgtggcacaacaactggcgggcaaacagtc |
| | | | gttgctgattggcgttgccacctccagtctggccctgcacg |
| | | | cgccgtcgcaaattgtcgcggcgattaaatctcgcgccgat |
| | | | caactgggtgccagcgtggtggtgtcgatggtagaacgaag |
| | | | cggcgtcgaagcctgtaaagcggcggtgcacaatcttctcg |
| | | | cgcaacgcgtcagtgggctgatcattaactatccgctggat |
| | | | gaccaggatgccattgctgtggaagctgcctgcactaatgt |
| | | | tccggcgttatttcttgatgtctctgaccagacacccatca |
| | | | acagtattattttctcccatgaagacggtacgcgactgggc |
| | | | gtggagcatctggtcgcattgggtcaccagcaaatcgcgct |
| | | | gttagcgggcccattaagttctgtctcggcgcgtctgcgtc |
| | | | tggctggctggcataaatatctcactcgcaatcaaattcag |
| | | | ccgatagcggaacgggaaggcgactggagtgccatgtccgg |
| | | | ttttcaacaaaccatgcaaatgctgaatgagggcatcgttc |
| | | | ccactgcgatgctggttgccaacgatcagatggcgctgggc |
| | | | gcaatgcgcgccattaccgagtccgggctgcgcgttggtgc |
| | | | ggatatctcggtagtgggatacgacgataccgaagacagct |
| | | | catgttatatcccgccgttaaccaccatcaaacaggatttt |
| | | | cgcctgctggggcaaaccagcgtggaccgcttgctgcaact |
| | | | ctctcagggccaggcggtgaagggcaatcagctgttgcccg |
| | | | tctcactggtgaaaagaaaaaccaccctggcgcccaatacg |
| | | | caaaccgcctctccccgcgcgttggccgattcattaatgca |
| | | | gctggcacgacaggtttcccgactggaaagcgggcagtgag |
| | | | cgcaacgcaattaatgtaagttagctcactcattaggcacc |
| | | | gggatctcgaccgatgcccttgagagccttcaacccagtca |
| | | | gctccttccggtgggcgcggggcatgactatcgtcgccgca |
| | | | cttatgactgtcttctttatcatgcaactcgtaggacaggt |
| | | | gccggcagcgctctgggtcattttcggcgaggaccgctttc |
| | | | gctggagcgcgacgatgatcggcctgtcgcttgcggtattc |
| | | | ggaatcttgcacgccctcgctcaagccttcgtcactggtcc |
| | | | cgccaccaaacgtttcggcgagaagcaggccattatcgccg |
| | | | gcatggcggccccacgggtgcgcatgatcgtgctcctgtcg |
| | | | ttgaggacccggctaggctggcggggttgccttactggtta |
| | | | gcagaatgaatcaccgatacgcgagcgaacgtgaagcgact |
| | | | gctgctgcaaaacgtctgcgacctgagcaacaacatgaatg |
| | | | gtcttcggtttccgtgtttcgtaaagtctggaaacgcggaa |
| | | | gtcagcgccctgcaccattatgttccggatctgcatcgcag |
| | | | gatgctgctggctaccctgtggaacacctacatctgtatta |
| | | | acgaagcgctggcattgaccctgagtgatttttctctggtc |
| | | | ccgccgcatccataccgccagttgtttaccctcacaacgtt |
| | | | ccagtaaccgggcatgttcatcatcagtaacccgtatcgtg |
| | | | agcatcctctctcgtttcatcggtatcattacccccatgaa |
| | | | cagaaatcccccttacacggaggcatcagtgaccaaacagg |
| | | | aaaaaaccgcccttaacatggcccgctttatcagaagccag |
| | | | acattaacgcttctggagaaactcaacgagctggacgcgga |
| | | | tgaacaggcagacatctgtgaatcgcttcacgaccacgctg |
| | | | atgagctttaccgcagctgcctcgcgcgtttcggtgatgac |
| | | | ggtgaaaacctctgacacatgcagctcccggagacggtcac |
| | | | agcttgtctgtaagcggatgccgggagcagacaagcccgtc |
| | | | agggcgcgtcagcgggtgttggcgggtgtcggggcgcagcc |
| | | | atgacccagtcacgtagcgatagcggagtgtatactggctt |
| | | | aactatgcggcatcagagcagattgtactgagagtgcacca |
| | | | tatatgcggtgtgaaataccgcacagatgcgtaaggagaaa |
| | | | ataccgcatcaggcgctcttccgcttcctcgctcactgact |
| | | | cgctgcgctcggtcgttcggctgcggcgagcggtatcagct |
| | | | cactcaaaggcggtaatacggttatccacagaatcagggga |
| | | | taacgcaggaaagaacatgtgagcaaaaggccagcaaaagg |
| | | | ccaggaaccgtaaaaaggccgcgttgctggcgtttttccat |
| | | | aggctccgcccccctgacgagcatcacaaaaatcgacgctc |
| | | | aagtcagaggtggcgaaacccgacaggactataaagatacc |
| | | | aggcgtttccccctggaagctccctcgtgcgctctcctgtt |
| | | | ccgaccctgccgcttaccggatacctgtccgcctttctccc |
| | | | ttcgggaagcgtggcgctttctcatagctcacgctgtaggt |
| | | | atctcagttcggtgtaggtcgttcgctccaagctgggctgt |
| | | | gtgcacgaaccccccgttcagcccgaccgctgcgccttatc |
| | | | cggtaactatcgtcttgagtccaacccggtaagacacgact |
| | | | tatcgccactggcagcagccactggtaacaggattagcaga |
| | | | gcgaggtatgtaggcggtgctacagagttcttgaagtggtg |
| | | | gcctaactacggctacactagaaggacagtatttggtatct |
| | | | gcgctctgctgaagccagttaccttcggaaaaagagttggt |
| | | | agctcttgatccggcaaacaaaccaccgctggtagcggtgg |
| | | | tttttttgtttgcaagcagcagattacgcgcagaaaaaaag |
| | | | gatctcaagaagatcctttgatcttttctacggggtctgac |
| | | | gctcagtggaacgaaaactcacgttaagggattttggtcat |
| | | | gaacaataaaactgtctgcttacataaacagtaatacaagg |
| | | | ggtgttatgagccatattcaacgggaaacgtcttgctctag |
| | | | gccgcgattaaattccaacatggatgctgatttatatgggt |
| | | | ataaatgggctcgcgataatgtcgggcaatcaggtgcgaca |
| | | | atctatcgattgtatgggaagcccgatgcgccagagttgtt |
| | | | tctgaaacatggcaaaggtagcgttgccaatgatgttacag |
| | | | atgagatggtcagactaaactggctgacggaatttatgcct |
| | | | cttccgaccatcaagcattttatccgtactcctgatgatgc |
| | | | atggttactcaccactgcgatccccgggaaaacagcattcc |
| | | | aggtattagaagaatatcctgattcaggtgaaaatattgtt |
| | | | gatgcgctggcagtgttcctgcgccggttgcattcgattcc |
| | | | tgtttgtaattgtccttttaacagcgatcgcgtatttcgtc |
| | | | tcgctcaggcgcaatcacgaatgaataacggtttggttgat |
| | | | gcgagtgattttgatgacgagcgtaatggctggcctgttga |
| | | | acaagtctggaaagaaatgcataaacttttgccattctcac |
| | | | cggattcagtcgtcactcatggtgatttctcacttgataac |
| | | | cttatttttgacgaggggaaattaataggttgtattgatgt |
| | | | tggacgagtcggaatcgcagaccgataccaggatcttgcca |
| | | | tcctatggaactgcctcggtgagttttctccttcattacag |
| | | | aaacggctttttcaaaaatatggtattgataatcctgatat |
| | | | gaataaattgcagtttcatttgatgctcgatgagtttttct |
| | | | aagaattaattcatgagcggatacatatttgaatgtattta |
| | | | gaaaaataaacaaataggggttccgcgcacatttccccgaa |
| | | | aagtgccacctgaaattgtaaacgttaatattttgttaaaa |
| | | | ttcgcgttaaatttttgttaaatcagctcattttttaacca |
| | | | ataggccgaaatcggcaaaatcccttataaatcaaaagaat |
| | | | agaccgagatagggttgagtgttgttccagtttggaacaag |
| | | | agtccactattaaagaacgtggactccaacgtcaaagggcg |
| | | | aaaaaccgtctatcagggcgatggcccactacgtgaaccat |
| | | | caccctaatcaagttttttggggtcgaggtgccgtaaagca |
| | | | ctaaatcggaaccctaaagggagcccccgatttagagcttg |
| | | | acggggaaagccggcgaacgtggcgagaaaggaagggaaga |
| | | | aagcgaaaggagcgggcgctagggcgctggcaagtgtagcg |
| | | | gtcacgctgcgcgtaaccaccacacccgccgcgcttaatgc |
| | | | gccgctacagggcgcgtcccattcgcca |
| 6 | HPD1 deletion | Artificial DNA | ttttctctgggctgtgttggttttttcgcagcttcagtttg |
| | cassette | | tgggtgtttgtgggtgtttggtgattccaacagatcgggtt |
| | | | aaatgtcacaagcatttaagaaacggccacgccaactaagc |
| | | | ccaaacgccgacccatcctacccgaattgtccactctcatg |
| | | | gataccatagttgaataaccgtcacctctattgaagcagtg |
| | | | atattacaaaaaggaacagggccattttgctgccgtagaag |
| | | | ctttcgcaggtaaagtggggaaaacccccatgcagcgtgta |
| | | | actggcatgataacactgaccgagttttcttttgtttaagg |
| | | | caaattgagtatgggcgggtgttccatgttctctttttttt |
| | | | taactctctccacagaaacccagaatggaattgtatctacg |
| | | | gttgtttcggtatgacccccggggatctgacgggtacaacg |
| | | | agaattgtattgaattgatcaagaacatgatcttggtgtta |
| | | | cagaacatcaagttcttggaccagactgagaatgcacagat |
| | | | atacaaggcgtcatgtgataaaatggatgagatttatccac |
| | | | aattgaagaaagagtttatggaaagtggtcaaccagaagct |
| | | | aaacaggaagaagcaaacgaagaggtgaaacaagaagaaga |
| | | | aggtaaataagtattttgtattatataacaaacaaagtaag |
| | | | gaatacagatttatacaataaattgccatactagtcacgtg |
| | | | agatatctcatccattccccaactcccaagaaaaaaaaaaa |
| | | | gtgaaaaaaaaaatcaaacccaaagatcaacctccccatca |
| | | | tcatcgtcatcaaacccccagctcaattcgcaatggttagc |
| | | | acaaaaacatacacagaaagggcatcagcacacccctccna |
| | | | ggttgcccaacgtttattccgcttaatggagtccaaaaaga |
| | | | ccaacctctgcgcctcgatcgacgtgaccacaaccgccgag |
| | | | ttcctttcgctcatcgacaagctcggtccccacatctgtct |
| | | | cgtgaagacgcacatcgatntcatctcagacttcagctacg |
| | | | agggcacgattgagccgttgcttgtgcttgcagagcgccac |
| | | | gggttcttgatattcgaggacaggaagtttgctgatatcgg |
| | | | aaacaccgtgatgttgcagtacacctcgggggtataccgga |
| | | | tcgcggcgtggagtgacatcacgaacgcgcacggagtgact |
| | | | gggaagggcgtcgttgaagggttgaaacgcggtgcggaggg |
| | | | ggtagaaaaggaaaggggcgtgttgatgtnggcggagttgt |
| | | | cgagtaaaggctcgttggcgcatggtgaatatacccgtgag |
| | | | acgatcgagattgcgaagagtgatcgggagttcgtgattgg |
| | | | gttcatcgcgcagcgggacatggggggtagagaagaagggt |
| | | | ttgattggatcatcatgacgcctggtgtggggttggatgat |
| | | | aaaggcgatgcgttgggccagcagtataggactgttgatga |
| | | | ggtggttctgactggtaccgatgtgattattgtcgggagag |
| | | | ggttgtttggaaaaggaagagaccctgaggtggagggaaag |
| | | | agatacagggatgctggatggaaggcatacttgaagagaac |
| | | | tggtcagttagaataaatattgtaataaataggtctatata |
| | | | catacactaagcttctaggacgtcattgtagtcttcgaagt |
| | | | tgtctgctagtttagttctcatgatttcgaaaaccaataac |
| | | | gcaatggatgtagcagggatggtggttagtgcgttcctgac |
| | | | aaacccagagtacgccgcctcaaaccacgtcacattcgccc |
| | | | tttgcttcatccgcatcacttgcttgaaggtatccacgtac |
| | | | gagttgtaatacaccttgaagaacggcttcgtctacggtcg |
| | | | acgacgggtacaacgagaattgtattgaattgatcaagaac |
| | | | atgatcttggtgttacagaacatcaagttcttggaccagac |
| | | | tgagaatgcacagatatacaaggcgtcatgtgataaaatgg |
| | | | atgagatttatccacaattgaagaaagagtttatggaaagt |
| | | | ggtcaaccagaagctaaacaggaagaagcaaacgaagaggt |
| | | | gaaacaagaagaagaaggtaaataagtattttgtattatat |
| | | | aacaaacaaagtaaggaatacagatttatacaataaattgc |
| | | | catactagtcacgtgagatatctcatccattccccaactcc |
| | | | caagaaaaaaaaaaagtgaaaaaaaaaatcaaacccaaaga |
| | | | tcaacctccccatcatcatcgtcatcaaacccccagctcaa |
| | | | ttcgcagagctcggtaccaaatgggtcaacagaatccaatt |
| | | | cggtgtactcgtagcaacctgttctttcttatcgtgatagt |
| | | | tcattctgacaacttttctgatccatcttcttcttctgtag |
| | | | agctcattgttgctggccaacttctcaatctgatccaacga |
| | | | gagctcgttaatcgtatggtcatccgtgtcattaatatcat |
| | | | tactcgtattcttcgtgattatatcatatgcccatttctca |
| | | | tcatcatcgataatcacgagatcttggatcaagtttccctc |
| | | | cacccatgcgttgatattgaagtcaatcttccatttttctg |
| | | | aatccaaaaacttgtagttcgcaggaggattgacctccgtc |
| | | | aaggtatccctcttgttgagattcaaaagcttgacgtcgtc |
| | | | ttcctgctggtggtcttcatcgtgctgtctctctaa |
| 7 | Genomic | Candida sp. | ttttctctgggctgtgttggttttttcgcagcttcagtttg |
| | region | polynucleotide | tgggtgtttgtgggtgtttggtgattccaacagatcgggtt |
| | upstream of | | aaatgtcacaagcatttaagaaacggccacgccaactaagc |
| | the HPD1 gene | | ccaaacgccgacccatcctacccgaattgtccactctcatg |
| | | | gataccatagttgaataaccgtcacctctattgaagcagtg |
| | | | atattacaaaaaggaacagggccattttgctgccgtagaag |
| | | | ctttcgcaggtaaagtggggaaaacccccatgcagcgtgta |
| | | | actggcatgataacactgaccgagttttcttttgtttaagg |
| | | | caaattgagtatgggcgggtgttccatgttctctttttttt |
| | | | taactctctccacagaaacccagaatggaat |
| 8 | PCR primer | Artificial DNA | ttttctctgggctgtgttggtt |
| 9 | PCR primer | Artificial DNA | tcataccgaaacaaccgtagatacaattccattctgggttt |
| | | | ctgtggaga |
| 10 | Genomic | Candida sp. | tactcgtagcaacctgttctttcttatcgtgatagttcatt |
| | region | polynucleotide | ctgacaacttttctgatccatcttcttcttctgtagagctc |
| | downstream of | | attgttgctggccaacttctcaatctgatccaacgagagct |
| | the HPD1 gene | | cgttaatcgtatggtcatccgtgtcattaatatcattactc |
| | | | gtattcttcgtgattatatcatatgcccatttctcatcatc |
| | | | atcgataatcacgagatcttggatcaagtttccctccaccc |
| | | | atgcgttgatattgaagtcaatcttccatttttctgaatcc |
| | | | aaaaacttgtagttcgcaggaggattgacctccgtcaaggt |
| | | | atccctcttgttgagattcaaaagcttgacgtcgtcttcct |
| | | | gctggtggtcttcatcgtgctgtctctctaa |
| 11 | PCR primer | Artificial DNA | tctccacagaaacccagaatggaattgtatctacggttgtt |
| | | | tcggtatga |
| 12 | PCR primer | Artificial DNA | ttagagagacagcacgatgaaga |
| 13 | 2.0 kb | Artificial DNA | tgtatctacggttgtttcggtatgacccccggggatctgac |
| | Pura3URA3Tura | | gggtacaacgagaattgtattgaattgatcaagaacatgat |
| | 3Pura3 | | cttggtgttacagaacatcaagttcttggaccagactgaga |
| | cassette | | atgcacagatatacaaggcgtcatgtgataaaatggatgag |
| | | | atttatccacaattgaagaaagagtttatggaaagtggtca |
| | | | accagaagctaaacaggaagaagcaaacgaagaggtgaaac |
| | | | aagaagaagaaggtaaataagtattttgtattatataacaa |
| | | | acaaagtaaggaatacagatttatacaataaattgccatac |
| | | | tagtcacgtgagatatctcatccattccccaactcccaaga |
| | | | aaaaaaaaaagtgaaaaaaaaaatcaaacccaaagatcaac |
| | | | ctccccatcatcatcgtcatcaaacccccagctcaattcgc |
| | | | aatggttagcacaaaaacatacacagaaagggcatcagcac |
| | | | acccctccnaggttgcccaacgtttattccgcttaatggag |
| | | | tccaaaaagaccaacctctgcgcctcgatcgacgtgaccac |
| | | | aaccgccgagttcctttcgctcatcgacaagctcggtcccc |
| | | | acatctgtctcgtgaagacgcacatcgatntcatctcagac |
| | | | ttcagctacgagggcacgattgagccgttgcttgtgcttgc |
| | | | agagcgccacgggttcttgatattcgaggacaggaagtttg |
| | | | ctgatatcggaaacaccgtgatgttgcagtacacctcgggg |
| | | | gtataccggatcgcggcgtggagtgacatcacgaacgcgca |
| | | | cggagtgactgggaagggcgtcgttgaagggttgaaacgcg |
| | | | gtgcggagggggtagaaaaggaaaggggcgtgttgatgtng |
| | | | gcggagttgtcgagtaaaggctcgttggcgcatggtgaata |
| | | | tacccgtgagacgatcgagattgcgaagagtgatcgggagt |
| | | | tcgtgattgggttcatcgcgcagcgggacatggggggtaga |
| | | | gaagaagggtttgattggatcatcatgacgcctggtgtggg |
| | | | gttggatgataaaggcgatgcgttgggccagcagtatagga |
| | | | ctgttgatgaggtggttctgactggtaccgatgtgattatt |
| | | | gtcgggagagggttgtttggaaaaggaagagaccctgaggt |
| | | | ggagggaaagagatacagggatgctggatggaaggcatact |
| | | | tgaagagaactggtcagttagaataaatattgtaataaata |
| | | | ggtctatatacatacactaagcttctaggacgtcattgtag |
| | | | tcttcgaagttgtctgctagtttagttctcatgatttcgaa |
| | | | aaccaataacgcaatggatgtagcagggatggtggttagtg |
| | | | cgttcctgacaaacccagagtacgccgcctcaaaccacgtc |
| | | | acattcgccctttgcttcatccgcatcacttgcttgaaggt |
| | | | atccacgtacgagttgtaatacaccttgaagaacggcttcg |
| | | | tctacggtcgacgacgggtacaacgagaattgtattgaatt |
| | | | gatcaagaacatgatcttggtgttacagaacatcaagttct |
| | | | tggaccagactgagaatgcacagatatacaaggcgtcatgt |
| | | | gataaaatggatgagatttatccacaattgaagaaagagtt |
| | | | tatggaaagtggtcaaccagaagctaaacaggaagaagcaa |
| | | | acgaagaggtgaaacaagaagaagaaggtaaataagtattt |
| | | | tgtattatataacaaacaaagtaaggaatacagatttatac |
| | | | aataaattgccatactagtcacgtgagatatctcatccatt |
| | | | ccccaactcccaagaaaaaaaaaaagtgaaaaaaaaaatca |
| | | | aacccaaagatcaacctccccatcatcatcgtcatcaaacc |
| | | | cccagctcaattcgcagagctcggtaccaaatgggtcaaca |
| | | | gaatccaattcggtg |
| 14 | pAA1860 | Artificial DNA | aagggcgaattctgcagatatccatcacactggcggccgct |
| | plasmid | | cgagcatgcatctagagggcccaattcgccctatagtgagt |
| | | | cgtattacaattcactggccgtcgttttacaacgtcgtgac |
| | | | tgggaaaaccctggcgttacccaacttaatcgccttgcagc |
| | | | acatccccctttcgccagctggcgtaatagcgaagaggccc |
| | | | gcaccgatcgcccttcccaacagttgcgcagcctatacgta |
| | | | cggcagtttaaggtttacacctataaaagagagagccgtta |
| | | | tcgtctgtttgtggatgtacagagtgatattattgacacgc |
| | | | cggggcgacggatggtgatccccctggccagtgcacgtctg |
| | | | ctgtcagataaagtctcccgtgaactttacccggtggtgca |
| | | | tatcggggatgaaagctggcgcatgatgaccaccgatatgg |
| | | | ccagtgtgccggtctccgttatcggggaagaagtggctgat |
| | | | ctcagccaccgcgaaaatgacatcaaaaacgccattaacct |
| | | | gatgttctggggaatataaatgtcaggcatgagattatcaa |
| | | | aaaggatcttcacctagatccttttcacgtagaaagccagt |
| | | | ccgcagaaacggtgctgaccccggatgaatgtcagctactg |
| | | | ggctatctggacaagggaaaacgcaagcgcaaagagaaagc |
| | | | aggtagcttgcagtgggcttacatggcgatagctagactgg |
| | | | gcggttttatggacagcaagcgaaccggaattgccagctgg |
| | | | ggcgccctctggtaaggttgggaagccctgcaaagtaaact |
| | | | ggatggctttctcgccgccaaggatctgatggcgcagggga |
| | | | tcaagctctgatcaagagacaggatgaggatcgtttcgcat |
| | | | gattgaacaagatggattgcacgcaggttctccggccgctt |
| | | | gggtggagaggctattcggctatgactgggcacaacagaca |
| | | | atcggctgctctgatgccgccgtgttccggctgtcagcgca |
| | | | ggggcgcccggttctttttgtcaagaccgacctgtccggtg |
| | | | ccctgaatgaactgcaagacgaggcagcgcggctatcgtgg |
| | | | ctggccacgacgggcgttccttgcgcagctgtgctcgacgt |
| | | | tgtcactgaagcgggaagggactggctgctattgggcgaag |
| | | | tgccggggcaggatctcctgtcatctcaccttgctcctgcc |
| | | | gagaaagtatccatcatggctgatgcaatgcggcggctgca |
| | | | tacgcttgatccggctacctgcccattcgaccaccaagcga |
| | | | aacatcgcatcgagcgagcacgtactcggatggaagccggt |
| | | | cttgtcgatcaggatgatctggacgaagagcatcaggggct |
| | | | cgcgccagccgaactgttcgccaggctcaaggcgagcatgc |
| | | | ccgacggcgaggatctcgtcgtgacccatggcgatgcctgc |
| | | | ttgccgaatatcatggtggaaaatggccgcttttctggatt |
| | | | catcgactgtggccggctgggtgtggcggaccgctatcagg |
| | | | acatagcgttggctacccgtgatattgctgaagagcttggc |
| | | | ggcgaatgggctgaccgcttcctcgtgctttacggtatcgc |
| | | | cgctcccgattcgcagcgcatcgccttctatcgccttcttg |
| | | | acgagttcttctgaattattaacgcttacaatttcctgatg |
| | | | cggtattttctccttacgcatctgtgcggtatttcacaccg |
| | | | catacaggtggcacttttcggggaaatgtgcgcggaacccc |
| | | | tatttgtttatttttctaaatacattcaaatatgtatccgc |
| | | | tcatgagacaataaccctgataaatgcttcaataatagcac |
| | | | gtgaggagggccaccatggccaagttgaccagtgccgttcc |
| | | | ggtgctcaccgcgcgcgacgtcgccggagcggtcgagttct |
| | | | ggaccgaccggctcgggttctcccgggacttcgtggaggac |
| | | | gacttcgccggtgtggtccgggacgacgtgaccctgttcat |
| | | | cagcgcggtccaggaccaggtggtgccggacaacaccctgg |
| | | | cctgggtgtgggtgcgcggcctggacgagctgtacgccgag |
| | | | tggtcggaggtcgtgtccacgaacttccgggacgcctccgg |
| | | | gccggccatgaccgagatcggcgagcagccgtgggggcggg |
| | | | agttcgccctgcgcgacccggccggcaactgcgtgcacttc |
| | | | gtggccgaggagcaggactgacacgtgctaaaacttcattt |
| | | | ttaatttaaaaggatctaggtgaagatcctttttgataatc |
| | | | tcatgaccaaaatcccttaacgtgagttttcgttccactga |
| | | | gcgtcagaccccgtagaaaagatcaaaggatcttcttgaga |
| | | | tcctttttttctgcgcgtaatctgctgcttgcaaacaaaaa |
| | | | aaccaccgctaccagcggtggtttgtttgccggatcaagag |
| | | | ctaccaactctttttccgaaggtaactggcttcagcagagc |
| | | | gcagataccaaatactgtccttctagtgtagccgtagttag |
| | | | gccaccacttcaagaactctgtagcaccgcctacatacctc |
| | | | gctctgctaatcctgttaccagtggctgctgccagtggcga |
| | | | taagtcgtgtcttaccgggttggactcaagacgatagttac |
| | | | cggataaggcgcagcggtcgggctgaacggggggttcgtgc |
| | | | acacagcccagcttggagcgaacgacctacaccgaactgag |
| | | | atacctacagcgtgagctatgagaaagcgccacgcttcccg |
| | | | aagggagaaaggcggacaggtatccggtaagcggcagggtc |
| | | | ggaacaggagagcgcacgagggagcttccagggggaaacgc |
| | | | ctggtatctttatagtcctgtcgggtttcgccacctctgac |
| | | | ttgagcgtcgatttttgtgatgctcgtcaggggggcggagc |
| | | | ctatggaaaaacgccagcaacgcggcctttttacggttcct |
| | | | gggcttttgctggccttttgctcacatgttctttcctgcgt |
| | | | tatcccctgattctgtggataaccgtattaccgcctttgag |
| | | | tgagctgataccgctcgccgcagccgaacgaccgagcgcag |
| | | | cgagtcagtgagcgaggaagcggaagagcgcccaatacgca |
| | | | aaccgcctctccccgcgcgttggccgattcattaatgcagc |
| | | | tggcacgacaggtttcccgactggaaagcgggcagtgagcg |
| | | | caacgcaattaatgtgagttagctcactcattaggcacccc |
| | | | aggctttacactttatgcttccggctcgtatgttgtgtgga |
| | | | attgtgagcggataacaatttcacacaggaaacagctatga |
| | | | ccatgattacgccaagctatttaggtgacactatagaatac |
| | | | tcaagctatgcatcaagcttggtaccgagctcggatccact |
| | | | agtaacggccgccagtgtgctggaattcgcccttttgtctc |
| | | | gcatggatgcacgaatgaacgactcgcctccaagcatattt |
| | | | atagctttgtcgacgttcttgacgttcaacgggagatcgat |
| | | | ggccgctacacgcgggatatccattgaatgttcatctggtc |
| | | | tttccaactctggcatggtgatggatgaagtgttggttgtc |
| | | | tgagacagatgggcttgttttgattttttggtgattttttc |
| | | | tttttccagagagtacaaaactgtgcagccgacaagaatct |
| | | | ggcaggacagcaccagttggaaattttggcaacacagtttc |
| | | | aattgaccactggtggagtgttgctacaagggttggtgata |
| | | | ctaagcagtgactcaattgacaccaggctgtacttttagac |
| | | | attcaattgaactgctgcattgccgtggggcagactactag |
| | | | aagtgtcctctcaatagctcgaaccacttgaaacacattac |
| | | | atcgtggcttaactgtatctacggttgtttcggtatgaccc |
| | | | ccggggatctgacgggtacaacgagaattgtattgaattga |
| | | | tcaagaacatgatcttggtgttacagaacatcaagttcttg |
| | | | gaccagactgagaatgcacagatatacaaggcgtcatgtga |
| | | | taaaatggatgagatttatccacaattgaagaaagagttta |
| | | | tggaaagtggtcaaccagaagctaaacaggaagaagcaaac |
| | | | gaagaggtgaaacaagaagaagaaggtaaataagtattttg |
| | | | tattatataacaaacaaagtaaggaatacagatttatacaa |
| | | | taaattgccatactagtcacgtgagatatctcatccattcc |
| | | | ccaactcccaagaaaaaaaaaaagtgaaaaaaaaaatcaaa |
| | | | cccaaagatcaacctccccatcatcatcgtcatcaaacccc |
| | | | cagctcaattcgcaatggttagcacaaaaacatacacagaa |
| | | | agggcatcagcacacccctccnaggttgcccaacgtttatt |
| | | | ccgcttaatggagtccaaaaagaccaacctctgcgcctcga |
| | | | tcgacgtgaccacaaccgccgagttcctttcgctcatcgac |
| | | | aagctcggtccccacatctgtctcgtgaagacgcacatcga |
| | | | tntcatctcagacttcagctacgagggcacgattgagccgt |
| | | | tgcttgtgcttgcagagcgccacgggttcttgatattcgag |
| | | | gacaggaagtttgctgatatcggaaacaccgtgatgttgca |
| | | | gtacacctcgggggtataccggatcgcggcgtggagtgaca |
| | | | tcacgaacgcgcacggagtgactgggaagggcgtcgttgaa |
| | | | gggttgaaacgcggtgcggagggggtagaaaaggaaagggg |
| | | | cgtgttgatgtnggcggagttgtcgagtaaaggctcgttgg |
| | | | cgcatggtgaatatacccgtgagacgatcgagattgcgaag |
| | | | agtgatcgggagttcgtgattgggttcatcgcgcagcggga |
| | | | catggggggtagagaagaagggtttgattggatcatcatga |
| | | | cgcctggtgtggggttggatgataaaggcgatgcgttgggc |
| | | | cagcagtataggactgttgatgaggtggttctgactggtac |
| | | | cgatgtgattattgtcgggagagggttgtttggaaaaggaa |
| | | | gagaccctgaggtggagggaaagagatacagggatgctgga |
| | | | tggaaggcatacttgaagagaactggtcagttagaataaat |
| | | | attgtaataaataggtctatatacatacactaagcttctag |
| | | | gacgtcattgtagtcttcgaagttgtctgctagtttagttc |
| | | | tcatgatttcgaaaaccaataacgcaatggatgtagcaggg |
| | | | atggtggttagtgcgttcctgacaaacccagagtacgccgc |
| | | | ctcaaaccacgtcacattcgccctttgcttcatccgcatca |
| | | | cttgcttgaaggtatccacgtacgagttgtaatacaccttg |
| | | | aagaacggcttcgtctacggtcgacgacgggtacaacgaga |
| | | | attgtattgaattgatcaagaacatgatcttggtgttacag |
| | | | aacatcaagttcttggaccagactgagaatgcacagatata |
| | | | caaggcgtcatgtgataaaatggatgagatttatccacaat |
| | | | tgaagaaagagtttatggaaagtggtcaaccagaagctaaa |
| | | | caggaagaagcaaacgaagaggtgaaacaagaagaagaagg |
| | | | taaataagtattttgtattatataacaaacaaagtaaggaa |
| | | | tacagatttatacaataaattgccatactagtcacgtgaga |
| | | | tatctcatccattccccaactcccaagaaaaaaaaaaagtg |
| | | | aaaaaaaaaatcaaacccaaagatcaacctccccatcatca |
| | | | tcgtcatcaaacccccagctcaattcgcagagctcggtacc |
| | | | aaatgggtcaacagaatccaattcggtggtgacgaagttgt |
| | | | caaggctaaggatggtgctggttccgccactttgtccatgg |
| | | | ctcaagctggtgctagattcgccggtgccgtcttggacggt |
| | | | ttggctggtgaaaaggacgtcattgaatgtacctttgtcga |
| | | | ctccccattgttcaagaacgaaggtgtcgaattcttctcct |
| | | | ccaaggttaccttgggtgttgacggtgtcaagactgtccac |
| | | | ccagttggcaacatttctgagtacgaagaagctcaagtcaa |
| | | | ggaagccaaggacactttgatcaagaacatcaagaagggtg |
| | | | tcgactttgttgctcaaaacccataa |
| 15 | PCR primer | Artificial DNA | tgggtcaacagaatccaattcggtgtactcgtagcaacctg |
| | | | ttctttctt |
| 16 | PCR primer | Artificial DNA | aagaaagaacaggttgctacgagtacaccgaattggattct |
| | | | gttgaccca |
| 17 | malonate- | Candida sp. | atgttatccagagttcttttcaagactaaaccaagagttcc |
| | semialdehyde | polynucleotide | tactaaatcaatcaccgccatggccatcagaaacaaatcca |
| | dehydrogenase | | tcgtgactttatcctccaccacctccacatacccaaccgac |
| | (acetylating) | | cacacgaccccgtccacggagccatacatcacgccatcctt |
| | (EC 1.2.1.18) | | cgtgaacaacgagttcatcaagtcggactccaacacctggt |
| | | | tcgacgtgcacgacccggccacgaactacgtcgtgtccaag |
| | | | gtgccacagtcgacgccggaggagttggaagaggcgatcgc |
| | | | gtcggcccatgccgcgttccccaagtggcgcgacaccagca |
| | | | tcatcaagcgtcaggggatcgcgttcaagtttgtgcagttg |
| | | | ttgcgcgagaacatggacagaatcgcaagcgtcattgtctt |
| | | | ggaacagggtaagacgtttgtcgatgcccagggtgacgtga |
| | | | ctagaggattgcaggttgctgaggctgcgtgcaacatcact |
| | | | aatgacttgaagggtgagtcgttggaagtgtctactgatat |
| | | | ggagaccaagatgattagagaacctttgggtgttgtgggat |
| | | | ccatctgtccttttaacttcccagctatggtcccattgtgg |
| | | | tctttgcctttggttttggtcacgggtaacactgctgtgat |
| | | | taagccttccgagagagtcccgggcgcaagtatgattattt |
| | | | gtgaattggccgccaaggctggtgttccacctggtgtgttg |
| | | | aacattgtccacggtaagcacgacaccgtcaacaagttgat |
| | | | tgaggacccaagaatcaaggcattgacttttgttggtggtg |
| | | | acaaggccggtaagtacatttacgaaaagggttccagtttg |
| | | | ggcaagagagtgcaggccaacttgggtgctaagaaccactt |
| | | | ggttgtgttgccagacgcacacaagcagagttttgtcaatg |
| | | | ccgtcaacggtgccgctttcggtgctgctggacagagatgt |
| | | | atggctatttctgtcttggtcaccgtgggtaagaccaagga |
| | | | atgggtgcaggatgtcatcaaggacgccaagttgttgaaca |
| | | | ccggaagtggatttgacccaaagagtgacttgggtccagtc |
| | | | atcaacccagagtccttgactcgtgctgaagaaatcattgc |
| | | | tgattccgtggccaacggtgccgtgttggaattggacggaa |
| | | | gaggatacagaccagaagacgctagattcgccaagggtaac |
| | | | ttcttgggtccaaccatcttgaccaacgtcaagccaggctt |
| | | | gagagcatacgacgaagagattttcgctcctgttttgtctg |
| | | | tggttaacgtcgacaccattgacgaagccattgagttgatc |
| | | | aacaacaacaagtacggtaacggtgtttcattatttacttc |
| | | | ctccggtggctcagcccagtatttcaccaagagaatcgacg |
| | | | tcggtcaagtcggtatcaatgtcccaatccctgttccattg |
| | | | cctatgttctccttcactggttccagaggctccttcttggg |
| | | | tgacttgaacttctacggtaaggccggtatcaccttcttga |
| | | | ccaagccaaagaccatcactagtgcctggaagaccaacttg |
| | | | attgatgacgagatcttgaaaccatctacctcgatgcctgt |
| | | | ccaacagtaa |
| 18 | malonate- | Candida sp. | mlsrvlfktkprvptksitamairnksivtlssttstyptd |
| | semialdehyde | polypeptide | httpstepyitpsfvnnefiksdsntwfdvhdpatnyvvsk |
| | dehydrogenase | | vpgstpeeleeaiasahaafpkwrdtsiikrggiafkfvql |
| | (acetylating) | | lrenmdriasvivleggktfvdaggdvtrglqvaeaacnit |
| | (EC 1.2.1.18) | | ndlkgeslevstdmetkmireplgvvgsicpfnfpamvplw |
| | | | slplvlvtgntavikpservpgasmiicelaakagvppgvl |
| | | | nivhgkhdtvnkliedprikaltfvggdkagkyiyekgssl |
| | | | gkrvganlgaknhlvvlpdahkgsfvnavngaafgaaggrc |
| | | | maisvlvtvgktkewvqdvikdakllntgsgfdpksdlgpv |
| | | | inpesltraeeiiadsvangavleldgrgyrpedarfakgn |
| | | | flgptiltnvkpglraydeeifapvlsvvnvdtideaieli |
| | | | nnnkygngvslftssggsagyftkridvgqvginvpipvpl |
| | | | pmfsftgsrgsflgdlnfygkagitfltkpktitsawktnl |
| | | | iddeilkpstsmpvgq |
| 19 | ALD6 deletion | Artificial DNA | tatcacagcacacacgacctactcatcaaccacccagaatc |
| | cassette | | accgctagctggcaccgcgaactggaaggcattgggagata |
| | | | ataaggttgtattgtgggtgtcgggtattgttaagggtatg |
| | | | tacgtaaggtggggggagaagggtgtgtgtgtgcttcggtg |
| | | | cgtcgcccctccacccctcctttcttcccgttgctcggccg |
| | | | ttgatacccatggctaatatcctacccttttactatttgat |
| | | | ccccacaattgctcctatggaggctggtgcacacacgactg |
| | | | aaaattagagagagagagagaaggatttcgatatcctataa |
| | | | tttcacattcagtggttaagcgcctacctgtctctttccct |
| | | | ctcccgcaaaagtatttaaacaaccaacaatacctcttctc |
| | | | tgttttacctcttgtccgagtttttcacaaatacctcccga |
| | | | gttctgctgcaagtactactcttctttccatcatgttatcc |
| | | | agagttcttgtatctacggttgtttcggtatgacccccggg |
| | | | gatctgacgggtacaacgagaattgtattgaattgatcaag |
| | | | aacatgatcttggtgttacagaacatcaagttcttggacca |
| | | | gactgagaatgcacagatatacaaggcgtcatgtgataaaa |
| | | | tggatgagatttatccacaattgaagaaagagtttatggaa |
| | | | agtggtcaaccagaagctaaacaggaagaagcaaacgaaga |
| | | | ggtgaaacaagaagaagaaggtaaataagtattttgtatta |
| | | | tataacaaacaaagtaaggaatacagatttatacaataaat |
| | | | tgccatactagtcacgtgagatatctcatccattccccaac |
| | | | tcccaagaaaaaaaaaaagtgaaaaaaaaaatcaaacccaa |
| | | | agatcaacctccccatcatcatcgtcatcaaacccccagct |
| | | | caattcgcaatggttagcacaaaaacatacacagaaagggc |
| | | | atcagcacacccctccnaggttgcccaacgtttattccgct |
| | | | taatggagtccaaaaagaccaacctctgcgcctcgatcgac |
| | | | gtgaccacaaccgccgagttcctttcgctcatcgacaagct |
| | | | cggtccccacatctgtctcgtgaagacgcacatcgatntca |
| | | | tctcagacttcagctacgagggcacgattgagccgttgctt |
| | | | gtgcttgcagagcgccacgggttcttgatattcgaggacag |
| | | | gaagtttgctgatatcggaaacaccgtgatgttgcagtaca |
| | | | cctcgggggtataccggatcgcggcgtggagtgacatcacg |
| | | | aacgcgcacggagtgactgggaagggcgtcgttgaagggtt |
| | | | gaaacgcggtgcggagggggtagaaaaggaaaggggcgtgt |
| | | | tgatgtnggcggagttgtcgagtaaaggctcgttggcgcat |
| | | | ggtgaatatacccgtgagacgatcgagattgcgaagagtga |
| | | | tcgggagttcgtgattgggttcatcgcgcagcgggacatgg |
| | | | ggggtagagaagaagggtttgattggatcatcatgacgcct |
| | | | ggtgtggggttggatgataaaggcgatgcgttgggccagca |
| | | | gtataggactgttgatgaggtggttctgactggtaccgatg |
| | | | tgattattgtcgggagagggttgtttggaaaaggaagagac |
| | | | cctgaggtggagggaaagagatacagggatgctggatggaa |
| | | | ggcatacttgaagagaactggtcagttagaataaatattgt |
| | | | aataaataggtctatatacatacactaagcttctaggacgt |
| | | | cattgtagtcttcgaagttgtctgctagtttagttctcatg |
| | | | atttcgaaaaccaataacgcaatggatgtagcagggatggt |
| | | | ggttagtgcgttcctgacaaacccagagtacgccgcctcaa |
| | | | accacgtcacattcgccctttgcttcatccgcatcacttgc |
| | | | ttgaaggtatccacgtacgagttgtaatacaccttgaagaa |
| | | | cggcttcgtctacggtcgacgacgggtacaacgagaattgt |
| | | | attgaattgatcaagaacatgatcttggtgttacagaacat |
| | | | caagttcttggaccagactgagaatgcacagatatacaagg |
| | | | cgtcatgtgataaaatggatgagatttatccacaattgaag |
| | | | aaagagtttatggaaagtggtcaaccagaagctaaacagga |
| | | | agaagcaaacgaagaggtgaaacaagaagaagaaggtaaat |
| | | | aagtattttgtattatataacaaacaaagtaaggaatacag |
| | | | atttatacaataaattgccatactagtcacgtgagatatct |
| | | | catccattccccaactcccaagaaaaaaaaaaagtgaaaaa |
| | | | aaaaatcaaacccaaagatcaacctccccatcatcatcgtc |
| | | | atcaaacccccagctcaattcgcagagctcggtaccaaatg |
| | | | ggtcaacagaatccaattcggtggaccaacgtcaagccagg |
| | | | cttgagagcatacgacgaagagattttcgctcctgttttgt |
| | | | ctgtggttaacgtcgacaccattgacgaagccattgagttg |
| | | | atcaacaacaacaagtacggtaacggtgtttcattatttac |
| | | | ttcctccggtggctcagcccagtatttcaccaagagaatcg |
| | | | acgtcggtcaagtcggtatcaatgtcccaatccctgttcca |
| | | | ttgcctatgttctccttcactggttccagaggctccttctt |
| | | | gggtgacttgaacttctacggtaaggccggtatcaccttct |
| | | | tgaccaagccaaagaccatcactagtgcctggaagaccaac |
| | | | ttgattgatgacgagatcttgaaaccatctacctcgatgcc |
| | | | tgtccaacagtaa |
| 20 | Genomic | Candida sp. | tatcacagcacacacgacctactcatcaaccacccagaatc |
| | region | polynucleotide | accgctagctggcaccgcgaactggaaggcattgggagata |
| | upstream of | | ataaggttgtattgtgggtgtcgggtattgttaagggtatg |
| | the ALD6 gene | | tacgtaaggtggggggagaagggtgtgtgtgtgcttcggtg |
| | | | cgtcgcccctccacccctcctttcttcccgttgctcggccg |
| | | | ttgatacccatggctaatatcctacccttttactatttgat |
| | | | ccccacaattgctcctatggaggctggtgcacacacgactg |
| | | | aaaattagagagagagagagaaggatttcgatatcctataa |
| | | | tttcacattcagtggttaagcgcctacctgtctctttccct |
| | | | ctcccgcaaaagtatttaaacaaccaacaatacctcttctc |
| | | | tgttttacctcttgtccgagtttttcacaaatacctcccga |
| | | | gttctgctgcaagtactactcttctttccatcatgttatcc |
| | | | agagttct |
| 21 | PCR primer | Artificial DNA | tatcacagcacacacgacctactc |
| 22 | PCR primer | Artificial DNA | tcataccgaaacaaccgtagatacaagaactctggataaca |
| | | | tgatggaaa |
| 23 | Genomic | Candida sp. | gaccaacgtcaagccaggcttgagagcatacgacgaagaga |
| | region | polynucleotide | ttttcgctcctgttttgtctgtggttaacgtcgacaccatt |
| | upstream of | | gacgaagccattgagttgatcaacaacaacaagtacggtaa |
| | the ALD6 gene | | cggtgtttcattatttacttcctccggtggctcagcccagt |
| | | | atttcaccaagagaatcgacgtcggtcaagtcggtatcaat |
| | | | gtcccaatccctgttccattgcctatgttctccttcactgg |
| | | | ttccagaggctccttcttgggtgacttgaacttctacggta |
| | | | aggccggtatcaccttcttgaccaagccaaagaccatcact |
| | | | agtgcctggaagaccaacttgattgatgacgagatcttgaa |
| | | | accatctacctcgatgcctgtccaacagtaa |
| 24 | PCR primer | Artificial DNA | ccaaatgggtcaacagaatccaattcggtggaccaacgtca |
| | | | agccaggct |
| 25 | PCR primer | Artificial DNA | ttactgttggacaggcatcgagg |
| 26 | PCR primer | Artificial DNA | tttccatcatgttatccagagttcttgtatctacggttgtt |
| | | | tcggtatga |
| 27 | PCR primer | Artificial DNA | agcctggcttgacgttggtccaccgaattggattctgttga |
| | | | cccatttgg |
| 28 | NADPH | Candida sp. | atggctttagacaagttagatttgtatgtcatcataacatt |
| | cytochrome | polynucleotide | ggtggtcgctgtagccgcctattttgctaagaaccagttcc |
| | P450 | | ttgatcagccccaggacaccgggttcctcaacacggacagc |
| | reductase A | | ggaagcaactccagagacgtcttgctgacattgaagaagaa |
| | (EC 1.6.2.4) | | taataaaaacacgttgttgttgtttgggtcccagacgggta |
| | | | cggcagaagattacgccaacaaattgtccagagaattgcac |
| | | | tccagatttggcttgaaaacgatggttgcagatttcgctga |
| | | | ttacgattgggataacttcggagatatcaccgaagacatct |
| | | | tggtgtttttcattgttgccacctatggtgagggtgaacct |
| | | | accgataatgccgacgagttccacacctggttgactgaaga |
| | | | agctgacactttgagtaccttgaaatacaccgtgttcgggt |
| | | | tgggtaactccacgtacgagttcttcaatgccattggtaga |
| | | | aagtttgacagattgttgagcgagaaaggtggtgacaggtt |
| | | | tgctgaatacgctgaaggtgatgacggtactggcaccttgg |
| | | | acgaagatttcatggcctggaaggacaatgtctttgacgcc |
| | | | ttgaagaatgatttgaactttgaagaaaaggaattgaagta |
| | | | cgaaccaaacgtgaaattgactgagagagacgacttgtctg |
| | | | ctgctgactcccaagtttccttgggtgagccaaacaagaag |
| | | | tacatcaactccgagggcatcgacttgaccaagggtccatt |
| | | | cgaccacacccacccatacttggccagaatcaccgagacga |
| | | | gagagttgttcagctccaaggacagacactgtatccacgtt |
| | | | gaatttgacatttctgaatcgaacttgaaatacaccaccgg |
| | | | tgaccatctagctatctggccatccaactccgacgaaaaca |
| | | | ttaagcaatttgccaagtgtttcggattggaagataaactc |
| | | | gacactgttattgaattgaaggcgttggactccacttacac |
| | | | catcccattcccaaccccaattacctacggtgctgtcatta |
| | | | gacaccatttagaaatctccggtccagtctcgagacaattc |
| | | | tttttgtcaattgctgggtttgctcctgatgaagaaacaaa |
| | | | gaaggcttttaccagacttggtggtgacaagcaagaattcg |
| | | | ccgccaaggtcacccgcagaaagttcaacattgccgatgcc |
| | | | ttgttatattcctccaacaacgctccatggtccgatgttcc |
| | | | ttttgaattccttattgaaaacgttccacacttgactccac |
| | | | gttactactccatttcgtcttcgtcattgagtgaaaagcaa |
| | | | ctcatcaacgttactgcagttgttgaagccgaagaagaagc |
| | | | tgatggcagaccagtcactggtgttgtcaccaacttgttga |
| | | | agaacgttgaaattgtgcaaaacaagactggcgaaaagcca |
| | | | cttgtccactacgatttgagcggcccaagaggcaagttcaa |
| | | | caagttcaagttgccagtgcatgtgagaagatccaacttta |
| | | | agttgccaaagaactccaccaccccagttatcttgattggt |
| | | | ccaggtactggtgttgccccattgagaggttttgtcagaga |
| | | | aagagttcaacaagtcaagaatggtgtcaatgttggcaaga |
| | | | ctttgttgttttatggttgcagaaactccaacgaggacttt |
| | | | ttgtacaagcaagaatgggccgagtacgcttctgttttggg |
| | | | tgaaaactttgagatgttcaatgccttctccagacaagacc |
| | | | catccaagaaggtttacgtccaggataagattttagaaaac |
| | | | agccaacttgtgcacgagttgttgactgaaggtgccattat |
| | | | ctacgtctgtggtgatgccagtagaatggctagagacgtgc |
| | | | agaccacaatttccaagattgttgctaaaagcagagaaatt |
| | | | agtgaagacaaggctgctgaattggtcaagtcctggaaggt |
| | | | ccaaaatagataccaagaagatgtttggtag |
| 29 | NADPH | Candida sp. | maldkldlyviitivvavaayfaknqfldqpqdtgflntds |
| | cytochrome | polypeptide | gsnsrdvlstlkknnkntlllfgsqtgtaedyanklsrelh |
| | P450 | | srfglktmvadfadydwdnfgditedilvffivatygegep |
| | reductase A | | tdnadefhtwlteeadtlstlkytvfglgnstyeffnaigr |
| | (EC 1.6.2.4) | | kfdrllsekggdrfaeyaegddgtgtldedfmawkdnvfda |
| | | | lkndlnfeekelkyepnvklterddlsaadsqvslgepnkk |
| | | | yinsegidltkgpfdhthpylaritetrelfsskdrhcihv |
| | | | efdisesnlkyttgdhlaiwpsnsdenikqfakcfgledkl |
| | | | dtvielkaldstytipfptpitygavirhhleisgpvsrqf |
| | | | flsiagfapdeetkkaftrlggdkqefaakvtrrkfniada |
| | | | llyssnnapwsdvpfeflienvphltpryysisssslsekq |
| | | | linvtavveaeeeadgrpvtgvvtnllknveivqnktgekp |
| | | | lvhydlsgprgkfnkfklpvhvrrsnfklpknsttpvilig |
| | | | pgtgvaplrgfvrervqqvkngvnvgktllfygcrnsnedf |
| | | | lykqewaeyasvlgenfemfnafsrqdpskkvyvqdkilen |
| | | | sqlvhelltegaiiyvcgdasrmardvqttiskivaksrei |
| | | | sedkaaelvkswkvqnryqedvw |
| 30 | NADPH | Candida sp. | atggctttagacaagttagatttgtatgtcatcataacatt |
| | cytochrome | polynucleotide | ggtggtcgctgtggccgcctattttgctaagaaccagttcc |
| | P450 | | ttgatcagccccaggacaccgggttcctcaacacggacagc |
| | reductase B | | ggaagcaactccagagacgtcttgctgacattgaagaagaa |
| | (EC 1.6.2.4) | | taataaaaacacgttgttgttgtttgggtcccagaccggta |
| | | | cggcagaagattacgccaacaaattgtcaagagaattgcac |
| | | | tccagatttggcttgaaaaccatggttgcagatttcgctga |
| | | | ttacgattgggataacttcggagatatcaccgaagatatct |
| | | | tggtgtttttcatcgttgccacctacggtgagggtgaacct |
| | | | accgacaatgccgacgagttccacacctggttgactgaaga |
| | | | agctgacactttgagtactttgagatataccgtgttcgggt |
| | | | tgggtaactccacctacgagttcttcaatgctattggtaga |
| | | | aagtttgacagattgttgagtgagaaaggtggtgacagatt |
| | | | tgctgaatatgctgaaggtgacgacggcactggcaccttgg |
| | | | acgaagatttcatggcctggaaggataatgtctttgacgcc |
| | | | ttgaagaatgacttgaactttgaagaaaaggaattgaagta |
| | | | cgaaccaaacgtgaaattgactgagagagatgacttgtctg |
| | | | ctgccgactcccaagtttccttgggtgagccaaacaagaag |
| | | | tacatcaactccgagggcatcgacttgaccaagggtccatt |
| | | | cgaccacacccacccatacttggccaggatcaccgagacca |
| | | | gagagttgttcagctccaaggaaagacactgtattcacgtt |
| | | | gaatttgacatttctgaatcgaacttgaaatacaccaccgg |
| | | | tgaccatctagccatctggccatccaactccgacgaaaaca |
| | | | tcaagcaatttgccaagtgtttcggattggaagataaactc |
| | | | gacactgttattgaattgaaggcattggactccacttacac |
| | | | cattccattcccaactccaattacttacggtgctgtcatta |
| | | | gacaccatttagaaatctccggtccagtctcgagacaattc |
| | | | tttttgtcgattgctgggtttgctcctgatgaagaaacaaa |
| | | | gaagactttcaccagacttggtggtgacaaacaagaattcg |
| | | | ccaccaaggttacccgcagaaagttcaacattgccgatgcc |
| | | | ttgttatattcctccaacaacactccatggtccgatgttcc |
| | | | ttttgagttccttattgaaaacatccaacacttgactccac |
| | | | gttactactccatttcttcttcgtcgttgagtgaaaaacaa |
| | | | ctcatcaatgttactgcagtcgttgaggccgaagaagaagc |
| | | | cgatggcagaccagtcactggtgttgttaccaacttgttga |
| | | | agaacattgaaattgcgcaaaacaagactggcgaaaagcca |
| | | | cttgttcactacgatttgagcggcccaagaggcaagttcaa |
| | | | caagttcaagttgccagtgcacgtgagaagatccaacttta |
| | | | agttgccaaagaactccaccaccccagttatcttgattggt |
| | | | ccaggtactggtgttgccccattgagaggtttcgttagaga |
| | | | aagagttcaacaagtcaagaatggtgtcaatgttggcaaga |
| | | | ctttgttgttttatggttgcagaaactccaacgaggacttt |
| | | | ttgtacaagcaagaatgggccgagtacgcttctgttttggg |
| | | | tgaaaactttgagatgttcaatgccttctctagacaagacc |
| | | | catccaagaaggtttacgtccaggataagattttagaaaac |
| | | | agccaacttgtgcacgaattgttgaccgaaggtgccattat |
| | | | ctacgtctgtggtgacgccagtagaatggccagagacgtcc |
| | | | agaccacgatctccaagattgttgccaaaagcagagaaatc |
| | | | agtgaagacaaggccgctgaattggtcaagtcctggaaagt |
| | | | ccaaaatagataccaagaagatgtttgg |
| 31 | NADPH | Candida sp. | maldkldlyviitivvavaayfaknqfldqpqdtgflntds |
| | cytochrome | polypeptide | gsnsrdvlstlkknnkntlllfgsqtgtaedyanklsrelh |
| | P450 | | srfglktmvadfadydwdnfgditedilvffivatygegep |
| | reductase B | | tdnadefhtwlteeadtlstlrytvfglgnstyeffnaigr |
| | (EC 1.6.2.4) | | kfdrllsekggdrfaeyaegddgtgtldedfmawkdnvfda |
| | | | lkndlnfeekelkyepnvklterddlsaadsqvslgepnkk |
| | | | yinsegidltkgpfdhthpylaritetrelfsskerhcihv |
| | | | efdisesnlkyttgdhlaiwpsnsdenikqfakcfgledkl |
| | | | dtvielkaldstytipfptpitygavirhhleisgpvsrqf |
| | | | flsiagfapdeetkktftrlggdkqefatkvtrrkfniada |
| | | | llyssnntpwsdvpfeflienighltpryysisssslsekg |
| | | | linvtavveaeeeadgrpvtgvvtnllknieiaqnktgekp |
| | | | lvhydlsgprgkfnkfklpvhvrrsnfklpknsttpvilig |
| | | | pgtgvaplrgfvrervqqvkngvnvgktllfygcrnsnedf |
| | | | lykqewaeyasvlgenfemfnafsrqdpskkvyvqdkilen |
| | | | sqlvhelltegaiiyvcgdasrmardvqttiskivaksrei |
| | | | sedkaaelvkswkvqnryqedvw |
| 32 | Cytochrome P- | Candida sp. | atggccacacaagaaatcatcgattctgtacttccgtactt |
| | 450 | polynucleotide | gaccaaatggtacactgtgattactgcagcagtattagtct |
| | monooxygenase | | tccttatctccacaaacatcaagaactacgtcaaggcaaag |
| | CYP52A12 | | aaattgaaatgtgtcgatccaccatacttgaaggatgccgg |
| | (EC | | tctcactggtattctgtctttgatcgccgccatcaaggcca |
| | 1.14.14.1) | | agaacgacggtagattggctaactttgccgatgaagttttc |
| | | | gacgagtacccaaaccacaccttctacttgtctgttgccgg |
| | | | tgctttgaagattgtcatgactgttgacccagaaaacatca |
| | | | aggctgtcttggccacccaattcactgacttctccttgggt |
| | | | accagacacgcccactttgctcctttgttgggtgacggtat |
| | | | cttcaccttggacggagaaggttggaagcactccagagcta |
| | | | tgttgagaccacagtttgctagagaccagattggacacgtt |
| | | | aaagccttggaaccacacatccaaatcatggctaagcagat |
| | | | caagttgaaccagggaaagactttcgatatccaagaattgt |
| | | | tctttagatttaccgtcgacaccgctactgagttcttgttt |
| | | | ggtgaatccgttcactccttgtacgatgaaaaattgggcat |
| | | | cccaactccaaacgaaatcccaggaagagaaaactttgccg |
| | | | ctgctttcaacgtttcccaacactacttggccaccagaagt |
| | | | tactcccagactttttactttttgaccaaccctaaggaatt |
| | | | cagagactgtaacgccaaggtccaccacttggccaagtact |
| | | | ttgtcaacaaggccttgaactttactcctgaagaactcgaa |
| | | | gagaaatccaagtccggttacgttttcttgtacgaattggt |
| | | | taagcaaaccagagatccaaaggtcttgcaagatcaattgt |
| | | | tgaacattatggttgccggaagagacaccactgccggtttg |
| | | | ttgtcctttgctttgtttgaattggctagacacccagagat |
| | | | gtggtccaagttgagagaagaaatcgaagttaactttggtg |
| | | | ttggtgaagactcccgcgttgaagaaattaccttcgaagcc |
| | | | ttgaagagatgtgaatacttgaaggctatccttaacgaaac |
| | | | cttgcgtatgtacccatctgttcctgtcaactttagaaccg |
| | | | ccaccagagacaccactttgccaagaggtggtggtgctaac |
| | | | ggtaccgacccaatctacattcctaaaggctccactgttgc |
| | | | ttacgttgtctacaagacccaccgtttggaagaatactacg |
| | | | gtaaggacgctaacgacttcagaccagaaagatggtttgaa |
| | | | ccatctactaagaagttgggctgggcttatgttccattcaa |
| | | | cggtggtccaagagtctgcttgggtcaacaattcgccttga |
| | | | ctgaagcttcttatgtgatcactagattggcccagatgttt |
| | | | gaaactgtctcatctgatccaggtctcgaataccctccacc |
| | | | aaagtgtattcacttgaccatgagtcacaacgatggtgtct |
| | | | ttgtcaagatgtaa |
| 33 | Cytochrome P- | Candida sp. | matgeiidsvlpyltkwytvitaavlvflistniknyvkak |
| | 450 | polypeptide | klkcvdppylkdagltgissliaaikakndgrlanfadevf |
| | monooxygenase | | deypnhtfylsvagalkivmtvdpenikavlatqftdfslg |
| | CYP52A12 | | trhahfapllgdgiftldgegwkhsramlrpgfardgighv |
| | (EC | | kalephigimakqiklnqgktfdigelffrftvdtateflf |
| | 1.14.14.1) | | gesvhslydeklgiptpneipgrenfaaafnvsqhylatrs |
| | | | ysqtfyfltnpkefrdcnakvhhlakyfvnkalnftpeele |
| | | | eksksgyvflyelvkqtrdpkvlqdqllnimvagrdttagl |
| | | | lsfalfelarhpemwsklreeievnfgvgedsrveeitfea |
| | | | lkrceylkailnetlrmypsvpvnfrtatrdttlprgggan |
| | | | gtdpiyipkgstvayvvykthrleeyygkdandfrperwfe |
| | | | pstkklgwayvpfnggprvclgqgfalteasyvitrlagmf |
| | | | etvssdpgleypppkcihltmshndgvfvkm* |
| 34 | Cytochrome P- | Candida sp. | atgactgtacacgatattatcgccacatacttcaccaaatg |
| | 450 | polynucleotide | gtacgtgatagtaccactcgctttgattgcttatagagtcc |
| | monooxygenase | | tcgactacttctatggcagatacttgatgtacaagcttggt |
| | CYP52A13 | | gctaaaccatttttccagaaacagacagacggctgtttcgg |
| | (EC | | attcaaagctccgcttgaattgttgaagaagaagagcgacg |
| | 1.14.14.1) | | gtaccctcatagacttcacactccagcgtatccacgatctc |
| | | | gatcgtcccgatatcccaactttcacattcccggtcttttc |
| | | | catcaaccttgtcaatacccttgagccggagaacatcaagg |
| | | | ccatcttggccactcagttcaacgatttctccttgggtacc |
| | | | agacactcgcactttgctcctttgttgggtgatggtatctt |
| | | | tacgttggatggcgccggctggaagcacagcagatctatgt |
| | | | tgagaccacagtttgccagagaacagatttcccacgtcaag |
| | | | ttgttggagccacacgttcaggtgttcttcaaacacgtcag |
| | | | aaaggcacagggcaagacttttgacatccaggaattgtttt |
| | | | tcagattgaccgtcgactccgccaccgagtttttgtttggt |
| | | | gaatccgttgagtccttgagagatgaatctatcggcatgtc |
| | | | catcaatgcgcttgactttgacggcaaggctggctttgctg |
| | | | atgcttttaactattcgcagaattatttggcttcgagagcg |
| | | | gttatgcaacaattgtactgggtgttgaacgggaaaaagtt |
| | | | taaggagtgcaacgctaaagtgcacaagtttgctgactact |
| | | | acgtcaacaaggctttggacttgacgcctgaacaattggaa |
| | | | aagcaggatggttatgtgtttttgtacgaattggtcaagca |
| | | | aaccagagacaagcaagtgttgagagaccaattgttgaaca |
| | | | tcatggttgctggtagagacaccaccgccggtttgttgtcg |
| | | | tttgttttctttgaattggccagaaacccagaagttaccaa |
| | | | caagttgagagaagaaattgaggacaagtttggactcggtg |
| | | | agaatgctagtgttgaagacatttcctttgagtcgttgaag |
| | | | tcctgtgaatacttgaaggctgttctcaacgaaaccttgag |
| | | | attgtacccatccgtgccacagaatttcagagttgccacca |
| | | | agaacactaccctcccaagaggtggtggtaaggacgggttg |
| | | | tctcctgttttggtgagaaagggtcagaccgttatttacgg |
| | | | tgtctacgcagcccacagaaacccagctgtttacggtaagg |
| | | | acgctcttgagtttagaccagagagatggtttgagccagag |
| | | | acaaagaagcttggctgggccttcctcccattcaacggtgg |
| | | | tccaagaatctgtttgggacagcagtttgccttgacagaag |
| | | | cttcgtatgtcactgtcaggttgctccaggagtttgcacac |
| | | | ttgtctatggacccagacaccgaatatccacctaagaaaat |
| | | | gtcgcatttgaccatgtcgcttttcgacggtgccaatattg |
| | | | agatgtattag |
| 35 | Cytochrome P- | Candida sp. | mtvhdiiatyftkwyvivplaliayrvldyfygrylmyklg |
| | 450 | polypeptide | akpffqkqtdgcfgfkaplellkkksdgtlidftlgrihdl |
| | monooxygenase | | drpdiptftfpvfsinlvntlepenikailatqfndfslgt |
| | CYP52A13 | | rhshfapllgdgiftldgagwkhsrsmlrpgfaregishvk |
| | (EC | | llephvgvffkhvrkaggktfdigelffrltvdsateflfg |
| | 1.14.14.1) | | esveslrdesigmsinaldfdgkagfadafnysqnylasra |
| | | | vmqqlywvingkkfkecnakvhkfadyyvnkaldltpeqle |
| | | | kgdgyvflyelvkqtrdkqvirdqllnimvagrdttaglls |
| | | | fvffelarnpevtnklreeiedkfglgenasvedisfeslk |
| | | | sceylkavinetlrlypsvpqnfrvatknttlprgggkdgl |
| | | | spvlvrkgqtviygvyaahrnpavygkdalefrperwfepe |
| | | | tkklgwaflpfnggpriclgqqfalteasyvtvrllgefah |
| | | | lsmdpdteyppkkmshltmslfdganiemy* |
| 36 | Cytochrome P- | Candida sp. | atgactgcacaggatattatcgccacatacatcaccaaatg |
| | 450 | polynucleotide | gtacgtgatagtaccactcgctttgattgcttatagggtcc |
| | monooxygenase | | tcgactacttttacggcagatacttgatgtacaagcttggt |
| | CYP52A14 | | gctaaaccgtttttccagaaacaaacagacggttatttcgg |
| | (EC | | attcaaagctccacttgaattgttaaaaaagaagagtgacg |
| | 1.14.14.1) | | gtaccctcatagacttcactctcgagcgtatccaagcgctc |
| | | | aatcgtccagatatcccaacttttacattcccaatcttttc |
| | | | catcaaccttatcagcacccttgagccggagaacatcaagg |
| | | | ctatcttggccacccagttcaacgatttctccttgggcacc |
| | | | agacactcgcactttgctcctttgttgggcgatggtatctt |
| | | | taccttggacggtgccggctggaagcacagcagatctatgt |
| | | | tgagaccacagtttgccagagaacagatttcccacgtcaag |
| | | | ttgttggagccacacatgcaggtgttcttcaagcacgtcag |
| | | | aaaggcacagggcaagacttttgacatccaagaattgtttt |
| | | | tcagattgaccgtcgactccgccactgagtttttgtttggt |
| | | | gaatccgttgagtccttgagagatgaatctattgggatgtc |
| | | | catcaatgcacttgactttgacggcaaggctggctttgctg |
| | | | atgcttttaactactcgcagaactatttggcttcgagagcg |
| | | | gttatgcaacaattgtactgggtgttgaacgggaaaaagtt |
| | | | taaggagtgcaacgctaaagtgcacaagtttgctgactatt |
| | | | acgtcagcaaggctttggacttgacacctgaacaattggaa |
| | | | aagcaggatggttatgtgttcttgtacgagttggtcaagca |
| | | | aaccagagacaggcaagtgttgagagaccagttgttgaaca |
| | | | tcatggttgccggtagagacaccaccgccggtttgttgtcg |
| | | | tttgttttctttgaattggccagaaacccagaggtgaccaa |
| | | | caagttgagagaagaaatcgaggacaagtttggtcttggtg |
| | | | agaatgctcgtgttgaagacatttcctttgagtcgttgaag |
| | | | tcatgtgaatacttgaaggctgttctcaacgaaactttgag |
| | | | attgtacccatccgtgccacagaatttcagagttgccacca |
| | | | aaaacactacccttccaaggggaggtggtaaggacgggtta |
| | | | tctcctgttttggtcagaaagggtcaaaccgttatgtacgg |
| | | | tgtctacgctgcccacagaaacccagctgtctacggtaagg |
| | | | acgcccttgagtttagaccagagaggtggtttgagccagag |
| | | | acaaagaagcttggctgggccttccttccattcaacggtgg |
| | | | tccaagaatttgcttgggacagcagtttgccttgacagaag |
| | | | cttcgtatgtcactgtcagattgctccaagagtttggacac |
| | | | ttgtctatggaccccaacaccgaatatccacctaggaaaat |
| | | | gtcgcatttgaccatgtcccttttcgacggtgccaacattg |
| | | | agatgtattag |
| 37 | Cytochrome P- | Candida sp. | mtaqdiiatyitkwyvivplaliayrvldyfygrylmyklg |
| | 450 | polypeptide | akpffqkqtdgyfgfkaplellkkksdgtlidftlerigal |
| | monooxygenase | | nrpdiptftfpifsinlistlepenikailatqfndfslgt |
| | CYP52A14 | | rhshfapllgdgiftldgagwkhsrsmlrpgfaregishvk |
| | (EC | | llephmqvffkhvrkaggktfdigelffrltvdsateflfg |
| | 1.14.14.1) | | esveslrdesigmsinaldfdgkagfadafnysqnylasra |
| | | | vmqqlywvingkkfkecnakvhkfadyyvskaldltpeqle |
| | | | kgdgyvflyelvkqtrdrqvirdqllnimvagrdttaglls |
| | | | fvffelarnpevtnklreeiedkfglgenarvedisfeslk |
| | | | sceylkavinetlrlypsvpqnfrvatknttlprgggkdgl |
| | | | spvlvrkgqtvmygvyaahrnpavygkdalefrperwfepe |
| | | | tkklgwaflpfnggpriclgqqfalteasyvtvrllgefgh |
| | | | lsmdpnteypprkmshltmslfdganiemy* |
| 38 | Cytochrome P- | Candida sp. | atgtcgtcttctccatcgtttgcccaagaggttctcgctac |
| | 450 | polynucleotide | cactagtccttacatcgagtactttcttgacaactacacca |
| | monooxygenase | | gatggtactacttcatacctttggtgcttctttcgttgaac |
| | CYP52A15 | | tttataagtttgctccacacaaggtacttggaacgcaggtt |
| | (EC | | ccacgccaagccactcggtaactttgtcagggaccctacgt |
| | 1.14.14.1) | | ttggtatcgctactccgttgcttttgatctacttgaagtcg |
| | | | aaaggtacggtcatgaagtttgcttggggcctctggaacaa |
| | | | caagtacatcgtcagagacccaaagtacaagacaactgggc |
| | | | tcaggattgttggcctcccattgattgaaaccatggaccca |
| | | | gagaacatcaaggctgttttggctactcagttcaatgattt |
| | | | ctctttgggaaccagacacgatttcttgtactccttgttgg |
| | | | gtgacggtattttcaccttggacggtgctggctggaaacat |
| | | | agtagaactatgttgagaccacagtttgctagagaacaggt |
| | | | ttctcacgtcaagttgttggagccacacgttcaggtgttct |
| | | | tcaagcacgttagaaagcaccgcggtcaaacgttcgacatc |
| | | | caagaattgttcttcaggttgaccgtcgactccgccaccga |
| | | | gttcttgtttggtgagtctgctgaatccttgagggacgaat |
| | | | ctattggattgaccccaaccaccaaggatttcgatggcaga |
| | | | agagatttcgctgacgctttcaactattcgcagacttacca |
| | | | ggcctacagatttttgttgcaacaaatgtactggatcttga |
| | | | atggctcggaattcagaaagtcgattgctgtcgtgcacaag |
| | | | tttgctgaccactatgtgcaaaaggctttggagttgaccga |
| | | | cgatgacttgcagaaacaagacggctatgtgttcttgtacg |
| | | | agttggctaagcaaaccagagacccaaaggtcttgagagac |
| | | | cagttattgaacattttggttgccggtagagacacgaccgc |
| | | | cggtttgttgtcatttgttttctacgagttgtcaagaaacc |
| | | | ctgaggtgtttgctaagttgagagaggaggtggaaaacaga |
| | | | tttggactcggtgaagaagctcgtgttgaagagatctcgtt |
| | | | tgagtccttgaagtcttgtgagtacttgaaggctgtcatca |
| | | | atgaaaccttgagattgtacccatcggttccacacaacttt |
| | | | agagttgctaccagaaacactaccctcccaagaggtggtgg |
| | | | tgaagatggatactcgccaattgtcgtcaagaagggtcaag |
| | | | ttgtcatgtacactgttattgctacccacagagacccaagt |
| | | | atctacggtgccgacgctgacgtcttcagaccagaaagatg |
| | | | gtttgaaccagaaactagaaagttgggctgggcatacgttc |
| | | | cattcaatggtggtccaagaatctgtttgggtcaacagttt |
| | | | gccttgaccgaagcttcatacgtcactgtcagattgctcca |
| | | | ggagtttgcacacttgtctatggacccagacaccgaatatc |
| | | | caccaaaattgcagaacaccttgaccttgtcgctctttgat |
| | | | ggtgctgatgttagaatgtactaa |
| 39 | Cytochrome P- | Candida sp. | mssspsfagevlattspyieyfldnytrwyyfiplvllsln |
| | 450 | polypeptide | fisllhtrylerrfhakplgnfvrdptfgiatpllliylks |
| | monooxygenase | | kgtvmkfawglwnnkyivrdpkykttglrivglplietmdp |
| | CYP52A15 | | enikavlatqfndfslgtrhdflysllgdgiftldgagwkh |
| | (EC | | srtmlrpgfareqvshvkllephvgvffkhvrkhrgqtfdi |
| | 1.14.14.1) | | gelffrltvdsateflfgesaeslrdesigltpttkdfdgr |
| | | | rdfadafnysqtyqayrfllqqmywilngsefrksiavvhk |
| | | | fadhyvqkaleltdddlqkqdgyvflyelakqtrdpkvlrd |
| | | | qllnilvagrdttagllsfvfyelsrnpevfaklreevenr |
| | | | fglgeearveeisfeslksceylkavinetlrlypsvphnf |
| | | | rvatrnttlprgggedgyspivvkkgqvvmytviathrdps |
| | | | iygadadvfrperwfepetrklgwayvpfnggpriclgqqf |
| | | | alteasyvtvrllgefahlsmdpdteyppklqntltlslfd |
| | | | gadvrmy* |
| 40 | Cytochrome P- | Candida sp. | atgtcgtcttctccatcgtttgctcaggaggttctcgctac |
| | 450 | polynucleotide | cactagtccttacatcgagtactttcttgacaactacacca |
| | monooxygenase | | gatggtactacttcatccctttggtgcttctttcgttgaac |
| | CYP52A16 | | ttcatcagcttgctccacacaaagtacttggaacgcaggtt |
| | (EC | | ccacgccaagccgctcggtaacgtcgtgttggatcctacgt |
| | 1.14.14.1) | | ttggtatcgctactccgttgatcttgatctacttaaagtcg |
| | | | aaaggtacagtcatgaagtttgcctggagcttctggaacaa |
| | | | caagtacattgtcaaagacccaaagtacaagaccactggcc |
| | | | ttagaattgtcggcctcccattgattgaaaccatagaccca |
| | | | gagaacatcaaagctgtgttggctactcagttcaacgattt |
| | | | ctccttgggaactagacacgatttcttgtactccttgttgg |
| | | | gcgatggtatttttaccttggacggtgctggctggaaacac |
| | | | agtagaactatgttgagaccacagtttgctagagaacaggt |
| | | | ttcccacgtcaagttgttggaaccacacgttcaggtgttct |
| | | | tcaagcacgttagaaaacaccgcggtcagacttttgacatc |
| | | | caagaattgttcttcagattgaccgtcgactccgccaccga |
| | | | gttcttgtttggtgagtctgctgaatccttgagagacgact |
| | | | ctgttggtttgaccccaaccaccaaggatttcgaaggcaga |
| | | | ggagatttcgctgacgctttcaactactcgcagacttacca |
| | | | ggcctacagatttttgttgcaacaaatgtactggattttga |
| | | | atggcgcggaattcagaaagtcgattgccatcgtgcacaag |
| | | | tttgctgaccactatgtgcaaaaggctttggagttgaccga |
| | | | cgatgacttgcagaaacaagacggctatgtgttcttgtacg |
| | | | agttggctaagcaaactagagacccaaaggtcttgagagac |
| | | | cagttgttgaacattttggttgccggtagagacacgaccgc |
| | | | cggtttgttgtcgtttgtgttctacgagttgtcgagaaacc |
| | | | ctgaagtgtttgccaagttgagagaggaggtggaaaacaga |
| | | | tttggactcggcgaagaggctcgtgttgaagagatctcttt |
| | | | tgagtccttgaagtcctgtgagtacttgaaggctgtcatca |
| | | | atgaagccttgagattgtacccatctgttccacacaacttc |
| | | | agagttgccaccagaaacactacccttccaagaggcggtgg |
| | | | taaagacggatgctcgccaattgttgtcaagaagggtcaag |
| | | | ttgtcatgtacactgtcattggtacccacagagacccaagt |
| | | | atctacggtgccgacgccgacgtcttcagaccagaaagatg |
| | | | gttcgagccagaaactagaaagttgggctgggcatatgttc |
| | | | cattcaatggtggtccaagaatctgtttgggtcagcagttt |
| | | | gccttgactgaagcttcatacgtcactgtcagattgctcca |
| | | | agagtttggaaacttgtccctggatccaaacgctgagtacc |
| | | | caccaaaattgcagaacaccttgaccttgtcactctttgat |
| | | | ggtgctgacgttagaatgttctaa |
| 41 | Cytochrome P- | Candida sp. | mssspsfagevlattspyieyfldnytrwyyfiplvllsln |
| | 450 | polypeptide | fisllhtkylerrfhakplgnvvldptfgiatpliliylks |
| | monooxygenase | | kgtvmkfawsfwnnkyivkdpkykttglrivglplietidp |
| | CYP52A16 | | enikavlatqfndfslgtrhdflysllgdgiftldgagwkh |
| | (EC | | srtmlrpgfareqvshvkllephvgvffkhvrkhrgqtfdi |
| | 1.14.14.1) | | gelffrltvdsateflfgesaeslrddsvgltpttkdfegr |
| | | | gdfadafnysqtyqayrfllqqmywilngaefrksiaivhk |
| | | | fadhyvqkaleltdddlqkqdgyvflyelakqtrdpkvlrd |
| | | | qllnilvagrdttagllsfvfyelsrnpevfaklreevenr |
| | | | fglgeearveeisfeslksceylkavinealrlypsvphnf |
| | | | rvatrnttlprgggkdgcspivvkkgqvvmytvigthrdps |
| | | | iygadadvfrperwfepetrklgwayvpfnggpriclgqqf |
| | | | alteasyvtvrllgefgnlssdpnaeyppklqntltlslfd |
| | | | gadvrmf* |
| 42 | Cytochrome P- | Candida sp. | atgattgaacaactcctagaatattggtatgtcgttgtgcc |
| | 450 | polynucleotide | agtgttgtacatcatcaaacaactccttgcatacacaaaga |
| | monooxygenase | | ctcgcgtcttgatgaaaaagttgggtgctgctccagtcaca |
| | CYP52A17 | | aacaagttgtacgacaacgctttcggtatcgtcaatggatg |
| | (EC | | gaaggctctccagttcaagaaagagggcagggctcaagagt |
| | 1.14.14.1) | | acaacgattacaagtttgaccactccaagaacccaagcgtg |
| | | | ggcacctacgtcagtattcttttcggcaccaggatcgtcgt |
| | | | gaccaaagatccagagaatatcaaagctattttggcaaccc |
| | | | agtttggtgatttttctttgggcaagaggcacactcttttt |
| | | | aagcctttgttaggtgatgggatcttcacattggacggcga |
| | | | aggctggaagcacagcagagccatgttgagaccacagtttg |
| | | | ccagagaacaagttgctcatgtgacgtcgttggaaccacac |
| | | | ttccagttgttgaagaagcatattcttaagcacaagggtga |
| | | | atactttgatatccaggaattgttctttagatttaccgttg |
| | | | attcggccacggagttcttatttggtgagtccgtgcactcc |
| | | | ttaaaggacgaatctattggtatcaaccaagacgatataga |
| | | | ttttgctggtagaaaggactttgctgagtcgttcaacaaag |
| | | | cccaggaatacttggctattagaaccttggtgcagacgttc |
| | | | tactggttggtcaacaacaaggagtttagagactgtaccaa |
| | | | gctggtgcacaagttcaccaactactatgttcagaaagctt |
| | | | tggatgctagcccagaagagcttgaaaagcaaagtgggtat |
| | | | gtgttcttgtacgagcttgtcaagcagacaagagaccccaa |
| | | | tgtgttgcgtgaccagtctttgaacatcttgttggccggaa |
| | | | gagacaccactgctgggttgttgtcgtttgctgtctttgag |
| | | | ttggccagacacccagagatctgggccaagttgagagagga |
| | | | aattgaacaacagtttggtcttggagaagactctcgtgttg |
| | | | aagagattacctttgagagcttgaagagatgtgagtacttg |
| | | | aaagcgttccttaatgaaaccttgcgtatttacccaagtgt |
| | | | cccaagaaacttcagaatcgccaccaagaacacgacattgc |
| | | | caaggggcggtggttcagacggtacctcgccaatcttgatc |
| | | | caaaagggagaagctgtgtcgtatggtatcaactctactca |
| | | | tttggaccctgtctattacggccctgatgctgctgagttca |
| | | | gaccagagagatggtttgagccatcaaccaaaaagctcggc |
| | | | tgggcttacttgccattcaacggtggtccaagaatctgttt |
| | | | gggtcagcagtttgccttgacggaagctggctatgtgttgg |
| | | | ttagattggtgcaagagttctcccacgttaggctggaccca |
| | | | gacgaggtgtacccgccaaagaggttgaccaacttgaccat |
| | | | gtgtttgcaggatggtgctattgtcaagtttgactag |
| 43 | Cytochrome P- | Candida sp. | mieqlleywyvvvpvlyiikqllaytktrvlmkklgaapvt |
| | 450 | polypeptide | nklydnafgivngwkalqfkkegrageyndykfdhsknpsv |
| | monooxygenase | | gtyvsilfgtrivvtkdpenikailatqfgdfslgkrhtlf |
| | CYP52A17 | | kpllgdgiftldgegwkhsramlrpqfareqvahvtsleph |
| | (EC | | fqllkkhilkhkgeyfdigelffrftvdsateflfgesvhs |
| | 1.14.14.1) | | lkdesigingddidfagrkdfaesfnkageylairtivqtf |
| | | | ywlvnnkefrdctksvhkftnyyvqkaldaspeelekqsgy |
| | | | vflyelvkqtrdpnvirdqslnillagrdttagllsfavfe |
| | | | larhpeiwaklreeieqqfglgedsrveeitfeslkrceyl |
| | | | kaflnetlriypsvprnfriatknttlprgggsdgtspili |
| | | | qkgeaysyginsthldpvyygpdaaefrperwfepstkklg |
| | | | waylpfnggpriclgqqfalteagyvlvrlvqefshvrsdp |
| | | | devyppkrltnitmclqdgaivkfd* |
| 44 | Cytochrome P- | Candida sp. | atgattgaacaaatcctagaatattggtatattgttgtgcc |
| | 450 | polynucleotide | tgtgttgtacatcatcaaacaactcattgcctacagcaaga |
| | monooxygenase | | ctcgcgtcttgatgaaacagttgggtgctgctccaatcaca |
| | CYP52A18 | | aaccagttgtacgacaacgttttcggtatcgtcaacggatg |
| | (EC | | gaaggctctccagttcaagaaagagggcagagctcaagagt |
| | 1.14.14.1) | | acaacgatcacaagtttgacagctccaagaacccaagcgtc |
| | | | ggcacctatgtcagtattctttttggcaccaagattgtcgt |
| | | | gaccaaggatccagagaatatcaaagctattttggcaaccc |
| | | | agtttggcgatttttctttgggcaagagacacgctcttttt |
| | | | aaacctttgttaggtgatgggatcttcaccttggacggcga |
| | | | aggctggaagcatagcagatccatgttaagaccacagtttg |
| | | | ccagagaacaagttgctcatgtgacgtcgttggaaccacac |
| | | | ttccagttgttgaagaagcatatccttaaacacaagggtga |
| | | | gtactttgatatccaggaattgttctttagatttactgtcg |
| | | | actcggccacggagttcttatttggtgagtccgtgcactcc |
| | | | ttaaaggacgaaactatcggtatcaaccaagacgatataga |
| | | | ttttgctggtagaaaggactttgctgagtcgttcaacaaag |
| | | | cccaggagtatttgtctattagaattttggtgcagaccttc |
| | | | tactggttgatcaacaacaaggagtttagagactgtaccaa |
| | | | gctggtgcacaagtttaccaactactatgttcagaaagctt |
| | | | tggatgctaccccagaggaacttgaaaagcaaggcgggtat |
| | | | gtgttcttgtatgagcttgtcaagcagacgagagaccccaa |
| | | | ggtgttgcgtgaccagtctttgaacatcttgttggcaggaa |
| | | | gagacaccactgctgggttgttgtcctttgctgtgtttgag |
| | | | ttggccagaaacccacacatctgggccaagttgagagagga |
| | | | aattgaacagcagtttggtcttggagaagactctcgtgttg |
| | | | aagagattacctttgagagcttgaagagatgtgagtacttg |
| | | | aaagcgttccttaacgaaaccttgcgtgtttacccaagtgt |
| | | | cccaagaaacttcagaatcgccaccaagaatacaacattgc |
| | | | caaggggtggtggtccagacggtacccagccaatcttgatc |
| | | | caaaagggagaaggtgtgtcgtatggtatcaactctaccca |
| | | | cttagatcctgtctattatggccctgatgctgctgagttca |
| | | | gaccagagagatggtttgagccatcaaccagaaagctcggc |
| | | | tgggcttacttgccattcaacggtgggccacgaatctgttt |
| | | | gggtcagcagtttgccttgaccgaagctggttacgttttgg |
| | | | tcagattggtgcaagagttctcccacattaggctggaccca |
| | | | gatgaagtgtatccaccaaagaggttgaccaacttgaccat |
| | | | gtgtttgcaggatggtgctattgtcaagtttgactag |
| 45 | Cytochrome P- | Candida sp. | miegileywyivvpvlyiikgliaysktrvlmkqlgaapit |
| | 450 | polypeptide | nglydnvfgivngwkalqfkkegrageyndhkfdssknpsv |
| | monooxygenase | | gtyvsilfgtkivvtkdpenikailatqfgdfslgkrhalf |
| | CYP52A18 | | kpllgdgiftldgegwkhsrsmlrpgfareqvahvtsleph |
| | (EC | | fqllkkhilkhkgeyfdigelffrftvdsateflfgesvhs |
| | 1.14.14.1) | | lkdetigingddidfagrkdfaesfnkageylsirilvqtf |
| | | | ywlinnkefrdctksvhkftnyyvqkaldatpeelekqggy |
| | | | vflyelvkqtrdpkvlrdqslnillagrdttagllsfavfe |
| | | | larnphiwaklreeieqqfglgedsrveeitfeslkrceyl |
| | | | kaflnetlrvypsvprnfriatknttlprgggpdgtqpili |
| | | | qkgegvsyginsthldpvyygpdaaefrperwfepstrklg |
| | | | waylpfnggpriclgqqfalteagyvlvrlvqefshirsdp |
| | | | devyppkrltnitmclqdgaivkfd* |
| 46 | Cytochrome P- | Candida sp. | atgctcgatcagatcttacattactggtacattgtcttgcc |
| | 450 | polynucleotide | attgttggccattatcaaccagatcgtggctcatgtcagga |
| | monooxygenase | | ccaattatttgatgaagaaattgggtgctaagccattcaca |
| | CYP52A19 | | cacgtccaacgtgacgggtggttgggcttcaaattcggccg |
| | (EC | | tgaattcctcaaagcaaaaagtgctgggagactggttgatt |
| | 1.14.14.1) | | taatcatctcccgtttccacgataatgaggacactttctcc |
| | | | agctatgcttttggcaaccatgtggtgttcaccagggaccc |
| | | | cgagaatatcaaggcgcttttggcaacccagtttggtgatt |
| | | | tttcattgggcagcagggtcaagttcttcaaaccattattg |
| | | | gggtacggtatcttcacattggacgccgaaggctggaagca |
| | | | cagcagagccatgttgagaccacagtttgccagagaacaag |
| | | | ttgctcatgtgacgtcgttggaaccacacttccagttgttg |
| | | | aagaagcatatccttaaacacaagggtgagtactttgatat |
| | | | ccaggaattgttctttagatttactgtcgactcggccacgg |
| | | | agttcttatttggtgagtccgtgcactccttaaaggacgag |
| | | | gaaattggctacgacacgaaagacatgtctgaagaaagacg |
| | | | cagatttgccgacgcgttcaacaagtcgcaagtctacgtgg |
| | | | ccaccagagttgctttacagaacttgtactggttggtcaac |
| | | | aacaaagagttcaaggagtgcaatgacattgtccacaagtt |
| | | | taccaactactatgttcagaaagccttggatgctaccccag |
| | | | aggaacttgaaaagcaaggcgggtatgtgttcttgtatgag |
| | | | cttgtcaagcagacgagagaccccaaggtgttgcgtgacca |
| | | | gtctttgaacatcttgttggcaggaagagacaccactgctg |
| | | | ggttgttgtcctttgctgtgtttgagttggccagaaaccca |
| | | | cacatctgggccaagttgagagaggaaattgaacagcagtt |
| | | | tggtcttggagaagactctcgtgttgaagagattacctttg |
| | | | agagcttgaagagatgtgagtacttgaaggccgtgttgaac |
| | | | gaaactttgagattacacccaagtgtcccaagaaacgcaag |
| | | | atttgcgattaaagacacgactttaccaagaggcggtggcc |
| | | | ccaacggcaaggatcctatcttgatcaggaaggatgaggtg |
| | | | gtgcagtactccatctcggcaactcagacaaatcctgctta |
| | | | ttatggcgccgatgctgctgattttagaccggaaagatggt |
| | | | ttgaaccatcaactagaaacttgggatgggctttcttgcca |
| | | | ttcaacggtggtccaagaatctgtttgggacaacagtttgc |
| | | | tttgactgaagccggttacgttttggttagacttgttcagg |
| | | | agtttccaaacttgtcacaagaccccgaaaccaagtaccca |
| | | | ccacctagattggcacacttgacgatgtgcttgtttgacgg |
| | | | tgcacacgtcaagatgtcatag |
| 47 | Cytochrome P- | Candida sp. | mldgilhywyivlpllaiingivahvrtnylmkklgakpft |
| | 450 | polypeptide | hvgrdgwlgfkfgreflkaksagrsvdliisrfhdnedtfs |
| | monooxygenase | | syafgnhvvftrdpenikallatqfgdfslgsrvkffkpll |
| | CYP52A19 | | gygiftldaegwkhsramlrpgfareqvahvtslephfqll |
| | (EC | | kkhilkhkgeyfdigelffrftvdsateflfgesvhslkde |
| | 1.14.14.1) | | eigydtkdmseerrrfadafnksqvyvatrvalqnlywlvn |
| | | | nkefkecndivhkftnyyvqkaldatpeelekqggyvflye |
| | | | lvkqtrdpkvlrdqslnillagrdttagllsfavfelarnp |
| | | | hiwaklreeieqqfglgedsrveeitfeslkrceylkavin |
| | | | etlrlhpsvprnarfaikdttlprgggpngkdpilirkdev |
| | | | vqysisatqtnpayygadaadfrperwfepstrnlgwaflp |
| | | | fnggpriclgqqfalteagyvlvrlvqefpnlsqdpetkyp |
| | | | pprlahltmclfdgahvkms* |
| 48 | Cytochrome P- | Candida sp. | atgctcgaccagatcttccattactggtacattgtcttgcc |
| | 450 | polynucleotide | attgttggtcattatcaagcagatcgtggctcatgccagga |
| | monooxygenase | | ccaattatttgatgaagaagttgggcgctaagccattcaca |
| | CYP52A20 | | catgtccaactagacgggtggtttggcttcaaatttggccg |
| | (EC | | tgaattcctcaaagctaaaagtgctgggaggcaggttgatt |
| | 1.14.14.1) | | taatcatctcccgtttccacgataatgaggacactttctcc |
| | | | agctatgcttttggcaaccatgtggtgttcaccagggaccc |
| | | | cgagaatatcaaggcgcttttggcaacccagtttggtgatt |
| | | | tttcattgggaagcagggtcaaattcttcaaaccattgttg |
| | | | gggtacggtatcttcaccttggacggcgaaggctggaagca |
| | | | cagcagagccatgttgagaccacagtttgccagagagcaag |
| | | | ttgctcatgtgacgtcgttggaaccacatttccagttgttg |
| | | | aagaagcatattcttaagcacaagggtgaatactttgatat |
| | | | ccaggaattgttctttagatttaccgttgattcagcgacgg |
| | | | agttcttatttggtgagtccgtgcactccttaagggacgag |
| | | | gaaattggctacgatacgaaggacatggctgaagaaagacg |
| | | | caaatttgccgacgcgttcaacaagtcgcaagtctatttgt |
| | | | ccaccagagttgctttacagacattgtactggttggtcaac |
| | | | aacaaagagttcaaggagtgcaacgacattgtccacaagtt |
| | | | caccaactactatgttcagaaagccttggatgctaccccag |
| | | | aggaacttgaaaaacaaggcgggtatgtgttcttgtacgag |
| | | | cttgccaagcagacgaaagaccccaatgtgttgcgtgacca |
| | | | gtctttgaacatcttgttggctggaagggacaccactgctg |
| | | | ggttgttgtcctttgctgtgtttgagttggccaggaaccca |
| | | | cacatctgggccaagttgagagaggaaattgaatcacactt |
| | | | tgggctgggtgaggactctcgtgttgaagagattacctttg |
| | | | agagcttgaagagatgtgagtacttgaaagccgtgttgaac |
| | | | gaaacgttgagattacacccaagtgtcccaagaaacgcaag |
| | | | atttgcgattaaagacacgactttaccaagaggcggtggcc |
| | | | ccaacggcaaggatcctatcttgatcagaaagaatgaggtg |
| | | | gtgcaatactccatctcggcaactcagacaaatcctgctta |
| | | | ttatggcgccgatgctgctgattttagaccggaaagatggt |
| | | | ttgagccatcaactagaaacttgggatgggcttacttgcca |
| | | | ttcaacggtggtccaagaatctgcttgggacaacagtttgc |
| | | | tttgaccgaagccggttacgttttggttagacttgttcagg |
| | | | aattccctagcttgtcacaggaccccgaaactgagtaccca |
| | | | ccacctagattggcacacttgacgatgtgcttgtttgacgg |
| | | | ggcatacgtcaagatgcaatag |
| 49 | Cytochrome P- | Candida sp. | mldgifhywyivlpllviikqivahartnylmkklgakpft |
| | 450 | polypeptide | hvgldgwfgfkfgreflkaksagrqvdliisrfhdnedtfs |
| | monooxygenase | | syafgnhvvftrdpenikallatqfgdfslgsrvkffkpll |
| | CYP52A20 | | gygiftldgegwkhsramlrpgfareqvahvtslephfqll |
| | (EC | | kkhilkhkgeyfdigelffrftvdsateflfgesvhslrde |
| | 1.14.14.1) | | eigydtkdmaeerrkfadafnksqvylstrvalqtlywlvn |
| | | | nkefkecndivhkftnyyvqkaldatpeelekqggyvflye |
| | | | lakqtkdpnvirdqslnillagrdttagllsfavfelarnp |
| | | | hiwaklreeieshfgsgedsrveeitfeslkrceylkavin |
| | | | etlrlhpsvprnarfaikdttlprgggpngkdpilirknev |
| | | | vqysisatqtnpayygadaadfrperwfepstrnlgwaylp |
| | | | fnggpriclgqqfalteagyvlvrlvqefpslsqdpeteyp |
| | | | pprlahltmclfdgayvkmq* |
| 50 | Cytochrome P- | Candida sp. | atggctatatctagtttgctatcgtgggatgtgatctgtgt |
| | 450 | polynucleotide | cgtcttcatttgcgtttgtgtttatttcgggtatgaatatt |
| | monooxygenase | | gttatactaaatacttgatgcacaaacatggcgctcgagaa |
| | CYP52D2 | | atcgagaatgtgatcaacgatgggttctttgggttccgctt |
| | (EC | | acctttgctactcatgcgagccagcaatgagggccgactta |
| | 1.14.14.1) | | tcgagttcagtgtcaagagattcgagtcggcgccacatcca |
| | | | cagaacaagacattggtcaaccgggcattgagcgttcctgt |
| | | | gatactcaccaaggacccagtgaatatcaaagcgatgctat |
| | | | cgacccagtttgatgacttttcccttgggttgagactacac |
| | | | cagtttgcgccgttgttggggaaaggcatctttactttgga |
| | | | cggcccagagtggaagcagagccgatctatgttgcgtccgc |
| | | | aatttgccaaagatcgggtttctcatatcctggatctagaa |
| | | | ccgcattttgtgttgcttcggaagcacattgatggccacaa |
| | | | tggagactacttcgacatccaggagctctacttccggttct |
| | | | cgatggatgtggcgacggggtttttgtttggcgagtctgtg |
| | | | gggtcgttgaaagacgaagatgcgaggttcctggaagcatt |
| | | | caatgagtcgcagaagtatttggcaactagggcaacgttgc |
| | | | acgagttgtactttctttgtgacgggtttaggtttcgccag |
| | | | tacaacaaggttgtgcgaaagttctgcagccagtgtgtcca |
| | | | caaggcgttagatgttgcaccggaagacaccagcgagtacg |
| | | | tgtttctccgcgagttggtcaaacacactcgagatcccgtt |
| | | | gttttacaagaccaagcgttgaacgtcttgcttgctggacg |
| | | | cgacaccaccgcgtcgttattatcgtttgcaacatttgagc |
| | | | tagcccggaatgaccacatgtggaggaagctacgagaggag |
| | | | gttatcctgacgatgggaccgtccagtgatgaaataaccgt |
| | | | ggccgggttgaagagttgccgttacctcaaagcaatcctaa |
| | | | acgaaactcttcgactatacccaagtgtgcctaggaacgcg |
| | | | agatttgctacgaggaatacgacgcttcctcgtggcggagg |
| | | | tccagatggatcgtttccgattttgataagaaagggccagc |
| | | | cagtggggtatttcatttgtgctacacacttgaatgagaag |
| | | | gtatatgggaatgatagccatgtgtttcgaccggagagatg |
| | | | ggctgcgttagagggcaagagtttgggctggtcgtatcttc |
| | | | cattcaacggcggcccgagaagctgccttggtcagcagttt |
| | | | gcaatccttgaagcttcgtatgttttggctcgattgacaca |
| | | | gtgctacacgacgatacagcttagaactaccgagtacccac |
| | | | caaagaaactcgttcatctcacgatgagtcttctcaacggg |
| | | | gtgtacatccgaactagaact |
| 51 | Cytochrome P- | Candida sp. | maissllswdvicvvficvcvyfgyeycytkylmhkhgare |
| | 450 | polypeptide | ienvindgffgfrlplllmrasnegrliefsvkrfesaphp |
| | monooxygenase | | qnktivnralsvpviltkdpvnikamlstqfddfslglrlh |
| | CYP52D2 | | gfapllgkgiftldgpewkgsrsmlrpqfakdrvshisdle |
| | (EC | | phfvllrkhidghngdyfdigelyfrfsmdvatgflfgesv |
| | 1.14.14.1) | | gslkdedarfseafnesqkylatratlhelyflcdgfrfrq |
| | | | ynkvvrkfcsqcvhkaldvapedtseyvflrelvkhtrdpv |
| | | | vlqdgalnvllagrdttasllsfatfelarndhmwrklree |
| | | | vistmgpssdeitvaglkscrylkailnetlrlypsvprna |
| | | | rfatrnttlprgggpdgsfpilirkgqpvgyficathlnek |
| | | | vygndshvfrperwaalegkslgwsylpfnggprsclgqqf |
| | | | aileasyvlarltqcyttiqlrtteyppkklvhltmsllng |
| | | | vyirtrt* |
| 52 | Alcohol | Candida sp. | atgtctgctaatatcccaaaaactcaaaaagctgtcgtctt |
| | dehydrogenase | polynucleotide | tgagaagaacggtggtgaattagaatacaaagatatcccag |
| | ADH1-1 short | | tgccaaccccaaaggccaacgaattgctcatcaacgtcaaa |
| | (EC 1.1.1.1) | | tactcgggtgtctgccacactgatttgcacgcctggaaggg |
| | | | tgactggccattggccaccaagttgccattggttggtggtc |
| | | | acgaaggtgctggtgtcgttgtcggcatgggtgaaaacgtc |
| | | | aagggctggaagattggtgacttcgccggtatcaaatggtt |
| | | | gaacggttcctgtatgtcctgtgagttctgtcaacaaggtg |
| | | | ctgaaccaaactgtggtgaggccgacttgtctggttacacc |
| | | | cacgatggttctttcgaacaatacgccactgctgatgctgt |
| | | | tcaagccgccagaatcccagctggtactgatttggccgaag |
| | | | ttgccccaatcttgtgtgcgggtgtcaccgtctacaaagcc |
| | | | ttgaagactgccgacttggccgctggtcaatgggtcgctat |
| | | | ctccggtgctggtggtggtttgggttccttggctgtccaat |
| | | | acgccgtcgccatgggcttgagagtcgttgccattgacggt |
| | | | ggtgacgaaaagggtgcctttgtcaagtccttgggtgctga |
| | | | agcctacattgatttcctcaaggaaaaggacattgtctctg |
| | | | ctgtcaagaaggccaccgatggaggtccacacggtgctatc |
| | | | aatgtttccgtttccgaaaaagccattgaccaatccgtcga |
| | | | gtacgttagaccattgggtaaggttgttttggttggtttgc |
| | | | cagctggctccaaggtcactgctggtgttttcgaagccgtt |
| | | | gtcaagtccattgaaatcaagggttcctatgtcggtaacag |
| | | | aaaggataccgccgaagccgttgactttttctccagaggct |
| | | | tgatcaagtgtccaatcaagattgttggcttgagtgaattg |
| | | | ccacaggtcttcaagttgatggaagaaggtaagatcttggg |
| | | | tagatacgtcttggatacctccaaa |
| 53 | Alcohol | Candida sp. | msanipktqkavvfeknggeleykdipvptpkanellinvk |
| | dehydrogenase | polypeptide | ysgvchtdlhawkgdwplatklplvgghegagvvvgmgenv |
| | ADH1-1 short | | kgwkigdfagikwlngscmscefcqqgaepncgeadlsgyt |
| | (EC 1.1.1.1) | | hdgsfegyatadavgaaripagtdlaevapilcagvtvyka |
| | | | lktadlaaggwvaisgaggglgslavqyavamglrvvaidg |
| | | | gdekgafvkslgaeayidflkekdivsavkkatdggphgai |
| | | | nvsysekaidgsveyvrplgkvvlvglpagskvtagvfeav |
| | | | vksieikgsyvgnrkdtaeavdffsrglikcpikivglsel |
| | | | pqvfklmeegkilgryvldtsk |
| 54 | Alcohol | Candida sp. | atgtctgctaatatcccaaaaactcaaaaagctgtcgtctt |
| | dehydrogenase | polynucleotide | cgagaagaacggtggtgaattaaaatacaaagacatcccag |
| | ADH1-2 short | | tgccaaccccaaaggccaacgaattgctcatcaacgtcaag |
| | (EC 1.1.1.1) | | tactcgggtgtctgtcacactgatttgcacgcctggaaggg |
| | | | tgactggccattggacaccaaattgccattggttggtggtc |
| | | | acgaaggtgctggtgttgttgtcggcatgggtgaaaacgtc |
| | | | aagggctggaaaatcggtgatttcgccggtatcaaatggtt |
| | | | gaacggttcttgtatgtcctgtgagttctgtcagcaaggtg |
| | | | ctgaaccaaactgtggtgaagctgacttgtctggttacacc |
| | | | cacgatggttctttcgaacaatacgccactgctgatgctgt |
| | | | gcaagccgccagaatcccagctggcactgatttggccgaag |
| | | | ttgccccaatcttgtgtgctggtgtcaccgtctacaaagcc |
| | | | ttgaagactgccgacttggctgctggtcaatgggtcgctat |
| | | | ctccggtgctggtggtggtttgggctccttggctgtccaat |
| | | | acgccgtcgccatgggtttgagagtcgttgccattgacggt |
| | | | ggtgacgaaaagggtgactttgtcaagtccttgggtgctga |
| | | | agcctacattgatttcctcaaggaaaagggcattgttgctg |
| | | | ctgtcaagaaggccactgatggcggtccacacggtgctatc |
| | | | aatgtttccgtttccgaaaaagccattgaccaatctgtcga |
| | | | gtacgttagaccattgggtaaggttgttttggttggtttgc |
| | | | cagctggctccaaggtcactgctggtgttttcgaagccgtt |
| | | | gtcaagtccattgaaatcaagggttcttacgtcggtaacag |
| | | | aaaggatactgccgaagccgttgactttttctccagaggct |
| | | | tgatcaagtgtccaatcaagattgtgggcttgagtgaattg |
| | | | ccacaggtcttcaagttgatggaagaaggtaagatcttggg |
| | | | tagatacgtcttggatacctccaaa |
| 55 | Alcohol | Candida sp. | msanipktqkavvfeknggelkykdipvptpkanellinvk |
| | dehydrogenase | polypeptide | ysgvchtdlhawkgdwpldtklplvgghegagvvvgmgenv |
| | ADH1-2 short | | kgwkigdfagikwlngscmscefcqqgaepncgeadlsgyt |
| | (EC 1.1.1.1) | | hdgsfegyatadavgaaripagtdlaevapilcagvtvyka |
| | | | lktadlaaggwvaisgaggglgslavqyavamglrvvaidg |
| | | | gdekgdfvkslgaeayidflkekgivaavkkatdggphgai |
| | | | nvsysekaidgsveyvrplgkvvlvglpagskvtagvfeav |
| | | | vksieikgsyvgnrkdtaeavdffsrglikcpikivglsel |
| | | | pqvfklmeegkilgryvldtsk |
| 56 | Alcohol | Candida sp. | atgcatgcattattctcaaaatcagtttttctcaagtatgt |
| | dehydrogenase | polynucleotide | gagtctgcccactacctctgctatcccccattccctagaat |
| | ADH1-2 | | tcattgtctcccgaagctcctatttaaggagacgaattccc |
| | (EC 1.1.1.1) | | ccatatcttccacgttgctcccactttccttccttctatta |
| | | | ttcttcttcttcagtctacaccaagaaatcatttcacacaa |
| | | | tgtctgctaatatcccaaaaactcaaaaagctgtcgtcttc |
| | | | gagaagaacggtggtgaattaaaatacaaagacatcccagt |
| | | | gccaaccccaaaggccaacgaattgctcatcaacgtcaagt |
| | | | actcgggtgtctgtcacactgatttgcacgcctggaagggt |
| | | | gactggccattggacaccaaattgccattggttggtggtca |
| | | | cgaaggtgctggtgttgttgtcggcatgggtgaaaacgtca |
| | | | agggctggaaaatcggtgatttcgccggtatcaaatggttg |
| | | | aacggttcttgtatgtcctgtgagttctgtcagcaaggtgc |
| | | | tgaaccaaactgtggtgaagctgacttgtctggttacaccc |
| | | | acgatggttctttcgaacaatacgccactgctgatgctgtg |
| | | | caagccgccagaatcccagctggcactgatttggccgaagt |
| | | | tgccccaatcttgtgtgctggtgtcaccgtctacaaagcct |
| | | | tgaagactgccgacttggctgctggtcaatgggtcgctatc |
| | | | tccggtgctggtggtggtttgggctccttggctgtccaata |
| | | | cgccgtcgccatgggtttgagagtcgttgccattgacggtg |
| | | | gtgacgaaaagggtgactttgtcaagtccttgggtgctgaa |
| | | | gcctacattgatttcctcaaggaaaagggcattgttgctgc |
| | | | tgtcaagaaggccactgatggcggtccacacggtgctatca |
| | | | atgtttccgtttccgaaaaagccattgaccaatctgtcgag |
| | | | tacgttagaccattgggtaaggttgttttggttggtttgcc |
| | | | agctggctccaaggtcactgctggtgttttcgaagccgttg |
| | | | tcaagtccattgaaatcaagggttcttacgtcggtaacaga |
| | | | aaggatactgccgaagccgttgactttttctccagaggctt |
| | | | gatcaagtgtccaatcaagattgtgggcttgagtgaattgc |
| | | | cacaggtcttcaagttgatggaagaaggtaagatcttgggt |
| | | | agatacgtcttggatacctccaaa |
| 57 | Alcohol | Candida sp. | mhalfsksvflkyvsspttsaiphslefivsrssylrrrip |
| | dehydrogenase | polypeptide | pylprcshfpsfyyssssvytkksfhtmsanipktgkavvf |
| | ADH1-2 | | eknggelkykdipvptpkanellinvkysgvchtdlhawkg |
| | (EC 1.1.1.1) | | dwpldtklplvgghegagvvvgmgenvkgwkigdfagikwl |
| | | | ngscmscefcqqgaepncgeadlsgythdgsfeqyatadav |
| | | | qaaripagtdlaevapilcagvtvykalktadlaaggwvai |
| | | | sgaggglgslavqyavamglrvvaidggdekgdfvkslgae |
| | | | ayidflkekgivaavkkatdggphgainvsysekaidqsve |
| | | | yvrplgkvvlvglpagskvtagvfeavvksieikgsyvgnr |
| | | | kdtaeavdffsrglikcpikivglselpqvfklmeegkilg |
| | | | ryvldtsk |
| 58 | Alcohol | Candida sp. | atgtcaattccaactactcaaaaagctatcattttcgaaac |
| | dehydrogenase | polynucleotide | caacggtggaaaattagaatacaaggacatcccagttccaa |
| | ADH2a | | agccaaagccaaacgaattgctcatcaacgtcaagtactcc |
| | (EC 1.1.1.1) | | ggtgtctgccacactgatttacacgcctggaagggtgactg |
| | | | gccattggacaccaagttgccattggtgggtggtcacgaag |
| | | | gtgctggtgttgttgttgccattggtgacaatgtcaaggga |
| | | | tggaaggtcggtgatttggccggtgtcaagtggttgaacgg |
| | | | ttcctgtatgaactgtgagtactgtcaacagggtgccgaac |
| | | | caaactgtccacaggctgacttgtctggttacacccacgac |
| | | | ggttctttccagcaatacgccactgcagatgccgtgcaagc |
| | | | cgctagaattccagctggtactgatttagccaacgttgccc |
| | | | ccatcttgtgtgctggtgtcactgtttacaaggccttgaag |
| | | | accgccgacttgcagccaggtcaatgggtcgccatttccgg |
| | | | tgccgctggtggtttgggttctttggccgttcaatacgcca |
| | | | aggccatgggctacagagttgtcgccatcgatggtggtgcc |
| | | | gacaagggtgagttcgtcaagtctttgggcgctgaggtctt |
| | | | tgttgatttcctcaaggaaaaggacattgttggtgctgtca |
| | | | agaaggcaaccgatggtggcccacacggtgccgttaacgtt |
| | | | tccatctccgaaaaggccatcaaccaatctgtcgactacgt |
| | | | tagaaccttgggtaaggttgtcttggtcggtttgccagctg |
| | | | gctccaaggtttctgctccagtctttgactccgtcgtcaag |
| | | | tccatccaaatcaagggttcctatgtcggtaacagaaagga |
| | | | cactgccgaagctgttgactttttctccagaggcttgatca |
| | | | agtgtccaatcaaggttgtcggtttgagtgaattgccagaa |
| | | | gtctacaagttgatggaagaaggtaagatcttgggtagata |
| | | | cgtcttggacaactctaag |
| 59 | Alcohol | Candida sp. | msipttqkaiifetnggkleykdipvpkpkpnellinvkys |
| | dehydrogenase | polypeptide | gvchtdlhawkgdwpldtklplvgghegagvvvaigdnvkg |
| | ADH2a | | wkvgdlagvkwlngscmnceycqqgaepncpqadlsgythd |
| | (EC 1.1.1.1) | | gsfqqyatadavqaaripagtdlanvapilcagvtvykalk |
| | | | tadlqpgqwvaisgaagglgslavqyakamgyrvvaidgga |
| | | | dkgefvkslgaevfvdflkekdivgavkkatdggphgavnv |
| | | | sisekaingsvdyvrtlgkvvlvglpagskvsapvfdsvvk |
| | | | sigikgsyvgnrkdtaeavdffsrglikcpikvvglselpe |
| | | | vyklmeegkilgryvldnsk |
| 60 | Alcohol | Candida sp. | atgtcaattccaactacccaaaaagctgttatctacgaagc |
| | dehydrogenase | polynucleotide | caactctgctccattgcaatacaccgatatcccagttccag |
| | ADH2b | | tccctaagccaaacgaattgctcgtccacgtcaaatactcc |
| | (EC 1.1.1.1) | | ggtgtttgtcactcagatatacacgtctggaagggtgactg |
| | | | gttcccagcatcgaaattgcccgttgttggtggtcacgaag |
| | | | gtgccggtgttgtcgttgccattggtgaaaacgtccaaggc |
| | | | tggaaagtaggtgacttggcaggtataaagatgttgaatgg |
| | | | ttcctgtatgaactgtgaatactgtcaacaaggtgctgaac |
| | | | caaactgtccccacgctgatgtctcgggttactcccacgac |
| | | | ggtactttccaacagtacgctaccgccgatgctgttcaagc |
| | | | tgctaaattcccagctggttctgatttagctagcatcgcac |
| | | | ctatatcctgcgccggtgttactgtttacaaagcattgaaa |
| | | | actgcaggcttgcagccaggtcaatgggttgccatctctgg |
| | | | tgcagctggtggtttgggttctttggctgtgcaatacgcca |
| | | | aggccatgggtttgagagtcgtggccattgacggtggtgac |
| | | | gaaagaggagtgtttgtcaaatcgttgggtgctgaagtttt |
| | | | cgttgatttcaccaaagaggccaatgtctctgaggctatca |
| | | | tcaaggctaccgacggtggtgcccatggcgtcatcaacgtt |
| | | | tccatttctgaaaaagccatcaaccagtctgttgaatatgt |
| | | | tagaactttgggaactgttgtcttggttggtttgccagctg |
| | | | gtgcaaagctcgaagctcctatcttcaatgccgttgccaaa |
| | | | tccatccaaatcaaaggttcttacgtgggaaacagaagaga |
| | | | cactgctgaggctgttgatttcttcgctagaggtttggtca |
| | | | aatgtccaattaaggttgttgggttgagtgaattgccagag |
| | | | attttcaaattgttggaagagggtaagatcttgggtagata |
| | | | cgttgttgacactgccaag |
| 61 | Alcohol | Candida sp. | msipttgkaviyeansaplqytdipvpvpkpnellvhvkys |
| | dehydrogenase | polypeptide | gvchsdihvwkgdwfpasklpvvgghegagvvvaigenvqg |
| | ADH2b | | wkvgdlagikmlngscmnceycqqgaepncphadvsgyshd |
| | (EC 1.1.1.1) | | gtfqqyatadavqaakfpagsdlasiapiscagvtvykalk |
| | | | taglqpgqwvaisgaagglgslavqyakamglrvvaidggd |
| | | | ergvfvkslgaevfvdftkeanvseaiikatdggahgvinv |
| | | | sisekaingsveyvrtlgtvvlvglpagakleapifnavak |
| | | | siqikgsyvgnrrdtaeavdffarglvkcpikvvglselpe |
| | | | ifklleegkilgryvvdtak |
| 62 | Alcohol | Candida sp. | atgtcaactcaatcaggttacggatacgtgaaaggacaaaa |
| | dehydrogenase | polynucleotide | gaccattcagaaatacaccgacatcccgatccctacgccgg |
| | ADH3 | | gccccaacgaagtcttgttgaaagtcgaagctgccggcttg |
| | (EC 1.1.1.1) | | tgtctctcggatccacacacgttgatcgggggtcccattga |
| | | | gagcaagccgccgttgccgaacgccacgaagttcatcatgg |
| | | | gtcacgaaatcgcggggctgattagccaagtaggcgccaac |
| | | | ttggccaacgatccatactataaaaagggaggtaggttcgc |
| | | | cttgactatcgcgcaggcttgtgggatttgtgagaattgtc |
| | | | gtgatgggtatgatgcaaagtgtgagtctacgacgcaggct |
| | | | tatgggttgaacgaggacggtggattccagcaatacttgtt |
| | | | gattaagaacttgcgtacgatgttgcctatccctgagggtg |
| | | | tgagttacgaagaagccgctgtgtctactgactctgtgttg |
| | | | actccattccatgcgattcagaaggtcgctcatttgttgca |
| | | | cccaactactaaggtgttggttcagggttgtggtgggttag |
| | | | gcttcaacgctattcaaatattgaagagctacaattgttac |
| | | | attgttgccactgatgtcaaaccagagcttgaaaaattagc |
| | | | tttggagtatggtgccaacgaataccacactgatctcacca |
| | | | agtccaagcatgagccaatgtcgttcgatttgattttcgac |
| | | | cttgtgggaatccaacctacttttgatttgtccgacaggta |
| | | | catcaaagcaaggggtaagattcttatgattggcttaggca |
| | | | gatccaagttgtttattccaaattataaattgggtatccgt |
| | | | gaagtcgagatcattttcaattttggtggtacttcggccga |
| | | | gcaaattgagtgcatgaaatgggttgcaaaaggcttgatca |
| | | | aacctaatattcacgtggctgattttgcttccttgcctgag |
| | | | tacctcgaggacttggccaagggtaaactcactggtagaat |
| | | | tgtatttagaccaagtaagttg |
| 63 | Alcohol | Candida sp. | mstqsgygyvkgqktiqkytdipiptpgpnevllkveaagl |
| | dehydrogenase | polypeptide | clsdphtliggpieskpplpnatkfimgheiagsisqvgan |
| | ADH3 | | landpyykkggrfaltiaqacgicencrdgydakcesttqa |
| | (EC 1.1.1.1) | | yglnedggfqqylliknlrtmlpipegvsyeeaaystdsvl |
| | | | tpfhaigkvahllhpttkvlvggcgglgfnaigilksyncy |
| | | | ivatdvkpeleklaleyganeyhtdltkskhepmsfdlifd |
| | | | lvgiqptfdlsdryikargkilmiglgrsklfipnyklgir |
| | | | eveiifnfggtsaeqiecmkwvakglikpnihvadfaslpe |
| | | | yledlakgkltgrivfrpskl |
| 64 | Alcohol | Candida sp. | atgtcattatcaggaaagacctcattaattgctgctggtac |
| | dehydrogenase | polynucleotide | caagaacttgggtggtgcaagtgccaaagaattggccaaag |
| | ADH4 | | ccggctccaacctcttcttgcactacagatccaacccagac |
| | (EC 1.1.1.1) | | gaggctgaaaagttcaagcaagagatcctcaaggagttccc |
| | | | taacgtcaaggtcgaaacctaccaatccaaattggaccgtg |
| | | | ccgccgacctcaccaacttgtttgctgctgccaagaaggca |
| | | | ttccctagtggtattgacgtcgctgtcaactttgtcggtaa |
| | | | ggtcatcaagggcccaatcactgaggtcactgaagaacagt |
| | | | ttgacgagatggatgttgccaacaacaagattgcctttttc |
| | | | ttcatcaaggaggccgctatcaacttgaacaagaacggtag |
| | | | tatcatttccatcgttactagtttgctcccagcttacaccg |
| | | | attcttacggtttgtaccagggtactaaaggagctgttgaa |
| | | | tactattcgaaatctatcctgaaggagttgattccaaaggg |
| | | | tatcaccagtaactgtattggtcctggtcctgcttctactt |
| | | | cctttttgtttaattccgaaaccaaggagagtgttgagttc |
| | | | ttcaagaccgttgctattgaccaacgtttgactgaagacag |
| | | | cgacattgccccaattgtgttgttcctcgccactggaggtc |
| | | | gttgggcaactggtcaaactatttacgctagtggtggtttc |
| | | | actgctcgt |
| 65 | Alcohol | Candida sp. | mslsgktsliaagtknlggasakelakagsnlflhyrsnpd |
| | dehydrogenase | polypeptide | eaekfkqeilkefpnvkvetygskldraadltnlfaaakka |
| | ADH4 | | fpsgidvavnfvgkvikgpitevteeqfdemdvannkiaff |
| | (EC 1.1.1.1) | | fikeaainlnkngsiisivtsllpaytdsyglyqgtkgave |
| | | | yysksiskelipkgitsncigpgpastsflfnsetkesvef |
| | | | fktvaidgrltedsdiapivlflatggrwatgqtiyasggf |
| | | | tar |
| 66 | Alcohol | Candida sp. | atgtcacttgtcctcaagcgattacttccaatcagatctcc |
| | dehydrogenase | polynucleotide | tactttactcaattcgaagttcatacagttacaatctcaaa |
| | ADH5 | | ttcgcacaatggctatccccgctactcaaactggattcttc |
| | (EC 1.1.1.1) | | ttcaccaaacaagaaggtttaaactacagaaccgacattcc |
| | | | tgtccgcaagccacaagccggtcagttgttgttgaaggtca |
| | | | atgccgttggtctctgccactcggacttgcacgtgattgac |
| | | | aaggagcttgaatgtggtgacaactatgtcatgggccacga |
| | | | aattgccggtaccgttgctgaagttggtcccgaagttgaag |
| | | | gctacaaggttggcgaccgtgtcgcttgtgttggtcctaac |
| | | | gggtgcggtgtctgtaagcactgcttgactggtaacgacaa |
| | | | tgtctgtaagactgctttcctcgactggttcgggttgggct |
| | | | ccgatggtgggtacgaagagtacttgttggtgagaagacca |
| | | | agaaacttggttaaggtcccggacaacgtctcgattgagga |
| | | | ggctgctgctatcactgatgctgtgttgactccttaccatg |
| | | | ctgtcaagactgccaaggtcaagccaaccagtaacgttttg |
| | | | gttattggtgctggtggattaggtggtaacggtatccagat |
| | | | tgtcaaggcttttggcggtaaggttactgttgtcgataaga |
| | | | aggataaggcacgtgaccaagctaaggctttgggtgctgat |
| | | | gaagtctacagtgaaatcccagcaagtattgaaccgggtac |
| | | | ttttgatgtctgtcttgattttgtttccgtgcaagccacct |
| | | | atgatctctgccaaaagtactgtgagccaaagggtatcatt |
| | | | atcccagttgggttgggtgctaccaagctcaccattgattt |
| | | | ggcagatttggatctccgtgaaatcacggttactggtacct |
| | | | tctggggaactgccaatgacttgagagaggcgtttgatttg |
| | | | gttagtcaaggtaagatcaagccgattgtttcacatgcccc |
| | | | attgaaggagttgccaaactatatggagaagttgaagcagg |
| | | | gagcatatgaaggaagagttgtcttccaccca |
| 67 | Alcohol | Candida sp. | mslvlkrllpirsptllnskfiglqsgirtmaipatqtgff |
| | dehydrogenase | polypeptide | ftkqeglnyrtdipvrkpgagqlllkvnavglchsdlhvid |
| | ADH5 | | kelecgdnyvmgheiagtvaevgpevegykvgdrvacvgpn |
| | (EC 1.1.1.1) | | gcgvckhcltgndnvcktafldwfglgsdggyeeyllvrrp |
| | | | rnlvkvpdnvsieeaaaitdavltpyhavktakvkptsnvl |
| | | | vigagglggngigivkafggkvtvvdkkdkardqakalgad |
| | | | evyseipasiepgtfdvcldfvsvgatydlcqkycepkgii |
| | | | ipvglgatkltidladldlreitvtgtfwgtandlreafdl |
| | | | vsqgkikpivshaplkelpnymeklkqgayegrvvfhp |
| 68 | Alcohol | Candida sp. | atgactgttgacgcttcttctgttccagacaagttccaagg |
| | dehydrogenase | polynucleotide | gtttgcctccgacaagagagaaaactgggaacacccaaagt |
| | ADH7 | | tgatctcctacgacagaaagcaactcaatgaccacgacgtt |
| | (EC 1.1.1.1) | | gtcttgaagaacgagacctgtggtttgtgttactcggacat |
| | | | ccacaccttgcgttccacgtggggaccatacggcaccaatg |
| | | | agcttgtcgttggccacgaaatctgtggtaccgtcattgct |
| | | | gtcggtccaaaggtcactgagttcaaggtcggtgacagagc |
| | | | cggtattggtgctgcctcttcgtcttgtcgtcactgttcca |
| | | | gatgtacccacgataacgagcaatactgtaaggaacaagtc |
| | | | tccacttacaattctgttgatccaaaggccgctggttacgt |
| | | | caccaagggtggttactcctcccactccatcgctgacgaat |
| | | | tgtttgtcttcaaggttccagatgacttgccattcgagtac |
| | | | gcttccccattattctgtgctggtatcacaactttctcccc |
| | | | attgtaccgtaacttggttgggtccgataaagacgccactg |
| | | | gtaagaccgttggtatcattggtgttggtggtcttggtcac |
| | | | cttgccatccagtttgcgtctaaagctttgaacgctaaggt |
| | | | cgttgctttctccagatcctcctccaagaaggaagaagctc |
| | | | tcgaattgggtgctgctgagtttgtcgccaccaacgaagac |
| | | | aagaactggaccagcagatacgaggaccaattcgacctcat |
| | | | cttgaactgtgcgagcggtatcgatggcttgaacttgtctg |
| | | | actacttgagtgtcttgaaagtcgacaagaagtttgtctct |
| | | | gttggtttgccaccaatcgacgacgagttcaacgtctctcc |
| | | | tttcactttcttgaagcaaggtgccagtttcggtagttcct |
| | | | tgttgggatccaaggctgaagtcaacatcatgttggaattg |
| | | | gctgccaagcacaacatcagaccatggattgaaaaggtccc |
| | | | aatcagtgaggaaaacgtcgccaaggctttgaagagatgtt |
| | | | ttgaaggtgatgtcagatacagattcgtcttcactgagttt |
| | | | gacaaagcttttggcaat |
| 69 | Alcohol | Candida sp. | mtvdassvpdkfqgfasdkrenwehpklisydrkqlndhdv |
| | dehydrogenase | polypeptide | vlknetcglcysdihtlrstwgpygtnelvvgheicgtvia |
| | ADH7 | | vgpkvtefkvgdragigaassscrhcsrcthdnegyckeqv |
| | (EC 1.1.1.1) | | stynsvdpkaagyvtkggysshsiadelfvfkvpddlpfey |
| | | | asplfcagittfsplyrnlvgsdkdatgktvgiigvgglgh |
| | | | laiqfaskalnakvvafsrssskkeealelgaaefvatned |
| | | | knwtsryedqfdlilncasgidglnlsdylsvlkvdkkfvs |
| | | | vglppiddefnvspftflkqgasfgssllgskaevnimlel |
| | | | aakhnirpwiekvpiseenvakalkrcfegdvryrfvftef |
| | | | dkafgn |
| 70 | Alcohol | Candida sp. | atgtccgttccaactactcagaaagctgttatctttgaaac |
| | dehydrogenase | polynucleotide | caatggtggcaagttagaatacaaagacgtgccggtccctg |
| | ADH8 | | tccctaaacccaacgaattgcttgtcaacgtcaagtactcg |
| | (EC 1.1.1.1) | | ggtgtgtgtcattctgacttgcatgtctggaaaggcgactg |
| | | | gcccattcctgccaagttgcccttggtgggaggtcacgaag |
| | | | gtgctggtgtcgttgtcggcatgggtgacaacgtcaagggc |
| | | | tggaaggtgggggacttggctggtatcaagtggttgaatgg |
| | | | ttcgtgtatgaactgtgagttttgccaacagggcgcagaac |
| | | | ctaactgttcaagagccgacatgtctgggtatacccacgat |
| | | | ggaactttccaacaatacgccactgctgatgctgtccaagc |
| | | | tgccaagatcccagaaggcgccgacatggctagtatcgccc |
| | | | cgatcttgtgcgctggtgtgaccgtgtacaaggctttgaag |
| | | | aacgccgacttgttggctggccaatgggtggctatctctgg |
| | | | tgctggtggtggtttgggctccttgggtgtgcagtacgcta |
| | | | aagccatgggttacagagtgttggctatcgacggtggtgac |
| | | | gagagaggagagtttgtcaagtccttgggcgccgaagtgta |
| | | | cattgacttccttaaggaacaggacatcgttagtgctatca |
| | | | gaaaggcaactggtggtggtccacacggtgttattaacgtc |
| | | | tcagtgtccgaaaaggcaatcaaccagtcggtggagtacgt |
| | | | cagaactttggggaaagtggttttagttagcttgccggcag |
| | | | gtggtaaactcactgctcctcttttcgagtctgttgctaga |
| | | | tcaatccagattagaactacgtgtgttggcaacagaaagga |
| | | | tactactgaagctattgatttctttgttagagggttgatcg |
| | | | attgcccaattaaagtcgctggtttaagtgaagtgccagag |
| | | | atttttgacttgatggagcagggaaagatcttgggtagata |
| | | | tgtcgttgatacgtcaaag |
| 71 | Alcohol | Candida sp. | msvpttqkavifetnggkleykdvpvpvpkpnellvnvkys |
| | dehydrogenase | polypeptide | gvchsdlhvwkgdwpipaklplvgghegagvvvgmgdnvkg |
| | ADH8 | | wkvgdlagikwlngscmncefcqqgaepncsradmsgythd |
| | (EC 1.1.1.1) | | gtfqqyatadavqaakipegadmasiapilcagvtvykalk |
| | | | nadllagqwvaisgaggglgslgvqyakamgyrvlaidggd |
| | | | ergefvkslgaevyidflkeqdivsairkatgggphgvinv |
| | | | sysekaingsveyvrtlgkvvlvslpaggkltaplfesvar |
| | | | sigirttcvgnrkdtteaidffvrglidcpikvaglsevpe |
| | | | ifdlmeqgkilgryvvdtsk |
| 72 | Aldehyde | Candida sp. | atgtccccaccatctaaattagaagactcctcctccgcaac |
| | dehydrogenase | polynucleotide | caccgctgccgatacccttggcgactcctggtacaccaaag |
| | (EC 1.2.1.5) | | tgtccgacattgcgcctggcgtgcagagattgaccgagtca |
| | | | ttccacagggatcaaaagacgcacgacattcagttccgctt |
| | | | gaaccaattgcgtaacctttactttgcggtccaggacaatg |
| | | | ccgacgcgctctgtgctgccttggacaaggacttctaccgt |
| | | | ccccccagtgaaaccaagaacttggaactcgtgggtggctt |
| | | | gaatgagttggtgcacaccatttcgagcttgcatgagtgga |
| | | | tgaagccggaaaaagtcacggatttgccacttactttgagg |
| | | | tcaaacccgatttatattgaaagaatcccattgggggtcgt |
| | | | gttgatcatctcgcctttcaactaccctttcttcttgtcgt |
| | | | tttcggccgtcgtgggtgcgattgctggtggtaacgcggtt |
| | | | gttttgaagggctctgagttgacgccaaacttctccagttt |
| | | | gttctcaaagatcttgactaaggctttggaccctgatattt |
| | | | tctttgcagtcgatggtgctatccctgagacgaccgagttg |
| | | | ttggaacaaaagtttgacaagatcatgtatactggtaacaa |
| | | | caccgtgggtaagattattgccaagaaggctgctgagacct |
| | | | tgacgccagttatcttggaattgggtggtaagtcgccagct |
| | | | ttcatcttggacgacgtcaaggataaaaacttggaagtcat |
| | | | cgccagaagaatcgcatggggtagattcaccaacgccggtc |
| | | | aaacctgtgttgctgtcgactacgtcttggttccaaccaaa |
| | | | ctccacaagaagttcattgctgcgttgaccaaggtcttgag |
| | | | tcaagaattctaccctaacttgaccaaagacaccaagggct |
| | | | acacccacgtcatccacgaccgtgcattcaacaatttgtcc |
| | | | aagatcatcagcaccaccaagggtgacattgtctttggcgg |
| | | | cgacaccgatgccgccacccgcttcatcgcccccaccgtca |
| | | | tcgacaacgccacctgggaggattcttccatgaagggcgaa |
| | | | atctttggtcccatcttgcccgtcttgacctacgacaagct |
| | | | caccaccgccatcaggcaagttgtgtccacgcacgacacgc |
| | | | cattagcgcagtacatcttcaccagcgggtccacatcccgc |
| | | | aagtacaaccgccagctcgaccagatcttgactggtgtccg |
| | | | gtccgggggtgtgattgtcaacgatgtcttgatgcacgttg |
| | | | cgttgatcaatgcgccatttggcggcgttggtgactccggg |
| | | | tacggctcgtaccacggcaagttctcgttccgcagcttcac |
| | | | gcacgaacgtaccaccatggagcagaagttgtggaacgacg |
| | | | ggatggtcaaggtcagataccctccttataactccaacaag |
| | | | gacaagttgatccaggtctcccagcagaactacaacggcaa |
| | | | ggtctggttcgatagaaacggcgacgtgcctgtgaatggac |
| | | | caggtgcgttgtttagcgcttggactacgttcactggtgtc |
| | | | ttccatttgcttggtgagttcatcactaataagcaatag |
| 73 | Aldehyde | Candida sp. | msppskledsssattaadtlgdswytkvsdiapgvqrltes |
| | dehydrogenase | polypeptide | fhrdqkthdigfringlrnlyfavgdnadalcaaldkdfyr |
| | (EC 1.2.1.5) | | ppsetknlelvgglnelvhtisslhewmkpekvtdlpltlr |
| | | | snpiyieriplgvvliispfnypfflsfsavvgaiaggnav |
| | | | vlkgseltpnfsslfskiltkaldpdiffavdgaipettel |
| | | | leqkfdkimytgnntvgkiiakkaaetltpvilelggkspa |
| | | | filddvkdknleviarriawgrftnagqtcvavdyvlvptk |
| | | | lhkkfiaaltkvlsgefypnitkdtkgythvihdrafnnls |
| | | | kiisttkgdivfggdtdaatrfiaptvidnatwedssmkge |
| | | | ifgpilpvltydklttairqvvsthdtplagyiftsgstsr |
| | | | kynrqldgiltgvrsggvivndvlmhvalinapfggvgdsg |
| | | | ygsyhgkfsfrsftherttmeqklwndgmvkvryppynsnk |
| | | | dkliqvsqqnyngkvwfdrngdvpvngpgalfsawttftgv |
| | | | fhllgefitnkq |
| 74 | Long chain | Candida sp. | atgtcaggattagaaatagccgctgctgccatccttggtag |
| | fatty acid- | polynucleotide | tcagttattggaagccaaatatttaattgccgacgacgtgc |
| | CoA ligase | | tgttagccaagacagtcgctgtcaatgccctcccatacttg |
| | (EC 6.2.1.3) | | tggaaagccagcagaggtaaggcatcatactggtacttttt |
| | | | cgagcagtccgtgttcaagaacccaaacaacaaggcgttgg |
| | | | cgttcccaagaccaagaaagaatgcccccacccccaagacc |
| | | | gacgccgaggggttccagatctacgacgaccagtttgacct |
| | | | agaagaatacacctacaaggaattgtacgatatggttttga |
| | | | agtactcgtacatcttgaagaacgagtacggtgtcactgcc |
| | | | aacgacaccattggtgtttcttgtatgaacaagccgctttt |
| | | | cattgtgttgtggttggcattgtggaacattggtgccttgc |
| | | | ctgcgttcttgaacttcaacaccaaggacaagccattgatc |
| | | | cactgtcttaagattgtcaacgcttcgcaagttttcgttga |
| | | | cccggactgtgattccccaatcagagataccgaggctcaga |
| | | | tcagagaggaattgccacatgtgcaaataaactacattgac |
| | | | gagtttgccttgtttgacagattgagactcaagtcgactcc |
| | | | aaaacacagagccgaggacaagaccagaagaccaaccgata |
| | | | ctgactcctcggcttgtgcattgatttacacctcgggtacc |
| | | | accggtttgccaaaagccggtatcatgtcctggagaaaagc |
| | | | cttcatggcctcggttttctttggccacatcatgaagattg |
| | | | actcgaaatcgaacgtcttgaccgccatgcccttgtaccac |
| | | | tccaccgcggccatgttggggttgtgtcctaccttgattgt |
| | | | cggtggctgtgtctcggtgtcccagaaattctccgctactt |
| | | | cgttctggacccaggccagattatgtggtgccacccacgtg |
| | | | caatacgtcggtgaggtctgtcgttacttgttgaactccaa |
| | | | gcctcatccagaccaagacagacacaatgtcagaattgcct |
| | | | acggtaacgggttgcgtccagatatatggtctgagttcaag |
| | | | cgcagattccacattgaaggtatcggtgagttctacgccgc |
| | | | caccgagtcccctatcgccaccaccaacttgcagtacggtg |
| | | | agtacggtgtcggcgcctgtcgtaagtacgggtccctcatc |
| | | | agcttgttattgtctacccagcagaaattggccaagatgga |
| | | | cccagaagacgagagtgaaatctacaaggaccccaagaccg |
| | | | ggttctgtaccgaggccgcttacaacgagccaggtgagttg |
| | | | ttgatgagaatcttgaaccctaacgacgtgcagaaatcctt |
| | | | ccagggttattacggtaacaagtccgccaccaacagcaaaa |
| | | | tcctcaccaatgttttcaaaaaaggtgacgcgtggtacaga |
| | | | tccggtgacttgttgaagatggacgagaacaaattgttgta |
| | | | ctttgtcgacagattaggtgacacgttccgttggaagtccg |
| | | | aaaacgtctccgccaccgaggtcgagaacgaattgatgggc |
| | | | tccaaggccttgaagcagtccgtcgttgtcggtgtcaaggt |
| | | | gccaaaccacgaaggtagagcctgttttgccgtctgtgaag |
| | | | ccaaggacgagttgagccatgaagaaatcttgaaattgatt |
| | | | cactctcacgtgaccaagtctttgcctgtgtatgctcaacc |
| | | | tgcgttcatcaagattggcaccattgaggcttcgcacaacc |
| | | | acaaggttcctaagaaccaattcaagaaccaaaagttacca |
| | | | aagggtgaagacggcaaggatttgatctactggttgaatgg |
| | | | cgacaagtaccaggagttgactgaagacgattggtctttga |
| | | | tttgtaccggtaaagccaaattggaatag |
| 75 | Long chain | Candida sp. | msgleiaaaailgsqlleakyliaddvslaktvavnalpyl |
| | fatty acid- | polypeptide | wkasrgkasywyffeqsvfknpnnkalafprprknaptpkt |
| | CoA ligase | | daegfqiyddqfdleeytykelydmvlkysyilkneygvta |
| | (EC 6.2.1.3) | | ndtigvscmnkplfivlwlalwnigalpaflnfntkdkpli |
| | | | hclkivnasqvfvdpdcdspirdteagireelphvginyid |
| | | | efalfdrlrlkstpkhraedktrrptdtdssacaliytsgt |
| | | | tglpkagimswrkafmasvffghimkidsksnvltamplyh |
| | | | staamlglcptlivggcvsysqkfsatsfwtqarlcgathv |
| | | | qyvgevcryllnskphpdqdrhnvriaygnglrpdiwsefk |
| | | | rrfhiegigefyaatespiattnlqygeygvgacrkygsli |
| | | | slllstqqklakmdpedeseiykdpktgfcteaaynepgel |
| | | | lmrilnpndvqksfqgyygnksatnskiltnvfkkgdawyr |
| | | | sgdllkmdenkllyfvdrlgdtfrwksenvsatevenelmg |
| | | | skalkqsvvvgvkvpnhegracfavceakdelsheeilkli |
| | | | hshvtkslpvyagpafikigtieashnhkvpknqfknqklp |
| | | | kgedgkdliywingdkygelteddwslictgkakle |
| 76 | Acyl-CoA | Candida sp. | atgggtgcccctttaacagtcgccgttggcgaagcaaaacc |
| | synthetase | polynucleotide | aggcgaaaccgctccaagaagaaaagccgctcaaaaaatgg |
| | (EC 6.2.1.3) | | cctctgtcgaacgcccaacagactcaaaggcaaccactttg |
| | | | ccagacttcattgaagagtgttttgccagaaacggcaccag |
| | | | agatgccatggcctggagagacttggtcgaaatccacgtcg |
| | | | aaaccaaacaggttaccaaaatcattgacggcgaacagaaa |
| | | | aaggtcgataaggactggatctactacgaaatgggtcctta |
| | | | caactacatatcctaccccaagttgttgacgttggtcaaga |
| | | | actactccaagggtttgttggagttgggcttggccccagat |
| | | | caagaatccaagttgatgatctttgccagtacctcccacaa |
| | | | gtggatgcagaccttcttagcctccagtttccaaggtatcc |
| | | | ccgttgtcaccgcctacgacaccttgggtgagtcgggcttg |
| | | | acccactccttggtgcaaaccgaatccgatgccgtgttcac |
| | | | cgacaaccaattgttgtcctccttgattcgtcctttggaga |
| | | | aggccacctccgtcaagtatgtcatccacggggaaaagatt |
| | | | gaccctaacgacaagagacagggcggcaaaatctaccagga |
| | | | tgcggaaaaggccaaggagaagattttacaaattagaccag |
| | | | atattaaatttatttctttcgacgaggttgttgcattgggt |
| | | | gaacaatcgtccaaagaattgcatttcccaaaaccagaaga |
| | | | cccaatctgtatcatgtacacctcgggttccaccggtgctc |
| | | | caaagggtgtggttatcaccaatgccaacattgttgccgcc |
| | | | gtgggtggtatctccaccaatgctactagagacttggttag |
| | | | aactgtcgacagagtgattgcatttttgccattggcccaca |
| | | | ttttcgagttggcctttgagttggttaccttctggtggggg |
| | | | gctccattgggttacgccaatgtcaagactttgaccgaagc |
| | | | ctcctgcagaaactgtcagccagacttgattgaattcaaac |
| | | | caaccatcatggttggtgttgctgccgtttgggaatcggtc |
| | | | agaaagggtgtcttgtctaaattgaaacaggcttctccaat |
| | | | ccaacaaaagatcttctgggctgcattcaatgccaagtcta |
| | | | ctttgaaccgttatggcttgccaggcggtgggttgtttgac |
| | | | gctgtcttcaagaaggttaaagccgccactggtggccaatt |
| | | | gcgttatgtgttgaatggtgggtccccaatctctgttgatg |
| | | | cccaagtgtttatctccaccttgcttgcgccaatgttgttg |
| | | | ggttacggtttgactgaaacctgtgccaataccaccattgt |
| | | | cgaacacacgcgcttccagattggtactttgggtaccttgg |
| | | | ttggatctgtcactgccaagttggttgatgttgctgatgct |
| | | | ggatactacgccaagaacaaccagggtgaaatctggttgaa |
| | | | aggcggtccagttgtcaaggaatactacaagaacgaagaag |
| | | | aaaccaaggctgcattcaccgaagatggctggttcaagact |
| | | | ggtgatattggtgaatggaccgccgacggtggtttgaacat |
| | | | cattgaccgtaagaagaacttggtcaagactttgaatggtg |
| | | | aatacattgctttggagaaattggaaagtatttacagatcc |
| | | | aaccacttgattttgaacttgtgtgtttacgctgaccaaac |
| | | | caaggtcaagccaattgctattgtcttgccaattgaagcca |
| | | | acttgaagtctatgttgaaggacgaaaagattatcccagat |
| | | | gctgattcacaagaattgagcagcttggttcacaacaagaa |
| | | | ggttgcccaagctgtcttgagacacttgctccaaaccggta |
| | | | aacaacaaggtttgaaaggtattgaattgttgcagaatgtt |
| | | | gtcttgttggatgacgagtggaccccacagaatggttttgt |
| | | | tacttctgcccaaaagttgcagagaaagaagattttagaaa |
| | | | gttgtaaaaaagaagttgaagaggcatacaagtcgtct |
| 77 | Acyl-CoA | Candida sp. | mgapltvavgeakpgetaprrkaaqkmasverptdskattl |
| | synthetase | polypeptide | pdfieecfarngtrdamawrdlveihvetkqvtkiidgeqk |
| | (EC 6.2.1.3) | | kvdkdwiyyemgpynyisypklltivknyskgllelglapd |
| | | | qesklmifastshkwmqtflassfqgipvvtaydtlgesgl |
| | | | thslvqtesdavftdnqllsslirplekatsvkyvihgeki |
| | | | dpndkrqggkiygdaekakekilgirpdikfisfdevvalg |
| | | | eqsskelhfpkpedpicimytsgstgapkgvvitnanivaa |
| | | | vggistnatrdlvrtvdrviaflplahifelafelvtfwwg |
| | | | aplgyanvktlteascrncqpdliefkptimvgvaavwesv |
| | | | rkgvlsklkgaspiqqkifwaafnakstlnryglpggglfd |
| | | | avfkkvkaatggqlryvinggspisvdaqvfistllapmll |
| | | | gygltetcanttivehtrfqigtlgtivgsvtaklvdvada |
| | | | gyyaknnggeiwlkggpvvkeyykneeetkaaftedgwfkt |
| | | | gdigewtadgglniidrkknlvktlngeyialeklesiyrs |
| | | | nhlilnlcvyadqtkvkpiaivlpieanlksmlkdekiipd |
| | | | adsgelsslvhnkkvagavlrhllgtgkqqglkgiellqnv |
| | | | vllddewtpqngfvtsagklqrkkilesckkeveeaykss |
| 78 | 3-ketoacyl- | Candida sp. | atggatagattaaaccaattaagcggccaattaaagccaaa |
| | CoA thiolase | polynucleotide | cgccaaacaatccatcttgcaaaaaaacccagacgacgtcg |
| | (beta- | | ttatcgttgctgcatacagaaccgccatcggtaaaggtttc |
| | ketothiolase) | | aaaggttccttcagaagcgtccgctctgaattcatcttgac |
| | POT1-1 | | tgagttcttgaaagaattcattaaaaagaccaacatcgacc |
| | (EC 2.3.1.16) | | catctttgattgaagatgtcgctatcggtaacgtcttgaac |
| | | | caggccgccggtgccaccgaacacagaggtgcttgtttggc |
| | | | tgccggtatcccatacaccgccgctttcatcgccgtcaaca |
| | | | gattctgctcatccggtttgatggccatctccgacattgcc |
| | | | aacaagatcaagactggtgaaatcgagtgtggtttggctgg |
| | | | tggtgccgaatccatgtccaccaactaccgtgatcctagag |
| | | | ttgccccaagaatcgacccacacttggctgacgacgcccaa |
| | | | atggaaaagtgtttgattcctatgggtatcaccaacgaaaa |
| | | | cgttgctaaccaattcaacatctccagagaaagacaagacg |
| | | | agttcgccgccaagtcctacaacaaggctgccaaggctgtt |
| | | | gccgctggtgctttcaagagcgaaatcttgccaatcagatc |
| | | | catcatcagaaactctgacggtaccgaaaaggaaatcattg |
| | | | tcgacactgacgaaggtccaagagaaggtgtcaccgctgaa |
| | | | tccttgggcaagttgagaccagctttcgacggtaccaccac |
| | | | tgccggtaacgcttcccaagtctctgacggtgctgccgccg |
| | | | tcttgttgatgaagagaagcttggctgaagccaagggatac |
| | | | ccaatcattggtaagtacgtcctttgttccaccgccggtgt |
| | | | tcctccagaaattatgggtgttggtccagcctacgctatcc |
| | | | cagaagtcttgaagagaactggtttgactgttgacgacatt |
| | | | gatgttttcgaaatcaacgaagcctttgctgctcaatgtct |
| | | | ctactctgctgaacaagtcaatgtgcctgaagagaagttga |
| | | | acatcaacggtggtgccattgccttgggccacccattgggt |
| | | | gaaaccggtgctcgtcaatacgccaccatcatcccattgtt |
| | | | aaaaccaggtcaaattggattgacttcaatgtgtattggtt |
| | | | ctggtatgggttctgcttctattttggttagagaatag |
| 79 | 3-ketoacyl- | Candida sp. | mdrinqlsgqlkpnakgsilqknpddvvivaayrtaigkgf |
| | CoA thiolase | polypeptide | kgsfrsvrsefilteflkefikktnidpsliedvaignvin |
| | (beta- | | qaagatehrgaclaagipytaafiavnrfcssglmaisdia |
| | ketothiolase) | | nkiktgeiecglaggaesmstnyrdprvapridphladdaq |
| | POT1-1 | | mekclipmgitnenvanqfnisrerqdefaaksynkaakav |
| | (EC 2.3.1.16) | | aagafkseilpirsiirnsdgtekeiivdtdegpregvtae |
| | | | slgklrpafdgtttagnasqvsdgaaavllmkrslaeakgy |
| | | | piigkyvlcstagvppeimgvgpayaipevlkrtgltvddi |
| | | | dvfeineafaagclysaegvnvpeeklninggaialghplg |
| | | | etgarqyatiipllkpgqigltsmcigsgmgsasilvre |
| 80 | 3-ketoacyl- | Candida sp. | atggatagattaaaccaattaagcggccaattaaagccaaa |
| | CoA thiolase | polynucleotide | cgctaaacaatccatcttgcaaaaaaacccagacgacgtcg |
| | (beta- | | ttatcgttgctgcatacagaaccgccatcggtaagggtttc |
| | ketothiolase) | | aaaggttccttcagaaacgtccactctgaattcatcttgac |
| | POT1-2 | | tgagttcttgaaagaatttatcaaaaagaccaacatcgacc |
| | (EC 2.3.1.16) | | catctttgattgaagatgtcgctatcggtaacgtcttgaac |
| | | | caggccgcaggtgccaccgaacacagaggtgcttgtttggc |
| | | | tgccggtatcccatacaccgccgccttcatcgctgtcaaca |
| | | | gattctgttcctccggtttgatggccatctccgacattgcc |
| | | | aacaagatcaagactggtgaaatcgagtgtggtttggctgg |
| | | | tggtgccgaatccatgtccaccaactaccgtgacccaagag |
| | | | ttgccccaagaatcgacccacatttggctgacgacgcccaa |
| | | | atggaaaagtgtttgattcctatgggtatcaccaacgaaaa |
| | | | cgttgctaaccaattcaacatctccagagaaagacaagacg |
| | | | agtttgccgccaagtcctacaacaaggctgccaaggcggtt |
| | | | gcctctggtgctttcaagagtgaaatcttgccaatcagatc |
| | | | catcatcagaaactctgacggtaccgaaaaggaaatcattg |
| | | | tcgacactgacgaaggtccaagagaaggtgtcaccgctgaa |
| | | | tctttgggcaagttgagaccagctttcgacggtaccaccac |
| | | | tgcaggtaacgcttctcaagtctctgacggtgccgccgccg |
| | | | tcttgttgatgaagagaagcttggctgaagccaagggatac |
| | | | ccaatcattggtaagtacgtcctttgttccaccgccggtgt |
| | | | tccaccagaaatcatgggtgttggtccagccttcgctatcc |
| | | | cagaagtcttgaagagaactggcttgactgttgacgacatt |
| | | | gatgttttcgaaatcaacgaagcctttgccgctcaatgtct |
| | | | ttactctgctgaacaagtcaatgtgcctgaagaaaagttga |
| | | | acatcaacggtggtgccattgccttgggccatccattgggt |
| | | | gaaaccggtgctcgtcaatacgccaccatcatcccattgtt |
| | | | aaagccaggtcaaattggattgacttcaatgtgtattggtt |
| | | | ctggtatgggttctgcttctattttggttagagaatag |
| 81 | 3-ketoacyl- | Candida sp. | mdrinqlsgqlkpnakgsilqknpddvvivaayrtaigkgf |
| | CoA thiolase | polypeptide | kgsfrnvhsefilteflkefikktnidpsliedvaignvin |
| | (beta- | | qaagatehrgaclaagipytaafiavnrfcssglmaisdia |
| | ketothiolase) | | nkiktgeiecglaggaesmstnyrdprvapridphladdaq |
| | POT1-2 | | mekclipmgitnenvanqfnisrerqdefaaksynkaakav |
| | (EC 2.3.1.16) | | asgafkseilpirsiirnsdgtekeiivdtdegpregvtae |
| | | | slgklrpafdgtttagnasqvsdgaaavllmkrslaeakgy |
| | | | piigkyvlcstagvppeimgvgpafaipevlkrtgltvddi |
| | | | dvfeineafaagclysaegvnvpeeklninggaialghplg |
| | | | etgarqyatiipllkpgqigltsmcigsgmgsasilvre |
| 82 | 3-ketoacyl- | Candida sp. | atgtcagttaaaagcaagcttgccgaaaaatccccagacga |
| | CoA thiolase | polynucleotide | tgttgtcgtcgttgcagcatacagaactgcccaaaccaaag |
| | (beta- | | gtggtaagggtggcttcagaaacgtcggctccgactttctt |
| | ketothiolase) | | ttgtactccttaaccaaagaattcttgaagaagaccggcat |
| | FOX3-1 | | cgacccatccatcatccaagacgctgccatcggtaacgtct |
| | (EC 2.3.1.16) | | tgaacagaagatccggtgatttcgaacacagaggtgccttg |
| | | | ttggctgccggtatcccacacaccacccctttcatcgccat |
| | | | caacagacagtgttcctctggtttgatggccatctcccaga |
| | | | tcgccaacaagatcaagactggtgaaatcgagtgtggtttg |
| | | | gctggtggtgctgaaagcatgaccaagaactacggtccaga |
| | | | tgcattggtccaaatcgacccggcctacgctgaaaacccag |
| | | | aattcatcaagaacggtattcctatgggtatcaccaacgag |
| | | | aatgtctgtgccaagttcaacgttgccagagacgctcaaga |
| | | | tcaatttgctgctgaatcctaccaaaaggctgaaaaggctc |
| | | | aaaaggaaggtaagtttgacgacgaaatcttgccaattgaa |
| | | | gtctaccaagaagacgacgacgatgaagatgaagacgaaga |
| | | | cgccgagccaaaggaaatcaaggtcaccgtcagcaaagatg |
| | | | acggaatcagaggtggtgtcaccaaggaaaaattggccaag |
| | | | atcaagcctgccttcaaagacgacggtgtttccaccgccgg |
| | | | taactcctcccaagtttccgacggtgctgctttggtcttgt |
| | | | tgatgaagcgttcctttgctgaacaacacggcttcaagcca |
| | | | ttggccaagtacatttcttgtgccattgctggtgttccacc |
| | | | tgaactcatgggtattggtccagctgttgccattccaaagg |
| | | | tcttgaaacaaaacggcttgaacgttaacgacattgatgtt |
| | | | tacgaaattaatgaagcctttgctggtcaatgtttgtactc |
| | | | tattgaaagctgtggcattgacagatccaaggtcaacatca |
| | | | acggtggtgccattgctttgggccatccattgggtgtcacc |
| | | | ggtgctcgtcaatacgctaccatcttgagattgatgaagcc |
| | | | aggccaagttggtcttacttctatgtgtattggtactggta |
| | | | tgggtgctgct tctgttttggttaaagagtag |
| 83 | 3-ketoacyl- | Candida sp. | msvksklaekspddvvvvaayrtaqtkggkggfrnvgsdfl |
| | CoA thiolase | polypeptide | lysltkeflkktgidpsiiqdaaignvinrrsgdfehrgal |
| | (beta- | | laagiphttpfiainrqcssglmaisgiankiktgeiecgl |
| | ketothiolase) | | aggaesmtknygpdalvqidpayaenpefikngipmgitne |
| | FOX3-1 | | nvcakfnvardagdgfaaesyqkaekagkegkfddeilpie |
| | (EC 2.3.1.16) | | vyqeddddedededaepkeikvtvskddgirggvtkeklak |
| | | | ikpafkddgvstagnssqvsdgaalvllmkrsfaeqhgfkp |
| | | | lakyiscaiagvppelmgigpavaipkvlkqnglnvndidv |
| | | | yeineafaggclysiescgidrskvninggaialghplgvt |
| | | | garqyatilrlmkpgqvgltsmcigtgmgaasvlvke |
| 84 | 3-ketoacyl- | Candida sp. | atgtcagttaaaagcaagcttgccgaaaaatccccagacga |
| | CoA thiolase | polynucleotide | tgttgtcgtcgttgcagcatacagaaccgcccaaaccaaag |
| | (beta- | | gtggtaagggtggcttcagaaacgtcggctctgactttctt |
| | ketothiolase) | | ttgtactccataaccaaagaattcttgaagaagaccggcgt |
| | FOX3-2 | | cgacccatccatcatccaagacgctgccatcggtaacgtct |
| | (EC 2.3.1.16) | | tgaacagaagatccggtgatttcgaacacagaggtgccttg |
| | | | ttggctgccggtgtcccacacaccaccccattcatcgccat |
| | | | caacagacaatgttcctctggtttgatggccatctcccaga |
| | | | tcgccaacaagatcaagactggtgaaatcgagtgtggtttg |
| | | | gctggtggtgctgaaagtatgaccaagaactacggtccaga |
| | | | cgcattggtccaaatcgacccggcctacgctgaaaacccag |
| | | | aattcatcaagaacggtattcctatgggtatcaccaacgag |
| | | | aatgtctgtgccaagttcaacgttgccagagacgctcagga |
| | | | tcaatttgctgccgaatcctaccaaaaggctgaaaaggctc |
| | | | aaaaggaaggtaagtttgacgacgaaatcttgccaattgaa |
| | | | gtctaccaagaagacgacgacgacgaagatgaagacgaaga |
| | | | tgccgaaccaaaagaaatcaaggtcaccatcagcaaagatg |
| | | | acggaatcagaggtggtgtcaccaaggaaaaattggccaag |
| | | | atcaagccagccttcaaagacgacggtgtttccaccgctgg |
| | | | taactcctcccaagtttccgacggtgctgctttggtcttgt |
| | | | tgatgaagcgttcctttgctgaacaacacggcttcaagcca |
| | | | ttggccaagtacatttcttgtgccattgctggtgttccacc |
| | | | tgaactcatgggtattggtccagctgttgccattccaaagg |
| | | | tcttgaaacaaaacggcttgaacgttaacgacattgatgtt |
| | | | tacgaaattaatgaagcctttgctggtcaatgtttgtactc |
| | | | cattgaaagctgtggcattgacagatccaaggtcaacatca |
| | | | acggtggtgccattgctttgggccacccattgggtgtcacc |
| | | | ggtgctcgtcaatacgctaccatcttgagattgttgaagcc |
| | | | aggccaagttggtcttacttctatgtgtattggtactggta |
| | | | tgggtgctgct tctgttttggttagagaatag |
| 85 | 3-ketoacyl- | Candida sp. | msvksklaekspddvvvvaayrtaqtkggkggfrnvgsdfl |
| | CoA thiolase | polypeptide | lysitkeflkktgvdpsiiqdaaignvinrrsgdfehrgal |
| | (beta- | | laagvphttpfiainrqcssglmaisgiankiktgeiecgl |
| | ketothiolase) | | aggaesmtknygpdalvqidpayaenpefikngipmgitne |
| | FOX3-2 | | nvcakfnvardagdgfaaesyqkaekagkegkfddeilpie |
| | (EC 2.3.1.16) | | vyqeddddedededaepkeikvtiskddgirggvtkeklak |
| | | | ikpafkddgvstagnssqvsdgaalvllmkrsfaeqhgfkp |
| | | | lakyiscaiagvppelmgigpavaipkvlkqnglnvndidv |
| | | | yeineafaggclysiescgidrskvninggaialghplgvt |
| | | | gargyatilrllkpgqvgltsmcigtgmgaasvlvre |
| 86 | Propionyl-CoA | E. Coli K-12 | atgtcttttagcgaattttatcagcgttcgattaacgaacc |
| | synthetase | MG1655 sp. | ggagcagttctgggccgagcaggcccggcgtattgactggc |
| | PrpE | polynucleotide | agacgccctttacgcaaacgctcgatcacagcaatccgccg |
| | (EC 6.2.1.17) | | tttgcccgttggttttgtgaaggccgaaccaacttgtgcca |
| | | | caacgccatcgaccgctggctggagaaacagccagaggcgc |
| | | | tggcgctgattgccgtctcttcggaaacagaagaagagcgc |
| | | | acctttacctttcgtcagctgcatgacgaagtgaacgcggt |
| | | | ggcctcaatgttgcgttcattgggtgtgcagcgcggcgatc |
| | | | gggtgctggtgtatatgccgatgattgccgaagcgcatatt |
| | | | actctgctggcctgcgcgcgcattggcgctattcactcggt |
| | | | ggtgtttggtggatttgcctcgcacagcgtggcggcgcgaa |
| | | | ttgatgacgctaaaccggtgctgattgtctcggctgatgcc |
| | | | ggagcgcgcggtggcaaaatcattccctataaaaaattgct |
| | | | cgacgatgcgataagtcaggcgcagcaccagccacgccatg |
| | | | ttttgctggtggatcgcgggctggcgaaaatggcgcgcgtc |
| | | | agcgggcgggatgtcgatttcgcgtcgttgcgccatcaaca |
| | | | catcggcgcgcgggtaccggtggcgtggctggaatccaacg |
| | | | aaacctcctgcattctctacacttccggcacgaccggcaaa |
| | | | cctaaaggcgtgcagcgtgacgtcggcggatatgcggtggc |
| | | | gctggcgacctcgatggacaccatttttggcggcaaagcgg |
| | | | gcagcgtgttcttttgcgcatcggatatcggctgggtggtg |
| | | | gggcattcgtatatcgtttacgcgccgctgctggcggggat |
| | | | ggcgactatcgtttacgaaggattgccgacctggccggact |
| | | | gcggcgtgtggtggacaatcgtcgagaaatatcaggttagc |
| | | | cggatgttctcagcgccgaccgccattcgcgtgctgaaaaa |
| | | | attccctaccgctgaaattcgcaaacacgatctctcgtcgc |
| | | | tggaagtgctctatctggctggagaaccgctggacgagccg |
| | | | accgccagttgggtgagcaatacgctggatgtgccggtcat |
| | | | cgacaactactggcagaccgaatccggctggccgattatgg |
| | | | cgattgctcgcggtctggacgacaggccgacgcgtctggga |
| | | | agccccggtgtgccgatgtatggctataacgtgcagttgct |
| | | | taatgaagtcaccggcgaaccgtgtggcgtcaacgagaaag |
| | | | ggatgctggtggtggaagggccgctgccgccggggtgtatt |
| | | | cagaccatctggggcgacgacggccgctttgtgaagactta |
| | | | ctggtcgctgttttcccgcccggtgtacgccacctttgact |
| | | | ggggcatccgtgacgctgacggttatcactttattctcggg |
| | | | cgcactgacgatgtaattaacgttgccgggcatcggctggg |
| | | | gacgcgcgagattgaagagagtatctccagccatccgggcg |
| | | | ttgccgaagtggcggtggttggggtgaaagatgcgctgaaa |
| | | | gggcaggtggcggtggcgtttgtcattccgaaagagagcga |
| | | | cagtctggaagatcgtgatgtggcgcactcgcaagagaagg |
| | | | cgattatggcgctggtggacagccagattggcaactttggc |
| | | | cgcccggcgcacgtctggtttgtctcgcaattgccaaaaac |
| | | | gcgatccggaaaaatgctgcgccgcacgatccaggcgattt |
| | | | gcgaaggacgcgatcctggagatctgacgaccattgatgat |
| | | | cctgcgtcgttggatcagatccgccaggcgatggaagagta |
| | | | g |
| 87 | Propionyl-CoA | E. Coli K-12 | msfsefyqrsinepeqfwaegarridwqtpftqtldhsnpp |
| | synthetase | MG1655 sp. | farwfcegrtnlchnaidrwlekqpealaliaysseteeer |
| | PrpE | polypeptide | tftfrqlhdevnavasmlrslgvqrgdrvlvympmiaeahi |
| | (EC 6.2.1.17) | | tllacarigaihsvvfggfashsvaariddakpvlivsada |
| | | | garggkiipykkllddaisqaqhqprhvllvdrglakmary |
| | | | sgrdvdfaslrhqhigarvpvawlesnetscilytsgttgk |
| | | | pkgvqrdvggyavalatsmdtifggkagsvffcasdigwvv |
| | | | ghsyivyapllagmativyeglptwpdcgvwwtivekyqvs |
| | | | rmfsaptairvlkkfptaeirkhdlsslevlylagepldep |
| | | | taswvsntldvpvidnywqtesgwpimaiarglddrptrlg |
| | | | spgvpmygynvqllnevtgepcgvnekgmlvvegplppgci |
| | | | qtiwgddgrfvktywslfsrpvyatfdwgirdadgyhfilg |
| | | | rtddvinvaghrlgtreieesisshpgvaevavvgvkdalk |
| | | | gqvavafvipkesdsledrdvahsgekaimalvdsgignfg |
| | | | rpahvwfvsqlpktrsgkmlrrtigaicegrdpgdlttidd |
| | | | pasldgirgamee |
| 88 | Propionyl-CoA | Metallosphaera | atgtttatgcgatatattatggttgaggaacagaccctgaa |
| | synthetase | sedula sp. | gaccgggtcacaggaactagaggagaaggcagactataaca |
| | Msed_1456 | polynucleotide | tgagatattacgctcacctcatgaagttgagtaaggaaaaa |
| | (EC 6.2.1.17) | | cctgcagagttctggggatctctagcacaggacctgctaga |
| | | | ctggtatgagccttggaaggagaccatgagacaggaagacc |
| | | | cgatgacaaggtggttcataggaggtaagataaatgcctcg |
| | | | tacaacgctgtcgacagacacctcaacggccccagaaagtt |
| | | | caaggctgcggtcatctgggaaagtgagttaggggaaagga |
| | | | agatcgtgacgtatcaggacatgttctatgaggttaatagg |
| | | | tgggccaatgcgctcagatccctaggagttggtaaagggga |
| | | | tagggtgaccatatacatgcccctgaccccagagggaatag |
| | | | ctgcaatgctggcctcggccaggataggtgcaattcatagc |
| | | | gtaatatttgccggctttggttcgcaagccatagccgacag |
| | | | ggttgaggacgccaaggcgaaggtagtgatcactgctgacg |
| | | | cctatcccagaaggggaaaggttgtggagttaaagaagact |
| | | | gtcgacgaggccttaaactcccttggagaaaggagcccagt |
| | | | acagcacgtgctcgtgtataggaggatgaaaacggatgtaa |
| | | | acatgaaggagggaagagacgttttcttcgacgaggtcggc |
| | | | aagtacaggtacgtggagcctgaaaggatggactccaatga |
| | | | tccactcttcattctctacacctctgggaccaccggtaaac |
| | | | ctaagggaattatgcactctaccggtggttatctgaccggg |
| | | | acagccgttatgctactgtggagctacggccttagccagga |
| | | | gaacgacgttctcttcaacacctcagatattggttggatag |
| | | | ttggccactcctacattacctattccccccttatcatgggg |
| | | | agaacggttgtcatttacgagagcgccccagactatcccta |
| | | | cccagacaagtgggctgagattattgagagatacagggcaa |
| | | | ccactttcggcacctcagctacagccttgcgttacttcatg |
| | | | aagtatggggacgaatacgtgaagaaccacgatctctcgtc |
| | | | catcaggataattgtgacgaacggggaagtgcttaactact |
| | | | ctccgtggaagtgggggctagaagtgttaggtggaggaaag |
| | | | gtattcatgtcccatcagtggtggcaaactgagacaggcgc |
| | | | accgaacctgggctaccttccgggtataatttacatgccaa |
| | | | tgaagtcgggtccagcctcaggcttccctctacccggtaac |
| | | | ttcgtggaggttctggacgagaacggaaatccctctgcccc |
| | | | tagagtgagaggataccttgtaatgaggccacccttcccgc |
| | | | ctaacatgatgatggggatgtggaacgataatggggagagg |
| | | | ttgaagaagacgtactttagcaagttcggttccctgtatta |
| | | | tccaggagacttcgccatggtggatgaggatggatacatct |
| | | | gggtgttgggtagggcagacgagactctaaaaattgcagcc |
| | | | cacagaattggagctggggaagtggaatcagcaatcacttc |
| | | | tcacccatcggttgccgaggcagcagtcataggcgtgccag |
| | | | actcagtgaaaggagaagaggttcacgcgttcgttgtgcta |
| | | | aagcaaggttacgctccttcctctgaactggctaaggacat |
| | | | acagtcacacgttaggaaggtcatggggcccattgttagtc |
| | | | cgcagattcatttcgtggataagttgcctaagacaaggtct |
| | | | gggaaggtcatgagaagggtgataaaggcagtgatgatggg |
| | | | ttcgagtgctggcgacttaaccaccatagaggacgaagcat |
| | | | caatggacgaaataaagaaggctgtcgaggaactaaagaag |
| | | | gagttaaagacctcctag |
| 89 | Propionyl-CoA | Metallosphaera | mfmryimveegtlktgsgeleekadynmryyahlmklskek |
| | synthetase | sedula sp. | paefwgslagdlldwyepwketmrgedpmtrwfiggkinas |
| | Msed_1456 | polypeptide | ynavdrhingprkfkaaviweselgerkivtygdmfyevnr |
| | (EC 6.2.1.17) | | wanalrslgvgkgdrvtiympltpegiaamlasarigaihs |
| | | | vifagfgsgaiadrvedakakvvitadayprrgkvvelkkt |
| | | | vdealnslgerspvqhvlvyrrmktdvnmkegrdvffdevg |
| | | | kyryvepermdsndplfilytsgttgkpkgimhstggyltg |
| | | | tavmllwsyglsgendvlfntsdigwivghsyitysplimg |
| | | | rtvviyesapdypypdkwaeiieryrattfgtsatalryfm |
| | | | kygdeyvknhdlssiriivtngevinyspwkwglevlgggk |
| | | | vfmshqwwqtetgapnlgylpgiiympmksgpasgfplpgn |
| | | | fvevldengnpsaprvrgylvmrppfppnmmmgmwndnger |
| | | | lkktyfskfgslyypgdfamvdedgyiwvlgradetlkiaa |
| | | | hrigagevesaitshpsvaeaavigvpdsvkgeevhafvvl |
| | | | kggyapsselakdigshvrkvmgpivspqihfvdklpktrs |
| | | | gkvmrrvikavmmgssagdlttiedeasmdeikkaveelkk |
| | | | elkts |
| 90 | Propionyl-CoA | Salmonella | atgtcttttagcgaattttatcagcgttccattaacgaacc |
| | synthetase | typhimurium | ggaggcgttctgggccgagcaggcccggcgtatcgactggc |
| | PrpE | sp. | gacagccgtttacgcagacgctggatcatagccgtccaccg |
| | (EC 6.2.1.17) | polynucleotide | tttgcccgctggttttgcggcggcaccactaacttatgtca |
| | | | taacgccgtcgaccgctggcgggataaacagccggaggcgc |
| | | | tggcgctgattgccgtctcatcagagaccgatgaagagcgc |
| | | | acatttaccttcagccagttgcatgatgaagtcaacgctgt |
| | | | ggccgctatgctgctgtcgctgggcgtgcagcgtggcgatc |
| | | | gcgtattggtctatatgccgatgattgccgaagcgcagata |
| | | | accctgctggcctgtgcgcgcattggcgcgatccattcggt |
| | | | ggtctttggcggttttgcctcgcacagcgtggcggcgcgca |
| | | | ttgacgatgccagaccggcgctgattgtgtcggcggatgcc |
| | | | ggagcgcgtggcggtaaaattctgccgtataaaaagctgct |
| | | | tgatgacgctattgcgcaggcgcagcatcagccgaaacacg |
| | | | ttctgctggtggacagagggctggcgaaaatgtcgtgggtg |
| | | | gatgggcgcgatctggatttttccacgttgcgccagcagta |
| | | | tctcggcgcgagcgtgccggtggcgtggctggaatccaatg |
| | | | aaacctcgtgcattctttacacctccggcactaccggcaaa |
| | | | ccgaaaggcgtccagcgcgacgtcggcggttatgcggtggc |
| | | | gctggcaacctcgatggacaccatttttggcggcaaggcgg |
| | | | gcggcgtattcttttgcgcatcggatatcggctgggtcgtc |
| | | | ggccactcctatatcgtttacgcgccgctgctggcaggcat |
| | | | ggcgactattgtttacgaaggactgccgacgtacccggact |
| | | | gcggggtctggtggaaaattgtcgagaaataccaggttaac |
| | | | cggatgttttccgccccgaccgcgattcgcgtgctgaaaaa |
| | | | attcccgacggcgcaaatccgcaatcacgatctctcctcgc |
| | | | tggaggcgctttatctggccggtgagccgctggacgagccg |
| | | | acggccagttgggtgacggagacgctgggcgtaccggtcat |
| | | | cgacaattattggcagacggagtccggctggccgatcatgg |
| | | | cgctggcccgcgcgctggacgacaggccgtcgcgtctggga |
| | | | agtcccggggtgccgatgtacggttataacgtccagctact |
| | | | caatgaagtcaccggcgaaccctgcggcataaatgaaaaag |
| | | | gcatgctggtgatcgaagggccgctgccgccgggttgtatt |
| | | | cagactatttggggcgacgatgcgcgttttgtgaagactta |
| | | | ctggtcgctgtttaaccgtcaggtttatgccactttcgact |
| | | | ggggaatccgcgacgccgaggggtattactttattctgggc |
| | | | cgtaccgatgatgtgattaatattgcgggtcatcggctggg |
| | | | gacgcgagaaatagaagaaagtatctccggttacccgaacg |
| | | | tagcggaagtggcggtggtggggataaaagacgctctgaaa |
| | | | gggcaggtggcggtggcgtttgtcattccgaagcagagcga |
| | | | tacgctggcggatcgcgaggcggcgcgcgacgaggaaaaag |
| | | | cgattatggcgctggtggataaccagatcggtcactttggt |
| | | | cgtccggcgcatgtctggtttgtttcgcagctccccaaaac |
| | | | gcgttccggaaagatgcttcgccgcacgatccaggcgatct |
| | | | gcgaaggccgtgatccgggcgatctgacaaccattgacgat |
| | | | cccgcgtcgttgcagcaaattcgccaggcgatcgaggaata |
| | | | g |
| 91 | Propionyl-CoA | Salmonella | msfsefyqrsinepeafwaegarridwruftqtldhsrpp |
| | synthetase | typhimurium | farwfcggttnlchnavdrwrdkuealaliayssetdeer |
| | PrpE | sp. | tftfsqlhdevnavaamllslgvqrgdrvlvympmiaeaqi |
| | (EC 6.2.1.17) | polypeptide | tllacarigaihsvvfggfashsvaariddarpalivsada |
| | | | garggkilpykkllddaiagaqhqpkhvllvdrglakmswv |
| | | | dgrdldfstlrqqylgasvpvawlesnetscilytsgttgk |
| | | | pkgvqrdvggyavalatsmdtifggkaggvffcasdigwvv |
| | | | ghsyivyapllagmativyeglptypdcgvwwkivekyqvn |
| | | | rmfsaptairvlkkfptaqirnhdlsslealylagepldep |
| | | | taswvtetlgvpvidnywqtesgwpimalaralddrpsrlg |
| | | | spgvpmygynvqllnevtgepcginekgmlviegplppgci |
| | | | qtiwgddarfvktywslfnrqvyatfdwgirdaegyyfilg |
| | | | rtddviniaghrlgtreieesisgypnvaevavvgikdalk |
| | | | gqvavafvipkgsdtladreaardeekaimalvdngighfg |
| | | | rpahvwfvsqlpktrsgkmlrrtigaicegrdpgdlttidd |
| | | | paslqqirgaiee |
| 92 | Acyl-CoA | Pseudomonas | atgctggtaaatgacgagcaacaacagatcgccgacgcggt |
| | dehydrogenase | putida KT2440 | acgtgcgttcgcccaggaacgcctgaagccgtttgccgagc |
| | PP_2216 | sp. | aatgggacaaggaccatcgcttcccgaaagaggccatcgac |
| | (EC 1.3.8.7) | polynucleotide | gagatggccgaactgggcctgttcggcatgctggtgccgga |
| | | | gcagtggggcggtagcgacaccggttatgtggcctatgcca |
| | | | tggccttggaggaaatcgctgcgggcgatggcgcctgctcg |
| | | | accatcatgagcgtgcacaactcggtgggttgcgtgccgat |
| | | | cctgcgcttcggcaacgagcagcagaaagagcagttcctca |
| | | | ccccgctggcgacaggtgcgatgctcggtgctttcgccctg |
| | | | accgagccgcaggctggctccgatgccagcagcctgaagac |
| | | | ccgcgcacgcctggaaggcgaccattacgtgctcaatggca |
| | | | gcaagcagttcattacctcggggcagaacgccggcgtagtg |
| | | | atcgtgtttgcggtcaccgacccggaggccggcaagcgtgg |
| | | | catcagcgccttcatcgtgccgaccgattcgccgggctacc |
| | | | aggtagcgcgggtggaggacaaactcggccagcacgcctcc |
| | | | gacacctgccagatcgttttcgacaatgtgcaagtgccagt |
| | | | ggccaaccggctgggggcggagggtgaaggctacaagatcg |
| | | | ccctggccaaccttgaaggcggccgtatcggcatcgcctcg |
| | | | caagcggtgggtatggcccgcgcggcgttcgaagtggcgcg |
| | | | ggactatgccaacgagcgccagagctttggcaaaccgctga |
| | | | tcgagcaccaggccgtggcgtttcgcctggccgacatggca |
| | | | acgaaaatttccgttgcccggcagatggtattgcacgccgc |
| | | | tgcccttcgtgatgcggggcgcccggcgctggtggaagcgt |
| | | | cgatggccaagctgttcgcctcggaaatggccgaaaaggtc |
| | | | tgttcggacgccttgcagaccctgggcggttatggctatct |
| | | | gagtgacttcccgctggagcggatctaccgcgacgttcggg |
| | | | tttgccagatctacgaaggcaccagcgacattcagcgcatg |
| | | | gtcattgcgcgcaatctttga |
| 93 | Acyl-CoA | Pseudomonas | mlvndeqqqiadavrafagerlkpfaeqwdkdhrfpkeaid |
| | dehydrogenase | putida KT2440 | emaelglfgmlvpeqwggsdtgyvayamaleeiaagdgacs |
| | PP_2216 | sp. | timsvhnsvgcvpilrfgneggkegfltplatgamlgafal |
| | (EC 1.3.8.7) | polypeptide | tepgagsdasslktrarlegdhyvingskqfitsgqnagvv |
| | | | ivfavtdpeagkrgisafivptdspgyqvarvedklgqhas |
| | | | dtcgivfdnvqvpvanrlgaegegykialanleggrigias |
| | | | gavgmaraafevardyanergsfgkpliehgavafrladma |
| | | | tkisvarqmvlhaaalrdagrpalveasmaklfasemaekv |
| | | | csdalgtlggygylsdfpleriyrdvrvcqiyegtsdigrm |
| | | | viarnl |
| 94 | Acyl-CoA | Pseudomonas | atgcccgagaccctgctcagcccccgcaacctggcctttga |
| | dehydrogenase | putida H8234 | gctctacgaagtgctcgacgcccaagccctcacccaacgcc |
| | PP_2216 | sp. | cgcgctttgccgagcacagccgcgaaaccttcgacgcggca |
| | (EC 1.3.8.1) | polynucleotide | ctgaccaccgcgcgcaccatcgccgaaaagtacttcgcccc |
| | | | gcacaaccgcaaggccgacgaaaacgagccgcgctacgtgg |
| | | | acggccgcgctgaactgatccccgaggtcaagcctgccgtc |
| | | | gacgcattcctcgaagccggcttcctcaacgccaaccggga |
| | | | cttcgagttcggcggcatgcagctgcccagcctggtttcgc |
| | | | aggcctgcttcgctcacttccaggctgccaacgccggcacc |
| | | | acggcctacccgttcctgaccatgggcgcagccaacctgat |
| | | | cgaaagtttcggcacagaggaacagaagcgtctgttcctgc |
| | | | agccaatgatcgagggccgctacttcggcaccatggcgctg |
| | | | accgagccccacgctggctcgtctctggccgacatccgcac |
| | | | ccgtgccgaacctgcgggcgacggcagctaccggctcaagg |
| | | | gcaacaagatcttcatctccggtggcgaccacgaactgtcg |
| | | | gaaaacatcgtgcacatggtgctggccaagctgccggacgc |
| | | | accgcctggggtgaaaggcatctcgctgttcatcgtgccca |
| | | | agtacaacgtcaaccccgacggcagccgtggcccgcgcaac |
| | | | gacgtgctgctggccgggctgttccacaagatgggctggcg |
| | | | cggtaccacctccaccgcgctgaacttcggcgacaacgacc |
| | | | agtgcgtcggctacctggtcggccagccgcaccaaggcctg |
| | | | gcctgcatgttccagatgatgaacgaggcgcgtatcggcgt |
| | | | tggcatgggcgcggtgatgctcggatacgccggctacctgt |
| | | | attcgctggaatatgcccgccaacggccgcaaggccggctg |
| | | | ccggacaacaaagacccgctcagcccggcggtgccgatcat |
| | | | cgcgcacaccgatgtgaaacgtatgctgctggcacagaagg |
| | | | cgtacgtggaaggcgccttcgacctgggcctttacgccgcg |
| | | | cgcctgttcgacgatacccacaccgccgatgacgaaacgtc |
| | | | ccgcacacaagcgcaggcgctgctcgacctgctgaccccgt |
| | | | tcgtcaagtcgtggccctcgacgttctgcctcaaggccaac |
| | | | gaactggcgatccagattctcggtggccacggctatacccg |
| | | | cgaatacccggtggaacagtactaccgcgacaatcgcctga |
| | | | acccgatccacgagggcaccgaaggcattcagtcgctcgac |
| | | | ttgctcggccgcaagctggcacagaaccatggtgccggcct |
| | | | caagcaactgatccgcctgatcgccaccaccggcgaacgtg |
| | | | caagccaccaccccaaactcgacccactgcgccagccactg |
| | | | gagcaactggtcaaccgcctgcagggcgtgacactggccct |
| | | | gctcggcgacatggcccaaggcgaagtcgctggtgccttgg |
| | | | caaactcggccttgtacctcaaggccttcggccattgcgtg |
| | | | atcggctggcgctggctggaacaggccattcacgccgagct |
| | | | tggcctgcagaaaggtcaccctgccgatcgcgacttctatc |
| | | | agggcaagctgcaggccgcgcgttatttcctgacctgggaa |
| | | | gtaccgggctgccataatgagctggcattgctagaggcgcg |
| | | | cgacaacacttgcctcaccatgcaggacgagtggttctaa |
| 95 | Acyl-CoA | Pseudomonas | mpetllsprnlafelyevldagaltgrprfaehsretfdaa |
| | dehydrogenase | putida H8234 | lttartiaekyfaphnrkadenepryvdgraelipevkpav |
| | PP_2216 | sp. | dafleagflnanrdfefggmqlpslvsgacfahfgaanagt |
| | (EC 1.3.8.1) | polypeptide | taypfltmgaanliesfgteeqkrlflqpmiegryfgtmal |
| | | | tephagssladirtraepagdgsyrlkgnkifisggdhels |
| | | | enivhmvlaklpdappgvkgislfivpkynvnpdgsrgprn |
| | | | dvllaglfhkmgwrgttstalnfgdndqcvgylvgqphqgl |
| | | | acmfgmmnearigvgmgavmlgyagylysleyargrpqgrl |
| | | | pdnkdplspavpiiahtdvkrmllagkayvegafdlglyaa |
| | | | rlfddthtaddetsrtgagalldlltpfvkswpstfclkan |
| | | | elaigilgghgytreypvegyyrdnrinpihegtegigsld |
| | | | llgrklagnhgaglkqlirliattgerashhpkldplrqpl |
| | | | eqlvnrlqgvtlallgdmaggevagalansalylkafghcv |
| | | | igwrwlegaihaelglqkghpadrdfyggklqaaryfltwe |
| | | | vpgchnelalleardntcltmgdewf |
| 96 | Acyl-CoA | Candida sp. | atgacttttacaaagaaaaacgttagtgtatcacaaggtcc |
| | oxidase POX4 | polynucleotide | tgaccctagatcatccatccaaaaggaaagagacagctcca |
| | (EC 1.3.3.6) | | aatggaaccctcaacaaatgaactacttcttggaaggctcc |
| | | | gtcgaaagaagtgagttgatgaaggctttggcccaacaaat |
| | | | ggaaagagacccaatcttgttcacagacggctcctactacg |
| | | | acttgaccaaggaccaacaaagagaattgaccgccgtcaag |
| | | | atcaacagaatcgccagatacagagaacaagaatccatcga |
| | | | cactttcaacaagagattgtccttgattggtatctttgacc |
| | | | cacaggtcggtaccagaattggtgtcaacctcggtttgttc |
| | | | ctttcttgtatcagaggtaacggtaccacttcccaattgaa |
| | | | ctactgggctaacgaaaaggaaaccgctgacgttaaaggta |
| | | | tctacggttgtttcggtatgaccgaattggcccacggttcc |
| | | | aacgttgctggtttggaaaccaccgccacatttgacaagga |
| | | | atctgacgagtttgtcatcaacaccccacacattggtgcca |
| | | | ccaagtggtggattggtggtgctgctcactccgccacccac |
| | | | tgttctgtctacgccagattgattgttgacggtcaagatta |
| | | | cggtgtcaagacttttgttgtcccattgagagactccaacc |
| | | | acgacctcatgccaggtgtcactgttggtgacattggtgcc |
| | | | aagatgggtagagatggtatcgataacggttggatccaatt |
| | | | ctccaacgtcagaatcccaagattctttatgttgcaaaagt |
| | | | tctgtaaggtttctgctgaaggtgaagtcaccttgccacct |
| | | | ttggaacaattgtcttactccgccttgttgggtggtagagt |
| | | | catgatggttttggactcctacagaatgttggctagaatgt |
| | | | ccaccattgccttgagatacgccattggtagaagacaattc |
| | | | aagggtgacaatgtcgatccaaaagatccaaacgctttgga |
| | | | aacccaattgatagattacccattgcaccaaaagagattgt |
| | | | tcccatacttggctgctgcctacgtcatctccgctggtgcc |
| | | | ctcaaggttgaagacaccatccataacaccttggctgaatt |
| | | | ggacgctgccgttgaaaagaacgacaccaaggctatcttta |
| | | | agtctattgacgacatgaagtcattgtttgttgactctggt |
| | | | tccttgaagtccactgccacttggttgggtgctgaagccat |
| | | | tgaccaatgtagacaagcctgtggtggtcacggttactcgt |
| | | | cctacaacggcttcggtaaagcctacaacgattgggttgtc |
| | | | caatgtacttgggaaggtgacaacaatgtcttggccatgag |
| | | | tgttggtaagccaattgtcaagcaagttatcagcattgaag |
| | | | atgccggcaagaccgtcagaggttccaccgctttcttgaac |
| | | | caattgaaggactacactggttccaacagctccaaggttgt |
| | | | tttgaacactgttgctgacttggacgacatcaagactgtca |
| | | | tcaaggctattgaagttgccatcatcagattgtcccaagaa |
| | | | gctgcttctattgtcaagaaggaatctttcgactatgtcgg |
| | | | cgctgaattggttcaactctccaagttgaaggctcaccact |
| | | | acttgttgactgaatacatcagaagaattgacacctttgac |
| | | | caaaaggacttggttccatacttgatcaccctcggtaagtt |
| | | | gtacgctgccactattgtcttggacagatttgccggtgtct |
| | | | tcttgactttcaacgttgcctccaccgaagccatcactgct |
| | | | ttggcctctgtgcaaattccaaagttgtgtgctgaagtcag |
| | | | accaaacgttgttgcttacaccgactccttccaacaatccg |
| | | | acatgattgtcaattctgctattggtagatacgatggtgac |
| | | | atctatgagaactactttgacttggtcaagttgcagaaccc |
| | | | accatccaagaccaaggctccttactctgatgctttggaag |
| | | | ccatgttgaacagaccaaccttggacgaaagagaaagattt |
| | | | gaaaagtctgatgaaaccgctgctatcttgtccaagtaa |
| 97 | Acyl-CoA | Candida sp. | mtftkknvsysqgpdprssigkerdsskwnpqqmnyflegs |
| | oxidase POX4 | polypeptide | verselmkalaqqmerdpilftdgsyydltkdqqreltavk |
| | (EC 1.3.3.6) | | inriaryreqesidtfnkrlsligifdpqvgtrigvnlglf |
| | | | lscirgngttsqlnywaneketadvkgiygcfgmtelahgs |
| | | | nvaglettatfdkesdefvintphigatkwwiggaahsath |
| | | | csvyarlivdggdygvktfvvplrdsnhdlmpgvtvgdiga |
| | | | kmgrdgidngwiqfsnvriprffmlqkfckvsaegevtlpp |
| | | | leqlsysallggrvmmvldsyrmlarmstialryaigrrqf |
| | | | kgdnvdpkdpnaletqlidyplhqkrlfpylaaayvisaga |
| | | | lkvedtihntlaeldaavekndtkaifksiddmkslfvdsg |
| | | | slkstatwlgaeaidgcrqacgghgyssyngfgkayndwvv |
| | | | qctwegdnnvlamsvgkpivkqvisiedagktvrgstafln |
| | | | qlkdytgsnsskvvintvadlddiktvikaievaiirlsge |
| | | | aasivkkesfdyvgaelvqlsklkahhyllteyirridtfd |
| | | | qkdlvpylitlgklyaativldrfagvfltfnvasteaita |
| | | | lasvgipklcaevrpnvvaytdsfqqsdmivnsaigrydgd |
| | | | iyenyfdlvklqnppsktkapysdaleamlnrptldererf |
| | | | eksdetaailsk |
| 98 | Acyl-CoA | Candida sp. | atgcctaccgaacttcaaaaagaaagagaactcaccaagtt |
| | oxidase POX5 | polynucleotide | caacccaaaggagttgaactacttcttggaaggttcccaag |
| | (EC 1.3.3.6) | | aaagatccgagatcatcagcaacatggtcgaacaaatgcaa |
| | | | aaagaccctatcttgaaggtcgacgcttcatactacaactt |
| | | | gaccaaagaccaacaaagagaagtcaccgccaagaagattg |
| | | | ccagactctccagatactttgagcacgagtacccagaccaa |
| | | | caggcccagagattgtcgatcctcggtgtctttgacccaca |
| | | | agtcttcaccagaatcggtgtcaacttgggtttgtttgttt |
| | | | cctgtgtccgtggtaacggtaccaactcccagttcttctac |
| | | | tggaccataaataagggtatcgacaagttgagaggtatcta |
| | | | tggttgttttggtatgactgagttggcccacggttccaacg |
| | | | tccaaggtattgaaaccaccgccacttttgacgaagacact |
| | | | gacgagtttgtcatcaacaccccacacattggtgccaccaa |
| | | | gtggtggatcggtggtgctgcgcactccgccacccactgct |
| | | | ccgtctacgccagattgaaggtcaaaggaaaggactacggt |
| | | | gtcaagacctttgttgtcccattgagagactccaaccacga |
| | | | cctcgagccaggtgtgactgttggtgacattggtgccaaga |
| | | | tgggtagagacggtatcgataacggttggatccagttctcc |
| | | | aacgtcagaatcccaagattctttatgttgcaaaagtactg |
| | | | taaggtttcccgtctgggtgaagtcaccatgccaccatctg |
| | | | aacaattgtcttactcggctttgattggtggtagagtcacc |
| | | | atgatgatggactcctacagaatgaccagtagattcatcac |
| | | | cattgccttgagatacgccatccacagaagacaattcaaga |
| | | | agaaggacaccgataccattgaaaccaagttgattgactac |
| | | | ccattgcatcaaaagagattgttcccattcttggctgccgc |
| | | | ttacttgttctcccaaggtgccttgtacttagaacaaacca |
| | | | tgaacgcaaccaacgacaagttggacgaagctgtcagtgct |
| | | | ggtgaaaaggaagccattgacgctgccattgtcgaatccaa |
| | | | gaaattgttcgtcgcttccggttgtttgaagtccacctgta |
| | | | cctggttgactgctgaagccattgacgaagctcgtcaagct |
| | | | tgtggtggtcacggttactcgtcttacaacggtttcggtaa |
| | | | agcctactccgactgggttgtccaatgtacctgggaaggtg |
| | | | acaacaacatcttggccatgaacgttgccaagccaatggtt |
| | | | agagacttgttgaaggagccagaacaaaagggattggttct |
| | | | ctccagcgttgccgacttggacgacccagccaagttggtta |
| | | | aggctttcgaccacgccctttccggcttggccagagacatt |
| | | | ggtgctgttgctgaagacaagggtttcgacattaccggtcc |
| | | | aagtttggttttggtttccaagttgaacgctcacagattct |
| | | | tgattgacggtttcttcaagcgtatcaccccagaatggtct |
| | | | gaagtcttgagacctttgggtttcttgtatgccgactggat |
| | | | cttgaccaactttggtgccaccttcttgcagtacggtatca |
| | | | ttaccccagatgtcagcagaaagatttcctccgagcacttc |
| | | | ccagccttgtgtgccaaggttagaccaaacgttgttggttt |
| | | | gactgatggtttcaacttgactgacatgatgaccaatgctg |
| | | | ctattggtagatatgatggtaacgtctacgaacactacttc |
| | | | gaaactgtcaaggctttgaacccaccagaaaacaccaaggc |
| | | | tccatactccaaggctttggaagacatgttgaaccgtccag |
| | | | accttgaagtcagagaaagaggtgaaaagtccgaagaagct |
| | | | gctgaaatcttgtccagttaa |
| 99 | Acyl-CoA | Candida sp. | mptelgkereltkfnpkelnyflegsgerseiisnmvegmq |
| | oxidase POX5 | polypeptide | kdpilkvdasyynitkdqgrevtakkiarlsryfeheypdg |
| | (EC 1.3.3.6) | | qaqrlsilgvfdpqvftrigvnlglfvscvrgngtnsqffy |
| | | | wtinkgidklrgiygcfgmtelahgsnvqgiettatfdedt |
| | | | defvintphigatkwwiggaahsathcsvyarlkvkgkdyg |
| | | | vktfvvplrdsnhdlepgvtvgdigakmgrdgidngwiqfs |
| | | | nvriprffmlqkyckvsrsgevtmppseqlsysaliggrvt |
| | | | mmmdsyrmtsrfitialryaihrrqfkkkdtdtietklidy |
| | | | plhqkrlfpflaaaylfsggalylegtmnatndkldeaysa |
| | | | gekeaidaaiveskklfvasgclkstctwltaeaidearga |
| | | | cgghgyssyngfgkaysdwvvqctwegdnnilamnvakpmv |
| | | | rdllkepeqkglvlssvadlddpaklvkafdhalsglardi |
| | | | gavaedkgfditgpslvlvsklnahrflidgffkritpews |
| | | | evlrplgflyadwiltnfgatflqygiitpdvsrkissehf |
| | | | palcakvrpnvvgltdgfnitdmmtnaaigrydgnvyehyf |
| | | | etvkalnppentkapyskaledmlnrpdlevrergekseea |
| | | | aeilss |
| 100 | Enoyl-CoA | Candida sp. | atgtctccagttgattttaaagataaagttgtgatcattac |
| | hydratase | polynucleotide | cggtgccggtggtggtttgggtaaatactactccctcgaat |
| | FOX2/HDE | | ttgccaagttgggcgccaaagtcgtcgttaacgacttgggt |
| | (EC 4.2.1.17) | | ggtgccttgaacggtcaaggtggaaactccaaggccgccga |
| | | | cgttgtcgttgacgaaattgtcaagaacggtggtgttgccg |
| | | | ttgccgattacaacaacgtcttggacggtgacaagattgtc |
| | | | gaaaccgccgtcaagaactttggtactgtccacgttatcat |
| | | | caacaatgccggtatcttgagagatgcctccatgaagaaga |
| | | | tgactgaaaaagactacaaattggtcattgacgtgcacttg |
| | | | aacggtgcctttgccgtcaccaaggctgcttggccatactt |
| | | | ccaaaagcaaaaatacggtagaattgtcaacacatcctccc |
| | | | cagctggtttgtacggtaactttggtcaagccaactacgcc |
| | | | tccgccaagtctgctttgttgggattcgctgaaaccttggc |
| | | | caaggaaggtgccaaatacaacatcaaggccaacgccattg |
| | | | ctccgttggccagatcaagaatgactgaatctatcttgcca |
| | | | cctccaatgttggaaaaattgggccctgaaaaggttgcccc |
| | | | attggtcttgtatttgtcgtcagctgaaaacgaattgactg |
| | | | gtcaattctttgaagttgctgctggcttttacgctcagatc |
| | | | agatgggaaagatccggtggtgtcttgttcaagccagatca |
| | | | atccttcaccgctgaggttgttgctaagagattctctgaaa |
| | | | tccttgattatgacgactctaggaagccagaatacttgaag |
| | | | aaccaatacccattcatgttgaacgactacgccactttgac |
| | | | caacgaagctagaaagttgccagctaacgatgcttctggtg |
| | | | ctccaactgtctccttgaaggacaaggttgttttgatcacc |
| | | | ggtgccggtgctggtttgggtaaagaatacgccaagtggtt |
| | | | cgccaagtacggtgccaaggttgttgttaacgacttcaagg |
| | | | atgctaccaagaccgttgacgaaatcaaagccgctggtggt |
| | | | gaagcttggccagatcaacacgatgttgccaaggactccga |
| | | | agctatcatcaagaatgtcattgacaagtacggtaccattg |
| | | | atatcttggtcaacaacgccggtatcttgagagacagatcc |
| | | | tttgccaagatgtccaagcaagaatgggactctgtccaaca |
| | | | agtccacttgattggtactttcaacttgagcagattggcat |
| | | | ggccatactttgttgaaaaacaatttggtagaatcatcaac |
| | | | attacctccaccagtggtatctacggtaactttggtcaagc |
| | | | caactactcgtcttctaaggctggtatcttgggtttgtcca |
| | | | agaccatggccattgaaggtgctaagaataacattaaggtc |
| | | | aacattgttgctccacacgctgaaactgccatgaccttgac |
| | | | catcttcagagaacaagacaagaacttgtaccacgctgacc |
| | | | aagttgctccattgttggtctacttgggtactgacgatgtc |
| | | | ccagtcaccggtgaaactttcgaaatcggtggtggttggat |
| | | | cggtaacaccagatggcaaagagccaagggtgctgtctccc |
| | | | acgacgaacacaccactgttgaattcatcaaggagcacttg |
| | | | aacgaaatcactgacttcaccactgacactgaaaatccaaa |
| | | | atctaccaccgaatcctccatggctatcttgtctgccgttg |
| | | | gtggtgatgacgatgatgatgacgaagacgaagaagaagac |
| | | | gaaggtgatgaagaagaagacgaagaagacgaagaagaaga |
| | | | cgatccagtctggagattcgacgacagagatgttatcttgt |
| | | | acaacattgcccttggtgccaccaccaagcaattgaagtac |
| | | | gtctacgaaaacgactctgacttccaagtcattccaacctt |
| | | | tggtcacttgatcaccttcaactctggtaagtcacaaaact |
| | | | cctttgccaagttgttgcgtaacttcaacccaatgttgttg |
| | | | ttgcacggtgaacactacttgaaggtgcacagctggccacc |
| | | | accaaccgaaggtgaaatcaagaccactttcgaaccaattg |
| | | | ccactactccaaagggtaccaacgttgttattgttcacggt |
| | | | tccaaatctgttgacaacaagtctggtgaattgatttactc |
| | | | caacgaagccacttacttcatcagaaactgtcaagccgaca |
| | | | acaaggtctacgctgaccgtccagcattcgccaccaaccaa |
| | | | ttcttggcaccaaagagagccccagactaccaagttgacgt |
| | | | tccagtcagtgaagacttggctgctttgtaccgtttgtctg |
| | | | gtgacagaaacccattgcacattgatccaaactttgctaaa |
| | | | ggtgccaagttccctaagccaatcttacacggtatgtgcac |
| | | | ttatggtttgagtgctaaggctttgattgacaagtttggta |
| | | | tgttcaacgaaatcaaggccagattcaccggtattgtcttc |
| | | | ccaggtgaaaccttgagagtcttggcatggaaggaaagcga |
| | | | tgacactattgtcttccaaactcatgttgttgatagaggta |
| | | | ctattgccattaacaacgctgctattaagttagtcggtgac |
| | | | aaagcaaagatc |
| 101 | Enoyl-CoA | Candida sp. | mspvdfkdkvviitgaggglgkyyslefaklgakvvvndlg |
| | hydratase | polypeptide | galngqggnskaadvvvdeivknggvavadynnvldgdkiv |
| | FOX2/HDE | | etavknfgtvhviinnagilrdasmkkmtekdyklvidvhl |
| | (EC 4.2.1.17) | | ngafavtkaawpyfqkqkygrivntsspaglygnfgqanya |
| | | | saksallgfaetlakegakynikanaiaplarsrmtesilp |
| | | | ppmleklgpekvaplvlylssaeneltgqffevaagfyaqi |
| | | | rwersggvlfkpdqsftaevvakrfseildyddsrkpeylk |
| | | | nqypfmlndyatltnearklpandasgaptvslkdkvvlit |
| | | | gagaglgkeyakwfakygakvvvndfkdatktvdeikaagg |
| | | | eawpdqhdvakdseaiiknvidkygtidilvnnagilrdrs |
| | | | fakmskqewdsvqqvhligtfnlsrlawpyfvekqfgriin |
| | | | itstsgiygnfgqanyssskagilglsktmaiegaknnikv |
| | | | nivaphaetamtltifreqdknlyhadqvapllvylgtddv |
| | | | pvtgetfeigggwigntrwqrakgayshdehttvefikehl |
| | | | neitdfttdtenpksttessmailsavggddddddedeeed |
| | | | egdeeedeedeeeddpvwrfddrdvilynialgattkqlky |
| | | | vyendsdfqviptfghlitfnsgksqnsfakllrnfnpmll |
| | | | lhgehylkvhswppptegeikttfepiattpkgtnvvivhg |
| | | | sksvdnksgeliysneatyfirncqadnkvyadrpafatnq |
| | | | flapkrapdyqvdvpvsedlaalyrlsgdrnplhidpnfak |
| | | | gakfpkpilhgmctyglsakalidkfgmfneikarftgivf |
| | | | pgetlrvlawkesddtivfqthvvdrgtiainnaaiklvgd |
| | | | kaki |
| 102 | 3- | Candida sp. | atgattcgcttcactgtttcttcaattagacccatcaactg |
| | hydroxypropionyl- | polynucleotide | tgctacaaggagatccatatcactactacaatcaagaatgt |
| | CoA | | catccagtgtatcgacaaacccaactgccgggggcgaagaa |
| | hydrolase | | gagccagttgtcttgacctccaccaagaaccatgccagaat |
| | (EC 3.1.2.4) | | catcaccctcaacagagtcagaaagttgaattcgttaaaca |
| | | | ccgaaatgattgaactaatgacaccacctgtcttggagtac |
| | | | gccaaagagaatgtcaacaacgtcaccatcttgacttcgaa |
| | | | ctcccctaaggcattgtgtgccggtggtgatgttgctgaat |
| | | | gtgcaattcaaatcagaaagggcaacccgggatacggcgct |
| | | | gatttttttgataaggaatacaacctcaattacattatttc |
| | | | caccttgccaaagccttacatttcccttatggatggcatca |
| | | | cgtttggtggtggtgttgggttgtctgttcacgctccattt |
| | | | agagttgccacggagaagaccaagttagccatgccggagat |
| | | | ggacattggattcttccctgatgtcggtaccactttcttct |
| | | | tgccaagattggacgacaagattggttactacgttgcgttg |
| | | | actgggtctgttttgccaggtttggatgcctatttggcggg |
| | | | atttgcaacccactatatcaagtcggaaaaaatccctctgt |
| | | | tgatcaagagattggctggcttgcaaccacctgaaattgaa |
| | | | ggcgaaatcacggttatttctggaaacaatcagtacttcaa |
| | | | ccaggtgaatgacattttgaacgagtttagtgagaagaagt |
| | | | tgcctcaggactacaggttcttcctttccccagatgatata |
| | | | gccgttatcaacaaggcattctcgcaagactcaatcgacgg |
| | | | tgtgttcaagtacttgaaagaggaaggttctccatttgcaa |
| | | | agaagacccttgacactttgtccaagaagccaaggagttcg |
| | | | ttggccgttgcatttgcgttgttgaaccagggtgataagaa |
| | | | cacgatcagagaacaatttgagttggaaatggttgctgcaa |
| | | | ccaacattatgagcatccctgctgaacgtaacgactttgct |
| | | | aaaggtgtcattcacaaattggtcgacaagataaaggaccc |
| | | | attcttcccacaatggaacgacccaagcacagtcacgccag |
| | | | agtttgtcaaaaacatactcagtttgtccaagaacaccgac |
| | | | aagtacttgaagaagccatacgtcaagcaatggtttggtgt |
| | | | tgacttcacccagtaccctcaccaattcggggtgccaacca |
| | | | accgcgaagttgaagcatacattgctggcaccgacggctcc |
| | | | aacagaacctacttgccaactccaagcgaagtgttcaagca |
| | | | tttcaagatcaagacgggcgacaagttgggtgttgaagcca |
| | | | agattcaacagattttggacttgcatggcgagactgcaaag |
| | | | tatgataacaagtatgtcacctggaaagacgaaccaaccaa |
| | | | a |
| 103 | 3- | Candida sp. | mirftvssirpincatrrsisllqsrmsssystnptaggee |
| | hydroxypropionyl- | polypeptide | epvvltstknhariitlnrvrklnslntemielmtppvley |
| | CoA | | akenvnnvtiltsnspkalcaggdvaecaiqirkgnpgyga |
| | hydrolase | | dffdkeynlnyiistlpkpyislmdgitfgggvglsvhapf |
| | (EC 3.1.2.4) | | rvatektklampemdigffpdvgttfflprlddkigyyval |
| | | | tgsvlpgldaylagfathyiksekipslikrlaglqppeie |
| | | | geitvisgnnqyfnqvndilnefsekklpgdyrfflspddi |
| | | | avinkafsqdsidgvfkylkeegspfakktldtlskkprss |
| | | | lavafallnqgdkntireqfelemvaatnimsipaerndfa |
| | | | kgvihklvdkikdpffpqwndpstvtpefvknilslskntd |
| | | | kylkkpyvkqwfgvdftqyphqfgvptnreveayiagtdgs |
| | | | nrtylptpsevfkhfkiktgdklgveakiggildlhgetak |
| | | | ydnkyvtwkdeptk |

### Example 19: Examples of Certain Non-Limiting Embodiments

Listed hereafter are non-limiting examples of certain embodiments of the technology.

A1. A genetically modified yeast, comprising a genetic modification that reduces or abolishes the activity of 3-hydroxypropionate dehydrogenase (HPD1) and/or malonate semialdehyde dehydrogenase (acetylating) (ALD6), wherein the yeast is of a strain selected from among Yarrowia yeast, Candida revkaufi, Candida pulcherrima, Candida tropicalis, Candida maltosa, Candida utilis, Candida viswanathii, Candida strain ATCC20336, Rhodotorula yeast, Rhodosporidium yeast, Saccharomyces yeast, Cryptococcus yeast, Trichosporon yeast, Pichia yeast, Kluyveromyces yeast and Lipomyces yeast.

A1.1 The genetically modified yeast of embodiment A1, wherein the genetic modification comprises:

(a) a disruption, deletion or knockout of (i) a polynucleotide that encodes a HPD1 polypeptide, or (ii) a promoter operably linked to a polynucleotide that encodes a HPD1 polypeptide, whereby HPD1 activity is reduced or abolished; and/or

(b) a disruption, deletion or knockout of (i) a polynucleotide that encodes a ALD6 polypeptide or (ii) a promoter operably linked to a polynucleotide that encodes a ALD6 polypeptide, whereby ALD6 activity is reduced or abolished.

A1.3 A genetically modified yeast, comprising a genetic modification that reduces or abolishes the activity of 3-hydroxypropionate dehydrogenase (HPD1) and increases the activity of malonate semialdehyde dehydrogenase (acetylating) (ALD6).

A1.4 The genetically modified embodiment of embodiment A1.3, wherein the yeast is of a strain selected from among Yarrowia yeast, Candida albicans, Candida revkaufi, Candida pulcherrima, Candida tropicalis, Candida maltosa, Candida utilis, Candida strain ATCC20336, Candida viswanathii, Rhodotorula yeast, Rhodosporidium yeast, Saccharomyces yeast, Cryptococcus yeast, Trichosporon yeast, Pichia yeast, Kluyveromyces yeast and Lipomyces yeast

A2. The genetically modified yeast of any of embodiments A1 to A1.4, further comprising a genetic modification that increases the activity of one or more enzymes selected from among a cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase and 3-hydroxypropionyl-CoA hydrolase.

A3. The genetically modified yeast of any one of embodiments A1 to A2, wherein the yeast is of a Candida tropicalis strain or a Candida strain ATCC20336.

A4. The genetically modified yeast of embodiment A3, wherein the yeast is a Candida strain ATCC20336.

A5. The genetically modified yeast of any one of embodiments A1 to A4, wherein the genetic modification comprises a disruption, deletion or knockout of (i) a polynucleotide that encodes a HPD1 polypeptide, or (ii) a promoter operably linked to a polynucleotide that encodes a HPD1 polypeptide, whereby HPD1 activity is reduced or abolished.

A5.1. The genetically modified yeast of any one of embodiments A1 to A4, wherein the genetic modification comprises a disruption, deletion or knockout of (i) a polynucleotide that encodes a ALD6 polypeptide or (ii) a promoter operably linked to a polynucleotide that encodes a ALD6 polypeptide, whereby ALD6 activity is reduced or abolished.

A6. The genetically modified yeast of any one of embodiments A1 to A4, wherein the genetic modification comprises:

a disruption, deletion or knockout of (i) a polynucleotide that encodes a HPD1 polypeptide, or (ii) a promoter operably linked to a polynucleotide that encodes a HPD1 polypeptide, whereby HPD1 activity is reduced or abolished; and.

a disruption, deletion or knockout of (i) a polynucleotide that encodes a ALD6 polypeptide or (ii) a promoter operably linked to a polynucleotide that encodes a ALD6 polypeptide, whereby ALD6 activity is reduced or abolished.

A7. The genetically modified yeast of embodiment A4, wherein the yeast strain is selected from among sAA5405, sAA5526, sAA5600, AA5679, sAA5710 and sAA5733.

A8. The genetically modified yeast of embodiment A7, wherein the yeast strain is sAA5600.

A9. The genetically modified yeast of embodiment A7, wherein the yeast strain is sAA5733.

A10. The genetically modified yeast of any one of embodiments A1 to A6, wherein the HPDI polypeptide comprises a polypeptide 70% or more identical to SEQ ID NO: 1.

All. The genetically modified yeast of embodiment A10, wherein the HPD1 polypeptide comprises a polypeptide 80% or more identical to SEQ ID NO: 1.

A12. The genetically modified yeast of any one of embodiments A1 to A6, wherein the ALD6 polypeptide comprises a polypeptide 70% or more identical to SEQ ID NO: 17.

A13. The genetically modified yeast of embodiment A12, wherein the ALD6 polypeptide comprises a polypeptide 80% or more identical to SEQ ID NO: 17.

A14. The genetically modified yeast of any one of embodiments A1 to A8 and A10 to A13, wherein the HPD1 activity is abolished.

A15. The genetically modified yeast of any one of embodiments A1 to A7 and A9 to A13, wherein the ALD6 activity is abolished.

A16. The genetically modified yeast of any one of embodiments A1 to A15, wherein the yeast is capable of producing 3-hydroxypropionic acid, 3-hydroxypropionate or a salt thereof from a feedstock comprising one or more alkane hydrocarbons with odd carbon numbered alkane chains.

A17. The genetically modified yeast of embodiment A16, wherein the source of the feedstock comprises one or more of petroleum, plants, chemically synthesized alkane hydrocarbons or alkane hydrocarbons produced by fermentation of a microorganism.

A18. The genetically modified yeast of embodiments A16 or A17, wherein the number of carbon atoms in the one or more alkane hydrocarbons is an odd number between three carbon atoms to thirty-five carbon atoms.

A19. The genetically modified yeast of any one of embodiments A16 to A18, wherein the feedstock comprises one or more alkane hydrocarbons selected from among propane, n-pentane, n-heptane or n-nonane.

A20. The genetically modified yeast of embodiment A19, wherein the feedstock comprises propane.

A21. The genetically modified yeast of embodiment A19 or A20, wherein the feedstock comprises n-pentane.

A22. The genetically modified yeast of any one of embodiments A19 to A21, wherein the feedstock comprises n-nonane.

A23. The genetically modified yeast of embodiment A20, wherein the feedstock consists of propane.

A24. The genetically modified yeast of embodiment A21, wherein the feedstock consists of n-pentane.

A25. The genetically modified yeast of embodiment A22, wherein the feedstock consists of n-nonane.

A26. The genetically modified yeast of any one of embodiments of A16 to A25, wherein the yield or titer of 3-hydroxypropionic acid, 3-hydroxypropionate or a salt thereof is between about 0.1 g/L to about 25 g/L.

B1. An isolated nucleic acid, comprising the polynucleotide set forth in SEQ ID NO:6.

B2. An isolated nucleic acid, comprising the polynucleotide set forth in SEQ ID NO:19.

C1. An expression vector, comprising the nucleic acid of embodiment B1.

C2. An expression vector, comprising the nucleic acid of embodiment B2.

C3. An expression vector, comprising the nucleic acids of embodiments B1 and B2.

D1. A cell, comprising a nucleic acid of embodiment B1 and/or B2.

D2. A cell, comprising an expression vector of any one of embodiments C1 to C3.

D3. The cell of embodiment D1 or D2, which is a bacterium.

D4. The cell of embodiment D1 or D2, which is a yeast.

D5. The cell of embodiment D4, wherein the yeast is selected from among Yarrowia yeast, Candida revkaufi, Candida pulcherrima, Candida tropicalis, Candida maltosa, Candida utilis, Candida viswanathii, Candida strain ATCC20336, Rhodotorula yeast, Rhodosporidium yeast, Saccharomyces yeast, Cryptococcus yeast, Trichosporon yeast, Pichia yeast, Kluyveromyces yeast and Lipomyces yeast.

D6. The cell of embodiment D5, wherein the yeast is Candida tropicalis or Candida strain ATCC20336.

D7. The cell of embodiment D6, wherein the yeast is a genetically modified ATCC20336 yeast.

E1. A method for producing 3-hydroxypropionic acid, 3-hydroxypropionate or a salt thereof, comprising: contacting a genetically modified yeast with a feedstock comprising one or more alkane hydrocarbons with odd carbon numbered alkane chains; and culturing the yeast under conditions in which the 3-hydroxypropionic acid, 3-hydroxypropionate or salt thereof is produced from the feedstock, wherein the yeast comprises a genetic modification that reduces or abolishes the activity of HPD1 and/or ALD6.

E1.1 The method of embodiment E1, wherein the genetically modified yeast comprises: (a) a disruption, deletion or knockout of (i) a polynucleotide that encodes a HPD1 polypeptide, or (ii) a promoter operably linked to a polynucleotide that encodes a HPD1 polypeptide, whereby HPDI activity is reduced or abolished, and/or (b) a disruption, deletion or knockout of (i) a polynucleotide that encodes a ALD6 polypeptide or (ii) a promoter operably linked to a polynucleotide that encodes a ALD6 polypeptide, whereby ALD6 activity is reduced or abolished.

E2. The method of embodiment E1 or E1.1, wherein the yeast is of a strain selected from among Yarrowia yeast, Candida yeast, Rhodotorula yeast, Rhodosporidium yeast, Saccharomyces yeast, Cryptococcus yeast, Trichosporon yeast, Pichia yeast, Kluyveromyces yeast and Lipomyces yeast.

E3. A method for producing 3-hydroxypropionic acid, 3-hydroxypropionate or a salt thereof, comprising: contacting the genetically modified yeast of any of embodiments A1 to A26 with a feedstock comprising one or more alkane hydrocarbons with odd carbon numbered alkane chains; and culturing the yeast under conditions in which the 3-hydroxypropionic acid, 3-hydroxypropionate or salt thereof is produced from the feedstock.

E4. A method for producing 3-hydroxypropionic acid, 3-hydroxypropionate or a salt thereof, comprising: contacting the cell of any of embodiments D1 to D7 with a feedstock comprising one or more alkane hydrocarbons with odd carbon numbered alkane chains; and culturing the cell under conditions in which the 3-hydroxypropionic acid, 3-hydroxypropionate or salt thereof is produced from the feedstock.

E5. The method of any of embodiments E1 to E4, wherein the source of the feedstock comprises one or more of petroleum, plants, chemically synthesized alkane hydrocarbons or alkane hydrocarbons produced by fermentation of a microorganism.

E6. The method of any of embodiments E1 to E5, wherein the number of carbon atoms in the one or more alkane hydrocarbons is an odd number between three carbon atoms to thirty-five carbon atoms.

E7. The method of any one of embodiments E1 to E6, wherein the feedstock comprises one or more alkane hydrocarbons selected from among propane, n-pentane, n-heptane or n-nonane.

E8. The method of embodiment E7, wherein the feedstock comprises propane.

E9. The method of embodiment E7 or E8, wherein the feedstock comprises n-pentane.

E10. The method of any one of embodiments E7 to E9, wherein the feedstock comprises n-nonane.

E11. The method of embodiment E8, wherein the feedstock consists of propane.

E12. The method of embodiment E9, wherein the feedstock consists of n-pentane.

E13. The method of embodiment E10, wherein the feedstock consists of n-nonane.

E14. The method of any one of embodiments E1 to E3 and E5 to E13, wherein the genetically modified yeast further comprises an increased activity of one or more enzymes selected from among a cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase and 3-hydroxypropionyl-CoA hydrolase.

E15. The method of any one of embodiments E1 to E3 and E5 to E14, wherein the genetically modified yeast is of a Candida tropicalis strain or a Candida strain ATCC20336.

E16. The method of embodiment E15, wherein the genetically modified yeast is of a Candida ATCC20336 strain.

E17. The method of any one of embodiments E1 to E3 and E5 to E16, comprising a disruption, deletion or knockout of (i) a polynucleotide that encodes a 3-hydroxypropionate dehydrogenase polypeptide, or (ii) a promoter operably linked to a polynucleotide that encodes a 3-hydroxypropionate dehydrogenase polypeptide, whereby 3-hydroxypropionate dehydrogenase (HPD1) activity is reduced or abolished.

E18. The method of any one of embodiments E1 to E3 and E5 to E17, comprising a disruption, deletion or knockout of (i) a polynucleotide that encodes a malonate semialdehyde dehydrogenase polypeptide or (ii) a promoter operably linked to a polynucleotide that encodes a malonate semialdehyde dehydrogenase polypeptide, whereby malonate semialdehyde dehydrogenase (ALD6) activity is reduced or abolished.

E19. The method of embodiment E16, wherein the yeast strain is selected from among sAA5405, sAA5526, sAA5600, AA5679, sAA5710 and sAA5733.

E20. The method of embodiment E19, wherein the yeast strain is sAA5600.

E21. The method of embodiment E19, wherein the yeast strain is sAA5733.

E22. The method of any one of embodiments E1 to E3 and E5 to E18, wherein the 3-hydroxypropionate dehydrogenase polypeptide comprises a polypeptide 70% or more identical to SEQ ID NO: 1.

E23. The method of embodiment E22, wherein the 3-hydroxypropionate dehydrogenase polypeptide comprises a polypeptide 80% or more identical to SEQ ID NO: 1.

E24. The method of any one of embodiments E1 to E3, E5 to E18, E22 and E23, wherein the malonate semialdehyde dehydrogenase polypeptide comprises a polypeptide 70% or more identical to SEQ ID NO: 17.

E25. The method of embodiment E24, wherein the malonate semialdehyde dehydrogenase polypeptide comprises a polypeptide 80% or more identical to SEQ ID NO: 17.

E26. The method of any one of embodiments E1 to E3, E5 to E18 and E22 to E25, wherein the 3-hydroxypropionate dehydrogenase activity is abolished in the genetically modified yeast.

E27. The method of any one of embodiments E1 to E3, E5 to E18 and E22 to E26, wherein the malonate semialdehyde dehydrogenase (ALD6) activity is abolished in the genetically modified yeast.

E28. The method of any one of embodiments E1 to E27, wherein the yield or titer of 3-hydroxypropionic acid or a salt thereof is between about 0.1 g/L to about 25 g/L.

E29. The method of any one of embodiments E1 to E28, further comprising isolating the 3-hydroxypropionic acid, 3-hydroxypropionate or salt thereof.

F1. A method for producing 3-hydroxypropionic acid, 3-hydroxypropionate or a salt thereof, comprising: contacting a genetically modified yeast with a feedstock comprising one or more odd chain fatty acids or esters thereof and culturing the yeast under conditions in which the 3-hydroxypropionic acid, 3-hydroxypropionate or salt thereof is produced from the feedstock, wherein the yeast comprises a genetic modification that reduces or abolishes the activity of HPD1 and/or ALD6.

F2. A method for producing 3-hydroxypropionic acid, 3-hydroxypropionate or a salt thereof, comprising: contacting a genetically modified yeast with a feedstock comprising one or more odd chain fatty acids or esters thereof, wherein the yeast is of a strain selected from among Yarrowia yeast, Candida revkaufi, Candida pulcherrima, Candida tropicalis, Candida maltosa, Candida utilis, Candida viswanathii, Candida strain ATCC20336, Rhodotorula yeast, Rhodosporidium yeast, Saccharomyces yeast, Cryptococcus yeast, Trichosporon yeast, Pichia yeast, Kluyveromyces yeast and Lipomyces yeast; and culturing the yeast under conditions in which the 3-hydroxypropionic acid, 3-hydroxypropionate or salt thereof is produced from the feedstock, wherein the yeast comprises a genetic modification that reduces or abolishes the activity of HPD1 and/or ALD6.

F3. The method of embodiment F1 or F2, wherein the genetically modified yeast comprises: (a) a disruption, deletion or knockout of (i) a polynucleotide that encodes a HPD1 polypeptide, or (ii) a promoter operably linked to a polynucleotide that encodes a HPD1 polypeptide, whereby HPDI activity is reduced or abolished, and/or (b) a disruption, deletion or knockout of (i) a polynucleotide that encodes a ALD6 polypeptide or (ii) a promoter operably linked to a polynucleotide that encodes a ALD6 polypeptide, whereby ALD6 activity is reduced or abolished.

F5. The method of any one of embodiments F1 to F4, wherein the yeast is of a strain selected from among Yarrowia yeast, Candida yeast, Rhodotorula yeast, Rhodosporidium yeast, Saccharomyces yeast, Cryptococcus yeast, Trichosporon yeast, Pichia yeast, Kluyveromyces yeast and Lipomyces yeast.

F6. A method for producing 3-hydroxypropionic acid, 3-hydroxypropionate or a salt thereof, comprising: contacting the genetically modified yeast of any of embodiments A1 to A26 with a feedstock comprising one or more odd chain fatty acids; and culturing the yeast under conditions in which the 3-hydroxypropionic acid, 3-hydroxypropionate or salt thereof is produced from the feedstock.

F7. A method for producing 3-hydroxypropionic acid, 3-hydroxypropionate or a salt thereof, comprising: contacting the cell of any of embodiments D1 to D7 with a feedstock comprising one or more odd chain fatty acids or esters thereof; and culturing the cell under conditions in which the 3-hydroxypropionic acid, 3-hydroxypropionate or salt thereof is produced from the feedstock.

F8. The method of any one of embodiments F1 to F7, further comprising isolating the 3-hydroxypropionic acid, 3-hydroxypropionate or a salt thereof.

F9. The method of any of embodiments F1 to F8, wherein the source of the feedstock comprises one or more of animals, microorganisms, plants, plant oils, chemically synthesized fatty acids or fatty acids produced by fermentation of a microorganism.

F10. The method of embodiment F9, wherein the animals, microorganisms or plants are genetically engineered to produce odd chain fatty acids or esters thereof.

F11. The method of any one of embodiments F1 to F10, wherein the number of carbon atoms in the one or more odd chain fatty acids or esters thereof is an odd number between three carbon atoms to thirty-five carbon atoms.

F11. The method of embodiment F11, wherein the fatty acid/ester thereof is selected from among propionic acid/propionate, valeric acid/valerate, heptanoic acid/heptanoate, nonanoic acid/nonanoate, undecanoic acid/undecanoate, tridecanoic acid/tridecanoate, pentadecanoic acid/pentadecanoate, heptadecanoic acid/heptadecanoate, nonadecanoic acid/nonadecanoate, heneicosanoic acid/heneisocanoate, tricosanoic acid/tricosanoate, pentacosanoic acid/pentacosanoate, heptacosanoic acid/heptacosanoate, nonacosanoic acid/nonacosanoate and hentriacontanoic acid/hentriacontanoate.

F12. The method of any of embodiments F1 to F10, wherein the number of carbon atoms in the one or more odd chain fatty acids or esters thereof is an odd number between seven carbon atoms to thirty-five carbon atoms.

F13. The method of any one of embodiments F1 to F12, wherein the feedstock comprises pentadecanoic acid or a pentadecanoate.

F14. The method of embodiment F13, wherein the feedstock comprises a pentadecanoate, and the pentadecanoate is methyl-pentadecanoate.

F15. The method of embodiment F14, wherein the feedstock consists of methyl-pentadecanoate.

F16. The method of any one of embodiments F1 to F6 and F8 to F15, wherein the genetically modified yeast further comprises an increased activity of one or more enzymes selected from among a cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase and 3-hydroxypropionyl-CoA hydrolase.

F17. The method of any one of embodiments F1 to F6 and F8 to F16, wherein the genetically modified yeast is of a Candida tropicalis strain or a Candida strain ATCC20336.

F18. The method of embodiment F17, wherein the genetically modified yeast is of a Candida ATCC20336 strain.

F19. The method of any one of embodiments F1 to F6 and F8 to F18, comprising a disruption, deletion or knockout of (i) a polynucleotide that encodes a 3-hydroxypropionate dehydrogenase polypeptide, or (ii) a promoter operably linked to a polynucleotide that encodes a 3-hydroxypropionate dehydrogenase polypeptide, whereby 3-hydroxypropionate dehydrogenase (HPD1) activity is reduced or abolished.

F20. The method of any one of embodiments F1 to F6 and F8 to F19, comprising a disruption, deletion or knockout of (i) a polynucleotide that encodes a malonate semialdehyde dehydrogenase polypeptide or (ii) a promoter operably linked to a polynucleotide that encodes a malonate semialdehyde dehydrogenase polypeptide, whereby malonate semialdehyde dehydrogenase (ALD6) activity is reduced or abolished.

F21. The method of embodiment F18, wherein the yeast strain is selected from among sAA5405, sAA5526, sAA5600, AA5679, sAA5710 and sAA5733.

F22. The method of embodiment F21, wherein the yeast strain is sAA5600.

F23. The method of embodiment F21, wherein the yeast strain is sAA5733.

F24. The method of any one of embodiments F1 to F6 and F8 to F20, wherein the 3-hydroxypropionate dehydrogenase polypeptide comprises a polypeptide 70% or more identical to SEQ ID NO: 1.

F25. The method of embodiment F24, wherein the 3-hydroxypropionate dehydrogenase polypeptide comprises a polypeptide 80% or more identical to SEQ ID NO: 1.

F26. The method of any one of embodiments F1 to F6, F8 to F20, F24 and F25, wherein the malonate semialdehyde dehydrogenase polypeptide comprises a polypeptide 70% or more identical to SEQ ID NO: 17.

F27. The method of embodiment F26, wherein the malonate semialdehyde dehydrogenase polypeptide comprises a polypeptide 80% or more identical to SEQ ID NO: 17.

F28. The method of any one of embodiments F1 to F6, F8 to F20 and F24 to F27, wherein the 3-hydroxypropionate dehydrogenase activity is abolished in the genetically modified yeast.

F29. The method of any one of embodiments F1 to F6, F8 to F20 and F24 to F28, wherein the malonate semialdehyde dehydrogenase (ALD6) activity is abolished in the genetically modified yeast.

F30. The method of any one of embodiments F1 to F29, wherein the yield or titer of 3-hydroxypropionic acid or a salt thereof is between about 0.1 g/L to about 25 g/L.

G1. A method for producing acrylic acid, acrylate or a salt or derivative thereof, comprising: performing the method of any one of embodiments F1 to F30, whereby 3-hydroxypropionic acid, 3-hydroxypropionate or a salt thereof is produced; and subjecting the 3-hydroxypropionic acid, 3-hydroxypropionate or salt thereof to conditions under which acrylic acid, acrylate or a salt or derivative thereof is produced.

G2. The method of embodiment F1, wherein the conditions comprise dehydration of the 3-hydroxypropionic acid, 3-hydroxypropionate or salt thereof.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A genetically modified yeast, comprising (a) one or more genetic modifications that reduce or abolish the activity of 3-hydroxypropionate dehydrogenase (HPD1) or malonate semialdehyde dehydrogenase (acetylating) (ALD6), and (b) one or more genetic modifications that increases the activity of one or more enzymes selected from among a cytochrome P-450 monooxygenase, a cytochrome P-450 reductase, an aldehyde dehydrogenase, an alcohol dehydrogenase, an acyl-CoA transferase, a long-chain-fatty-acid CoA ligase, an acyl-CoA synthetase, an acetyl-CoA C-acyltransferase, a propionyl-CoA synthetase, an acyl-CoA oxidase, an acyl-CoA dehydrogenase, an enoyl-CoA hydratase, and 3-hydroxypropionyl-CoA hydrolase.

2. The genetically modified yeast of claim 1, wherein the one or more genetic modifications reduce or abolish the activity of 3-hydroxypropionate dehydrogenase (HPD1) and increase the activity of malonate semialdehyde dehydrogenase (acetylating) (ALD6).

3. The genetically modified yeast of claim 1, wherein the HPD1 activity is reduced or abolished, and wherein the one or more genetic modifications comprise a disruption, deletion or knockout of (i) a polynucleotide that encodes a HPD1 polypeptide, or (ii) a promoter operably linked to a polynucleotide that encodes a HPD1 polypeptide.

4. The genetically modified yeast of claim 1, wherein the ALD6 activity is reduced or abolished, and wherein the one or more genetic modifications comprise a disruption, deletion or knockout of (i) a polynucleotide that encodes a ALD6 polypeptide or (ii) a promoter operably linked to a polynucleotide that encodes a ALD6 polypeptide.

5. (canceled)

6. The genetically modified yeast of claim 1, wherein the genetically modified yeast is of a strain selected from the group consisting of Yarrowia yeast, Candida albicans, Candida revkaufi, Candida pulcherrima, Candida tropicalis, Candida maltosa, Candida utilis, Candida viswanathii, Candida strain ATCC20336, Rhodotorula yeast, Rhodosporidium yeast, Saccharomyces yeast, Cryptococcus yeast, Trichosporon yeast, Pichia yeast, Kluyveromyces yeast and Lipomyces yeast.

7. The genetically modified yeast of claim 6, wherein the genetically modified yeast is a Candida tropicalis strain or a Candida strain ATCC20336.

8. (canceled)

9. The genetically modified yeast of claim 8, wherein the genetically modified yeast is selected from the group consisting of sAA5405, sAA5526, sAA5600, AA5679, sAA5710 and sAA5733.

10. (canceled)

11. (canceled)

12. The genetically modified yeast of claim 3, wherein the HPD1 polypeptide comprises a polypeptide 70% or more identical to SEQ ID NO: 1.

13. (canceled)

14. The genetically modified yeast of claim 3, wherein the ALD6 polypeptide comprises a polypeptide 70% or more identical to SEQ ID NO: 17.

15. (canceled)

16. The genetically modified yeast of claim 1, wherein the HPD1 or ALD6 activity is abolished.

17. The genetically modified yeast of claim 1, wherein the genetically modified yeast is adapted to produce 3-hydroxypropionic acid, 3-hydroxypropionate or a salt thereof from a feedstock.

18. The genetically modified yeast of claim 17, wherein the feedstock comprises one or more alkane hydrocarbons with odd carbon numbered chains or one or more fatty acids or esters with odd carbon numbered chains.

19. (canceled)

20. The genetically modified yeast of claim 17, wherein the odd carbon numbered chains have three carbon atoms to thirty-five carbon atoms.

21. (canceled)

22. The genetically modified yeast of claim 18, wherein the feedstock comprises propane, n-pentane, or n-nonane.

23. The genetically modified yeast of claim 18, wherein the feedstock comprises pentadecanoic acid or pentadecanoate.

24. The genetically modified yeast of claim 23, wherein the pentadecanoate is methyl-pentadecanoate.

25. The genetically modified yeast of claim 18, wherein source of the feedstock comprises one or more of petroleum, plants, chemically synthesized alkane hydrocarbons, alkane hydrocarbons produced by fermentation of a microorganism, animals, microorganisms, plants, plant oils, chemically synthesized fatty acids or fatty acids produced by fermentation of a microorganism.

26. The genetically modified yeast of claim 18, wherein yield or titer of 3-hydroxypropionic acid, 3-hydroxypropionate or a salt thereof is about 0.1 g/L to about 25 g/L.

27. An expression vector, comprising one or more genetic modifications that reduce or abolish the activity of 3-hydroxypropionate dehydrogenase (HPD1) or malonate semialdehyde dehydrogenase (acetylating) (ALD6).

28.-36. (canceled)

37. A method for producing 3-hydroxypropionic acid, 3-hydroxypropionate or a salt thereof, comprising: (a) contacting a genetically modified yeast that comprises one or more genetic modifications that reduce or abolish the activity of 3-hydroxypropionate dehydrogenase (HPD1) or malonate semialdehyde dehydrogenase (acetylating) (ALD6) with a feedstock; and (b) culturing the genetically modified yeast under a condition in which the 3-hydroxypropionic acid, 3-hydroxypropionate or salt thereof is produced.

38.-40. (canceled)


SEQUENCE LISTING (116 sequences)

SEQ ID NO: 1 - DNA, 1029 bp, Candida viswanathii
  Candida viswanathii (ATCC20336)DNA sequencing encoding a 3-hydroxypropionate dehydrogenase (EC 1.1.1.59)
atgttgagatcttcagtccgtactttctccacccagtccagagtattagccaactacggtttcgtaggcttgggtctcatgggccagcacatggccagacacgtctacaaccagttgcagccagcagacaagttgtatgtccacgacgtcaacccccagcacaccacccagttcgtcaccgacgtgaccacccagaagccacagaacgccacacaattgacgcccttgtcctccttgaaagagttcaccaccgagccagagtcccagttggacttcatcgtcaccatggtccccgagggcaagcacgtcaaggccgttgtctccgagctagtcgaccactacaatgcgtcgggaaaatacgacccatccaagaagttgacctttgtggactcctccaccatcgacatccccacctccagggaggtccaccagctcgttgccgacaagttacaaggcgccacgttcatcgacgccccggtttcgggtggtgtcgctggtgccaggaacggaaccttgtcgttcatggtgtcgcgggacaccaaggaagacgtcgaccctaacctcgtcacgcttttgaactacatgggcagcaacatcttcccatgtggtggaacccacgggaccggcttggctgccaagttggcaaacaactacttgttggcgatcacgaacatcgccgtcgcagatagcttccagttggcaaactcgttcgggttgaacttgcagaactacgccaagttggtgtcgacctccacaggtaagtcctgggctagtgtcgataactgcccaatccccggtgtctaccctgaaaagaacttgacttgtgataacggatacaagggtgggtttgtcacgaagttgacgagaaaggatgtcgtcttggctacggagtctgctaaggctaacaaccagttccttatgcttggcgaagtcggtagatactggtacgacaaggcttgtgaagatgaaaagtacgccaacagagacttgtctgttcttttcgaattcttgggtgatcttaaaaaataa

SEQ ID NO: 2 - protein, 342 aa, Candida viswanathii
  Candida viswanathii (ATCC20336)3- hydroxypropionate dehydrogenase (EC 1.1.1.59)
MLRSSVRTFSTQSRVLANYGFVGLGLMGQHMARHVYNQLQPADKLYVHDVNPQHTTQFVTDVTTQKPQNATQLTPLSSLKEFTTEPESQLDFIVTMVPEGKHVKAVVSELVDHYNASGKYDPSKKLTFVDSSTIDIPTSREVHQLVADKLQGATFIDAPVSGGVAGARNGTLSFMVSRDTKEDVDPNLVTLLNYMGSNIFPCGGTHGTGLAAKLANNYLLAITNIAVADSFQLANSFGLNLQNYAKLVSTSTGKSWASVDNCPIPGVYPEKNLTCDNGYKGGFVTKLTRKDVVLATESAKANNQFLMLGEVGRYWYDKACEDEKYANRDLSVLFEFLGDLKK

SEQ ID NO: 3 - DNA, 29 bp, Artificial Sequence
  Description of Artificial Sequence Synthetic primer
tacccatatgttgagatcttcagtccgta

SEQ ID NO: 4 - DNA, 33 bp, Artificial Sequence
  Description of Artificial Sequence Synthetic primer
taccctcgagttttttaagatcacccaagaatt

SEQ ID NO: 5 - DNA, 6260 bp, Artificial Sequence
  Description of Artificial Sequence Synthetic pAA1753 plasmid comprising the Candida viswanathii (ATCC20336) 3- hydroxypropionate dehydrogenase gene expressed from a T7 promoter
atccggatatagttcctcctttcagcaaaaaacccctcaagacccgtttagaggccccaaggggttatgctagttattgctcagcggtggcagcagccaactcagcttcctttcgggctttgttagcagccggatctcagtggtggtggtggtggtgctcgagttttttaagatcacccaagaattcgaaaagaacagacaagtctctgttggcgtacttttcatcttcacaagccttgtcgtaccagtatctaccgacttcgccaagcataaggaactggttgttagccttagcagactccgtagccaagacgacatcctttctcgtcaacttcgtgacaaacccacccttgtatccgttatcacaagtcaagttcttttcagggtagacaccggggattgggcagttatcgacactagcccaggacttacctgtggaggtcgacaccaacttggcgtagttctgcaagttcaacccgaacgagtttgccaactggaagctatctgcgacggcgatgttcgtgatcgccaacaagtagttgtttgccaacttggcagccaagccggtcccgtgggttccaccacatgggaagatgttgctgcccatgtagttcaaaagcgtgacgaggttagggtcgacgtcttccttggtgtcccgcgacaccatgaacgacaaggttccgttcctggcaccagcgacaccacccgaaaccggggcgtcgatgaacgtggcgccttgtaacttgtcggcaacgagctggtggacctccctggaggtggggatgtcgatggtggaggagtccacaaaggtcaacttcttggatgggtcgtattttcccgacgcattgtagtggtcgactagctcggagacaacggccttgacgtgcttgccctcggggaccatggtgacgatgaagtccaactgggactctggctcggtggtgaactctttcaaggaggacaagggcgtcaattgtgtggcgttctgtggcttctgggtggtcacgtcggtgacgaactgggtggtgtgctgggggttgacgtcgtggacatacaacttgtctgctggctgcaactggttgtagacgtgtctggccatgtgctggcccatgagacccaagcctacgaaaccgtagttggctaatactctggactgggtggagaaagtacggactgaagatctcaacatatgtatatctccttcttaaagttaaacaaaattatttctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcagcgccctgcaccattatgttccggatctgcatcgcaggatgctgctggctaccctgtggaacacctacatctgtattaacgaagcgctggcattgaccctgagtgatttttctctggtcccgccgcatccataccgccagttgtttaccctcacaacgttccagtaaccgggcatgttcatcatcagtaacccgtatcgtgagcatcctctctcgtttcatcggtatcattacccccatgaacagaaatcccccttacacggaggcatcagtgaccaaacaggaaaaaaccgcccttaacatggcccgctttatcagaagccagacattaacgcttctggagaaactcaacgagctggacgcggatgaacaggcagacatctgtgaatcgcttcacgaccacgctgatgagctttaccgcagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctctaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaagaattaattcatgagcggatacatatttgaatgtatttagaaaaataaacaaataggggttccgcgcacatttccccgaaaagtgccacctgaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgcca

SEQ ID NO: 6 - DNA, 2783 bp, Artificial Sequence
  Description of Artificial Sequence Synthetic HPD1 deletion cassette
  modified_base (901)..(901): a, c, t, g, unknown or other
  modified_base (1045)..(1045): a, c, t, g, unknown or other
  modified_base (1301)..(1301): a, c, t, g, unknown or other
ttttctctgggctgtgttggttttttcgcagcttcagtttgtgggtgtttgtgggtgtttggtgattccaacagatcgggttaaatgtcacaagcatttaagaaacggccacgccaactaagcccaaacgccgacccatcctacccgaattgtccactctcatggataccatagttgaataaccgtcacctctattgaagcagtgatattacaaaaaggaacagggccattttgctgccgtagaagctttcgcaggtaaagtggggaaaacccccatgcagcgtgtaactggcatgataacactgaccgagttttcttttgtttaaggcaaattgagtatgggcgggtgttccatgttctctttttttttaactctctccacagaaacccagaatggaattgtatctacggttgtttcggtatgacccccggggatctgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcaatggttagcacaaaaacatacacagaaagggcatcagcacacccctccnaggttgcccaacgtttattccgcttaatggagtccaaaaagaccaacctctgcgcctcgatcgacgtgaccacaaccgccgagttcctttcgctcatcgacaagctcggtccccacatctgtctcgtgaagacgcacatcgatntcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgtnggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctacggtcgacgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcagagctcggtaccaaatgggtcaacagaatccaattcggtgtactcgtagcaacctgttctttcttatcgtgatagttcattctgacaacttttctgatccatcttcttcttctgtagagctcattgttgctggccaacttctcaatctgatccaacgagagctcgttaatcgtatggtcatccgtgtcattaatatcattactcgtattcttcgtgattatatcatatgcccatttctcatcatcatcgataatcacgagatcttggatcaagtttccctccacccatgcgttgatattgaagtcaatcttccatttttctgaatccaaaaacttgtagttcgcaggaggattgacctccgtcaaggtatccctcttgttgagattcaaaagcttgacgtcgtcttcctgctggtggtcttcatcgtgctgtctctctaa

SEQ ID NO: 7 - DNA, 400 bp, Candida viswanathii
  Candida viswanathii (ATCC20336); Genomic region upstream of the HPD1 gene
ttttctctgggctgtgttggttttttcgcagcttcagtttgtgggtgtttgtgggtgtttggtgattccaacagatcgggttaaatgtcacaagcatttaagaaacggccacgccaactaagcccaaacgccgacccatcctacccgaattgtccactctcatggataccatagttgaataaccgtcacctctattgaagcagtgatattacaaaaaggaacagggccattttgctgccgtagaagctttcgcaggtaaagtggggaaaacccccatgcagcgtgtaactggcatgataacactgaccgagttttcttttgtttaaggcaaattgagtatgggcgggtgttccatgttctctttttttttaactctctccacagaaacccagaatggaat

SEQ ID NO: 8 - DNA, 22 bp, Artificial Sequence
  Description of Artificial Sequence Synthetic primer
ttttctctgggctgtgttggtt

SEQ ID NO: 9 - DNA, 50 bp, Artificial Sequence
  Description of Artificial Sequence Synthetic primer
tcataccgaaacaaccgtagatacaattccattctgggtttctgtggaga

SEQ ID NO: 10 - DNA, 400 bp, Candida viswanathii
  Candida viswanathii (ATCC20336); Genomic region downstream of the HPD1 gene
tactcgtagcaacctgttctttcttatcgtgatagttcattctgacaacttttctgatccatcttcttcttctgtagagctcattgttgctggccaacttctcaatctgatccaacgagagctcgttaatcgtatggtcatccgtgtcattaatatcattactcgtattcttcgtgattatatcatatgcccatttctcatcatcatcgataatcacgagatcttggatcaagtttccctccacccatgcgttgatattgaagtcaatcttccatttttctgaatccaaaaacttgtagttcgcaggaggattgacctccgtcaaggtatccctcttgttgagattcaaaagcttgacgtcgtcttcctgctggtggtcttcatcgtgctgtctctctaa

SEQ ID NO: 11 - DNA, 50 bp, Artificial Sequence
  Description of Artificial Sequence Synthetic primer
tctccacagaaacccagaatggaattgtatctacggttgtttcggtatga

SEQ ID NO: 12 - DNA, 23 bp, Artificial Sequence
  Description of Artificial Sequence Synthetic primer
ttagagagacagcacgatgaaga

SEQ ID NO: 13 - DNA, 1983 bp, Artificial Sequence
  Description of Artificial Sequence Synthetic 2.0 kb Pura3URA3Tura3Pura3 cassette
  modified_base (501)..(501): a, c, t, g, unknown or other
  modified_base (645)..(645): a, c, t, g, unknown or other
  modified_base (901)..(901): a, c, t, g, unknown or other
tgtatctacggttgtttcggtatgacccccggggatctgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcaatggttagcacaaaaacatacacagaaagggcatcagcacacccctccnaggttgcccaacgtttattccgcttaatggagtccaaaaagaccaacctctgcgcctcgatcgacgtgaccacaaccgccgagttcctttcgctcatcgacaagctcggtccccacatctgtctcgtgaagacgcacatcgatntcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgtnggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctacggtcgacgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcagagctcggtaccaaatgggtcaacagaatccaattcggtg

SEQ ID NO: 14 - DNA, 6299 bp, Artificial Sequence
  Description of Artificial Sequence Synthetic pAA1860 plasmid containing the 2.0 kb Pura3URA3Tura3Pura3 cassette used for gene deletions in Candida viswanathii (ATCC20336)
  modified_base (4491)..(4491): a, c, t, g, unknown or other
  modified_base (4635)..(4635): a, c, t, g, unknown or other
  modified_base (4891)..(4891): a, c, t, g, unknown or other
aagggcgaattctgcagatatccatcacactggcggccgctcgagcatgcatctagagggcccaattcgccctatagtgagtcgtattacaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctatacgtacggcagtttaaggtttacacctataaaagagagagccgttatcgtctgtttgtggatgtacagagtgatattattgacacgccggggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggcatgagattatcaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgaggagggccaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctatttaggtgacactatagaatactcaagctatgcatcaagcttggtaccgagctcggatccactagtaacggccgccagtgtgctggaattcgcccttttgtctcgcatggatgcacgaatgaacgactcgcctccaagcatatttatagctttgtcgacgttcttgacgttcaacgggagatcgatggccgctacacgcgggatatccattgaatgttcatctggtctttccaactctggcatggtgatggatgaagtgttggttgtctgagacagatgggcttgttttgattttttggtgattttttctttttccagagagtacaaaactgtgcagccgacaagaatctggcaggacagcaccagttggaaattttggcaacacagtttcaattgaccactggtggagtgttgctacaagggttggtgatactaagcagtgactcaattgacaccaggctgtacttttagacattcaattgaactgctgcattgccgtggggcagactactagaagtgtcctctcaatagctcgaaccacttgaaacacattacatcgtggcttaactgtatctacggttgtttcggtatgacccccggggatctgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcaatggttagcacaaaaacatacacagaaagggcatcagcacacccctccnaggttgcccaacgtttattccgcttaatggagtccaaaaagaccaacctctgcgcctcgatcgacgtgaccacaaccgccgagttcctttcgctcatcgacaagctcggtccccacatctgtctcgtgaagacgcacatcgatntcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgtnggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctacggtcgacgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcagagctcggtaccaaatgggtcaacagaatccaattcggtggtgacgaagttgtcaaggctaaggatggtgctggttccgccactttgtccatggctcaagctggtgctagattcgccggtgccgtcttggacggtttggctggtgaaaaggacgtcattgaatgtacctttgtcgactccccattgttcaagaacgaaggtgtcgaattcttctcctccaaggttaccttgggtgttgacggtgtcaagactgtccacccagttggcaacatttctgagtacgaagaagctcaagtcaaggaagccaaggacactttgatcaagaacatcaagaagggtgtcgactttgttgctcaaaacccataa

SEQ ID NO: 15 - DNA, 50 bp, Artificial Sequence
  Description of Artificial Sequence Synthetic primer
tgggtcaacagaatccaattcggtgtactcgtagcaacctgttctttctt

SEQ ID NO: 16 - DNA, 50 bp, Artificial Sequence
  Description of Artificial Sequence Synthetic primer
aagaaagaacaggttgctacgagtacaccgaattggattctgttgaccca

SEQ ID NO: 17 - DNA, 1650 bp, Candida viswanathii
  Candida viswanathii (ATCC20336); DNA sequencing encoding a malonate-semialdehyde dehydrogenase (acetylating)(EC 1.2.1.18)
atgttatccagagttcttttcaagactaaaccaagagttcctactaaatcaatcaccgccatggccatcagaaacaaatccatcgtgactttatcctccaccacctccacatacccaaccgaccacacgaccccgtccacggagccatacatcacgccatccttcgtgaacaacgagttcatcaagtcggactccaacacctggttcgacgtgcacgacccggccacgaactacgtcgtgtccaaggtgccacagtcgacgccggaggagttggaagaggcgatcgcgtcggcccatgccgcgttccccaagtggcgcgacaccagcatcatcaagcgtcaggggatcgcgttcaagtttgtgcagttgttgcgcgagaacatggacagaatcgcaagcgtcattgtcttggaacagggtaagacgtttgtcgatgcccagggtgacgtgactagaggattgcaggttgctgaggctgcgtgcaacatcactaatgacttgaagggtgagtcgttggaagtgtctactgatatggagaccaagatgattagagaacctttgggtgttgtgggatccatctgtccttttaacttcccagctatggtcccattgtggtctttgcctttggttttggtcacgggtaacactgctgtgattaagccttccgagagagtcccgggcgcaagtatgattatttgtgaattggccgccaaggctggtgttccacctggtgtgttgaacattgtccacggtaagcacgacaccgtcaacaagttgattgaggacccaagaatcaaggcattgacttttgttggtggtgacaaggccggtaagtacatttacgaaaagggttccagtttgggcaagagagtgcaggccaacttgggtgctaagaaccacttggttgtgttgccagacgcacacaagcagagttttgtcaatgccgtcaacggtgccgctttcggtgctgctggacagagatgtatggctatttctgtcttggtcaccgtgggtaagaccaaggaatgggtgcaggatgtcatcaaggacgccaagttgttgaacaccggaagtggatttgacccaaagagtgacttgggtccagtcatcaacccagagtccttgactcgtgctgaagaaatcattgctgattccgtggccaacggtgccgtgttggaattggacggaagaggatacagaccagaagacgctagattcgccaagggtaacttcttgggtccaaccatcttgaccaacgtcaagccaggcttgagagcatacgacgaagagattttcgctcctgttttgtctgtggttaacgtcgacaccattgacgaagccattgagttgatcaacaacaacaagtacggtaacggtgtttcattatttacttcctccggtggctcagcccagtatttcaccaagagaatcgacgtcggtcaagtcggtatcaatgtcccaatccctgttccattgcctatgttctccttcactggttccagaggctccttcttgggtgacttgaacttctacggtaaggccggtatcaccttcttgaccaagccaaagaccatcactagtgcctggaagaccaacttgattgatgacgagatcttgaaaccatctacctcgatgcctgtccaacagtaa

SEQ ID NO: 18 - protein, 549 aa, Candida viswanathii
  Candida viswanathii (ATCC20336); malonate- semialdehyde dehydrogenase (acetylating)(EC 1.2.1.18)
MLSRVLFKTKPRVPTKSITAMAIRNKSIVTLSSTTSTYPTDHTTPSTEPYITPSFVNNEFIKSDSNTWFDVHDPATNYVVSKVPQSTPEELEEAIASAHAAFPKWRDTSIIKRQGIAFKFVQLLRENMDRIASVIVLEQGKTFVDAQGDVTRGLQVAEAACNITNDLKGESLEVSTDMETKMIREPLGVVGSICPFNFPAMVPLWSLPLVLVTGNTAVIKPSERVPGASMIICELAAKAGVPPGVLNIVHGKHDTVNKLIEDPRIKALTFVGGDKAGKYIYEKGSSLGKRVQANLGAKNHLVVLPDAHKQSFVNAVNGAAFGAAGQRCMAISVLVTVGKTKEWVQDVIKDAKLLNTGSGFDPKSDLGPVINPESLTRAEEIIADSVANGAVLELDGRGYRPEDARFAKGNFLGPTILTNVKPGLRAYDEEIFAPVLSVVNVDTIDEAIELINNNKYGNGVSLFTSSGGSAQYFTKRIDVGQVGINVPIPVPLPMFSFTGSRGSFLGDLNFYGKAGITFLTKPKTITSAWKTNLIDDEILKPSTSMPVQQ

SEQ ID NO: 19 - DNA, 2883 bp, Artificial Sequence
  Description of Artificial Sequence Synthetic ALD6 deletion cassette
  modified_base (1001)..(1001): a, c, t, g, unknown or other
  modified_base (1145)..(1145): a, c, t, g, unknown or other
  modified_base (1401)..(1401): a, c, t, g, unknown or other
tatcacagcacacacgacctactcatcaaccacccagaatcaccgctagctggcaccgcgaactggaaggcattgggagataataaggttgtattgtgggtgtcgggtattgttaagggtatgtacgtaaggtggggggagaagggtgtgtgtgtgcttcggtgcgtcgcccctccacccctcctttcttcccgttgctcggccgttgatacccatggctaatatcctacccttttactatttgatccccacaattgctcctatggaggctggtgcacacacgactgaaaattagagagagagagagaaggatttcgatatcctataatttcacattcagtggttaagcgcctacctgtctctttccctctcccgcaaaagtatttaaacaaccaacaatacctcttctctgttttacctcttgtccgagtttttcacaaatacctcccgagttctgctgcaagtactactcttctttccatcatgttatccagagttcttgtatctacggttgtttcggtatgacccccggggatctgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcaatggttagcacaaaaacatacacagaaagggcatcagcacacccctccnaggttgcccaacgtttattccgcttaatggagtccaaaaagaccaacctctgcgcctcgatcgacgtgaccacaaccgccgagttcctttcgctcatcgacaagctcggtccccacatctgtctcgtgaagacgcacatcgatntcatctcagacttcagctacgagggcacgattgagccgttgcttgtgcttgcagagcgccacgggttcttgatattcgaggacaggaagtttgctgatatcggaaacaccgtgatgttgcagtacacctcgggggtataccggatcgcggcgtggagtgacatcacgaacgcgcacggagtgactgggaagggcgtcgttgaagggttgaaacgcggtgcggagggggtagaaaaggaaaggggcgtgttgatgtnggcggagttgtcgagtaaaggctcgttggcgcatggtgaatatacccgtgagacgatcgagattgcgaagagtgatcgggagttcgtgattgggttcatcgcgcagcgggacatggggggtagagaagaagggtttgattggatcatcatgacgcctggtgtggggttggatgataaaggcgatgcgttgggccagcagtataggactgttgatgaggtggttctgactggtaccgatgtgattattgtcgggagagggttgtttggaaaaggaagagaccctgaggtggagggaaagagatacagggatgctggatggaaggcatacttgaagagaactggtcagttagaataaatattgtaataaataggtctatatacatacactaagcttctaggacgtcattgtagtcttcgaagttgtctgctagtttagttctcatgatttcgaaaaccaataacgcaatggatgtagcagggatggtggttagtgcgttcctgacaaacccagagtacgccgcctcaaaccacgtcacattcgccctttgcttcatccgcatcacttgcttgaaggtatccacgtacgagttgtaatacaccttgaagaacggcttcgtctacggtcgacgacgggtacaacgagaattgtattgaattgatcaagaacatgatcttggtgttacagaacatcaagttcttggaccagactgagaatgcacagatatacaaggcgtcatgtgataaaatggatgagatttatccacaattgaagaaagagtttatggaaagtggtcaaccagaagctaaacaggaagaagcaaacgaagaggtgaaacaagaagaagaaggtaaataagtattttgtattatataacaaacaaagtaaggaatacagatttatacaataaattgccatactagtcacgtgagatatctcatccattccccaactcccaagaaaaaaaaaaagtgaaaaaaaaaatcaaacccaaagatcaacctccccatcatcatcgtcatcaaacccccagctcaattcgcagagctcggtaccaaatgggtcaacagaatccaattcggtggaccaacgtcaagccaggcttgagagcatacgacgaagagattttcgctcctgttttgtctgtggttaacgtcgacaccattgacgaagccattgagttgatcaacaacaacaagtacggtaacggtgtttcattatttacttcctccggtggctcagcccagtatttcaccaagagaatcgacgtcggtcaagtcggtatcaatgtcccaatccctgttccattgcctatgttctccttcactggttccagaggctccttcttgggtgacttgaacttctacggtaaggccggtatcaccttcttgaccaagccaaagaccatcactagtgcctggaagaccaacttgattgatgacgagatcttgaaaccatctacctcgatgcctgtccaacagtaa

SEQ ID NO: 20 - DNA, 500 bp, Candida viswanathii
  Candida viswanathii (ATCC20336); Genomic region upstream of the ALD6 gene
tatcacagcacacacgacctactcatcaaccacccagaatcaccgctagctggcaccgcgaactggaaggcattgggagataataaggttgtattgtgggtgtcgggtattgttaagggtatgtacgtaaggtggggggagaagggtgtgtgtgtgcttcggtgcgtcgcccctccacccctcctttcttcccgttgctcggccgttgatacccatggctaatatcctacccttttactatttgatccccacaattgctcctatggaggctggtgcacacacgactgaaaattagagagagagagagaaggatttcgatatcctataatttcacattcagtggttaagcgcctacctgtctctttccctctcccgcaaaagtatttaaacaaccaacaatacctcttctctgttttacctcttgtccgagtttttcacaaatacctcccgagttctgctgcaagtactactcttctttccatcatgttatccagagttct

SEQ ID NO: 21 - DNA, 24 bp, Artificial Sequence
  Description of Artificial Sequence Synthetic primer
tatcacagcacacacgacctactc

SEQ ID NO: 22 - DNA, 50 bp, Artificial Sequence
  Description of Artificial Sequence Synthetic primer
tcataccgaaacaaccgtagatacaagaactctggataacatgatggaaa

SEQ ID NO: 23 - DNA, 400 bp, Candida viswanathii (ATCC20336)
  Candida viswanathii (ATCC20336); Genomic region downstream of the ALD6 gene
gaccaacgtcaagccaggcttgagagcatacgacgaagagattttcgctcctgttttgtctgtggttaacgtcgacaccattgacgaagccattgagttgatcaacaacaacaagtacggtaacggtgtttcattatttacttcctccggtggctcagcccagtatttcaccaagagaatcgacgtcggtcaagtcggtatcaatgtcccaatccctgttccattgcctatgttctccttcactggttccagaggctccttcttgggtgacttgaacttctacggtaaggccggtatcaccttcttgaccaagccaaagaccatcactagtgcctggaagaccaacttgattgatgacgagatcttgaaaccatctacctcgatgcctgtccaacagtaa

SEQ ID NO: 24 - DNA, 50 bp, Artificial Sequence
  Description of Artificial Sequence Synthetic primer
ccaaatgggtcaacagaatccaattcggtggaccaacgtcaagccaggct

SEQ ID NO: 25 - DNA, 23 bp, Artificial Sequence
  Description of Artificial Sequence Synthetic primer
ttactgttggacaggcatcgagg

SEQ ID NO: 26 - DNA, 50 bp, Artificial Sequence
  Description of Artificial Sequence Synthetic primer
tttccatcatgttatccagagttcttgtatctacggttgtttcggtatga

SEQ ID NO: 27 - DNA, 50 bp, Artificial Sequence
  Description of Artificial Sequence Synthetic primer
agcctggcttgacgttggtccaccgaattggattctgttgacccatttgg

SEQ ID NO: 28 - DNA, 2040 bp, Candida sp.
  Candida sp.; NADPH cytochrome P450 reductase A (EC 1.6.2.4)
atggctttagacaagttagatttgtatgtcatcataacattggtggtcgctgtagccgcctattttgctaagaaccagttccttgatcagccccaggacaccgggttcctcaacacggacagcggaagcaactccagagacgtcttgctgacattgaagaagaataataaaaacacgttgttgttgtttgggtcccagacgggtacggcagaagattacgccaacaaattgtccagagaattgcactccagatttggcttgaaaacgatggttgcagatttcgctgattacgattgggataacttcggagatatcaccgaagacatcttggtgtttttcattgttgccacctatggtgagggtgaacctaccgataatgccgacgagttccacacctggttgactgaagaagctgacactttgagtaccttgaaatacaccgtgttcgggttgggtaactccacgtacgagttcttcaatgccattggtagaaagtttgacagattgttgagcgagaaaggtggtgacaggtttgctgaatacgctgaaggtgatgacggtactggcaccttggacgaagatttcatggcctggaaggacaatgtctttgacgccttgaagaatgatttgaactttgaagaaaaggaattgaagtacgaaccaaacgtgaaattgactgagagagacgacttgtctgctgctgactcccaagtttccttgggtgagccaaacaagaagtacatcaactccgagggcatcgacttgaccaagggtccattcgaccacacccacccatacttggccagaatcaccgagacgagagagttgttcagctccaaggacagacactgtatccacgttgaatttgacatttctgaatcgaacttgaaatacaccaccggtgaccatctagctatctggccatccaactccgacgaaaacattaagcaatttgccaagtgtttcggattggaagataaactcgacactgttattgaattgaaggcgttggactccacttacaccatcccattcccaaccccaattacctacggtgctgtcattagacaccatttagaaatctccggtccagtctcgagacaattctttttgtcaattgctgggtttgctcctgatgaagaaacaaagaaggcttttaccagacttggtggtgacaagcaagaattcgccgccaaggtcacccgcagaaagttcaacattgccgatgccttgttatattcctccaacaacgctccatggtccgatgttccttttgaattccttattgaaaacgttccacacttgactccacgttactactccatttcgtcttcgtcattgagtgaaaagcaactcatcaacgttactgcagttgttgaagccgaagaagaagctgatggcagaccagtcactggtgttgtcaccaacttgttgaagaacgttgaaattgtgcaaaacaagactggcgaaaagccacttgtccactacgatttgagcggcccaagaggcaagttcaacaagttcaagttgccagtgcatgtgagaagatccaactttaagttgccaaagaactccaccaccccagttatcttgattggtccaggtactggtgttgccccattgagaggttttgtcagagaaagagttcaacaagtcaagaatggtgtcaatgttggcaagactttgttgttttatggttgcagaaactccaacgaggactttttgtacaagcaagaatgggccgagtacgcttctgttttgggtgaaaactttgagatgttcaatgccttctccagacaagacccatccaagaaggtttacgtccaggataagattttagaaaacagccaacttgtgcacgagttgttgactgaaggtgccattatctacgtctgtggtgatgccagtagaatggctagagacgtgcagaccacaatttccaagattgttgctaaaagcagagaaattagtgaagacaaggctgctgaattggtcaagtcctggaaggtccaaaatagataccaagaagatgtttggtag

SEQ ID NO: 29 - protein, 679 aa, Candida sp.
  Candida sp.; NADPH cytochrome P450 reductase A (EC 1.6.2.4)
MALDKLDLYVIITLVVAVAAYFAKNQFLDQPQDTGFLNTDSGSNSRDVLSTLKKNNKNTLLLFGSQTGTAEDYANKLSRELHSRFGLKTMVADFADYDWDNFGDITEDILVFFIVATYGEGEPTDNADEFHTWLTEEADTLSTLKYTVFGLGNSTYEFFNAIGRKFDRLLSEKGGDRFAEYAEGDDGTGTLDEDFMAWKDNVFDALKNDLNFEEKELKYEPNVKLTERDDLSAADSQVSLGEPNKKYINSEGIDLTKGPFDHTHPYLARITETRELFSSKDRHCIHVEFDISESNLKYTTGDHLAIWPSNSDENIKQFAKCFGLEDKLDTVIELKALDSTYTIPFPTPITYGAVIRHHLEISGPVSRQFFLSIAGFAPDEETKKAFTRLGGDKQEFAAKVTRRKFNIADALLYSSNNAPWSDVPFEFLIENVPHLTPRYYSISSSSLSEKQLINVTAVVEAEEEADGRPVTGVVTNLLKNVEIVQNKTGEKPLVHYDLSGPRGKFNKFKLPVHVRRSNFKLPKNSTTPVILIGPGTGVAPLRGFVRERVQQVKNGVNVGKTLLFYGCRNSNEDFLYKQEWAEYASVLGENFEMFNAFSRQDPSKKVYVQDKILENSQLVHELLTEGAIIYVCGDASRMARDVQTTISKIVAKSREISEDKAAELVKSWKVQNRYQEDVW

SEQ ID NO: 30 - DNA, 2037 bp, Candida sp.
  Candida sp.; NADPH cytochrome P450 reductase B (EC 1.6.2.4)
atggctttagacaagttagatttgtatgtcatcataacattggtggtcgctgtggccgcctattttgctaagaaccagttccttgatcagccccaggacaccgggttcctcaacacggacagcggaagcaactccagagacgtcttgctgacattgaagaagaataataaaaacacgttgttgttgtttgggtcccagaccggtacggcagaagattacgccaacaaattgtcaagagaattgcactccagatttggcttgaaaaccatggttgcagatttcgctgattacgattgggataacttcggagatatcaccgaagatatcttggtgtttttcatcgttgccacctacggtgagggtgaacctaccgacaatgccgacgagttccacacctggttgactgaagaagctgacactttgagtactttgagatataccgtgttcgggttgggtaactccacctacgagttcttcaatgctattggtagaaagtttgacagattgttgagtgagaaaggtggtgacagatttgctgaatatgctgaaggtgacgacggcactggcaccttggacgaagatttcatggcctggaaggataatgtctttgacgccttgaagaatgacttgaactttgaagaaaaggaattgaagtacgaaccaaacgtgaaattgactgagagagatgacttgtctgctgccgactcccaagtttccttgggtgagccaaacaagaagtacatcaactccgagggcatcgacttgaccaagggtccattcgaccacacccacccatacttggccaggatcaccgagaccagagagttgttcagctccaaggaaagacactgtattcacgttgaatttgacatttctgaatcgaacttgaaatacaccaccggtgaccatctagccatctggccatccaactccgacgaaaacatcaagcaatttgccaagtgtttcggattggaagataaactcgacactgttattgaattgaaggcattggactccacttacaccattccattcccaactccaattacttacggtgctgtcattagacaccatttagaaatctccggtccagtctcgagacaattctttttgtcgattgctgggtttgctcctgatgaagaaacaaagaagactttcaccagacttggtggtgacaaacaagaattcgccaccaaggttacccgcagaaagttcaacattgccgatgccttgttatattcctccaacaacactccatggtccgatgttccttttgagttccttattgaaaacatccaacacttgactccacgttactactccatttcttcttcgtcgttgagtgaaaaacaactcatcaatgttactgcagtcgttgaggccgaagaagaagccgatggcagaccagtcactggtgttgttaccaacttgttgaagaacattgaaattgcgcaaaacaagactggcgaaaagccacttgttcactacgatttgagcggcccaagaggcaagttcaacaagttcaagttgccagtgcacgtgagaagatccaactttaagttgccaaagaactccaccaccccagttatcttgattggtccaggtactggtgttgccccattgagaggtttcgttagagaaagagttcaacaagtcaagaatggtgtcaatgttggcaagactttgttgttttatggttgcagaaactccaacgaggactttttgtacaagcaagaatgggccgagtacgcttctgttttgggtgaaaactttgagatgttcaatgccttctctagacaagacccatccaagaaggtttacgtccaggataagattttagaaaacagccaacttgtgcacgaattgttgaccgaaggtgccattatctacgtctgtggtgacgccagtagaatggccagagacgtccagaccacgatctccaagattgttgccaaaagcagagaaatcagtgaagacaaggccgctgaattggtcaagtcctggaaagtccaaaatagataccaagaagatgtttgg

SEQ ID NO: 31 - protein, 679 aa, Candida sp.
  Candida sp.; NADPH cytochrome P450 reductase B (EC 1.6.2.4)
MALDKLDLYVIITLVVAVAAYFAKNQFLDQPQDTGFLNTDSGSNSRDVLSTLKKNNKNTLLLFGSQTGTAEDYANKLSRELHSRFGLKTMVADFADYDWDNFGDITEDILVFFIVATYGEGEPTDNADEFHTWLTEEADTLSTLRYTVFGLGNSTYEFFNAIGRKFDRLLSEKGGDRFAEYAEGDDGTGTLDEDFMAWKDNVFDALKNDLNFEEKELKYEPNVKLTERDDLSAADSQVSLGEPNKKYINSEGIDLTKGPFDHTHPYLARITETRELFSSKERHCIHVEFDISESNLKYTTGDHLAIWPSNSDENIKQFAKCFGLEDKLDTVIELKALDSTYTIPFPTPITYGAVIRHHLEISGPVSRQFFLSIAGFAPDEETKKTFTRLGGDKQEFATKVTRRKFNIADALLYSSNNTPWSDVPFEFLIENIQHLTPRYYSISSSSLSEKQLINVTAVVEAEEEADGRPVTGVVTNLLKNIEIAQNKTGEKPLVHYDLSGPRGKFNKFKLPVHVRRSNFKLPKNSTTPVILIGPGTGVAPLRGFVRERVQQVKNGVNVGKTLLFYGCRNSNEDFLYKQEWAEYASVLGENFEMFNAFSRQDPSKKVYVQDKILENSQLVHELLTEGAIIYVCGDASRMARDVQTTISKIVAKSREISEDKAAELVKSWKVQNRYQEDVW

SEQ ID NO: 32 - DNA, 1572 bp, Candida sp.
  Candida sp.; Cytochrome P-450 monooxygenase CYP52A12 (EC 1.14.14.1)
atggccacacaagaaatcatcgattctgtacttccgtacttgaccaaatggtacactgtgattactgcagcagtattagtcttccttatctccacaaacatcaagaactacgtcaaggcaaagaaattgaaatgtgtcgatccaccatacttgaaggatgccggtctcactggtattctgtctttgatcgccgccatcaaggccaagaacgacggtagattggctaactttgccgatgaagttttcgacgagtacccaaaccacaccttctacttgtctgttgccggtgctttgaagattgtcatgactgttgacccagaaaacatcaaggctgtcttggccacccaattcactgacttctccttgggtaccagacacgcccactttgctcctttgttgggtgacggtatcttcaccttggacggagaaggttggaagcactccagagctatgttgagaccacagtttgctagagaccagattggacacgttaaagccttggaaccacacatccaaatcatggctaagcagatcaagttgaaccagggaaagactttcgatatccaagaattgttctttagatttaccgtcgacaccgctactgagttcttgtttggtgaatccgttcactccttgtacgatgaaaaattgggcatcccaactccaaacgaaatcccaggaagagaaaactttgccgctgctttcaacgtttcccaacactacttggccaccagaagttactcccagactttttactttttgaccaaccctaaggaattcagagactgtaacgccaaggtccaccacttggccaagtactttgtcaacaaggccttgaactttactcctgaagaactcgaagagaaatccaagtccggttacgttttcttgtacgaattggttaagcaaaccagagatccaaaggtcttgcaagatcaattgttgaacattatggttgccggaagagacaccactgccggtttgttgtcctttgctttgtttgaattggctagacacccagagatgtggtccaagttgagagaagaaatcgaagttaactttggtgttggtgaagactcccgcgttgaagaaattaccttcgaagccttgaagagatgtgaatacttgaaggctatccttaacgaaaccttgcgtatgtacccatctgttcctgtcaactttagaaccgccaccagagacaccactttgccaagaggtggtggtgctaacggtaccgacccaatctacattcctaaaggctccactgttgcttacgttgtctacaagacccaccgtttggaagaatactacggtaaggacgctaacgacttcagaccagaaagatggtttgaaccatctactaagaagttgggctgggcttatgttccattcaacggtggtccaagagtctgcttgggtcaacaattcgccttgactgaagcttcttatgtgatcactagattggcccagatgtttgaaactgtctcatctgatccaggtctcgaataccctccaccaaagtgtattcacttgaccatgagtcacaacgatggtgtctttgtcaagatgtaa

SEQ ID NO: 33 - protein, 523 aa, Candida sp.
  Candida sp.; Cytochrome P-450 monooxygenase CYP52A12 (EC 1.14.14.1)
MATQEIIDSVLPYLTKWYTVITAAVLVFLISTNIKNYVKAKKLKCVDPPYLKDAGLTGISSLIAAIKAKNDGRLANFADEVFDEYPNHTFYLSVAGALKIVMTVDPENIKAVLATQFTDFSLGTRHAHFAPLLGDGIFTLDGEGWKHSRAMLRPQFARDQIGHVKALEPHIQIMAKQIKLNQGKTFDIQELFFRFTVDTATEFLFGESVHSLYDEKLGIPTPNEIPGRENFAAAFNVSQHYLATRSYSQTFYFLTNPKEFRDCNAKVHHLAKYFVNKALNFTPEELEEKSKSGYVFLYELVKQTRDPKVLQDQLLNIMVAGRDTTAGLLSFALFELARHPEMWSKLREEIEVNFGVGEDSRVEEITFEALKRCEYLKAILNETLRMYPSVPVNFRTATRDTTLPRGGGANGTDPIYIPKGSTVAYVVYKTHRLEEYYGKDANDFRPERWFEPSTKKLGWAYVPFNGGPRVCLGQQFALTEASYVITRLAQMFETVSSDPGLEYPPPKCIHLTMSHNDGVFVKM

SEQ ID NO: 34 - DNA, 1569 bp, Candida sp.
  Candida sp.; Cytochrome P-450 monooxygenase CYP52A13 (EC 1.14.14.1)
atgactgtacacgatattatcgccacatacttcaccaaatggtacgtgatagtaccactcgctttgattgcttatagagtcctcgactacttctatggcagatacttgatgtacaagcttggtgctaaaccatttttccagaaacagacagacggctgtttcggattcaaagctccgcttgaattgttgaagaagaagagcgacggtaccctcatagacttcacactccagcgtatccacgatctcgatcgtcccgatatcccaactttcacattcccggtcttttccatcaaccttgtcaatacccttgagccggagaacatcaaggccatcttggccactcagttcaacgatttctccttgggtaccagacactcgcactttgctcctttgttgggtgatggtatctttacgttggatggcgccggctggaagcacagcagatctatgttgagaccacagtttgccagagaacagatttcccacgtcaagttgttggagccacacgttcaggtgttcttcaaacacgtcagaaaggcacagggcaagacttttgacatccaggaattgtttttcagattgaccgtcgactccgccaccgagtttttgtttggtgaatccgttgagtccttgagagatgaatctatcggcatgtccatcaatgcgcttgactttgacggcaaggctggctttgctgatgcttttaactattcgcagaattatttggcttcgagagcggttatgcaacaattgtactgggtgttgaacgggaaaaagtttaaggagtgcaacgctaaagtgcacaagtttgctgactactacgtcaacaaggctttggacttgacgcctgaacaattggaaaagcaggatggttatgtgtttttgtacgaattggtcaagcaaaccagagacaagcaagtgttgagagaccaattgttgaacatcatggttgctggtagagacaccaccgccggtttgttgtcgtttgttttctttgaattggccagaaacccagaagttaccaacaagttgagagaagaaattgaggacaagtttggactcggtgagaatgctagtgttgaagacatttcctttgagtcgttgaagtcctgtgaatacttgaaggctgttctcaacgaaaccttgagattgtacccatccgtgccacagaatttcagagttgccaccaagaacactaccctcccaagaggtggtggtaaggacgggttgtctcctgttttggtgagaaagggtcagaccgttatttacggtgtctacgcagcccacagaaacccagctgtttacggtaaggacgctcttgagtttagaccagagagatggtttgagccagagacaaagaagcttggctgggccttcctcccattcaacggtggtccaagaatctgtttgggacagcagtttgccttgacagaagcttcgtatgtcactgtcaggttgctccaggagtttgcacacttgtctatggacccagacaccgaatatccacctaagaaaatgtcgcatttgaccatgtcgcttttcgacggtgccaatattgagatgtattag

SEQ ID NO: 35 - protein, 522 aa, Candida sp.
  Candida sp.; Cytochrome P-450 monooxygenase CYP52A13 (EC 1.14.14.1)
MTVHDIIATYFTKWYVIVPLALIAYRVLDYFYGRYLMYKLGAKPFFQKQTDGCFGFKAPLELLKKKSDGTLIDFTLQRIHDLDRPDIPTFTFPVFSINLVNTLEPENIKAILATQFNDFSLGTRHSHFAPLLGDGIFTLDGAGWKHSRSMLRPQFAREQISHVKLLEPHVQVFFKHVRKAQGKTFDIQELFFRLTVDSATEFLFGESVESLRDESIGMSINALDFDGKAGFADAFNYSQNYLASRAVMQQLYWVLNGKKFKECNAKVHKFADYYVNKALDLTPEQLEKQDGYVFLYELVKQTRDKQVLRDQLLNIMVAGRDTTAGLLSFVFFELARNPEVTNKLREEIEDKFGLGENASVEDISFESLKSCEYLKAVLNETLRLYPSVPQNFRVATKNTTLPRGGGKDGLSPVLVRKGQTVIYGVYAAHRNPAVYGKDALEFRPERWFEPETKKLGWAFLPFNGGPRICLGQQFALTEASYVTVRLLQEFAHLSMDPDTEYPPKKMSHLTMSLFDGANIEMY

SEQ ID NO: 36 - DNA, 1569 bp, Candida sp.
  Candida sp.; Cytochrome P-450 monooxygenase CYP52A14 (EC 1.14.14.1)
atgactgcacaggatattatcgccacatacatcaccaaatggtacgtgatagtaccactcgctttgattgcttatagggtcctcgactacttttacggcagatacttgatgtacaagcttggtgctaaaccgtttttccagaaacaaacagacggttatttcggattcaaagctccacttgaattgttaaaaaagaagagtgacggtaccctcatagacttcactctcgagcgtatccaagcgctcaatcgtccagatatcccaacttttacattcccaatcttttccatcaaccttatcagcacccttgagccggagaacatcaaggctatcttggccacccagttcaacgatttctccttgggcaccagacactcgcactttgctcctttgttgggcgatggtatctttaccttggacggtgccggctggaagcacagcagatctatgttgagaccacagtttgccagagaacagatttcccacgtcaagttgttggagccacacatgcaggtgttcttcaagcacgtcagaaaggcacagggcaagacttttgacatccaagaattgtttttcagattgaccgtcgactccgccactgagtttttgtttggtgaatccgttgagtccttgagagatgaatctattgggatgtccatcaatgcacttgactttgacggcaaggctggctttgctgatgcttttaactactcgcagaactatttggcttcgagagcggttatgcaacaattgtactgggtgttgaacgggaaaaagtttaaggagtgcaacgctaaagtgcacaagtttgctgactattacgtcagcaaggctttggacttgacacctgaacaattggaaaagcaggatggttatgtgttcttgtacgagttggtcaagcaaaccagagacaggcaagtgttgagagaccagttgttgaacatcatggttgccggtagagacaccaccgccggtttgttgtcgtttgttttctttgaattggccagaaacccagaggtgaccaacaagttgagagaagaaatcgaggacaagtttggtcttggtgagaatgctcgtgttgaagacatttcctttgagtcgttgaagtcatgtgaatacttgaaggctgttctcaacgaaactttgagattgtacccatccgtgccacagaatttcagagttgccaccaaaaacactacccttccaaggggaggtggtaaggacgggttatctcctgttttggtcagaaagggtcaaaccgttatgtacggtgtctacgctgcccacagaaacccagctgtctacggtaaggacgcccttgagtttagaccagagaggtggtttgagccagagacaaagaagcttggctgggccttccttccattcaacggtggtccaagaatttgcttgggacagcagtttgccttgacagaagcttcgtatgtcactgtcagattgctccaagagtttggacacttgtctatggaccccaacaccgaatatccacctaggaaaatgtcgcatttgaccatgtcccttttcgacggtgccaacattgagatgtattag

SEQ ID NO: 37 - protein, 522 aa, Candida sp.
  Candida sp.; Cytochrome P-450 monooxygenase CYP52A14 (EC 1.14.14.1)
MTAQDIIATYITKWYVIVPLALIAYRVLDYFYGRYLMYKLGAKPFFQKQTDGYFGFKAPLELLKKKSDGTLIDFTLERIQALNRPDIPTFTFPIFSINLISTLEPENIKAILATQFNDFSLGTRHSHFAPLLGDGIFTLDGAGWKHSRSMLRPQFAREQISHVKLLEPHMQVFFKHVRKAQGKTFDIQELFFRLTVDSATEFLFGESVESLRDESIGMSINALDFDGKAGFADAFNYSQNYLASRAVMQQLYWVLNGKKFKECNAKVHKFADYYVSKALDLTPEQLEKQDGYVFLYELVKQTRDRQVLRDQLLNIMVAGRDTTAGLLSFVFFELARNPEVTNKLREEIEDKFGLGENARVEDISFESLKSCEYLKAVLNETLRLYPSVPQNFRVATKNTTLPRGGGKDGLSPVLVRKGQTVMYGVYAAHRNPAVYGKDALEFRPERWFEPETKKLGWAFLPFNGGPRICLGQQFALTEASYVTVRLLQEFGHLSMDPNTEYPPRKMSHLTMSLFDGANIEMY

SEQ ID NO: 38 - DNA, 1623 bp, Candida sp.
  Candida sp.; Cytochrome P-450 monooxygenase CYP52A15 (EC 1.14.14.1)
atgtcgtcttctccatcgtttgcccaagaggttctcgctaccactagtccttacatcgagtactttcttgacaactacaccagatggtactacttcatacctttggtgcttctttcgttgaactttataagtttgctccacacaaggtacttggaacgcaggttccacgccaagccactcggtaactttgtcagggaccctacgtttggtatcgctactccgttgcttttgatctacttgaagtcgaaaggtacggtcatgaagtttgcttggggcctctggaacaacaagtacatcgtcagagacccaaagtacaagacaactgggctcaggattgttggcctcccattgattgaaaccatggacccagagaacatcaaggctgttttggctactcagttcaatgatttctctttgggaaccagacacgatttcttgtactccttgttgggtgacggtattttcaccttggacggtgctggctggaaacatagtagaactatgttgagaccacagtttgctagagaacaggtttctcacgtcaagttgttggagccacacgttcaggtgttcttcaagcacgttagaaagcaccgcggtcaaacgttcgacatccaagaattgttcttcaggttgaccgtcgactccgccaccgagttcttgtttggtgagtctgctgaatccttgagggacgaatctattggattgaccccaaccaccaaggatttcgatggcagaagagatttcgctgacgctttcaactattcgcagacttaccaggcctacagatttttgttgcaacaaatgtactggatcttgaatggctcggaattcagaaagtcgattgctgtcgtgcacaagtttgctgaccactatgtgcaaaaggctttggagttgaccgacgatgacttgcagaaacaagacggctatgtgttcttgtacgagttggctaagcaaaccagagacccaaaggtcttgagagaccagttattgaacattttggttgccggtagagacacgaccgccggtttgttgtcatttgttttctacgagttgtcaagaaaccctgaggtgtttgctaagttgagagaggaggtggaaaacagatttggactcggtgaagaagctcgtgttgaagagatctcgtttgagtccttgaagtcttgtgagtacttgaaggctgtcatcaatgaaaccttgagattgtacccatcggttccacacaactttagagttgctaccagaaacactaccctcccaagaggtggtggtgaagatggatactcgccaattgtcgtcaagaagggtcaagttgtcatgtacactgttattgctacccacagagacccaagtatctacggtgccgacgctgacgtcttcagaccagaaagatggtttgaaccagaaactagaaagttgggctgggcatacgttccattcaatggtggtccaagaatctgtttgggtcaacagtttgccttgaccgaagcttcatacgtcactgtcagattgctccaggagtttgcacacttgtctatggacccagacaccgaatatccaccaaaattgcagaacaccttgaccttgtcgctctttgatggtgctgatgttagaatgtactaa

SEQ ID NO: 39 - protein, 540 aa, Candida sp.
  Candida sp.; Cytochrome P-450 monooxygenase CYP52A15 (EC 1.14.14.1)
MSSSPSFAQEVLATTSPYIEYFLDNYTRWYYFIPLVLLSLNFISLLHTRYLERRFHAKPLGNFVRDPTFGIATPLLLIYLKSKGTVMKFAWGLWNNKYIVRDPKYKTTGLRIVGLPLIETMDPENIKAVLATQFNDFSLGTRHDFLYSLLGDGIFTLDGAGWKHSRTMLRPQFAREQVSHVKLLEPHVQVFFKHVRKHRGQTFDIQELFFRLTVDSATEFLFGESAESLRDESIGLTPTTKDFDGRRDFADAFNYSQTYQAYRFLLQQMYWILNGSEFRKSIAVVHKFADHYVQKALELTDDDLQKQDGYVFLYELAKQTRDPKVLRDQLLNILVAGRDTTAGLLSFVFYELSRNPEVFAKLREEVENRFGLGEEARVEEISFESLKSCEYLKAVINETLRLYPSVPHNFRVATRNTTLPRGGGEDGYSPIVVKKGQVVMYTVIATHRDPSIYGADADVFRPERWFEPETRKLGWAYVPFNGGPRICLGQQFALTEASYVTVRLLQEFAHLSMDPDTEYPPKLQNTLTLSLFDGADVRMY

SEQ ID NO: 40 - DNA, 1623 bp, Candida sp.
  Candida sp.; Cytochrome P-450 monooxygenase CYP52A16 (EC 1.14.14.1)
atgtcgtcttctccatcgtttgctcaggaggttctcgctaccactagtccttacatcgagtactttcttgacaactacaccagatggtactacttcatccctttggtgcttctttcgttgaacttcatcagcttgctccacacaaagtacttggaacgcaggttccacgccaagccgctcggtaacgtcgtgttggatcctacgtttggtatcgctactccgttgatcttgatctacttaaagtcgaaaggtacagtcatgaagtttgcctggagcttctggaacaacaagtacattgtcaaagacccaaagtacaagaccactggccttagaattgtcggcctcccattgattgaaaccatagacccagagaacatcaaagctgtgttggctactcagttcaacgatttctccttgggaactagacacgatttcttgtactccttgttgggcgatggtatttttaccttggacggtgctggctggaaacacagtagaactatgttgagaccacagtttgctagagaacaggtttcccacgtcaagttgttggaaccacacgttcaggtgttcttcaagcacgttagaaaacaccgcggtcagacttttgacatccaagaattgttcttcagattgaccgtcgactccgccaccgagttcttgtttggtgagtctgctgaatccttgagagacgactctgttggtttgaccccaaccaccaaggatttcgaaggcagaggagatttcgctgacgctttcaactactcgcagacttaccaggcctacagatttttgttgcaacaaatgtactggattttgaatggcgcggaattcagaaagtcgattgccatcgtgcacaagtttgctgaccactatgtgcaaaaggctttggagttgaccgacgatgacttgcagaaacaagacggctatgtgttcttgtacgagttggctaagcaaactagagacccaaaggtcttgagagaccagttgttgaacattttggttgccggtagagacacgaccgccggtttgttgtcgtttgtgttctacgagttgtcgagaaaccctgaagtgtttgccaagttgagagaggaggtggaaaacagatttggactcggcgaagaggctcgtgttgaagagatctcttttgagtccttgaagtcctgtgagtacttgaaggctgtcatcaatgaagccttgagattgtacccatctgttccacacaacttcagagttgccaccagaaacactacccttccaagaggcggtggtaaagacggatgctcgccaattgttgtcaagaagggtcaagttgtcatgtacactgtcattggtacccacagagacccaagtatctacggtgccgacgccgacgtcttcagaccagaaagatggttcgagccagaaactagaaagttgggctgggcatatgttccattcaatggtggtccaagaatctgtttgggtcagcagtttgccttgactgaagcttcatacgtcactgtcagattgctccaagagtttggaaacttgtccctggatccaaacgctgagtacccaccaaaattgcagaacaccttgaccttgtcactctttgatggtgctgacgttagaatgttctaa

SEQ ID NO: 41 - protein, 540 aa, Candida sp.
  Candida sp.; Cytochrome P-450 monooxygenase CYP52A16 (EC 1.14.14.1)
MSSSPSFAQEVLATTSPYIEYFLDNYTRWYYFIPLVLLSLNFISLLHTKYLERRFHAKPLGNVVLDPTFGIATPLILIYLKSKGTVMKFAWSFWNNKYIVKDPKYKTTGLRIVGLPLIETIDPENIKAVLATQFNDFSLGTRHDFLYSLLGDGIFTLDGAGWKHSRTMLRPQFAREQVSHVKLLEPHVQVFFKHVRKHRGQTFDIQELFFRLTVDSATEFLFGESAESLRDDSVGLTPTTKDFEGRGDFADAFNYSQTYQAYRFLLQQMYWILNGAEFRKSIAIVHKFADHYVQKALELTDDDLQKQDGYVFLYELAKQTRDPKVLRDQLLNILVAGRDTTAGLLSFVFYELSRNPEVFAKLREEVENRFGLGEEARVEEISFESLKSCEYLKAVINEALRLYPSVPHNFRVATRNTTLPRGGGKDGCSPIVVKKGQVVMYTVIGTHRDPSIYGADADVFRPERWFEPETRKLGWAYVPFNGGPRICLGQQFALTEASYVTVRLLQEFGNLSSDPNAEYPPKLQNTLTLSLFDGADVRMF

SEQ ID NO: 42 - DNA, 1554 bp, Candida sp.
  Candida sp.; Cytochrome P-450 monooxygenase CYP52A17 (EC 1.14.14.1)
atgattgaacaactcctagaatattggtatgtcgttgtgccagtgttgtacatcatcaaacaactccttgcatacacaaagactcgcgtcttgatgaaaaagttgggtgctgctccagtcacaaacaagttgtacgacaacgctttcggtatcgtcaatggatggaaggctctccagttcaagaaagagggcagggctcaagagtacaacgattacaagtttgaccactccaagaacccaagcgtgggcacctacgtcagtattcttttcggcaccaggatcgtcgtgaccaaagatccagagaatatcaaagctattttggcaacccagtttggtgatttttctttgggcaagaggcacactctttttaagcctttgttaggtgatgggatcttcacattggacggcgaaggctggaagcacagcagagccatgttgagaccacagtttgccagagaacaagttgctcatgtgacgtcgttggaaccacacttccagttgttgaagaagcatattcttaagcacaagggtgaatactttgatatccaggaattgttctttagatttaccgttgattcggccacggagttcttatttggtgagtccgtgcactccttaaaggacgaatctattggtatcaaccaagacgatatagattttgctggtagaaaggactttgctgagtcgttcaacaaagcccaggaatacttggctattagaaccttggtgcagacgttctactggttggtcaacaacaaggagtttagagactgtaccaagctggtgcacaagttcaccaactactatgttcagaaagctttggatgctagcccagaagagcttgaaaagcaaagtgggtatgtgttcttgtacgagcttgtcaagcagacaagagaccccaatgtgttgcgtgaccagtctttgaacatcttgttggccggaagagacaccactgctgggttgttgtcgtttgctgtctttgagttggccagacacccagagatctgggccaagttgagagaggaaattgaacaacagtttggtcttggagaagactctcgtgttgaagagattacctttgagagcttgaagagatgtgagtacttgaaagcgttccttaatgaaaccttgcgtatttacccaagtgtcccaagaaacttcagaatcgccaccaagaacacgacattgccaaggggcggtggttcagacggtacctcgccaatcttgatccaaaagggagaagctgtgtcgtatggtatcaactctactcatttggaccctgtctattacggccctgatgctgctgagttcagaccagagagatggtttgagccatcaaccaaaaagctcggctgggcttacttgccattcaacggtggtccaagaatctgtttgggtcagcagtttgccttgacggaagctggctatgtgttggttagattggtgcaagagttctcccacgttaggctggacccagacgaggtgtacccgccaaagaggttgaccaacttgaccatgtgtttgcaggatggtgctattgtcaagtttgactag

SEQ ID NO: 43 - protein, 517 aa, Candida sp.
  Candida sp.; Cytochrome P-450 monooxygenase CYP52A17 (EC 1.14.14.1)
MIEQLLEYWYVVVPVLYIIKQLLAYTKTRVLMKKLGAAPVTNKLYDNAFGIVNGWKALQFKKEGRAQEYNDYKFDHSKNPSVGTYVSILFGTRIVVTKDPENIKAILATQFGDFSLGKRHTLFKPLLGDGIFTLDGEGWKHSRAMLRPQFAREQVAHVTSLEPHFQLLKKHILKHKGEYFDIQELFFRFTVDSATEFLFGESVHSLKDESIGINQDDIDFAGRKDFAESFNKAQEYLAIRTLVQTFYWLVNNKEFRDCTKSVHKFTNYYVQKALDASPEELEKQSGYVFLYELVKQTRDPNVLRDQSLNILLAGRDTTAGLLSFAVFELARHPEIWAKLREEIEQQFGLGEDSRVEEITFESLKRCEYLKAFLNETLRIYPSVPRNFRIATKNTTLPRGGGSDGTSPILIQKGEAVSYGINSTHLDPVYYGPDAAEFRPERWFEPSTKKLGWAYLPFNGGPRICLGQQFALTEAGYVLVRLVQEFSHVRSDPDEVYPPKRLTNLTMCLQDGAIVKFD

SEQ ID NO: 44 - DNA, 1554 bp, Candida sp.
  Candida sp.; Cytochrome P-450 monooxygenase CYP52A18 (EC 1.14.14.1)
atgattgaacaaatcctagaatattggtatattgttgtgcctgtgttgtacatcatcaaacaactcattgcctacagcaagactcgcgtcttgatgaaacagttgggtgctgctccaatcacaaaccagttgtacgacaacgttttcggtatcgtcaacggatggaaggctctccagttcaagaaagagggcagagctcaagagtacaacgatcacaagtttgacagctccaagaacccaagcgtcggcacctatgtcagtattctttttggcaccaagattgtcgtgaccaaggatccagagaatatcaaagctattttggcaacccagtttggcgatttttctttgggcaagagacacgctctttttaaacctttgttaggtgatgggatcttcaccttggacggcgaaggctggaagcatagcagatccatgttaagaccacagtttgccagagaacaagttgctcatgtgacgtcgttggaaccacacttccagttgttgaagaagcatatccttaaacacaagggtgagtactttgatatccaggaattgttctttagatttactgtcgactcggccacggagttcttatttggtgagtccgtgcactccttaaaggacgaaactatcggtatcaaccaagacgatatagattttgctggtagaaaggactttgctgagtcgttcaacaaagcccaggagtatttgtctattagaattttggtgcagaccttctactggttgatcaacaacaaggagtttagagactgtaccaagctggtgcacaagtttaccaactactatgttcagaaagctttggatgctaccccagaggaacttgaaaagcaaggcgggtatgtgttcttgtatgagcttgtcaagcagacgagagaccccaaggtgttgcgtgaccagtctttgaacatcttgttggcaggaagagacaccactgctgggttgttgtcctttgctgtgtttgagttggccagaaacccacacatctgggccaagttgagagaggaaattgaacagcagtttggtcttggagaagactctcgtgttgaagagattacctttgagagcttgaagagatgtgagtacttgaaagcgttccttaacgaaaccttgcgtgtttacccaagtgtcccaagaaacttcagaatcgccaccaagaatacaacattgccaaggggtggtggtccagacggtacccagccaatcttgatccaaaagggagaaggtgtgtcgtatggtatcaactctacccacttagatcctgtctattatggccctgatgctgctgagttcagaccagagagatggtttgagccatcaaccagaaagctcggctgggcttacttgccattcaacggtgggccacgaatctgtttgggtcagcagtttgccttgaccgaagctggttacgttttggtcagattggtgcaagagttctcccacattaggctggacccagatgaagtgtatccaccaaagaggttgaccaacttgaccatgtgtttgcaggatggtgctattgtcaagtttgactag

SEQ ID NO: 45 - protein, 517 aa, Candida sp.
  Candida sp.; Cytochrome P-450 monooxygenase CYP52A18 (EC 1.14.14.1)
MIEQILEYWYIVVPVLYIIKQLIAYSKTRVLMKQLGAAPITNQLYDNVFGIVNGWKALQFKKEGRAQEYNDHKFDSSKNPSVGTYVSILFGTKIVVTKDPENIKAILATQFGDFSLGKRHALFKPLLGDGIFTLDGEGWKHSRSMLRPQFAREQVAHVTSLEPHFQLLKKHILKHKGEYFDIQELFFRFTVDSATEFLFGESVHSLKDETIGINQDDIDFAGRKDFAESFNKAQEYLSIRILVQTFYWLINNKEFRDCTKSVHKFTNYYVQKALDATPEELEKQGGYVFLYELVKQTRDPKVLRDQSLNILLAGRDTTAGLLSFAVFELARNPHIWAKLREEIEQQFGLGEDSRVEEITFESLKRCEYLKAFLNETLRVYPSVPRNFRIATKNTTLPRGGGPDGTQPILIQKGEGVSYGINSTHLDPVYYGPDAAEFRPERWFEPSTRKLGWAYLPFNGGPRICLGQQFALTEAGYVLVRLVQEFSHIRSDPDEVYPPKRLTNLTMCLQDGAIVKFD

SEQ ID NO: 46 - DNA, 1539 bp, Candida sp.
  Candida sp.; Cytochrome P-450 monooxygenase CYP52A19 (EC 1.14.14.1)
atgctcgatcagatcttacattactggtacattgtcttgccattgttggccattatcaaccagatcgtggctcatgtcaggaccaattatttgatgaagaaattgggtgctaagccattcacacacgtccaacgtgacgggtggttgggcttcaaattcggccgtgaattcctcaaagcaaaaagtgctgggagactggttgatttaatcatctcccgtttccacgataatgaggacactttctccagctatgcttttggcaaccatgtggtgttcaccagggaccccgagaatatcaaggcgcttttggcaacccagtttggtgatttttcattgggcagcagggtcaagttcttcaaaccattattggggtacggtatcttcacattggacgccgaaggctggaagcacagcagagccatgttgagaccacagtttgccagagaacaagttgctcatgtgacgtcgttggaaccacacttccagttgttgaagaagcatatccttaaacacaagggtgagtactttgatatccaggaattgttctttagatttactgtcgactcggccacggagttcttatttggtgagtccgtgcactccttaaaggacgaggaaattggctacgacacgaaagacatgtctgaagaaagacgcagatttgccgacgcgttcaacaagtcgcaagtctacgtggccaccagagttgctttacagaacttgtactggttggtcaacaacaaagagttcaaggagtgcaatgacattgtccacaagtttaccaactactatgttcagaaagccttggatgctaccccagaggaacttgaaaagcaaggcgggtatgtgttcttgtatgagcttgtcaagcagacgagagaccccaaggtgttgcgtgaccagtctttgaacatcttgttggcaggaagagacaccactgctgggttgttgtcctttgctgtgtttgagttggccagaaacccacacatctgggccaagttgagagaggaaattgaacagcagtttggtcttggagaagactctcgtgttgaagagattacctttgagagcttgaagagatgtgagtacttgaaggccgtgttgaacgaaactttgagattacacccaagtgtcccaagaaacgcaagatttgcgattaaagacacgactttaccaagaggcggtggccccaacggcaaggatcctatcttgatcaggaaggatgaggtggtgcagtactccatctcggcaactcagacaaatcctgcttattatggcgccgatgctgctgattttagaccggaaagatggtttgaaccatcaactagaaacttgggatgggctttcttgccattcaacggtggtccaagaatctgtttgggacaacagtttgctttgactgaagccggttacgttttggttagacttgttcaggagtttccaaacttgtcacaagaccccgaaaccaagtacccaccacctagattggcacacttgacgatgtgcttgtttgacggtgcacacgtcaagatgtcatag

SEQ ID NO: 47 - protein, 512 aa, Candida sp.
  Candida sp.; Cytochrome P-450 monooxygenase CYP52A19 (EC 1.14.14.1)
MLDQILHYWYIVLPLLAIINQIVAHVRTNYLMKKLGAKPFTHVQRDGWLGFKFGREFLKAKSAGRSVDLIISRFHDNEDTFSSYAFGNHVVFTRDPENIKALLATQFGDFSLGSRVKFFKPLLGYGIFTLDAEGWKHSRAMLRPQFAREQVAHVTSLEPHFQLLKKHILKHKGEYFDIQELFFRFTVDSATEFLFGESVHSLKDEEIGYDTKDMSEERRRFADAFNKSQVYVATRVALQNLYWLVNNKEFKECNDIVHKFTNYYVQKALDATPEELEKQGGYVFLYELVKQTRDPKVLRDQSLNILLAGRDTTAGLLSFAVFELARNPHIWAKLREEIEQQFGLGEDSRVEEITFESLKRCEYLKAVLNETLRLHPSVPRNARFAIKDTTLPRGGGPNGKDPILIRKDEVVQYSISATQTNPAYYGADAADFRPERWFEPSTRNLGWAFLPFNGGPRICLGQQFALTEAGYVLVRLVQEFPNLSQDPETKYPPPRLAHLTMCLFDGAHVKMS

SEQ ID NO: 48 - DNA, 1539 bp, Candida sp.
  Candida sp.; Cytochrome P-450 monooxygenase CYP52A20 (EC 1.14.14.1)
atgctcgaccagatcttccattactggtacattgtcttgccattgttggtcattatcaagcagatcgtggctcatgccaggaccaattatttgatgaagaagttgggcgctaagccattcacacatgtccaactagacgggtggtttggcttcaaatttggccgtgaattcctcaaagctaaaagtgctgggaggcaggttgatttaatcatctcccgtttccacgataatgaggacactttctccagctatgcttttggcaaccatgtggtgttcaccagggaccccgagaatatcaaggcgcttttggcaacccagtttggtgatttttcattgggaagcagggtcaaattcttcaaaccattgttggggtacggtatcttcaccttggacggcgaaggctggaagcacagcagagccatgttgagaccacagtttgccagagagcaagttgctcatgtgacgtcgttggaaccacatttccagttgttgaagaagcatattcttaagcacaagggtgaatactttgatatccaggaattgttctttagatttaccgttgattcagcgacggagttcttatttggtgagtccgtgcactccttaagggacgaggaaattggctacgatacgaaggacatggctgaagaaagacgcaaatttgccgacgcgttcaacaagtcgcaagtctatttgtccaccagagttgctttacagacattgtactggttggtcaacaacaaagagttcaaggagtgcaacgacattgtccacaagttcaccaactactatgttcagaaagccttggatgctaccccagaggaacttgaaaaacaaggcgggtatgtgttcttgtacgagcttgccaagcagacgaaagaccccaatgtgttgcgtgaccagtctttgaacatcttgttggctggaagggacaccactgctgggttgttgtcctttgctgtgtttgagttggccaggaacccacacatctgggccaagttgagagaggaaattgaatcacactttgggctgggtgaggactctcgtgttgaagagattacctttgagagcttgaagagatgtgagtacttgaaagccgtgttgaacgaaacgttgagattacacccaagtgtcccaagaaacgcaagatttgcgattaaagacacgactttaccaagaggcggtggccccaacggcaaggatcctatcttgatcagaaagaatgaggtggtgcaatactccatctcggcaactcagacaaatcctgcttattatggcgccgatgctgctgattttagaccggaaagatggtttgagccatcaactagaaacttgggatgggcttacttgccattcaacggtggtccaagaatctgcttgggacaacagtttgctttgaccgaagccggttacgttttggttagacttgttcaggaattccctagcttgtcacaggaccccgaaactgagtacccaccacctagattggcacacttgacgatgtgcttgtttgacggggcatacgtcaagatgcaatag

SEQ ID NO: 49 - protein, 512 aa, Candida sp.
  Candida sp.; Cytochrome P-450 monooxygenase CYP52A20 (EC 1.14.14.1)
MLDQIFHYWYIVLPLLVIIKQIVAHARTNYLMKKLGAKPFTHVQLDGWFGFKFGREFLKAKSAGRQVDLIISRFHDNEDTFSSYAFGNHVVFTRDPENIKALLATQFGDFSLGSRVKFFKPLLGYGIFTLDGEGWKHSRAMLRPQFAREQVAHVTSLEPHFQLLKKHILKHKGEYFDIQELFFRFTVDSATEFLFGESVHSLRDEEIGYDTKDMAEERRKFADAFNKSQVYLSTRVALQTLYWLVNNKEFKECNDIVHKFTNYYVQKALDATPEELEKQGGYVFLYELAKQTKDPNVLRDQSLNILLAGRDTTAGLLSFAVFELARNPHIWAKLREEIESHFGSGEDSRVEEITFESLKRCEYLKAVLNETLRLHPSVPRNARFAIKDTTLPRGGGPNGKDPILIRKNEVVQYSISATQTNPAYYGADAADFRPERWFEPSTRNLGWAYLPFNGGPRICLGQQFALTEAGYVLVRLVQEFPSLSQDPETEYPPPRLAHLTMCLFDGAYVKMQ

SEQ ID NO: 50 - DNA, 1497 bp, Candida sp.
  Candida sp.; Cytochrome P-450 monooxygenase CYP52D2 (EC 1.14.14.1)
atggctatatctagtttgctatcgtgggatgtgatctgtgtcgtcttcatttgcgtttgtgtttatttcgggtatgaatattgttatactaaatacttgatgcacaaacatggcgctcgagaaatcgagaatgtgatcaacgatgggttctttgggttccgcttacctttgctactcatgcgagccagcaatgagggccgacttatcgagttcagtgtcaagagattcgagtcggcgccacatccacagaacaagacattggtcaaccgggcattgagcgttcctgtgatactcaccaaggacccagtgaatatcaaagcgatgctatcgacccagtttgatgacttttcccttgggttgagactacaccagtttgcgccgttgttggggaaaggcatctttactttggacggcccagagtggaagcagagccgatctatgttgcgtccgcaatttgccaaagatcgggtttctcatatcctggatctagaaccgcattttgtgttgcttcggaagcacattgatggccacaatggagactacttcgacatccaggagctctacttccggttctcgatggatgtggcgacggggtttttgtttggcgagtctgtggggtcgttgaaagacgaagatgcgaggttcctggaagcattcaatgagtcgcagaagtatttggcaactagggcaacgttgcacgagttgtactttctttgtgacgggtttaggtttcgccagtacaacaaggttgtgcgaaagttctgcagccagtgtgtccacaaggcgttagatgttgcaccggaagacaccagcgagtacgtgtttctccgcgagttggtcaaacacactcgagatcccgttgttttacaagaccaagcgttgaacgtcttgcttgctggacgcgacaccaccgcgtcgttattatcgtttgcaacatttgagctagcccggaatgaccacatgtggaggaagctacgagaggaggttatcctgacgatgggaccgtccagtgatgaaataaccgtggccgggttgaagagttgccgttacctcaaagcaatcctaaacgaaactcttcgactatacccaagtgtgcctaggaacgcgagatttgctacgaggaatacgacgcttcctcgtggcggaggtccagatggatcgtttccgattttgataagaaagggccagccagtggggtatttcatttgtgctacacacttgaatgagaaggtatatgggaatgatagccatgtgtttcgaccggagagatgggctgcgttagagggcaagagtttgggctggtcgtatcttccattcaacggcggcccgagaagctgccttggtcagcagtttgcaatccttgaagcttcgtatgttttggctcgattgacacagtgctacacgacgatacagcttagaactaccgagtacccaccaaagaaactcgttcatctcacgatgagtcttctcaacggggtgtacatccgaactagaact

SEQ ID NO: 51 - protein, 499 aa, Candida sp.
  Candida sp.; Cytochrome P-450 monooxygenase CYP52D2 (EC 1.14.14.1)
MAISSLLSWDVICVVFICVCVYFGYEYCYTKYLMHKHGAREIENVINDGFFGFRLPLLLMRASNEGRLIEFSVKRFESAPHPQNKTLVNRALSVPVILTKDPVNIKAMLSTQFDDFSLGLRLHQFAPLLGKGIFTLDGPEWKQSRSMLRPQFAKDRVSHISDLEPHFVLLRKHIDGHNGDYFDIQELYFRFSMDVATGFLFGESVGSLKDEDARFSEAFNESQKYLATRATLHELYFLCDGFRFRQYNKVVRKFCSQCVHKALDVAPEDTSEYVFLRELVKHTRDPVVLQDQALNVLLAGRDTTASLLSFATFELARNDHMWRKLREEVISTMGPSSDEITVAGLKSCRYLKAILNETLRLYPSVPRNARFATRNTTLPRGGGPDGSFPILIRKGQPVGYFICATHLNEKVYGNDSHVFRPERWAALEGKSLGWSYLPFNGGPRSCLGQQFAILEASYVLARLTQCYTTIQLRTTEYPPKKLVHLTMSLLNGVYIRTRT

SEQ ID NO: 52 - DNA, 1050 bp, Candida sp.
  Candida sp.; Alcohol dehydrogenase ADH1-1 short (EC 1.1.1.1)
atgtctgctaatatcccaaaaactcaaaaagctgtcgtctttgagaagaacggtggtgaattagaatacaaagatatcccagtgccaaccccaaaggccaacgaattgctcatcaacgtcaaatactcgggtgtctgccacactgatttgcacgcctggaagggtgactggccattggccaccaagttgccattggttggtggtcacgaaggtgctggtgtcgttgtcggcatgggtgaaaacgtcaagggctggaagattggtgacttcgccggtatcaaatggttgaacggttcctgtatgtcctgtgagttctgtcaacaaggtgctgaaccaaactgtggtgaggccgacttgtctggttacacccacgatggttctttcgaacaatacgccactgctgatgctgttcaagccgccagaatcccagctggtactgatttggccgaagttgccccaatcttgtgtgcgggtgtcaccgtctacaaagccttgaagactgccgacttggccgctggtcaatgggtcgctatctccggtgctggtggtggtttgggttccttggctgtccaatacgccgtcgccatgggcttgagagtcgttgccattgacggtggtgacgaaaagggtgcctttgtcaagtccttgggtgctgaagcctacattgatttcctcaaggaaaaggacattgtctctgctgtcaagaaggccaccgatggaggtccacacggtgctatcaatgtttccgtttccgaaaaagccattgaccaatccgtcgagtacgttagaccattgggtaaggttgttttggttggtttgccagctggctccaaggtcactgctggtgttttcgaagccgttgtcaagtccattgaaatcaagggttcctatgtcggtaacagaaaggataccgccgaagccgttgactttttctccagaggcttgatcaagtgtccaatcaagattgttggcttgagtgaattgccacaggtcttcaagttgatggaagaaggtaagatcttgggtagatacgtcttggatacctccaaa

SEQ ID NO: 53 - protein, 350 aa, Candida sp.
  Candida sp.; Alcohol dehydrogenase ADH1-1 short (EC 1.1.1.1)
MSANIPKTQKAVVFEKNGGELEYKDIPVPTPKANELLINVKYSGVCHTDLHAWKGDWPLATKLPLVGGHEGAGVVVGMGENVKGWKIGDFAGIKWLNGSCMSCEFCQQGAEPNCGEADLSGYTHDGSFEQYATADAVQAARIPAGTDLAEVAPILCAGVTVYKALKTADLAAGQWVAISGAGGGLGSLAVQYAVAMGLRVVAIDGGDEKGAFVKSLGAEAYIDFLKEKDIVSAVKKATDGGPHGAINVSVSEKAIDQSVEYVRPLGKVVLVGLPAGSKVTAGVFEAVVKSIEIKGSYVGNRKDTAEAVDFFSRGLIKCPIKIVGLSELPQVFKLMEEGKILGRYVLDTSK

SEQ ID NO: 54 - DNA, 1050 bp, Candida sp.
  Candida sp.; Alcohol dehydrogenase ADH1-2 short (EC 1.1.1.1)
atgtctgctaatatcccaaaaactcaaaaagctgtcgtcttcgagaagaacggtggtgaattaaaatacaaagacatcccagtgccaaccccaaaggccaacgaattgctcatcaacgtcaagtactcgggtgtctgtcacactgatttgcacgcctggaagggtgactggccattggacaccaaattgccattggttggtggtcacgaaggtgctggtgttgttgtcggcatgggtgaaaacgtcaagggctggaaaatcggtgatttcgccggtatcaaatggttgaacggttcttgtatgtcctgtgagttctgtcagcaaggtgctgaaccaaactgtggtgaagctgacttgtctggttacacccacgatggttctttcgaacaatacgccactgctgatgctgtgcaagccgccagaatcccagctggcactgatttggccgaagttgccccaatcttgtgtgctggtgtcaccgtctacaaagccttgaagactgccgacttggctgctggtcaatgggtcgctatctccggtgctggtggtggtttgggctccttggctgtccaatacgccgtcgccatgggtttgagagtcgttgccattgacggtggtgacgaaaagggtgactttgtcaagtccttgggtgctgaagcctacattgatttcctcaaggaaaagggcattgttgctgctgtcaagaaggccactgatggcggtccacacggtgctatcaatgtttccgtttccgaaaaagccattgaccaatctgtcgagtacgttagaccattgggtaaggttgttttggttggtttgccagctggctccaaggtcactgctggtgttttcgaagccgttgtcaagtccattgaaatcaagggttcttacgtcggtaacagaaaggatactgccgaagccgttgactttttctccagaggcttgatcaagtgtccaatcaagattgtgggcttgagtgaattgccacaggtcttcaagttgatggaagaaggtaagatcttgggtagatacgtcttggatacctccaaa

SEQ ID NO: 55 - protein, 350 aa, Candida sp.
  Candida sp.; Alcohol dehydrogenase ADH1-2 short (EC 1.1.1.1)
MSANIPKTQKAVVFEKNGGELKYKDIPVPTPKANELLINVKYSGVCHTDLHAWKGDWPLDTKLPLVGGHEGAGVVVGMGENVKGWKIGDFAGIKWLNGSCMSCEFCQQGAEPNCGEADLSGYTHDGSFEQYATADAVQAARIPAGTDLAEVAPILCAGVTVYKALKTADLAAGQWVAISGAGGGLGSLAVQYAVAMGLRVVAIDGGDEKGDFVKSLGAEAYIDFLKEKGIVAAVKKATDGGPHGAINVSVSEKAIDQSVEYVRPLGKVVLVGLPAGSKVTAGVFEAVVKSIEIKGSYVGNRKDTAEAVDFFSRGLIKCPIKIVGLSELPQVFKLMEEGKILGRYVLDTSK

SEQ ID NO: 56 - DNA, 1254 bp, Candida sp.
  Candida sp.; Alcohol dehydrogenase ADH1-2 (EC 1.1.1.1)
atgcatgcattattctcaaaatcagtttttctcaagtatgtgagtctgcccactacctctgctatcccccattccctagaattcattgtctcccgaagctcctatttaaggagacgaattcccccatatcttccacgttgctcccactttccttccttctattattcttcttcttcagtctacaccaagaaatcatttcacacaatgtctgctaatatcccaaaaactcaaaaagctgtcgtcttcgagaagaacggtggtgaattaaaatacaaagacatcccagtgccaaccccaaaggccaacgaattgctcatcaacgtcaagtactcgggtgtctgtcacactgatttgcacgcctggaagggtgactggccattggacaccaaattgccattggttggtggtcacgaaggtgctggtgttgttgtcggcatgggtgaaaacgtcaagggctggaaaatcggtgatttcgccggtatcaaatggttgaacggttcttgtatgtcctgtgagttctgtcagcaaggtgctgaaccaaactgtggtgaagctgacttgtctggttacacccacgatggttctttcgaacaatacgccactgctgatgctgtgcaagccgccagaatcccagctggcactgatttggccgaagttgccccaatcttgtgtgctggtgtcaccgtctacaaagccttgaagactgccgacttggctgctggtcaatgggtcgctatctccggtgctggtggtggtttgggctccttggctgtccaatacgccgtcgccatgggtttgagagtcgttgccattgacggtggtgacgaaaagggtgactttgtcaagtccttgggtgctgaagcctacattgatttcctcaaggaaaagggcattgttgctgctgtcaagaaggccactgatggcggtccacacggtgctatcaatgtttccgtttccgaaaaagccattgaccaatctgtcgagtacgttagaccattgggtaaggttgttttggttggtttgccagctggctccaaggtcactgctggtgttttcgaagccgttgtcaagtccattgaaatcaagggttcttacgtcggtaacagaaaggatactgccgaagccgttgactttttctccagaggcttgatcaagtgtccaatcaagattgtgggcttgagtgaattgccacaggtcttcaagttgatggaagaaggtaagatcttgggtagatacgtcttggatacctccaaa

SEQ ID NO: 57 - protein, 418 aa, Candida sp.
  Candida sp.; Alcohol dehydrogenase ADH1-2 (EC 1.1.1.1)
MHALFSKSVFLKYVSSPTTSAIPHSLEFIVSRSSYLRRRIPPYLPRCSHFPSFYYSSSSVYTKKSFHTMSANIPKTQKAVVFEKNGGELKYKDIPVPTPKANELLINVKYSGVCHTDLHAWKGDWPLDTKLPLVGGHEGAGVVVGMGENVKGWKIGDFAGIKWLNGSCMSCEFCQQGAEPNCGEADLSGYTHDGSFEQYATADAVQAARIPAGTDLAEVAPILCAGVTVYKALKTADLAAGQWVAISGAGGGLGSLAVQYAVAMGLRVVAIDGGDEKGDFVKSLGAEAYIDFLKEKGIVAAVKKATDGGPHGAINVSVSEKAIDQSVEYVRPLGKVVLVGLPAGSKVTAGVFEAVVKSIEIKGSYVGNRKDTAEAVDFFSRGLIKCPIKIVGLSELPQVFKLMEEGKILGRYVLDTSK

SEQ ID NO: 58 - DNA, 1044 bp, Candida sp.
  Candida sp.; Alcohol dehydrogenase ADH2a (EC 1.1.1.1)
atgtcaattccaactactcaaaaagctatcattttcgaaaccaacggtggaaaattagaatacaaggacatcccagttccaaagccaaagccaaacgaattgctcatcaacgtcaagtactccggtgtctgccacactgatttacacgcctggaagggtgactggccattggacaccaagttgccattggtgggtggtcacgaaggtgctggtgttgttgttgccattggtgacaatgtcaagggatggaaggtcggtgatttggccggtgtcaagtggttgaacggttcctgtatgaactgtgagtactgtcaacagggtgccgaaccaaactgtccacaggctgacttgtctggttacacccacgacggttctttccagcaatacgccactgcagatgccgtgcaagccgctagaattccagctggtactgatttagccaacgttgcccccatcttgtgtgctggtgtcactgtttacaaggccttgaagaccgccgacttgcagccaggtcaatgggtcgccatttccggtgccgctggtggtttgggttctttggccgttcaatacgccaaggccatgggctacagagttgtcgccatcgatggtggtgccgacaagggtgagttcgtcaagtctttgggcgctgaggtctttgttgatttcctcaaggaaaaggacattgttggtgctgtcaagaaggcaaccgatggtggcccacacggtgccgttaacgtttccatctccgaaaaggccatcaaccaatctgtcgactacgttagaaccttgggtaaggttgtcttggtcggtttgccagctggctccaaggtttctgctccagtctttgactccgtcgtcaagtccatccaaatcaagggttcctatgtcggtaacagaaaggacactgccgaagctgttgactttttctccagaggcttgatcaagtgtccaatcaaggttgtcggtttgagtgaattgccagaagtctacaagttgatggaagaaggtaagatcttgggtagatacgtcttggacaactctaag

SEQ ID NO: 59 - protein, 348 aa, Candida sp.
  Candida sp.; Alcohol dehydrogenase ADH2a (EC 1.1.1.1)
MSIPTTQKAIIFETNGGKLEYKDIPVPKPKPNELLINVKYSGVCHTDLHAWKGDWPLDTKLPLVGGHEGAGVVVAIGDNVKGWKVGDLAGVKWLNGSCMNCEYCQQGAEPNCPQADLSGYTHDGSFQQYATADAVQAARIPAGTDLANVAPILCAGVTVYKALKTADLQPGQWVAISGAAGGLGSLAVQYAKAMGYRVVAIDGGADKGEFVKSLGAEVFVDFLKEKDIVGAVKKATDGGPHGAVNVSISEKAINQSVDYVRTLGKVVLVGLPAGSKVSAPVFDSVVKSIQIKGSYVGNRKDTAEAVDFFSRGLIKCPIKVVGLSELPEVYKLMEEGKILGRYVLDNSK

SEQ ID NO: 60 - DNA, 1044 bp, Candida sp.
  Candida sp.; Alcohol dehydrogenase ADH2b (EC 1.1.1.1)
atgtcaattccaactacccaaaaagctgttatctacgaagccaactctgctccattgcaatacaccgatatcccagttccagtccctaagccaaacgaattgctcgtccacgtcaaatactccggtgtttgtcactcagatatacacgtctggaagggtgactggttcccagcatcgaaattgcccgttgttggtggtcacgaaggtgccggtgttgtcgttgccattggtgaaaacgtccaaggctggaaagtaggtgacttggcaggtataaagatgttgaatggttcctgtatgaactgtgaatactgtcaacaaggtgctgaaccaaactgtccccacgctgatgtctcgggttactcccacgacggtactttccaacagtacgctaccgccgatgctgttcaagctgctaaattcccagctggttctgatttagctagcatcgcacctatatcctgcgccggtgttactgtttacaaagcattgaaaactgcaggcttgcagccaggtcaatgggttgccatctctggtgcagctggtggtttgggttctttggctgtgcaatacgccaaggccatgggtttgagagtcgtggccattgacggtggtgacgaaagaggagtgtttgtcaaatcgttgggtgctgaagttttcgttgatttcaccaaagaggccaatgtctctgaggctatcatcaaggctaccgacggtggtgcccatggcgtcatcaacgtttccatttctgaaaaagccatcaaccagtctgttgaatatgttagaactttgggaactgttgtcttggttggtttgccagctggtgcaaagctcgaagctcctatcttcaatgccgttgccaaatccatccaaatcaaaggttcttacgtgggaaacagaagagacactgctgaggctgttgatttcttcgctagaggtttggtcaaatgtccaattaaggttgttgggttgagtgaattgccagagattttcaaattgttggaagagggtaagatcttgggtagatacgttgttgacactgccaag

SEQ ID NO: 61 - protein, 348 aa, Candida sp.
  Candida sp.; Alcohol dehydrogenase ADH2b (EC 1.1.1.1)
MSIPTTQKAVIYEANSAPLQYTDIPVPVPKPNELLVHVKYSGVCHSDIHVWKGDWFPASKLPVVGGHEGAGVVVAIGENVQGWKVGDLAGIKMLNGSCMNCEYCQQGAEPNCPHADVSGYSHDGTFQQYATADAVQAAKFPAGSDLASIAPISCAGVTVYKALKTAGLQPGQWVAISGAAGGLGSLAVQYAKAMGLRVVAIDGGDERGVFVKSLGAEVFVDFTKEANVSEAIIKATDGGAHGVINVSISEKAINQSVEYVRTLGTVVLVGLPAGAKLEAPIFNAVAKSIQIKGSYVGNRRDTAEAVDFFARGLVKCPIKVVGLSELPEIFKLLEEGKILGRYVVDTAK

SEQ ID NO: 62 - DNA, 1047 bp, Candida sp.
  Candida sp.; Alcohol dehydrogenase ADH3 (EC 1.1.1.1)
atgtcaactcaatcaggttacggatacgtgaaaggacaaaagaccattcagaaatacaccgacatcccgatccctacgccgggccccaacgaagtcttgttgaaagtcgaagctgccggcttgtgtctctcggatccacacacgttgatcgggggtcccattgagagcaagccgccgttgccgaacgccacgaagttcatcatgggtcacgaaatcgcggggctgattagccaagtaggcgccaacttggccaacgatccatactataaaaagggaggtaggttcgccttgactatcgcgcaggcttgtgggatttgtgagaattgtcgtgatgggtatgatgcaaagtgtgagtctacgacgcaggcttatgggttgaacgaggacggtggattccagcaatacttgttgattaagaacttgcgtacgatgttgcctatccctgagggtgtgagttacgaagaagccgctgtgtctactgactctgtgttgactccattccatgcgattcagaaggtcgctcatttgttgcacccaactactaaggtgttggttcagggttgtggtgggttaggcttcaacgctattcaaatattgaagagctacaattgttacattgttgccactgatgtcaaaccagagcttgaaaaattagctttggagtatggtgccaacgaataccacactgatctcaccaagtccaagcatgagccaatgtcgttcgatttgattttcgaccttgtgggaatccaacctacttttgatttgtccgacaggtacatcaaagcaaggggtaagattcttatgattggcttaggcagatccaagttgtttattccaaattataaattgggtatccgtgaagtcgagatcattttcaattttggtggtacttcggccgagcaaattgagtgcatgaaatgggttgcaaaaggcttgatcaaacctaatattcacgtggctgattttgcttccttgcctgagtacctcgaggacttggccaagggtaaactcactggtagaattgtatttagaccaagtaagttg

SEQ ID NO: 63 - protein, 349 aa, Candida sp.
  Candida sp.; Alcohol dehydrogenase ADH3 (EC 1.1.1.1)
MSTQSGYGYVKGQKTIQKYTDIPIPTPGPNEVLLKVEAAGLCLSDPHTLIGGPIESKPPLPNATKFIMGHEIAGSISQVGANLANDPYYKKGGRFALTIAQACGICENCRDGYDAKCESTTQAYGLNEDGGFQQYLLIKNLRTMLPIPEGVSYEEAAVSTDSVLTPFHAIQKVAHLLHPTTKVLVQGCGGLGFNAIQILKSYNCYIVATDVKPELEKLALEYGANEYHTDLTKSKHEPMSFDLIFDLVGIQPTFDLSDRYIKARGKILMIGLGRSKLFIPNYKLGIREVEIIFNFGGTSAEQIECMKWVAKGLIKPNIHVADFASLPEYLEDLAKGKLTGRIVFRPSKL

SEQ ID NO: 64 - DNA, 747 bp, Candida sp.
  Candida sp.; Alcohol dehydrogenase ADH4 (EC 1.1.1.1)
atgtcattatcaggaaagacctcattaattgctgctggtaccaagaacttgggtggtgcaagtgccaaagaattggccaaagccggctccaacctcttcttgcactacagatccaacccagacgaggctgaaaagttcaagcaagagatcctcaaggagttccctaacgtcaaggtcgaaacctaccaatccaaattggaccgtgccgccgacctcaccaacttgtttgctgctgccaagaaggcattccctagtggtattgacgtcgctgtcaactttgtcggtaaggtcatcaagggcccaatcactgaggtcactgaagaacagtttgacgagatggatgttgccaacaacaagattgcctttttcttcatcaaggaggccgctatcaacttgaacaagaacggtagtatcatttccatcgttactagtttgctcccagcttacaccgattcttacggtttgtaccagggtactaaaggagctgttgaatactattcgaaatctatcctgaaggagttgattccaaagggtatcaccagtaactgtattggtcctggtcctgcttctacttcctttttgtttaattccgaaaccaaggagagtgttgagttcttcaagaccgttgctattgaccaacgtttgactgaagacagcgacattgccccaattgtgttgttcctcgccactggaggtcgttgggcaactggtcaaactatttacgctagtggtggtttcactgctcgt

SEQ ID NO: 65 - protein, 249 aa, Candida sp.
  Candida sp.; Alcohol dehydrogenase ADH4 (EC 1.1.1.1)
MSLSGKTSLIAAGTKNLGGASAKELAKAGSNLFLHYRSNPDEAEKFKQEILKEFPNVKVETYQSKLDRAADLTNLFAAAKKAFPSGIDVAVNFVGKVIKGPITEVTEEQFDEMDVANNKIAFFFIKEAAINLNKNGSIISIVTSLLPAYTDSYGLYQGTKGAVEYYSKSISKELIPKGITSNCIGPGPASTSFLFNSETKESVEFFKTVAIDQRLTEDSDIAPIVLFLATGGRWATGQTIYASGGFTAR

SEQ ID NO: 66 - DNA, 1098 bp, Candida sp.
  Candida sp.; Alcohol dehydrogenase ADH5 (EC 1.1.1.1)
atgtcacttgtcctcaagcgattacttccaatcagatctcctactttactcaattcgaagttcatacagttacaatctcaaattcgcacaatggctatccccgctactcaaactggattcttcttcaccaaacaagaaggtttaaactacagaaccgacattcctgtccgcaagccacaagccggtcagttgttgttgaaggtcaatgccgttggtctctgccactcggacttgcacgtgattgacaaggagcttgaatgtggtgacaactatgtcatgggccacgaaattgccggtaccgttgctgaagttggtcccgaagttgaaggctacaaggttggcgaccgtgtcgcttgtgttggtcctaacgggtgcggtgtctgtaagcactgcttgactggtaacgacaatgtctgtaagactgctttcctcgactggttcgggttgggctccgatggtgggtacgaagagtacttgttggtgagaagaccaagaaacttggttaaggtcccggacaacgtctcgattgaggaggctgctgctatcactgatgctgtgttgactccttaccatgctgtcaagactgccaaggtcaagccaaccagtaacgttttggttattggtgctggtggattaggtggtaacggtatccagattgtcaaggcttttggcggtaaggttactgttgtcgataagaaggataaggcacgtgaccaagctaaggctttgggtgctgatgaagtctacagtgaaatcccagcaagtattgaaccgggtacttttgatgtctgtcttgattttgtttccgtgcaagccacctatgatctctgccaaaagtactgtgagccaaagggtatcattatcccagttgggttgggtgctaccaagctcaccattgatttggcagatttggatctccgtgaaatcacggttactggtaccttctggggaactgccaatgacttgagagaggcgtttgatttggttagtcaaggtaagatcaagccgattgtttcacatgccccattgaaggagttgccaaactatatggagaagttgaagcagggagcatatgaaggaagagttgtcttccaccca

SEQ ID NO: 67 - protein, 366 aa, Candida sp.
  Candida sp.; Alcohol dehydrogenase ADH5 (EC 1.1.1.1)
MSLVLKRLLPIRSPTLLNSKFIQLQSQIRTMAIPATQTGFFFTKQEGLNYRTDIPVRKPQAGQLLLKVNAVGLCHSDLHVIDKELECGDNYVMGHEIAGTVAEVGPEVEGYKVGDRVACVGPNGCGVCKHCLTGNDNVCKTAFLDWFGLGSDGGYEEYLLVRRPRNLVKVPDNVSIEEAAAITDAVLTPYHAVKTAKVKPTSNVLVIGAGGLGGNGIQIVKAFGGKVTVVDKKDKARDQAKALGADEVYSEIPASIEPGTFDVCLDFVSVQATYDLCQKYCEPKGIIIPVGLGATKLTIDLADLDLREITVTGTFWGTANDLREAFDLVSQGKIKPIVSHAPLKELPNYMEKLKQGAYEGRVVFHP

SEQ ID NO: 68 - DNA, 1125 bp, Candida sp.
  Candida sp.; Alcohol dehydrogenase ADH7 (EC 1.1.1.1)
atgactgttgacgcttcttctgttccagacaagttccaagggtttgcctccgacaagagagaaaactgggaacacccaaagttgatctcctacgacagaaagcaactcaatgaccacgacgttgtcttgaagaacgagacctgtggtttgtgttactcggacatccacaccttgcgttccacgtggggaccatacggcaccaatgagcttgtcgttggccacgaaatctgtggtaccgtcattgctgtcggtccaaaggtcactgagttcaaggtcggtgacagagccggtattggtgctgcctcttcgtcttgtcgtcactgttccagatgtacccacgataacgagcaatactgtaaggaacaagtctccacttacaattctgttgatccaaaggccgctggttacgtcaccaagggtggttactcctcccactccatcgctgacgaattgtttgtcttcaaggttccagatgacttgccattcgagtacgcttccccattattctgtgctggtatcacaactttctccccattgtaccgtaacttggttgggtccgataaagacgccactggtaagaccgttggtatcattggtgttggtggtcttggtcaccttgccatccagtttgcgtctaaagctttgaacgctaaggtcgttgctttctccagatcctcctccaagaaggaagaagctctcgaattgggtgctgctgagtttgtcgccaccaacgaagacaagaactggaccagcagatacgaggaccaattcgacctcatcttgaactgtgcgagcggtatcgatggcttgaacttgtctgactacttgagtgtcttgaaagtcgacaagaagtttgtctctgttggtttgccaccaatcgacgacgagttcaacgtctctcctttcactttcttgaagcaaggtgccagtttcggtagttccttgttgggatccaaggctgaagtcaacatcatgttggaattggctgccaagcacaacatcagaccatggattgaaaaggtcccaatcagtgaggaaaacgtcgccaaggctttgaagagatgttttgaaggtgatgtcagatacagattcgtcttcactgagtttgacaaagcttttggcaat

SEQ ID NO: 69 - protein, 375 aa, Candida sp.
  Candida sp.; Alcohol dehydrogenase ADH7 (EC 1.1.1.1)
MTVDASSVPDKFQGFASDKRENWEHPKLISYDRKQLNDHDVVLKNETCGLCYSDIHTLRSTWGPYGTNELVVGHEICGTVIAVGPKVTEFKVGDRAGIGAASSSCRHCSRCTHDNEQYCKEQVSTYNSVDPKAAGYVTKGGYSSHSIADELFVFKVPDDLPFEYASPLFCAGITTFSPLYRNLVGSDKDATGKTVGIIGVGGLGHLAIQFASKALNAKVVAFSRSSSKKEEALELGAAEFVATNEDKNWTSRYEDQFDLILNCASGIDGLNLSDYLSVLKVDKKFVSVGLPPIDDEFNVSPFTFLKQGASFGSSLLGSKAEVNIMLELAAKHNIRPWIEKVPISEENVAKALKRCFEGDVRYRFVFTEFDKAFGN

SEQ ID NO: 70 - DNA, 1044 bp, Candida sp.
  Candida sp.; Alcohol dehydrogenase ADH8 (EC 1.1.1.1)
atgtccgttccaactactcagaaagctgttatctttgaaaccaatggtggcaagttagaatacaaagacgtgccggtccctgtccctaaacccaacgaattgcttgtcaacgtcaagtactcgggtgtgtgtcattctgacttgcatgtctggaaaggcgactggcccattcctgccaagttgcccttggtgggaggtcacgaaggtgctggtgtcgttgtcggcatgggtgacaacgtcaagggctggaaggtgggggacttggctggtatcaagtggttgaatggttcgtgtatgaactgtgagttttgccaacagggcgcagaacctaactgttcaagagccgacatgtctgggtatacccacgatggaactttccaacaatacgccactgctgatgctgtccaagctgccaagatcccagaaggcgccgacatggctagtatcgccccgatcttgtgcgctggtgtgaccgtgtacaaggctttgaagaacgccgacttgttggctggccaatgggtggctatctctggtgctggtggtggtttgggctccttgggtgtgcagtacgctaaagccatgggttacagagtgttggctatcgacggtggtgacgagagaggagagtttgtcaagtccttgggcgccgaagtgtacattgacttccttaaggaacaggacatcgttagtgctatcagaaaggcaactggtggtggtccacacggtgttattaacgtctcagtgtccgaaaaggcaatcaaccagtcggtggagtacgtcagaactttggggaaagtggttttagttagcttgccggcaggtggtaaactcactgctcctcttttcgagtctgttgctagatcaatccagattagaactacgtgtgttggcaacagaaaggatactactgaagctattgatttctttgttagagggttgatcgattgcccaattaaagtcgctggtttaagtgaagtgccagagatttttgacttgatggagcagggaaagatcttgggtagatatgtcgttgatacgtcaaag

SEQ ID NO: 71 - protein, 348 aa, Candida sp.
  Candida sp.; Alcohol dehydrogenase ADH8 (EC 1.1.1.1)
MSVPTTQKAVIFETNGGKLEYKDVPVPVPKPNELLVNVKYSGVCHSDLHVWKGDWPIPAKLPLVGGHEGAGVVVGMGDNVKGWKVGDLAGIKWLNGSCMNCEFCQQGAEPNCSRADMSGYTHDGTFQQYATADAVQAAKIPEGADMASIAPILCAGVTVYKALKNADLLAGQWVAISGAGGGLGSLGVQYAKAMGYRVLAIDGGDERGEFVKSLGAEVYIDFLKEQDIVSAIRKATGGGPHGVINVSVSEKAINQSVEYVRTLGKVVLVSLPAGGKLTAPLFESVARSIQIRTTCVGNRKDTTEAIDFFVRGLIDCPIKVAGLSEVPEIFDLMEQGKILGRYVVDTSK

SEQ ID NO: 72 - DNA, 1638 bp, Candida sp.
atgtccccaccatctaaattagaagactcctcctccgcaaccaccgctgccgatacccttggcgactcctggtacaccaaagtgtccgacattgcgcctggcgtgcagagattgaccgagtcattccacagggatcaaaagacgcacgacattcagttccgcttgaaccaattgcgtaacctttactttgcggtccaggacaatgccgacgcgctctgtgctgccttggacaaggacttctaccgtccccccagtgaaaccaagaacttggaactcgtgggtggcttgaatgagttggtgcacaccatttcgagcttgcatgagtggatgaagccggaaaaagtcacggatttgccacttactttgaggtcaaacccgatttatattgaaagaatcccattgggggtcgtgttgatcatctcgcctttcaactaccctttcttcttgtcgttttcggccgtcgtgggtgcgattgctggtggtaacgcggttgttttgaagggctctgagttgacgccaaacttctccagtttgttctcaaagatcttgactaaggctttggaccctgatattttctttgcagtcgatggtgctatccctgagacgaccgagttgttggaacaaaagtttgacaagatcatgtatactggtaacaacaccgtgggtaagattattgccaagaaggctgctgagaccttgacgccagttatcttggaattgggtggtaagtcgccagctttcatcttggacgacgtcaaggataaaaacttggaagtcatcgccagaagaatcgcatggggtagattcaccaacgccggtcaaacctgtgttgctgtcgactacgtcttggttccaaccaaactccacaagaagttcattgctgcgttgaccaaggtcttgagtcaagaattctaccctaacttgaccaaagacaccaagggctacacccacgtcatccacgaccgtgcattcaacaatttgtccaagatcatcagcaccaccaagggtgacattgtctttggcggcgacaccgatgccgccacccgcttcatcgcccccaccgtcatcgacaacgccacctgggaggattcttccatgaagggcgaaatctttggtcccatcttgcccgtcttgacctacgacaagctcaccaccgccatcaggcaagttgtgtccacgcacgacacgccattagcgcagtacatcttcaccagcgggtccacatcccgcaagtacaaccgccagctcgaccagatcttgactggtgtccggtccgggggtgtgattgtcaacgatgtcttgatgcacgttgcgttgatcaatgcgccatttggcggcgttggtgactccgggtacggctcgtaccacggcaagttctcgttccgcagcttcacgcacgaacgtaccaccatggagcagaagttgtggaacgacgggatggtcaaggtcagataccctccttataactccaacaaggacaagttgatccaggtctcccagcagaactacaacggcaaggtctggttcgatagaaacggcgacgtgcctgtgaatggaccaggtgcgttgtttagcgcttggactacgttcactggtgtcttccatttgcttggtgagttcatcactaataagcaatag

SEQ ID NO: 73 - protein, 545 aa, Candida sp.
  Candida sp.; Aldehyde dehydrogenase (EC 1.2.1.5)
MSPPSKLEDSSSATTAADTLGDSWYTKVSDIAPGVQRLTESFHRDQKTHDIQFRLNQLRNLYFAVQDNADALCAALDKDFYRPPSETKNLELVGGLNELVHTISSLHEWMKPEKVTDLPLTLRSNPIYIERIPLGVVLIISPFNYPFFLSFSAVVGAIAGGNAVVLKGSELTPNFSSLFSKILTKALDPDIFFAVDGAIPETTELLEQKFDKIMYTGNNTVGKIIAKKAAETLTPVILELGGKSPAFILDDVKDKNLEVIARRIAWGRFTNAGQTCVAVDYVLVPTKLHKKFIAALTKVLSQEFYPNLTKDTKGYTHVIHDRAFNNLSKIISTTKGDIVFGGDTDAATRFIAPTVIDNATWEDSSMKGEIFGPILPVLTYDKLTTAIRQVVSTHDTPLAQYIFTSGSTSRKYNRQLDQILTGVRSGGVIVNDVLMHVALINAPFGGVGDSGYGSYHGKFSFRSFTHERTTMEQKLWNDGMVKVRYPPYNSNKDKLIQVSQQNYNGKVWFDRNGDVPVNGPGALFSAWTTFTGVFHLLGEFITNKQ

SEQ ID NO: 74 - DNA, 1956 bp, Candida sp.
  Candida sp.; Long chain fatty acid-CoA ligase (EC 6.2.1.3)
atgtcaggattagaaatagccgctgctgccatccttggtagtcagttattggaagccaaatatttaattgccgacgacgtgctgttagccaagacagtcgctgtcaatgccctcccatacttgtggaaagccagcagaggtaaggcatcatactggtactttttcgagcagtccgtgttcaagaacccaaacaacaaggcgttggcgttcccaagaccaagaaagaatgcccccacccccaagaccgacgccgaggggttccagatctacgacgaccagtttgacctagaagaatacacctacaaggaattgtacgatatggttttgaagtactcgtacatcttgaagaacgagtacggtgtcactgccaacgacaccattggtgtttcttgtatgaacaagccgcttttcattgtgttgtggttggcattgtggaacattggtgccttgcctgcgttcttgaacttcaacaccaaggacaagccattgatccactgtcttaagattgtcaacgcttcgcaagttttcgttgacccggactgtgattccccaatcagagataccgaggctcagatcagagaggaattgccacatgtgcaaataaactacattgacgagtttgccttgtttgacagattgagactcaagtcgactccaaaacacagagccgaggacaagaccagaagaccaaccgatactgactcctcggcttgtgcattgatttacacctcgggtaccaccggtttgccaaaagccggtatcatgtcctggagaaaagccttcatggcctcggttttctttggccacatcatgaagattgactcgaaatcgaacgtcttgaccgccatgcccttgtaccactccaccgcggccatgttggggttgtgtcctaccttgattgtcggtggctgtgtctcggtgtcccagaaattctccgctacttcgttctggacccaggccagattatgtggtgccacccacgtgcaatacgtcggtgaggtctgtcgttacttgttgaactccaagcctcatccagaccaagacagacacaatgtcagaattgcctacggtaacgggttgcgtccagatatatggtctgagttcaagcgcagattccacattgaaggtatcggtgagttctacgccgccaccgagtcccctatcgccaccaccaacttgcagtacggtgagtacggtgtcggcgcctgtcgtaagtacgggtccctcatcagcttgttattgtctacccagcagaaattggccaagatggacccagaagacgagagtgaaatctacaaggaccccaagaccgggttctgtaccgaggccgcttacaacgagccaggtgagttgttgatgagaatcttgaaccctaacgacgtgcagaaatccttccagggttattacggtaacaagtccgccaccaacagcaaaatcctcaccaatgttttcaaaaaaggtgacgcgtggtacagatccggtgacttgttgaagatggacgagaacaaattgttgtactttgtcgacagattaggtgacacgttccgttggaagtccgaaaacgtctccgccaccgaggtcgagaacgaattgatgggctccaaggccttgaagcagtccgtcgttgtcggtgtcaaggtgccaaaccacgaaggtagagcctgttttgccgtctgtgaagccaaggacgagttgagccatgaagaaatcttgaaattgattcactctcacgtgaccaagtctttgcctgtgtatgctcaacctgcgttcatcaagattggcaccattgaggcttcgcacaaccacaaggttcctaagaaccaattcaagaaccaaaagttaccaaagggtgaagacggcaaggatttgatctactggttgaatggcgacaagtaccaggagttgactgaagacgattggtctttgatttgtaccggtaaagccaaattggaatag

SEQ ID NO: 75 - protein, 651 aa, Candida sp.
  Candida sp.; Long chain fatty acid-CoA ligase (EC 6.2.1.3)
MSGLEIAAAAILGSQLLEAKYLIADDVSLAKTVAVNALPYLWKASRGKASYWYFFEQSVFKNPNNKALAFPRPRKNAPTPKTDAEGFQIYDDQFDLEEYTYKELYDMVLKYSYILKNEYGVTANDTIGVSCMNKPLFIVLWLALWNIGALPAFLNFNTKDKPLIHCLKIVNASQVFVDPDCDSPIRDTEAQIREELPHVQINYIDEFALFDRLRLKSTPKHRAEDKTRRPTDTDSSACALIYTSGTTGLPKAGIMSWRKAFMASVFFGHIMKIDSKSNVLTAMPLYHSTAAMLGLCPTLIVGGCVSVSQKFSATSFWTQARLCGATHVQYVGEVCRYLLNSKPHPDQDRHNVRIAYGNGLRPDIWSEFKRRFHIEGIGEFYAATESPIATTNLQYGEYGVGACRKYGSLISLLLSTQQKLAKMDPEDESEIYKDPKTGFCTEAAYNEPGELLMRILNPNDVQKSFQGYYGNKSATNSKILTNVFKKGDAWYRSGDLLKMDENKLLYFVDRLGDTFRWKSENVSATEVENELMGSKALKQSVVVGVKVPNHEGRACFAVCEAKDELSHEEILKLIHSHVTKSLPVYAQPAFIKIGTIEASHNHKVPKNQFKNQKLPKGEDGKDLIYWLNGDKYQELTEDDWSLICTGKAKLE

SEQ ID NO: 76 - DNA, 2088 bp, Candida sp.
  Candida sp.; Acyl-CoA synthetase (EC 6.2.1.3)
atgggtgcccctttaacagtcgccgttggcgaagcaaaaccaggcgaaaccgctccaagaagaaaagccgctcaaaaaatggcctctgtcgaacgcccaacagactcaaaggcaaccactttgccagacttcattgaagagtgttttgccagaaacggcaccagagatgccatggcctggagagacttggtcgaaatccacgtcgaaaccaaacaggttaccaaaatcattgacggcgaacagaaaaaggtcgataaggactggatctactacgaaatgggtccttacaactacatatcctaccccaagttgttgacgttggtcaagaactactccaagggtttgttggagttgggcttggccccagatcaagaatccaagttgatgatctttgccagtacctcccacaagtggatgcagaccttcttagcctccagtttccaaggtatccccgttgtcaccgcctacgacaccttgggtgagtcgggcttgacccactccttggtgcaaaccgaatccgatgccgtgttcaccgacaaccaattgttgtcctccttgattcgtcctttggagaaggccacctccgtcaagtatgtcatccacggggaaaagattgaccctaacgacaagagacagggcggcaaaatctaccaggatgcggaaaaggccaaggagaagattttacaaattagaccagatattaaatttatttctttcgacgaggttgttgcattgggtgaacaatcgtccaaagaattgcatttcccaaaaccagaagacccaatctgtatcatgtacacctcgggttccaccggtgctccaaagggtgtggttatcaccaatgccaacattgttgccgccgtgggtggtatctccaccaatgctactagagacttggttagaactgtcgacagagtgattgcatttttgccattggcccacattttcgagttggcctttgagttggttaccttctggtggggggctccattgggttacgccaatgtcaagactttgaccgaagcctcctgcagaaactgtcagccagacttgattgaattcaaaccaaccatcatggttggtgttgctgccgtttgggaatcggtcagaaagggtgtcttgtctaaattgaaacaggcttctccaatccaacaaaagatcttctgggctgcattcaatgccaagtctactttgaaccgttatggcttgccaggcggtgggttgtttgacgctgtcttcaagaaggttaaagccgccactggtggccaattgcgttatgtgttgaatggtgggtccccaatctctgttgatgcccaagtgtttatctccaccttgcttgcgccaatgttgttgggttacggtttgactgaaacctgtgccaataccaccattgtcgaacacacgcgcttccagattggtactttgggtaccttggttggatctgtcactgccaagttggttgatgttgctgatgctggatactacgccaagaacaaccagggtgaaatctggttgaaaggcggtccagttgtcaaggaatactacaagaacgaagaagaaaccaaggctgcattcaccgaagatggctggttcaagactggtgatattggtgaatggaccgccgacggtggtttgaacatcattgaccgtaagaagaacttggtcaagactttgaatggtgaatacattgctttggagaaattggaaagtatttacagatccaaccacttgattttgaacttgtgtgtttacgctgaccaaaccaaggtcaagccaattgctattgtcttgccaattgaagccaacttgaagtctatgttgaaggacgaaaagattatcccagatgctgattcacaagaattgagcagcttggttcacaacaagaaggttgcccaagctgtcttgagacacttgctccaaaccggtaaacaacaaggtttgaaaggtattgaattgttgcagaatgttgtcttgttggatgacgagtggaccccacagaatggttttgttacttctgcccaaaagttgcagagaaagaagattttagaaagttgtaaaaaagaagttgaagaggcatacaagtcgtct

SEQ ID NO: 77 - protein, 696 aa, Candida sp.
  Candida sp.; Acyl-CoA synthetase (EC 6.2.1.3)
MGAPLTVAVGEAKPGETAPRRKAAQKMASVERPTDSKATTLPDFIEECFARNGTRDAMAWRDLVEIHVETKQVTKIIDGEQKKVDKDWIYYEMGPYNYISYPKLLTLVKNYSKGLLELGLAPDQESKLMIFASTSHKWMQTFLASSFQGIPVVTAYDTLGESGLTHSLVQTESDAVFTDNQLLSSLIRPLEKATSVKYVIHGEKIDPNDKRQGGKIYQDAEKAKEKILQIRPDIKFISFDEVVALGEQSSKELHFPKPEDPICIMYTSGSTGAPKGVVITNANIVAAVGGISTNATRDLVRTVDRVIAFLPLAHIFELAFELVTFWWGAPLGYANVKTLTEASCRNCQPDLIEFKPTIMVGVAAVWESVRKGVLSKLKQASPIQQKIFWAAFNAKSTLNRYGLPGGGLFDAVFKKVKAATGGQLRYVLNGGSPISVDAQVFISTLLAPMLLGYGLTETCANTTIVEHTRFQIGTLGTLVGSVTAKLVDVADAGYYAKNNQGEIWLKGGPVVKEYYKNEEETKAAFTEDGWFKTGDIGEWTADGGLNIIDRKKNLVKTLNGEYIALEKLESIYRSNHLILNLCVYADQTKVKPIAIVLPIEANLKSMLKDEKIIPDADSQELSSLVHNKKVAQAVLRHLLQTGKQQGLKGIELLQNVVLLDDEWTPQNGFVTSAQKLQRKKILESCKKEVEEAYKSS

SEQ ID NO: 78 - DNA, 1227 bp, Candida sp.
  Candida sp.; 3-ketoacyl-CoA thiolase (beta- ketothiolase) (POT1-1) (EC 2.3.1.16)
atggatagattaaaccaattaagcggccaattaaagccaaacgccaaacaatccatcttgcaaaaaaacccagacgacgtcgttatcgttgctgcatacagaaccgccatcggtaaaggtttcaaaggttccttcagaagcgtccgctctgaattcatcttgactgagttcttgaaagaattcattaaaaagaccaacatcgacccatctttgattgaagatgtcgctatcggtaacgtcttgaaccaggccgccggtgccaccgaacacagaggtgcttgtttggctgccggtatcccatacaccgccgctttcatcgccgtcaacagattctgctcatccggtttgatggccatctccgacattgccaacaagatcaagactggtgaaatcgagtgtggtttggctggtggtgccgaatccatgtccaccaactaccgtgatcctagagttgccccaagaatcgacccacacttggctgacgacgcccaaatggaaaagtgtttgattcctatgggtatcaccaacgaaaacgttgctaaccaattcaacatctccagagaaagacaagacgagttcgccgccaagtcctacaacaaggctgccaaggctgttgccgctggtgctttcaagagcgaaatcttgccaatcagatccatcatcagaaactctgacggtaccgaaaaggaaatcattgtcgacactgacgaaggtccaagagaaggtgtcaccgctgaatccttgggcaagttgagaccagctttcgacggtaccaccactgccggtaacgcttcccaagtctctgacggtgctgccgccgtcttgttgatgaagagaagcttggctgaagccaagggatacccaatcattggtaagtacgtcctttgttccaccgccggtgttcctccagaaattatgggtgttggtccagcctacgctatcccagaagtcttgaagagaactggtttgactgttgacgacattgatgttttcgaaatcaacgaagcctttgctgctcaatgtctctactctgctgaacaagtcaatgtgcctgaagagaagttgaacatcaacggtggtgccattgccttgggccacccattgggtgaaaccggtgctcgtcaatacgccaccatcatcccattgttaaaaccaggtcaaattggattgacttcaatgtgtattggttctggtatgggttctgcttctattttggttagagaatag

SEQ ID NO: 79 - protein, 408 aa, Candida sp.
  Candida sp.; 3-ketoacyl-CoA thiolase (beta- ketothiolase) (POT1-1) (EC 2.3.1.16)
MDRLNQLSGQLKPNAKQSILQKNPDDVVIVAAYRTAIGKGFKGSFRSVRSEFILTEFLKEFIKKTNIDPSLIEDVAIGNVLNQAAGATEHRGACLAAGIPYTAAFIAVNRFCSSGLMAISDIANKIKTGEIECGLAGGAESMSTNYRDPRVAPRIDPHLADDAQMEKCLIPMGITNENVANQFNISRERQDEFAAKSYNKAAKAVAAGAFKSEILPIRSIIRNSDGTEKEIIVDTDEGPREGVTAESLGKLRPAFDGTTTAGNASQVSDGAAAVLLMKRSLAEAKGYPIIGKYVLCSTAGVPPEIMGVGPAYAIPEVLKRTGLTVDDIDVFEINEAFAAQCLYSAEQVNVPEEKLNINGGAIALGHPLGETGARQYATIIPLLKPGQIGLTSMCIGSGMGSASILVRE

SEQ ID NO: 80 - DNA, 1227 bp, Candida sp.
  Candida sp.; 3-ketoacyl-CoA thiolase (beta- ketothiolase) (POT1-2) (EC 2.3.1.16)
atggatagattaaaccaattaagcggccaattaaagccaaacgctaaacaatccatcttgcaaaaaaacccagacgacgtcgttatcgttgctgcatacagaaccgccatcggtaagggtttcaaaggttccttcagaaacgtccactctgaattcatcttgactgagttcttgaaagaatttatcaaaaagaccaacatcgacccatctttgattgaagatgtcgctatcggtaacgtcttgaaccaggccgcaggtgccaccgaacacagaggtgcttgtttggctgccggtatcccatacaccgccgccttcatcgctgtcaacagattctgttcctccggtttgatggccatctccgacattgccaacaagatcaagactggtgaaatcgagtgtggtttggctggtggtgccgaatccatgtccaccaactaccgtgacccaagagttgccccaagaatcgacccacatttggctgacgacgcccaaatggaaaagtgtttgattcctatgggtatcaccaacgaaaacgttgctaaccaattcaacatctccagagaaagacaagacgagtttgccgccaagtcctacaacaaggctgccaaggcggttgcctctggtgctttcaagagtgaaatcttgccaatcagatccatcatcagaaactctgacggtaccgaaaaggaaatcattgtcgacactgacgaaggtccaagagaaggtgtcaccgctgaatctttgggcaagttgagaccagctttcgacggtaccaccactgcaggtaacgcttctcaagtctctgacggtgccgccgccgtcttgttgatgaagagaagcttggctgaagccaagggatacccaatcattggtaagtacgtcctttgttccaccgccggtgttccaccagaaatcatgggtgttggtccagccttcgctatcccagaagtcttgaagagaactggcttgactgttgacgacattgatgttttcgaaatcaacgaagcctttgccgctcaatgtctttactctgctgaacaagtcaatgtgcctgaagaaaagttgaacatcaacggtggtgccattgccttgggccatccattgggtgaaaccggtgctcgtcaatacgccaccatcatcccattgttaaagccaggtcaaattggattgacttcaatgtgtattggttctggtatgggttctgcttctattttggttagagaatag

SEQ ID NO: 81 - protein, 408 aa, Candida sp.
  Candida sp.; 3-ketoacyl-CoA thiolase (beta- ketothiolase) (POT1-2) (EC 2.3.1.16)
MDRLNQLSGQLKPNAKQSILQKNPDDVVIVAAYRTAIGKGFKGSFRNVHSEFILTEFLKEFIKKTNIDPSLIEDVAIGNVLNQAAGATEHRGACLAAGIPYTAAFIAVNRFCSSGLMAISDIANKIKTGEIECGLAGGAESMSTNYRDPRVAPRIDPHLADDAQMEKCLIPMGITNENVANQFNISRERQDEFAAKSYNKAAKAVASGAFKSEILPIRSIIRNSDGTEKEIIVDTDEGPREGVTAESLGKLRPAFDGTTTAGNASQVSDGAAAVLLMKRSLAEAKGYPIIGKYVLCSTAGVPPEIMGVGPAFAIPEVLKRTGLTVDDIDVFEINEAFAAQCLYSAEQVNVPEEKLNINGGAIALGHPLGETGARQYATIIPLLKPGQIGLTSMCIGSGMGSASILVRE

SEQ ID NO: 82 - DNA, 1221 bp, Candida sp.
  Candida sp.; 3-ketoacyl-CoA thiolase (beta- ketothiolase) FOX3-1 (EC 2.3.1.16)
atgtcagttaaaagcaagcttgccgaaaaatccccagacgatgttgtcgtcgttgcagcatacagaactgcccaaaccaaaggtggtaagggtggcttcagaaacgtcggctccgactttcttttgtactccttaaccaaagaattcttgaagaagaccggcatcgacccatccatcatccaagacgctgccatcggtaacgtcttgaacagaagatccggtgatttcgaacacagaggtgccttgttggctgccggtatcccacacaccacccctttcatcgccatcaacagacagtgttcctctggtttgatggccatctcccagatcgccaacaagatcaagactggtgaaatcgagtgtggtttggctggtggtgctgaaagcatgaccaagaactacggtccagatgcattggtccaaatcgacccggcctacgctgaaaacccagaattcatcaagaacggtattcctatgggtatcaccaacgagaatgtctgtgccaagttcaacgttgccagagacgctcaagatcaatttgctgctgaatcctaccaaaaggctgaaaaggctcaaaaggaaggtaagtttgacgacgaaatcttgccaattgaagtctaccaagaagacgacgacgatgaagatgaagacgaagacgccgagccaaaggaaatcaaggtcaccgtcagcaaagatgacggaatcagaggtggtgtcaccaaggaaaaattggccaagatcaagcctgccttcaaagacgacggtgtttccaccgccggtaactcctcccaagtttccgacggtgctgctttggtcttgttgatgaagcgttcctttgctgaacaacacggcttcaagccattggccaagtacatttcttgtgccattgctggtgttccacctgaactcatgggtattggtccagctgttgccattccaaaggtcttgaaacaaaacggcttgaacgttaacgacattgatgtttacgaaattaatgaagcctttgctggtcaatgtttgtactctattgaaagctgtggcattgacagatccaaggtcaacatcaacggtggtgccattgctttgggccatccattgggtgtcaccggtgctcgtcaatacgctaccatcttgagattgatgaagccaggccaagttggtcttacttctatgtgtattggtactggtatgggtgctgcttctgttttggttaaagagtag

SEQ ID NO: 83 - protein, 406 aa, Candida sp.
  Candida sp.; 3-ketoacyl-CoA thiolase (beta- ketothiolase) FOX3-1 (EC 2.3.1.16)
MSVKSKLAEKSPDDVVVVAAYRTAQTKGGKGGFRNVGSDFLLYSLTKEFLKKTGIDPSIIQDAAIGNVLNRRSGDFEHRGALLAAGIPHTTPFIAINRQCSSGLMAISQIANKIKTGEIECGLAGGAESMTKNYGPDALVQIDPAYAENPEFIKNGIPMGITNENVCAKFNVARDAQDQFAAESYQKAEKAQKEGKFDDEILPIEVYQEDDDDEDEDEDAEPKEIKVTVSKDDGIRGGVTKEKLAKIKPAFKDDGVSTAGNSSQVSDGAALVLLMKRSFAEQHGFKPLAKYISCAIAGVPPELMGIGPAVAIPKVLKQNGLNVNDIDVYEINEAFAGQCLYSIESCGIDRSKVNINGGAIALGHPLGVTGARQYATILRLMKPGQVGLTSMCIGTGMGAASVLVKE

SEQ ID NO: 84 - DNA, 1221 bp, Candida sp.
  Candida sp.; 3-ketoacyl-CoA thiolase (beta- ketothiolase) FOX3-2 (EC 2.3.1.16)
atgtcagttaaaagcaagcttgccgaaaaatccccagacgatgttgtcgtcgttgcagcatacagaaccgcccaaaccaaaggtggtaagggtggcttcagaaacgtcggctctgactttcttttgtactccataaccaaagaattcttgaagaagaccggcgtcgacccatccatcatccaagacgctgccatcggtaacgtcttgaacagaagatccggtgatttcgaacacagaggtgccttgttggctgccggtgtcccacacaccaccccattcatcgccatcaacagacaatgttcctctggtttgatggccatctcccagatcgccaacaagatcaagactggtgaaatcgagtgtggtttggctggtggtgctgaaagtatgaccaagaactacggtccagacgcattggtccaaatcgacccggcctacgctgaaaacccagaattcatcaagaacggtattcctatgggtatcaccaacgagaatgtctgtgccaagttcaacgttgccagagacgctcaggatcaatttgctgccgaatcctaccaaaaggctgaaaaggctcaaaaggaaggtaagtttgacgacgaaatcttgccaattgaagtctaccaagaagacgacgacgacgaagatgaagacgaagatgccgaaccaaaagaaatcaaggtcaccatcagcaaagatgacggaatcagaggtggtgtcaccaaggaaaaattggccaagatcaagccagccttcaaagacgacggtgtttccaccgctggtaactcctcccaagtttccgacggtgctgctttggtcttgttgatgaagcgttcctttgctgaacaacacggcttcaagccattggccaagtacatttcttgtgccattgctggtgttccacctgaactcatgggtattggtccagctgttgccattccaaaggtcttgaaacaaaacggcttgaacgttaacgacattgatgtttacgaaattaatgaagcctttgctggtcaatgtttgtactccattgaaagctgtggcattgacagatccaaggtcaacatcaacggtggtgccattgctttgggccacccattgggtgtcaccggtgctcgtcaatacgctaccatcttgagattgttgaagccaggccaagttggtcttacttctatgtgtattggtactggtatgggtgctgcttctgttttggttagagaatag

SEQ ID NO: 85 - protein, 406 aa, Candida sp.
  Candida sp.; 3-ketoacyl-CoA thiolase (beta- ketothiolase) FOX3-2 (EC 2.3.1.16)
MSVKSKLAEKSPDDVVVVAAYRTAQTKGGKGGFRNVGSDFLLYSITKEFLKKTGVDPSIIQDAAIGNVLNRRSGDFEHRGALLAAGVPHTTPFIAINRQCSSGLMAISQIANKIKTGEIECGLAGGAESMTKNYGPDALVQIDPAYAENPEFIKNGIPMGITNENVCAKFNVARDAQDQFAAESYQKAEKAQKEGKFDDEILPIEVYQEDDDDEDEDEDAEPKEIKVTISKDDGIRGGVTKEKLAKIKPAFKDDGVSTAGNSSQVSDGAALVLLMKRSFAEQHGFKPLAKYISCAIAGVPPELMGIGPAVAIPKVLKQNGLNVNDIDVYEINEAFAGQCLYSIESCGIDRSKVNINGGAIALGHPLGVTGARQYATILRLLKPGQVGLTSMCIGTGMGAASVLVRE

SEQ ID NO: 86 - DNA, 1887 bp, Escherichia coli
  Escherichia coliK-12 MG1655 sp.; Propionyl-CoA synthetase PrpE
atgtcttttagcgaattttatcagcgttcgattaacgaaccggagcagttctgggccgagcaggcccggcgtattgactggcagacgccctttacgcaaacgctcgatcacagcaatccgccgtttgcccgttggttttgtgaaggccgaaccaacttgtgccacaacgccatcgaccgctggctggagaaacagccagaggcgctggcgctgattgccgtctcttcggaaacagaagaagagcgcacctttacctttcgtcagctgcatgacgaagtgaacgcggtggcctcaatgttgcgttcattgggtgtgcagcgcggcgatcgggtgctggtgtatatgccgatgattgccgaagcgcatattactctgctggcctgcgcgcgcattggcgctattcactcggtggtgtttggtggatttgcctcgcacagcgtggcggcgcgaattgatgacgctaaaccggtgctgattgtctcggctgatgccggagcgcgcggtggcaaaatcattccctataaaaaattgctcgacgatgcgataagtcaggcgcagcaccagccacgccatgttttgctggtggatcgcgggctggcgaaaatggcgcgcgtcagcgggcgggatgtcgatttcgcgtcgttgcgccatcaacacatcggcgcgcgggtaccggtggcgtggctggaatccaacgaaacctcctgcattctctacacttccggcacgaccggcaaacctaaaggcgtgcagcgtgacgtcggcggatatgcggtggcgctggcgacctcgatggacaccatttttggcggcaaagcgggcagcgtgttcttttgcgcatcggatatcggctgggtggtggggcattcgtatatcgtttacgcgccgctgctggcggggatggcgactatcgtttacgaaggattgccgacctggccggactgcggcgtgtggtggacaatcgtcgagaaatatcaggttagccggatgttctcagcgccgaccgccattcgcgtgctgaaaaaattccctaccgctgaaattcgcaaacacgatctctcgtcgctggaagtgctctatctggctggagaaccgctggacgagccgaccgccagttgggtgagcaatacgctggatgtgccggtcatcgacaactactggcagaccgaatccggctggccgattatggcgattgctcgcggtctggacgacaggccgacgcgtctgggaagccccggtgtgccgatgtatggctataacgtgcagttgcttaatgaagtcaccggcgaaccgtgtggcgtcaacgagaaagggatgctggtggtggaagggccgctgccgccggggtgtattcagaccatctggggcgacgacggccgctttgtgaagacttactggtcgctgttttcccgcccggtgtacgccacctttgactggggcatccgtgacgctgacggttatcactttattctcgggcgcactgacgatgtaattaacgttgccgggcatcggctggggacgcgcgagattgaagagagtatctccagccatccgggcgttgccgaagtggcggtggttggggtgaaagatgcgctgaaagggcaggtggcggtggcgtttgtcattccgaaagagagcgacagtctggaagatcgtgatgtggcgcactcgcaagagaaggcgattatggcgctggtggacagccagattggcaactttggccgcccggcgcacgtctggtttgtctcgcaattgccaaaaacgcgatccggaaaaatgctgcgccgcacgatccaggcgatttgcgaaggacgcgatcctggagatctgacgaccattgatgatcctgcgtcgttggatcagatccgccaggcgatggaagagtag

SEQ ID NO: 87 - protein, 628 aa, Escherichia coli [sequence omitted]

SEQ ID NO: 88 - DNA, 1986 bp, Metallosphaera sedula
  Metallosphaera sedula sp.; Propionyl-CoA synthetase Msed_1456 (EC 6.2.1.17)
atgtttatgcgatatattatggttgaggaacagaccctgaagaccgggtcacaggaactagaggagaaggcagactataacatgagatattacgctcacctcatgaagttgagtaaggaaaaacctgcagagttctggggatctctagcacaggacctgctagactggtatgagccttggaaggagaccatgagacaggaagacccgatgacaaggtggttcataggaggtaagataaatgcctcgtacaacgctgtcgacagacacctcaacggccccagaaagttcaaggctgcggtcatctgggaaagtgagttaggggaaaggaagatcgtgacgtatcaggacatgttctatgaggttaataggtgggccaatgcgctcagatccctaggagttggtaaaggggatagggtgaccatatacatgcccctgaccccagagggaatagctgcaatgctggcctcggccaggataggtgcaattcatagcgtaatatttgccggctttggttcgcaagccatagccgacagggttgaggacgccaaggcgaaggtagtgatcactgctgacgcctatcccagaaggggaaaggttgtggagttaaagaagactgtcgacgaggccttaaactcccttggagaaaggagcccagtacagcacgtgctcgtgtataggaggatgaaaacggatgtaaacatgaaggagggaagagacgttttcttcgacgaggtcggcaagtacaggtacgtggagcctgaaaggatggactccaatgatccactcttcattctctacacctctgggaccaccggtaaacctaagggaattatgcactctaccggtggttatctgaccgggacagccgttatgctactgtggagctacggccttagccaggagaacgacgttctcttcaacacctcagatattggttggatagttggccactcctacattacctattccccccttatcatggggagaacggttgtcatttacgagagcgccccagactatccctacccagacaagtgggctgagattattgagagatacagggcaaccactttcggcacctcagctacagccttgcgttacttcatgaagtatggggacgaatacgtgaagaaccacgatctctcgtccatcaggataattgtgacgaacggggaagtgcttaactactctccgtggaagtgggggctagaagtgttaggtggaggaaaggtattcatgtcccatcagtggtggcaaactgagacaggcgcaccgaacctgggctaccttccgggtataatttacatgccaatgaagtcgggtccagcctcaggcttccctctacccggtaacttcgtggaggttctggacgagaacggaaatccctctgcccctagagtgagaggataccttgtaatgaggccacccttcccgcctaacatgatgatggggatgtggaacgataatggggagaggttgaagaagacgtactttagcaagttcggttccctgtattatccaggagacttcgccatggtggatgaggatggatacatctgggtgttgggtagggcagacgagactctaaaaattgcagcccacagaattggagctggggaagtggaatcagcaatcacttctcacccatcggttgccgaggcagcagtcataggcgtgccagactcagtgaaaggagaagaggttcacgcgttcgttgtgctaaagcaaggttacgctccttcctctgaactggctaaggacatacagtcacacgttaggaaggtcatggggcccattgttagtccgcagattcatttcgtggataagttgcctaagacaaggtctgggaaggtcatgagaagggtgataaaggcagtgatgatgggttcgagtgctggcgacttaaccaccatagaggacgaagcatcaatggacgaaataaagaaggctgtcgaggaactaaagaaggagttaaagacctcctag

SEQ ID NO: 89 - protein, 661 aa, Metallosphaera sedula
  Metallosphaera sedula Propionyl-CoA synthetase Msed_1456 (EC 6.2.1.17)
MFMRYIMVEEQTLKTGSQELEEKADYNMRYYAHLMKLSKEKPAEFWGSLAQDLLDWYEPWKETMRQEDPMTRWFIGGKINASYNAVDRHLNGPRKFKAAVIWESELGERKIVTYQDMFYEVNRWANALRSLGVGKGDRVTIYMPLTPEGIAAMLASARIGAIHSVIFAGFGSQAIADRVEDAKAKVVITADAYPRRGKVVELKKTVDEALNSLGERSPVQHVLVYRRMKTDVNMKEGRDVFFDEVGKYRYVEPERMDSNDPLFILYTSGTTGKPKGIMHSTGGYLTGTAVMLLWSYGLSQENDVLFNTSDIGWIVGHSYITYSPLIMGRTVVIYESAPDYPYPDKWAEIIERYRATTFGTSATALRYFMKYGDEYVKNHDLSSIRIIVTNGEVLNYSPWKWGLEVLGGGKVFMSHQWWQTETGAPNLGYLPGIIYMPMKSGPASGFPLPGNFVEVLDENGNPSAPRVRGYLVMRPPFPPNMMMGMWNDNGERLKKTYFSKFGSLYYPGDFAMVDEDGYIWVLGRADETLKIAAHRIGAGEVESAITSHPSVAEAAVIGVPDSVKGEEVHAFVVLKQGYAPSSELAKDIQSHVRKVMGPIVSPQIHFVDKLPKTRSGKVMRRVIKAVMMGSSAGDLTTIEDEASMDEIKKAVEELKKELKTS

SEQ ID NO: 90 - DNA, 1887 bp, Salmonella typhimurium
  Salmonella typhimuriumPropionyl-CoA synthetase PrpE (EC 6.2.1.17)
atgtcttttagcgaattttatcagcgttccattaacgaaccggaggcgttctgggccgagcaggcccggcgtatcgactggcgacagccgtttacgcagacgctggatcatagccgtccaccgtttgcccgctggttttgcggcggcaccactaacttatgtcataacgccgtcgaccgctggcgggataaacagccggaggcgctggcgctgattgccgtctcatcagagaccgatgaagagcgcacatttaccttcagccagttgcatgatgaagtcaacgctgtggccgctatgctgctgtcgctgggcgtgcagcgtggcgatcgcgtattggtctatatgccgatgattgccgaagcgcagataaccctgctggcctgtgcgcgcattggcgcgatccattcggtggtctttggcggttttgcctcgcacagcgtggcggcgcgcattgacgatgccagaccggcgctgattgtgtcggcggatgccggagcgcgtggcggtaaaattctgccgtataaaaagctgcttgatgacgctattgcgcaggcgcagcatcagccgaaacacgttctgctggtggacagagggctggcgaaaatgtcgtgggtggatgggcgcgatctggatttttccacgttgcgccagcagtatctcggcgcgagcgtgccggtggcgtggctggaatccaatgaaacctcgtgcattctttacacctccggcactaccggcaaaccgaaaggcgtccagcgcgacgtcggcggttatgcggtggcgctggcaacctcgatggacaccatttttggcggcaaggcgggcggcgtattcttttgcgcatcggatatcggctgggtcgtcggccactcctatatcgtttacgcgccgctgctggcaggcatggcgactattgtttacgaaggactgccgacgtacccggactgcggggtctggtggaaaattgtcgagaaataccaggttaaccggatgttttccgccccgaccgcgattcgcgtgctgaaaaaattcccgacggcgcaaatccgcaatcacgatctctcctcgctggaggcgctttatctggccggtgagccgctggacgagccgacggccagttgggtgacggagacgctgggcgtaccggtcatcgacaattattggcagacggagtccggctggccgatcatggcgctggcccgcgcgctggacgacaggccgtcgcgtctgggaagtcccggggtgccgatgtacggttataacgtccagctactcaatgaagtcaccggcgaaccctgcggcataaatgaaaaaggcatgctggtgatcgaagggccgctgccgccgggttgtattcagactatttggggcgacgatgcgcgttttgtgaagacttactggtcgctgtttaaccgtcaggtttatgccactttcgactggggaatccgcgacgccgaggggtattactttattctgggccgtaccgatgatgtgattaatattgcgggtcatcggctggggacgcgagaaatagaagaaagtatctccggttacccgaacgtagcggaagtggcggtggtggggataaaagacgctctgaaagggcaggtggcggtggcgtttgtcattccgaagcagagcgatacgctggcggatcgcgaggcggcgcgcgacgaggaaaaagcgattatggcgctggtggataaccagatcggtcactttggtcgtccggcgcatgtctggtttgtttcgcagctccccaaaacgcgttccggaaagatgcttcgccgcacgatccaggcgatctgcgaaggccgtgatccgggcgatctgacaaccattgacgatcccgcgtcgttgcagcaaattcgccaggcgatcgaggaatag

SEQ ID NO: 91 - protein, 628 aa, Salmonella typhimurium
  Salmonella typhimuriumPropionyl-CoA synthetase PrpE (EC 6.2.1.17)
MSFSEFYQRSINEPEAFWAEQARRIDWRQPFTQTLDHSRPPFARWFCGGTTNLCHNAVDRWRDKQPEALALIAVSSETDEERTFTFSQLHDEVNAVAAMLLSLGVQRGDRVLVYMPMIAEAQITLLACARIGAIHSVVFGGFASHSVAARIDDARPALIVSADAGARGGKILPYKKLLDDAIAQAQHQPKHVLLVDRGLAKMSWVDGRDLDFSTLRQQYLGASVPVAWLESNETSCILYTSGTTGKPKGVQRDVGGYAVALATSMDTIFGGKAGGVFFCASDIGWVVGHSYIVYAPLLAGMATIVYEGLPTYPDCGVWWKIVEKYQVNRMFSAPTAIRVLKKFPTAQIRNHDLSSLEALYLAGEPLDEPTASWVTETLGVPVIDNYWQTESGWPIMALARALDDRPSRLGSPGVPMYGYNVQLLNEVTGEPCGINEKGMLVIEGPLPPGCIQTIWGDDARFVKTYWSLFNRQVYATFDWGIRDAEGYYFILGRTDDVINIAGHRLGTREIEESISGYPNVAEVAVVGIKDALKGQVAVAFVIPKQSDTLADREAARDEEKAIMALVDNQIGHFGRPAHVWFVSQLPKTRSGKMLRRTIQAICEGRDPGDLTTIDDPASLQQIRQAIEE

SEQ ID NO: 92 - DNA, 1128 bp, Pseudomonas putida
  Pseudomonas putida KT2440 sp.; Acyl-CoA dehydrogenase PP_2216 (EC 1.3.8.7)
atgctggtaaatgacgagcaacaacagatcgccgacgcggtacgtgcgttcgcccaggaacgcctgaagccgtttgccgagcaatgggacaaggaccatcgcttcccgaaagaggccatcgacgagatggccgaactgggcctgttcggcatgctggtgccggagcagtggggcggtagcgacaccggttatgtggcctatgccatggccttggaggaaatcgctgcgggcgatggcgcctgctcgaccatcatgagcgtgcacaactcggtgggttgcgtgccgatcctgcgcttcggcaacgagcagcagaaagagcagttcctcaccccgctggcgacaggtgcgatgctcggtgctttcgccctgaccgagccgcaggctggctccgatgccagcagcctgaagacccgcgcacgcctggaaggcgaccattacgtgctcaatggcagcaagcagttcattacctcggggcagaacgccggcgtagtgatcgtgtttgcggtcaccgacccggaggccggcaagcgtggcatcagcgccttcatcgtgccgaccgattcgccgggctaccaggtagcgcgggtggaggacaaactcggccagcacgcctccgacacctgccagatcgttttcgacaatgtgcaagtgccagtggccaaccggctgggggcggagggtgaaggctacaagatcgccctggccaaccttgaaggcggccgtatcggcatcgcctcgcaagcggtgggtatggcccgcgcggcgttcgaagtggcgcgggactatgccaacgagcgccagagctttggcaaaccgctgatcgagcaccaggccgtggcgtttcgcctggccgacatggcaacgaaaatttccgttgcccggcagatggtattgcacgccgctgcccttcgtgatgcggggcgcccggcgctggtggaagcgtcgatggccaagctgttcgcctcggaaatggccgaaaaggtctgttcggacgccttgcagaccctgggcggttatggctatctgagtgacttcccgctggagcggatctaccgcgacgttcgggtttgccagatctacgaaggcaccagcgacattcagcgcatggtcattgcgcgcaatctttga

SEQ ID NO: 93 - protein, 375 aa, Pseudomonas putida
  Pseudomonas putida%KT2440 sp.; Acyl-CoA dehydrogenase PP_2216 (EC 1.3.8.7)
MLVNDEQQQIADAVRAFAQERLKPFAEQWDKDHRFPKEAIDEMAELGLFGMLVPEQWGGSDTGYVAYAMALEEIAAGDGACSTIMSVHNSVGCVPILRFGNEQQKEQFLTPLATGAMLGAFALTEPQAGSDASSLKTRARLEGDHYVLNGSKQFITSGQNAGVVIVFAVTDPEAGKRGISAFIVPTDSPGYQVARVEDKLGQHASDTCQIVFDNVQVPVANRLGAEGEGYKIALANLEGGRIGIASQAVGMARAAFEVARDYANERQSFGKPLIEHQAVAFRLADMATKISVARQMVLHAAALRDAGRPALVEASMAKLFASEMAEKVCSDALQTLGGYGYLSDFPLERIYRDVRVCQIYEGTSDIQRMVIARNL

SEQ ID NO: 94 - DNA, 1803 bp, Pseudomonas putida
  Pseudomonas putida%H8234 sp.; Acyl-CoA dehydrogenase PP_2216 (EC 1.3.8.1)
atgcccgagaccctgctcagcccccgcaacctggcctttgagctctacgaagtgctcgacgcccaagccctcacccaacgcccgcgctttgccgagcacagccgcgaaaccttcgacgcggcactgaccaccgcgcgcaccatcgccgaaaagtacttcgccccgcacaaccgcaaggccgacgaaaacgagccgcgctacgtggacggccgcgctgaactgatccccgaggtcaagcctgccgtcgacgcattcctcgaagccggcttcctcaacgccaaccgggacttcgagttcggcggcatgcagctgcccagcctggtttcgcaggcctgcttcgctcacttccaggctgccaacgccggcaccacggcctacccgttcctgaccatgggcgcagccaacctgatcgaaagtttcggcacagaggaacagaagcgtctgttcctgcagccaatgatcgagggccgctacttcggcaccatggcgctgaccgagccccacgctggctcgtctctggccgacatccgcacccgtgccgaacctgcgggcgacggcagctaccggctcaagggcaacaagatcttcatctccggtggcgaccacgaactgtcggaaaacatcgtgcacatggtgctggccaagctgccggacgcaccgcctggggtgaaaggcatctcgctgttcatcgtgcccaagtacaacgtcaaccccgacggcagccgtggcccgcgcaacgacgtgctgctggccgggctgttccacaagatgggctggcgcggtaccacctccaccgcgctgaacttcggcgacaacgaccagtgcgtcggctacctggtcggccagccgcaccaaggcctggcctgcatgttccagatgatgaacgaggcgcgtatcggcgttggcatgggcgcggtgatgctcggatacgccggctacctgtattcgctggaatatgcccgccaacggccgcaaggccggctgccggacaacaaagacccgctcagcccggcggtgccgatcatcgcgcacaccgatgtgaaacgtatgctgctggcacagaaggcgtacgtggaaggcgccttcgacctgggcctttacgccgcgcgcctgttcgacgatacccacaccgccgatgacgaaacgtcccgcacacaagcgcaggcgctgctcgacctgctgaccccgttcgtcaagtcgtggccctcgacgttctgcctcaaggccaacgaactggcgatccagattctcggtggccacggctatacccgcgaatacccggtggaacagtactaccgcgacaatcgcctgaacccgatccacgagggcaccgaaggcattcagtcgctcgacttgctcggccgcaagctggcacagaaccatggtgccggcctcaagcaactgatccgcctgatcgccaccaccggcgaacgtgcaagccaccaccccaaactcgacccactgcgccagccactggagcaactggtcaaccgcctgcagggcgtgacactggccctgctcggcgacatggcccaaggcgaagtcgctggtgccttggcaaactcggccttgtacctcaaggccttcggccattgcgtgatcggctggcgctggctggaacaggccattcacgccgagcttggcctgcagaaaggtcaccctgccgatcgcgacttctatcagggcaagctgcaggccgcgcgttatttcctgacctgggaagtaccgggctgccataatgagctggcattgctagaggcgcgcgacaacacttgcctcaccatgcaggacgagtggttctaa

SEQ ID NO: 95 - protein, 600 aa, Pseudomonas putida
  Pseudomonas putida H8234 sp.
MPETLLSPRNLAFELYEVLDAQALTQRPRFAEHSRETFDAALTTARTIAEKYFAPHNRKADENEPRYVDGRAELIPEVKPAVDAFLEAGFLNANRDFEFGGMQLPSLVSQACFAHFQAANAGTTAYPFLTMGAANLIESFGTEEQKRLFLQPMIEGRYFGTMALTEPHAGSSLADIRTRAEPAGDGSYRLKGNKIFISGGDHELSENIVHMVLAKLPDAPPGVKGISLFIVPKYNVNPDGSRGPRNDVLLAGLFHKMGWRGTTSTALNFGDNDQCVGYLVGQPHQGLACMFQMMNEARIGVGMGAVMLGYAGYLYSLEYARQRPQGRLPDNKDPLSPAVPIIAHTDVKRMLLAQKAYVEGAFDLGLYAARLFDDTHTADDETSRTQAQALLDLLTPFVKSWPSTFCLKANELAIQILGGHGYTREYPVEQYYRDNRLNPIHEGTEGIQSLDLLGRKLAQNHGAGLKQLIRLIATTGERASHHPKLDPLRQPLEQLVNRLQGVTLALLGDMAQGEVAGALANSALYLKAFGHCVIGWRWLEQAIHAELGLQKGHPADRDFYQGKLQAARYFLTWEVPGCHNELALLEARDNTCLTMQDEWF

SEQ ID NO: 96 - DNA, 2130 bp, Candida sp.
  Candida sp.; Acyl-CoA oxidase POX4 (EC 1.3.3.6)
atgacttttacaaagaaaaacgttagtgtatcacaaggtcctgaccctagatcatccatccaaaaggaaagagacagctccaaatggaaccctcaacaaatgaactacttcttggaaggctccgtcgaaagaagtgagttgatgaaggctttggcccaacaaatggaaagagacccaatcttgttcacagacggctcctactacgacttgaccaaggaccaacaaagagaattgaccgccgtcaagatcaacagaatcgccagatacagagaacaagaatccatcgacactttcaacaagagattgtccttgattggtatctttgacccacaggtcggtaccagaattggtgtcaacctcggtttgttcctttcttgtatcagaggtaacggtaccacttcccaattgaactactgggctaacgaaaaggaaaccgctgacgttaaaggtatctacggttgtttcggtatgaccgaattggcccacggttccaacgttgctggtttggaaaccaccgccacatttgacaaggaatctgacgagtttgtcatcaacaccccacacattggtgccaccaagtggtggattggtggtgctgctcactccgccacccactgttctgtctacgccagattgattgttgacggtcaagattacggtgtcaagacttttgttgtcccattgagagactccaaccacgacctcatgccaggtgtcactgttggtgacattggtgccaagatgggtagagatggtatcgataacggttggatccaattctccaacgtcagaatcccaagattctttatgttgcaaaagttctgtaaggtttctgctgaaggtgaagtcaccttgccacctttggaacaattgtcttactccgccttgttgggtggtagagtcatgatggttttggactcctacagaatgttggctagaatgtccaccattgccttgagatacgccattggtagaagacaattcaagggtgacaatgtcgatccaaaagatccaaacgctttggaaacccaattgatagattacccattgcaccaaaagagattgttcccatacttggctgctgcctacgtcatctccgctggtgccctcaaggttgaagacaccatccataacaccttggctgaattggacgctgccgttgaaaagaacgacaccaaggctatctttaagtctattgacgacatgaagtcattgtttgttgactctggttccttgaagtccactgccacttggttgggtgctgaagccattgaccaatgtagacaagcctgtggtggtcacggttactcgtcctacaacggcttcggtaaagcctacaacgattgggttgtccaatgtacttgggaaggtgacaacaatgtcttggccatgagtgttggtaagccaattgtcaagcaagttatcagcattgaagatgccggcaagaccgtcagaggttccaccgctttcttgaaccaattgaaggactacactggttccaacagctccaaggttgttttgaacactgttgctgacttggacgacatcaagactgtcatcaaggctattgaagttgccatcatcagattgtcccaagaagctgcttctattgtcaagaaggaatctttcgactatgtcggcgctgaattggttcaactctccaagttgaaggctcaccactacttgttgactgaatacatcagaagaattgacacctttgaccaaaaggacttggttccatacttgatcaccctcggtaagttgtacgctgccactattgtcttggacagatttgccggtgtcttcttgactttcaacgttgcctccaccgaagccatcactgctttggcctctgtgcaaattccaaagttgtgtgctgaagtcagaccaaacgttgttgcttacaccgactccttccaacaatccgacatgattgtcaattctgctattggtagatacgatggtgacatctatgagaactactttgacttggtcaagttgcagaacccaccatccaagaccaaggctccttactctgatgctttggaagccatgttgaacagaccaaccttggacgaaagagaaagatttgaaaagtctgatgaaaccgctgctatcttgtccaagtaa

SEQ ID NO: 97 - protein, 709 aa, Candida sp.
  Candida sp.; Acyl-CoA oxidase POX4 (EC 1.3.3.6)
MTFTKKNVSVSQGPDPRSSIQKERDSSKWNPQQMNYFLEGSVERSELMKALAQQMERDPILFTDGSYYDLTKDQQRELTAVKINRIARYREQESIDTFNKRLSLIGIFDPQVGTRIGVNLGLFLSCIRGNGTTSQLNYWANEKETADVKGIYGCFGMTELAHGSNVAGLETTATFDKESDEFVINTPHIGATKWWIGGAAHSATHCSVYARLIVDGQDYGVKTFVVPLRDSNHDLMPGVTVGDIGAKMGRDGIDNGWIQFSNVRIPRFFMLQKFCKVSAEGEVTLPPLEQLSYSALLGGRVMMVLDSYRMLARMSTIALRYAIGRRQFKGDNVDPKDPNALETQLIDYPLHQKRLFPYLAAAYVISAGALKVEDTIHNTLAELDAAVEKNDTKAIFKSIDDMKSLFVDSGSLKSTATWLGAEAIDQCRQACGGHGYSSYNGFGKAYNDWVVQCTWEGDNNVLAMSVGKPIVKQVISIEDAGKTVRGSTAFLNQLKDYTGSNSSKVVLNTVADLDDIKTVIKAIEVAIIRLSQEAASIVKKESFDYVGAELVQLSKLKAHHYLLTEYIRRIDTFDQKDLVPYLITLGKLYAATIVLDRFAGVFLTFNVASTEAITALASVQIPKLCAEVRPNVVAYTDSFQQSDMIVNSAIGRYDGDIYENYFDLVKLQNPPSKTKAPYSDALEAMLNRPTLDERERFEKSDETAAILSK

SEQ ID NO: 98 - DNA, 1989 bp, Candida sp.
  Candida sp.; Acyl-CoA oxidase POX5 (EC 1.3.3.6)
atgcctaccgaacttcaaaaagaaagagaactcaccaagttcaacccaaaggagttgaactacttcttggaaggttcccaagaaagatccgagatcatcagcaacatggtcgaacaaatgcaaaaagaccctatcttgaaggtcgacgcttcatactacaacttgaccaaagaccaacaaagagaagtcaccgccaagaagattgccagactctccagatactttgagcacgagtacccagaccaacaggcccagagattgtcgatcctcggtgtctttgacccacaagtcttcaccagaatcggtgtcaacttgggtttgtttgtttcctgtgtccgtggtaacggtaccaactcccagttcttctactggaccataaataagggtatcgacaagttgagaggtatctatggttgttttggtatgactgagttggcccacggttccaacgtccaaggtattgaaaccaccgccacttttgacgaagacactgacgagtttgtcatcaacaccccacacattggtgccaccaagtggtggatcggtggtgctgcgcactccgccacccactgctccgtctacgccagattgaaggtcaaaggaaaggactacggtgtcaagacctttgttgtcccattgagagactccaaccacgacctcgagccaggtgtgactgttggtgacattggtgccaagatgggtagagacggtatcgataacggttggatccagttctccaacgtcagaatcccaagattctttatgttgcaaaagtactgtaaggtttcccgtctgggtgaagtcaccatgccaccatctgaacaattgtcttactcggctttgattggtggtagagtcaccatgatgatggactcctacagaatgaccagtagattcatcaccattgccttgagatacgccatccacagaagacaattcaagaagaaggacaccgataccattgaaaccaagttgattgactacccattgcatcaaaagagattgttcccattcttggctgccgcttacttgttctcccaaggtgccttgtacttagaacaaaccatgaacgcaaccaacgacaagttggacgaagctgtcagtgctggtgaaaaggaagccattgacgctgccattgtcgaatccaagaaattgttcgtcgcttccggttgtttgaagtccacctgtacctggttgactgctgaagccattgacgaagctcgtcaagcttgtggtggtcacggttactcgtcttacaacggtttcggtaaagcctactccgactgggttgtccaatgtacctgggaaggtgacaacaacatcttggccatgaacgttgccaagccaatggttagagacttgttgaaggagccagaacaaaagggattggttctctccagcgttgccgacttggacgacccagccaagttggttaaggctttcgaccacgccctttccggcttggccagagacattggtgctgttgctgaagacaagggtttcgacattaccggtccaagtttggttttggtttccaagttgaacgctcacagattcttgattgacggtttcttcaagcgtatcaccccagaatggtctgaagtcttgagacctttgggtttcttgtatgccgactggatcttgaccaactttggtgccaccttcttgcagtacggtatcattaccccagatgtcagcagaaagatttcctccgagcacttcccagccttgtgtgccaaggttagaccaaacgttgttggtttgactgatggtttcaacttgactgacatgatgaccaatgctgctattggtagatatgatggtaacgtctacgaacactacttcgaaactgtcaaggctttgaacccaccagaaaacaccaaggctccatactccaaggctttggaagacatgttgaaccgtccagaccttgaagtcagagaaagaggtgaaaagtccgaagaagctgctgaaatcttgtccagttaa

SEQ ID NO: 99 - protein, 662 aa, Candida sp.
  Candida sp.; Acyl-CoA oxidase POX5 (EC 1.3.3.6)
MPTELQKERELTKFNPKELNYFLEGSQERSEIISNMVEQMQKDPILKVDASYYNLTKDQQREVTAKKIARLSRYFEHEYPDQQAQRLSILGVFDPQVFTRIGVNLGLFVSCVRGNGTNSQFFYWTINKGIDKLRGIYGCFGMTELAHGSNVQGIETTATFDEDTDEFVINTPHIGATKWWIGGAAHSATHCSVYARLKVKGKDYGVKTFVVPLRDSNHDLEPGVTVGDIGAKMGRDGIDNGWIQFSNVRIPRFFMLQKYCKVSRSGEVTMPPSEQLSYSALIGGRVTMMMDSYRMTSRFITIALRYAIHRRQFKKKDTDTIETKLIDYPLHQKRLFPFLAAAYLFSQGALYLEQTMNATNDKLDEAVSAGEKEAIDAAIVESKKLFVASGCLKSTCTWLTAEAIDEARQACGGHGYSSYNGFGKAYSDWVVQCTWEGDNNILAMNVAKPMVRDLLKEPEQKGLVLSSVADLDDPAKLVKAFDHALSGLARDIGAVAEDKGFDITGPSLVLVSKLNAHRFLIDGFFKRITPEWSEVLRPLGFLYADWILTNFGATFLQYGIITPDVSRKISSEHFPALCAKVRPNVVGLTDGFNLTDMMTNAAIGRYDGNVYEHYFETVKALNPPENTKAPYSKALEDMLNRPDLEVRERGEKSEEAAEILSS

SEQ ID NO: 100 - DNA, 2718 bp, Candida sp.
  Candida sp.; Enoyl-CoA hydratase FOX2/HDE (EC 4.2.1.17)
atgtctccagttgattttaaagataaagttgtgatcattaccggtgccggtggtggtttgggtaaatactactccctcgaatttgccaagttgggcgccaaagtcgtcgttaacgacttgggtggtgccttgaacggtcaaggtggaaactccaaggccgccgacgttgtcgttgacgaaattgtcaagaacggtggtgttgccgttgccgattacaacaacgtcttggacggtgacaagattgtcgaaaccgccgtcaagaactttggtactgtccacgttatcatcaacaatgccggtatcttgagagatgcctccatgaagaagatgactgaaaaagactacaaattggtcattgacgtgcacttgaacggtgcctttgccgtcaccaaggctgcttggccatacttccaaaagcaaaaatacggtagaattgtcaacacatcctccccagctggtttgtacggtaactttggtcaagccaactacgcctccgccaagtctgctttgttgggattcgctgaaaccttggccaaggaaggtgccaaatacaacatcaaggccaacgccattgctccgttggccagatcaagaatgactgaatctatcttgccacctccaatgttggaaaaattgggccctgaaaaggttgccccattggtcttgtatttgtcgtcagctgaaaacgaattgactggtcaattctttgaagttgctgctggcttttacgctcagatcagatgggaaagatccggtggtgtcttgttcaagccagatcaatccttcaccgctgaggttgttgctaagagattctctgaaatccttgattatgacgactctaggaagccagaatacttgaagaaccaatacccattcatgttgaacgactacgccactttgaccaacgaagctagaaagttgccagctaacgatgcttctggtgctccaactgtctccttgaaggacaaggttgttttgatcaccggtgccggtgctggtttgggtaaagaatacgccaagtggttcgccaagtacggtgccaaggttgttgttaacgacttcaaggatgctaccaagaccgttgacgaaatcaaagccgctggtggtgaagcttggccagatcaacacgatgttgccaaggactccgaagctatcatcaagaatgtcattgacaagtacggtaccattgatatcttggtcaacaacgccggtatcttgagagacagatcctttgccaagatgtccaagcaagaatgggactctgtccaacaagtccacttgattggtactttcaacttgagcagattggcatggccatactttgttgaaaaacaatttggtagaatcatcaacattacctccaccagtggtatctacggtaactttggtcaagccaactactcgtcttctaaggctggtatcttgggtttgtccaagaccatggccattgaaggtgctaagaataacattaaggtcaacattgttgctccacacgctgaaactgccatgaccttgaccatcttcagagaacaagacaagaacttgtaccacgctgaccaagttgctccattgttggtctacttgggtactgacgatgtcccagtcaccggtgaaactttcgaaatcggtggtggttggatcggtaacaccagatggcaaagagccaagggtgctgtctcccacgacgaacacaccactgttgaattcatcaaggagcacttgaacgaaatcactgacttcaccactgacactgaaaatccaaaatctaccaccgaatcctccatggctatcttgtctgccgttggtggtgatgacgatgatgatgacgaagacgaagaagaagacgaaggtgatgaagaagaagacgaagaagacgaagaagaagacgatccagtctggagattcgacgacagagatgttatcttgtacaacattgcccttggtgccaccaccaagcaattgaagtacgtctacgaaaacgactctgacttccaagtcattccaacctttggtcacttgatcaccttcaactctggtaagtcacaaaactcctttgccaagttgttgcgtaacttcaacccaatgttgttgttgcacggtgaacactacttgaaggtgcacagctggccaccaccaaccgaaggtgaaatcaagaccactttcgaaccaattgccactactccaaagggtaccaacgttgttattgttcacggttccaaatctgttgacaacaagtctggtgaattgatttactccaacgaagccacttacttcatcagaaactgtcaagccgacaacaaggtctacgctgaccgtccagcattcgccaccaaccaattcttggcaccaaagagagccccagactaccaagttgacgttccagtcagtgaagacttggctgctttgtaccgtttgtctggtgacagaaacccattgcacattgatccaaactttgctaaaggtgccaagttccctaagccaatcttacacggtatgtgcacttatggtttgagtgctaaggctttgattgacaagtttggtatgttcaacgaaatcaaggccagattcaccggtattgtcttcccaggtgaaaccttgagagtcttggcatggaaggaaagcgatgacactattgtcttccaaactcatgttgttgatagaggtactattgccattaacaacgctgctattaagttagtcggtgacaaagcaaagatc

SEQ ID NO: 101 - protein, 906 aa, Candida sp.
  Candida sp.; Enoyl-CoA hydratase FOX2/HDE (EC 4.2.1.17)
MSPVDFKDKVVIITGAGGGLGKYYSLEFAKLGAKVVVNDLGGALNGQGGNSKAADVVVDEIVKNGGVAVADYNNVLDGDKIVETAVKNFGTVHVIINNAGILRDASMKKMTEKDYKLVIDVHLNGAFAVTKAAWPYFQKQKYGRIVNTSSPAGLYGNFGQANYASAKSALLGFAETLAKEGAKYNIKANAIAPLARSRMTESILPPPMLEKLGPEKVAPLVLYLSSAENELTGQFFEVAAGFYAQIRWERSGGVLFKPDQSFTAEVVAKRFSEILDYDDSRKPEYLKNQYPFMLNDYATLTNEARKLPANDASGAPTVSLKDKVVLITGAGAGLGKEYAKWFAKYGAKVVVNDFKDATKTVDEIKAAGGEAWPDQHDVAKDSEAIIKNVIDKYGTIDILVNNAGILRDRSFAKMSKQEWDSVQQVHLIGTFNLSRLAWPYFVEKQFGRIINITSTSGIYGNFGQANYSSSKAGILGLSKTMAIEGAKNNIKVNIVAPHAETAMTLTIFREQDKNLYHADQVAPLLVYLGTDDVPVTGETFEIGGGWIGNTRWQRAKGAVSHDEHTTVEFIKEHLNEITDFTTDTENPKSTTESSMAILSAVGGDDDDDDEDEEEDEGDEEEDEEDEEEDDPVWRFDDRDVILYNIALGATTKQLKYVYENDSDFQVIPTFGHLITFNSGKSQNSFAKLLRNFNPMLLLHGEHYLKVHSWPPPTEGEIKTTFEPIATTPKGTNVVIVHGSKSVDNKSGELIYSNEATYFIRNCQADNKVYADRPAFATNQFLAPKRAPDYQVDVPVSEDLAALYRLSGDRNPLHIDPNFAKGAKFPKPILHGMCTYGLSAKALIDKFGMFNEIKARFTGIVFPGETLRVLAWKESDDTIVFQTHVVDRGTIAINNAAIKLVGDKAKI

SEQ ID NO: 102 - DNA, 1518 bp, Candida sp.
  Candida sp.; 3-hydroxypropionyl-CoA hydrolase (EC 3.1.2.4)
atgattcgcttcactgtttcttcaattagacccatcaactgtgctacaaggagatccatatcactactacaatcaagaatgtcatccagtgtatcgacaaacccaactgccgggggcgaagaagagccagttgtcttgacctccaccaagaaccatgccagaatcatcaccctcaacagagtcagaaagttgaattcgttaaacaccgaaatgattgaactaatgacaccacctgtcttggagtacgccaaagagaatgtcaacaacgtcaccatcttgacttcgaactcccctaaggcattgtgtgccggtggtgatgttgctgaatgtgcaattcaaatcagaaagggcaacccgggatacggcgctgatttttttgataaggaatacaacctcaattacattatttccaccttgccaaagccttacatttcccttatggatggcatcacgtttggtggtggtgttgggttgtctgttcacgctccatttagagttgccacggagaagaccaagttagccatgccggagatggacattggattcttccctgatgtcggtaccactttcttcttgccaagattggacgacaagattggttactacgttgcgttgactgggtctgttttgccaggtttggatgcctatttggcgggatttgcaacccactatatcaagtcggaaaaaatccctctgttgatcaagagattggctggcttgcaaccacctgaaattgaaggcgaaatcacggttatttctggaaacaatcagtacttcaaccaggtgaatgacattttgaacgagtttagtgagaagaagttgcctcaggactacaggttcttcctttccccagatgatatagccgttatcaacaaggcattctcgcaagactcaatcgacggtgtgttcaagtacttgaaagaggaaggttctccatttgcaaagaagacccttgacactttgtccaagaagccaaggagttcgttggccgttgcatttgcgttgttgaaccagggtgataagaacacgatcagagaacaatttgagttggaaatggttgctgcaaccaacattatgagcatccctgctgaacgtaacgactttgctaaaggtgtcattcacaaattggtcgacaagataaaggacccattcttcccacaatggaacgacccaagcacagtcacgccagagtttgtcaaaaacatactcagtttgtccaagaacaccgacaagtacttgaagaagccatacgtcaagcaatggtttggtgttgacttcacccagtaccctcaccaattcggggtgccaaccaaccgcgaagttgaagcatacattgctggcaccgacggctccaacagaacctacttgccaactccaagcgaagtgttcaagcatttcaagatcaagacgggcgacaagttgggtgttgaagccaagattcaacagattttggacttgcatggcgagactgcaaagtatgataacaagtatgtcacctggaaagacgaaccaaccaaa

SEQ ID NO: 103 - protein, 506 aa, Candida sp.
  Candida sp.; 3-hydroxypropionyl-CoA hydrolase (EC 3.1.2.4)
MIRFTVSSIRPINCATRRSISLLQSRMSSSVSTNPTAGGEEEPVVLTSTKNHARIITLNRVRKLNSLNTEMIELMTPPVLEYAKENVNNVTILTSNSPKALCAGGDVAECAIQIRKGNPGYGADFFDKEYNLNYIISTLPKPYISLMDGITFGGGVGLSVHAPFRVATEKTKLAMPEMDIGFFPDVGTTFFLPRLDDKIGYYVALTGSVLPGLDAYLAGFATHYIKSEKIPSLIKRLAGLQPPEIEGEITVISGNNQYFNQVNDILNEFSEKKLPQDYRFFLSPDDIAVINKAFSQDSIDGVFKYLKEEGSPFAKKTLDTLSKKPRSSLAVAFALLNQGDKNTIREQFELEMVAATNIMSIPAERNDFAKGVIHKLVDKIKDPFFPQWNDPSTVTPEFVKNILSLSKNTDKYLKKPYVKQWFGVDFTQYPHQFGVPTNREVEAYIAGTDGSNRTYLPTPSEVFKHFKIKTGDKLGVEAKIQQILDLHGETAKYDNKYVTWKDEPTK

SEQ ID NO: 104 - protein, 9 aa, Artificial Sequence
  Description of Artificial Sequence Synthetic peptide
DYKDDDDKG

SEQ ID NO: 105 - protein, 14 aa, Artificial Sequence
  Description of Artificial Sequence Synthetic peptide
GKPIPNPLLGLDST

SEQ ID NO: 106 - protein, 10 aa, Artificial Sequence
  Description of Artificial Sequence Synthetic peptide
EQKLISEEDL

SEQ ID NO: 107 - protein, 11 aa, Artificial Sequence
  Description of Artificial Sequence Synthetic peptide
QPELAPEDPED

SEQ ID NO: 108 - protein, 9 aa, Artificial Sequence
  Description of Artificial Sequence Synthetic peptide
YPYDVPDYA

SEQ ID NO: 109 - protein, 11 aa, Artificial Sequence
  Description of Artificial Sequence Synthetic peptide
YTDIEMNRLGK

SEQ ID NO: 110 - protein, 6 aa, Artificial Sequence
  Description of Artificial Sequence Synthetic 6xHis tag
HHHHHH

SEQ ID NO: 111 - protein, 7 aa, Artificial Sequence
  Description of Artificial Sequence Synthetic peptide
  MOD_RES (3)..(5): Any amino acid
  MISC_FEATURE (3)..(5): This region may encompass 1-3 residues wherein some positions may be absent
CCXXXCC

SEQ ID NO: 112 - protein, 6 aa, Artificial Sequence
  Description of Artificial Sequence Synthetic peptide
CCPGCC

SEQ ID NO: 113 - protein, 6 aa, Artificial Sequence
  Description of Artificial Sequence Synthetic peptide
LVPRGS

SEQ ID NO: 114 - protein, 5 aa, Artificial Sequence
  Description of Artificial Sequence Synthetic peptide
DDDDK

SEQ ID NO: 115 - protein, 7 aa, Artificial Sequence
  Description of Artificial Sequence Synthetic peptide
ENLYFQG

SEQ ID NO: 116 - protein, 8 aa, Artificial Sequence
  Description of Artificial Sequence Synthetic peptide
LEVLFQGP